(19) United States
(12) Patent Application Publication
(10) Pub. No.: US 2025/0290086 A1
(43) Pub. Date: Sep. 18, 2025

(54) TISSUE-SPECIFIC PROMOTERS IN PLANTS

(71) Applicants: Altria Client Services LLC, Richmond, VA (US); University of Virginia Patent Foundation, Charlottesville, VA (US)

(72) Inventors: Chengalrayan Kudithipudi, Midlothian, VA (US); Yanxin Shen, Henrico, VA (US); Dongmei Xu, Glen Allen, VA (US); Michael Paul Timko, Charlottesville, VA (US); Roel Rabara, Charlottesville, VA (US)

(73) Assignees: Altria Client Services LLC, Richmond, VA (US); University of Virginia Patent Foundation, Charlottesville, VA (US)

(21) Appl. No.: 19/225,122

(22) Filed: Jun. 2, 2025

Related U.S. Application Data
(63) Continuation of application No. 18/449,849, filed on Aug. 15, 2023, now Pat. No. 12,351,809.
Application No. 18/449,849 is a continuation of application No. 17/591,188, filed on Feb. 2, 2022, now abandoned.
(60) Provisional application No. 63/145,259, filed on Feb. 3, 2021.
(60) Provisional application No. 63/145,262, filed on Feb. 3, 2021.
(60) Provisional application No. 63/145,263, filed on Feb. 3, 2021.

(51) Int. Cl. C12N 15/82; C07K 14/415; C12P 5/00

(52) U.S. Cl. CPC C12N 15/8262; C12N 15/8223; C12N 15/8243; C07K 14/415; C12N 2800/22; C12P 5/007

(57) ABSTRACT

The present disclosure relates to compositions and methods related to tissue-specific promoters and their uses in plants, including tobacco and cannabis. The provided trichome-specific promoters enable the expression of heterologous polynucleotides in trichome tissues.

### CROSS-REFERENCE TO RELATED APPLICATIONS

This application is a continuation of U.S. patent application Ser. No. 18/449,849, filed on Aug. 15, 2023, which is a continuation of U.S. patent application Ser. No. 17/591,188, filed on Feb. 2, 2022, which claims the benefit of: U.S. Provisional Patent Application No. 63/145,259, filed Feb. 3, 2021, U.S. Provisional Patent Application No. 63/145,262, filed Feb. 3, 2021, and U.S. Provisional Patent Application No. 63/145,263, filed Feb. 3, 2021, all of which are incorporated by reference herein in their entireties.

### INCORPORATION OF SEQUENCE LISTING

A Sequence Listing contained in the file named “P34834US02_SL.xml” which is 173,064 bytes in size (measured in MS-Windows®), created on May 30, 2025, and containing a total number of 102 sequences from SEQ ID NO: 1 to SEQ ID NO: 102, is filed electronically herewith and incorporated by reference in its entirety.

### FIELD

The present disclosure relates to trichome-preferred and trichome-specific promoters and their uses in plants, including tobacco and cannabis.

### SEQUENCES

Table 1 provides nucleic acid sequences and amino acid sequences used in this disclosure.

TABLE 1
Sequences for this disclosure

| SEQ | Sequence | Sequence | |
|---|---|---|---|
| ID NO | Description | Type | Sequence |
| 1 | NtPhy0.5kb- | Nucleic acid | GGGGACAAGTTTGTACAAAAAAGCAGGCTTATAAATACCT |
| | Fwd | | ATGAAAATTAAAT |
| 2 | NtPhy-Rev | Nucleic acid | GGGGACCACTTTGTACAAGAAAGCTGGGTAGGCAGTTATT |
| | | | GTTATATGTGATAG |
| 3 | NtCyc-0.5-Fwd | Nucleic acid | GGGGACAAGTTTGTACAAAAAAGCAGGCTGCAATAATTTT |
| | | | TTATATCTAT |
| 4 | NyCyc-Rev | Nucleic acid | GGGGACCACTTTGTACAAGAAAGCTGGGTACTCTCTCTCT |
| | | | TTCTCTCG |
| 5 | NtPSO-1.0-Fwd | Nucleic acid | GGGGACAAGTTTGTACAAAAAAGCAGGCTATTTACGAGTT |
| | | | TACTATACTT |
| 6 | NtPSO-Rev | Nucleic acid | GGGGACCACTTTGTACAAGAAAGCTGGGTATTTGGGAGGG |
| | | | AATTAAAG |
| 7 | NtRbcST11243bp- | Nucleic acid | GGGGACAAGTTTGTACAAAAAAGCAGGCTATTAGCATCAA |
| | Fwd | | CCGGGTTAGC |
| 8 | NtRbcST_436bp- | Nucleic acid | GGGGACAAGTTTGTACAAAAAAGCAGGCTTTCATGAATCT |
| | Fwd | | CAATATGGAGG |
| 9 | NtRbcST-Rev | Nucleic acid | GGGGACCACTTTGTACAAGAAAGCTGGGTAGTTCACCTTC |
| | | | ACTTTAAGCTAC |
| 10 | RbcsT promoter | Nucleic acid | ATTAGCATCAACCGGGTTAGCAATTGGGGCATTATTGCGA |
| | (1243 nt) | | TCAGCGGGAGGCACCTTGTTGCTGAGCACCAAATTATTCT |
| | | | TTTTGCCGTGATGGCCAGACTCAACGTCAACGTTCAAGTG |
| | | | AGCAGACCAAAAGTTTAACTTTTTTGGGATGGCTTGAAAT |
| | | | TAAGACTTTAAATAATAAGCATAAAATGAGGTGTTATGGA |
| | | | GATTCGGATCAAACCACCACTATTATCCCTAGCCCCACGG |
| | | | TTGATGCCAAACTGTTTATTTCAAAATCGGATAACAATTA |
| | | | CATTTGTATGCGGTTTTAAGAATATGTGATTTAATTCAAC |
| | | | ACGAACAATTAAGAATATCAAATAATCAAATTAGAGATAA |
| | | | AATAATCATCCAAACGAATTGCGATATTATGGCTAACCAT |
| | | | GAGCTTAGACTGACCGATGAGCTCGCTCTTGGTTGACCTT |
| | | | CGAGCCCAGCCGTGAATGAAGTACATAACAAATGAGCAAA |
| | | | AACTTTAACAATAGCTAAAAGGCAGAAAGTAAACTTGTAT |
| | | | TGCTTTGATTTACATATTACAACATGTGTTACAAAAGAAA |
| | | | AACTTCCACCCTTTATATAGTGGAGAGTTTCATCCCCAGT |
| | | | ATAAATCTAAAAAAGGTAAAAATCTTCCTTTGCTGGTAAT |
| | | | TACTAATTCATGATCGACATCGAGTGAGATTTGCACCGTA |
| | | | ATATCCGGTTGATTGCGATATCACGATCCTCTATCTGTCA |
| | | | TGTGTAACCGTTTATCATGCCTCCCGAGATCTTAGAATTC |
| | | | ATTCTTGGACCGGGGTGCATGCCCAATAACAGGCACATTG |
| | | | TTCGCCCTTCATGAATCTCAATATGGAGGGTCTTTAACCT |
| | | | CGATTATAATTTTGTGTATATGTACTCTTCCTCTATTTTC |
| | | | TCGTCGAAAAATCGGAGTAAACATTATCCCCGATTTTACC |
| | | | ATACACATACGTTTTGCCTAAATAAAATGACAAAAGAGAA |
| | | | AGGGTGACGAAAACTGTCACTATGCCTAACTAACCCTCGT |
| | | | AGATACCGTCTTCCGCGAACCCTACACACCCCTAAGGCCC |
| | | | TAACCCCTTCTTCACCTTACTTTATTAATTACCTCACTCA |
| | | | CTTTTGTCCTCTTATGTAACCGTTTCCAGTTTAATTACAA |
| | | | AAAAATTCAGATTACTTTGAAGCATCACTTCTCTCCCTTT |
| | | | CATATAAACTCAGTTTTTTTGTTCGAGTCTTTATACATAT |
| | | | GTCTTTATCATAACTAAAGTAGTAGCTTAAAGTGAAGGTG |
| | | | AAC |
| 11 | RbcsT promoter | Nucleic acid | TTCATGAATCTCAATATGGAGGGTCTTTAACCTCGATTAT |
| | (436 nt) | | AATTTTGTGTATATGTACTCTTCCTCTATTTTCTCGTCGA |
| | | | AAAATCGGAGTAAACATTATCCCCGATTTTACCATACACA |
| | | | TACGTTTTGCCTAAATAAAATGACAAAAGAGAAAGGGTGA |
| | | | CGAAAACTGTCACTATGCCTAACTAACCCTCGTAGATACC |
| | | | GTCTTCCGCGAACCCTACACACCCCTAAGGCCCTAACCCC |
| | | | TTCTTCACCTTACTTTATTAATTACCTCACTCACTTTTGT |
| | | | CCTCTTATGTAACCGTTTCCAGTTTAATTACAAAAAAATT |
| | | | CAGATTACTTTGAAGCATCACTTCTCTCCCTTTCATATAA |
| | | | ACTCAGTTTTTTTGTTCGAGTCTTTATACATATGTCTTTA |
| | | | TCATAACTAAAGTAGTAGCTTAAAGTGAAGGTGAAC |
| 12 | NtPSO 1.5 kb | Nucleic acid | AAAATATTTGAAAGAAAATATTGTAGCTAAATGAATATTT |
| | promoter | | TTCCTTCAAGTTATTAAAAGTTGTGGCAATATAGGTTAAA |
| | | | TTAGCCACATGTTTCTTGCTTTAATAGAATTTTGTAGCTA |
| | | | ATCATTAACTTTTACCACGAGTTGAAACTTAATATAACAA |
| | | | CAATAACCTTTTAACCATAATAAACCATTTAAATCAAATA |
| | | | TTACTAAATAAATAACTTTGCTTCAAGTTCTATAAAATCA |
| | | | TGGCAATAGTCATTACGATAAAATGATATAACCACGAATA |
| | | | TATTGCAACGATAAATTCTGTAACTAATCATTAGTTTTTG |
| | | | CGACGAGGTAAATTTTCCGTCACAGTAGCAATCTTCTAGG |
| | | | CACATTAAAAATTTGAAACAAAATTTTGTAGTCAAATAAA |
| | | | TATTTATCTTCTTATTTTAAGAAAATAAAAATAGTTAGAT |
| | | | AATAGTTACTACTATTTGTCATGAAAATATCAATAGATAC |
| | | | AAATTTAAAGTGACTATAAATTTACGAGTTTACTATACTT |
| | | | TAGTCGTACAGTTTGCAATAATAGTATTTTAACCACAATT |
| | | | AGTTATATGTACAAAATTTCATAAGTGAATAACTTTTTTT |
| | | | CAATGACAAAAATAAGAGTTGCTCAAACAATATCAAGTTA |
| | | | CAAAAATTTAATTTTAACTGTAAAAGTTATATTTTTCCAA |
| | | | AATAACATAAACTATAGTAATTATATATAGTTTGAAGTAT |
| | | | TAATAAAATTTAAATATGCAAAAGTTAATTTTAATAAACC |
| | | | ATTTGTATGCCTACTTGTAGCCTCTAAACTATTTTATTTG |
| | | | CTTTATTTATCAAACTCATATTTTATTTTATTGCACCTTG |
| | | | TTAGTTTTGGACGTTAATTATATATATTTGGTGTAAAATT |
| | | | TAAAATATATTAACATTTGTGGAGAATTTATGTATGCCTG |
| | | | GTTCTTAACTATTTTTTTTTTATATAACTGGTTAGAGTAA |
| | | | TTTCTTATATTTCAGTATTTATTTTTAAATAAGTCCTCAT |
| | | | AAATTGAAGACTTTAAAAGTTTTTGTGTCATTCCTCTTTT |
| | | | TATTTAAGAAATTGAAGAATTCCGCTAAATTTCATATTCC |
| | | | GCTGTTATTTAACTGTTTATTCCCTTGTTAATATAATTGG |
| | | | TAAGAAGTTTTAAAATAAAGGAGTTAATGATTTCTAGGTT |
| | | | CATGGCTTGCCTAGCTTCTACGAGTAAGCGCCATCACGAC |
| | | | TCCCGAGGATAAGGAAATCCGGGTCGTAGCATTCACTCAC |
| | | | AAAAATTACTAAAAACAAAGTTTACCCTTCTCCCAAAAGT |
| | | | AAATTTCATATTTGGCTCCACATAATGTGTTCAATGAGTC |
| | | | AAGTGAAGTACTTTTCATTGACAAAAAAAAGTTGCTGAAA |
| | | | AATGCATATCTCATATTTTTTTTTTTTAGAGAAATCCCAT |
| | | | TTCTTGCCTAAACGAAATGCCTATAAAAGAGCATATATTT |
| | | | GCAACAACAGTTTGCAGAAACTATCAAGTCAAATAATCCC |
| | | | CCCTTTAATTCCCTCCCAAA |
| 13 | NtPSO 1.0 kb | Nucleic acid | TTTACGAGTTTACTATACTTTAGTCGTACAGTTTGCAATA |
| | promoter | | ATAGTATTTTAACCACAATTAGTTATATGTACAAAATTTC |
| | | | ATAAGTGAATAACTTTTTTTCAATGACAAAAATAAGAGTT |
| | | | GCTCAAACAATATCAAGTTACAAAAATTTAATTTTAACTG |
| | | | TAAAAGTTATATTTTTCCAAAATAACATAAACTATAGTAA |
| | | | TTATATATAGTTTGAAGTATTAATAAAATTTAAATATGCA |
| | | | AAAGTTAATTTTAATAAACCATTTGTATGCCTACTTGTAG |
| | | | CCTCTAAACTATTTTATTTGCTTTATTTATCAAACTCATA |
| | | | TTTTATTTTATTGCACCTTGTTAGTTTTGGACGTTAATTA |
| | | | TATATATTTGGTGTAAAATTTAAAATATATTAACATTTGT |
| | | | GGAGAATTTATGTATGCCTGGTTCTTAACTATTTTTTTTT |
| | | | TATATAACTGGTTAGAGTAATTTCTTATATTTCAGTATTT |
| | | | ATTTTTAAATAAGTCCTCATAAATTGAAGACTTTAAAAGT |
| | | | TTTTGTGTCATTCCTCTTTTTATTTAAGAAATTGAAGAAT |
| | | | TCCGCTAAATTTCATATTCCGCTGTTATTTAACTGTTTAT |
| | | | TCCCTTGTTAATATAATTGGTAAGAAGTTTTAAAATAAAG |
| | | | GAGTTAATGATTTCTAGGTTCATGGCTTGCCTAGCTTCTA |
| | | | CGAGTAAGCGCCATCACGACTCCCGAGGATAAGGAAATCC |
| | | | GGGTCGTAGCATTCACTCACAAAAATTACTAAAAACAAAG |
| | | | TTTACCCTTCTCCCAAAAGTAAATTTCATATTTGGCTCCA |
| | | | CATAATGTGTTCAATGAGTCAAGTGAAGTACTTTTCATTG |
| | | | ACAAAAAAAAGTTGCTGAAAAATGCATATCTCATATTTTT |
| | | | TTTTTTTAGAGAAATCCCATTTCTTGCCTAAACGAAATGC |
| | | | CTATAAAAGAGCATATATTTGCAACAACAGTTTGCAGAAA |
| | | | CTATCAAGTCAAATAATCCCCCCTTTAATTCCCTCCCAAA |
| | | | ACCAGAATCACCATACAAAGCTATTTCCAGACTCGGAATC |
| | | | CCAAATAGACATCAATAGCACTGAAATGCACTTCAACCCA |
| | | | ATCGTATGAAATTCTTCCAAAAAGGCTAACTTCCACAATA |
| | | | AGTGCCAAAACGATCTCGGGTAATTCCAAACCCGATCTGG |
| | | | ACACACACCCAAGTCCAAAATAATCATACAAACCTGTTAA |
| | | | AACATTCAAATCCCGATTCCGAGGTCAATTACTGAAAAAT |
| | | | CCTATCCTAGTTAATTCTTCCAACTAAAAGCTTCCAAAAT |
| | | | GAGAATTCTATTTCCAAATAAACTCTGAACTTCCCGGAAT |
| | | | TCAATTTTGACTATGCGTACAAGTCATAATACCTGAATTG |
| | | | AAGCTGCAGATAGCCTCAAATCGCTAAACAACGTGCTAGG |
| | | | TCTCAAAACGCCCTGTCGGGTCGTTACATTATAGGTGATT |
| | | | AACTACGTATATACTTGTGCAAGTGTTCTATCATAATTCA |
| | | | GGCCAACTAGTAAGAGTAGAAACAATGAATGGCACATAAC |
| | | | AAACGATCACCACGAAATGTACATGATATAACTCACACAA |
| | | | GGTAGGCACGCTACTAGACAATTACCAATAACAACAATGC |
| 14 | NtPHY 1.5 kb | Nucleic acid | CTAGGACATCACAAGATATGACAAATCAATCCTTACTATC |
| | promoter | | ACGGTTGAGTTGTAACGTGCAAGAATATTTCACTCTTTTT |
| | | | AGGGCACTAAGATCACTCCACCAACATTTCAAGAGAATCC |
| | | | CTGGCACTGCTAAAAAGCCCTCTACACTGTAGTGAATTTT |
| | | | TCTTAGTTATCTAAAGTTAATTATTCATTTAGTATTCTTT |
| | | | ACATTAGGTTCCCCCTTCTAGGTCCTGCACGTAACTAGAT |
| | | | TGAATGGATTGGTCCACTCTATTATTATAGAGTAATTATT |
| | | | AAATTTTTATTTGACTAGGCATCACTAGTTGCACTATCAA |
| | | | CAAAGTATTAGTTCTAGCCTTCTGGGTACTTCATACCTAT |
| | | | GCAAATGATAATTTTATTAAAACAATAGATGTACATGGAT |
| | | | ATAAATACCTATGAAAATTAAATAAATTATAACTAAGAAA |
| | | | AAAACTTTAAAGTTCACTCCTAAGATATCGGGTTATTACA |
| | | | TGACCAAACACAATTTGTTTATCAAATACTTTCAAAAGAA |
| | | | TTTGCCAAACGTAAATTATTTTTCTCCAAAGTGACTTATG |
| | | | AATTACTATGTTGATAAAATACTTTTCAAAGTAACTAATG |
| | | | TTTAGAAGTCAAGGATGGGCTTCTTTTGATTATTGAAGTT |
| | | | TGTAGCAATTGTATGTAGTTATAGTCAGGGTGACCACCAG |
| | | | CATCTCATATAGCAATACACAAGTGGATTAGCGCATTTTA |
| | | | AATTTCAATTAGTTCATGCAAATATACACGTAATAGCATT |
| | | | ATAAGCCACTTTCACAACAGGCAGATTAGTGGTTTTGAAA |
| | | | TTTCAACCAATGATATATACTATAAATTGATCAAGCACAA |
| | | | ACCTTAATTGAGCAACACAATTTCTTACAGCAATAACTAT |
| | | | CACATATAACAATAACTGCC |
| 15 | NtPHY 0.5 kb | Nucleic acid | ATAAATACCTATGAAAATTAAATAAATTATAACTAAGAAA |
| | promoter | | AAAACTTTAAAGTTCACTCCTAAGATATCGGGTTATTACA |
| | | | TGACCAAACACAATTTGTTTATCAAATACTTTCAAAAGAA |
| | | | TTTGCCAAACGTAAATTATTTTTCTCCAAAGTGACTTATG |
| | | | AATTACTATGTTGATAAAATACTTTTCAAAGTAACTAATG |
| | | | TTTAGAAGTCAAGGATGGGCTTCTTTTGATTATTGAAGTT |
| | | | TGTAGCAATTGTATGTAGTTATAGTCAGGGTGACCACCAG |
| | | | CATCTCATATAGCAATACACAAGTGGATTAGCGCATTTTA |
| | | | AATTTCAATTAGTTCATGCAAATATACACGTAATAGCATT |
| | | | ATAAGCCACTTTCACAACAGGCAGATTAGTGGTTTTGAAA |
| | | | TTTCAACCAATGATATATACTATAAATTGATCAAGCACAA |
| | | | ACCTTAATTGAGCAACACAATTTCTTACAGCAATAACTAT |
| | | | CACATATAACAATAACTGCC |
| 16 | NtCYC 1.5 kb | Nucleic acid | TAATACTTATTTTGTAAGAATTGCAATATTGTTATTTTTG |
| | promoter | | TTATGGACTTAAATATTAACCATGTTATAATCTTAAGTTT |
| | | | ATATTATTAGAAAAACTTAGTTTTTGAAAGACTAATATGA |
| | | | ACATTAGTACTTATTTCAAAAATAAGCACCTAGATATATG |
| | | | AAATTACTTTAAGTACTTATTTAAAATAATTAAGTACCAC |
| | | | ACATACATACATATCTCTACAAACTGTTAAAGTTTTCTAT |
| | | | ATGAGTACTTATTTTAAAATAAGAGCTTAAATATAATAAA |
| | | | TTATGTTAAATTTCTTATTTAAAATAATAAAGGACCAAAC |
| | | | ATGCATAAAATAAAGTATGAGCTTAATAAGTCAAGAAGCT |
| | | | AATTGATAAGCATTGATGCCAAATGCACTTACTAACTTTT |
| | | | CTATATTGTAGGAAAAATCTAACTTTTATATTTAAAATTT |
| | | | ATTTTTGTAAAACTTCTCTAATTTTTGGACAAACTCTTAT |
| | | | ATTGATTTTTTAATCAAAGCCAAAATATTTATTTAACTAT |
| | | | GAAAAAATTTTAACAACTAATTTATTATGGTAAATAATAT |
| | | | TGATATGGTAACTTCAAGCACATGACAAAAATTATAACTA |
| | | | ACTGCAGAAGTTTATTGTCTCTCTGAATCTTGTGGCTATA |
| | | | TCATAACAAATACTTGTAGCTAATAAGCCAACGATGTTCT |
| | | | CGGTTTCATATAATTTGAATTTTAAAATAGTTTTTAAATT |
| | | | TAATATTTATTTCAAATCATTATTGTGGCTAACATGTTAT |
| | | | AATCGCAGTAATATTTGGAGATGCAATACTTATATTTAGC |
| | | | TACAAAATTTTATTGTATCAGAATAAGTTTGTAGCTATTA |
| | | | AGTTAGTTTTTGCCACAAATTTTTATAATTGAAGCAAAAA |
| | | | TACCTATTCAGCTACAGTATTTTGTATCGAGTAATATTTT |
| | | | GTGACTAGAAGATTAATATTGCTACAGTAATTTCAGACGT |
| | | | GTGGCAAAAACTCATAATTAGCTACAAAATATTGTCGTAG |
| | | | CAATAATTTTTTATATCTATTAATGCAATTATTACTACAT |
| | | | GCTTTTATAACTTGAGGCAAAAATATCTAATAGCTATAAC |
| | | | ATTTTGTTAGAAGTAATTTTTGTGGCTATAAAATTGGTAT |
| | | | TGCTACAGTAATTTCAAATGCGTGGCAAAAAAATACGATT |
| | | | AACTACGAAATTTTATTGTAGCAATAACNTATTGTAGCAA |
| | | | TAAATTTGTAGCTATTTGGGTAATATTGCTACGACAGTTA |
| | | | GCAATTATAGCAAAAATGCTAAATTAGCTTTGTTAATTTA |
| | | | ATTTTGTAGCTAAACTTTTTTATGAAATTTTAATTTTTGT |
| | | | GGCTATTGTTAGGTATTAGCTACAATTTTCATATATGTAG |
| | | | CTAAGAATTTGTAGCTATATATACATAATGTTGTAGTGGC |
| | | | AAATTCTAACATTGTACGCTTGGCTGCCCTTTTTTTTTTG |
| | | | GCTACAAAACTCTAAAGTAAAGGAACTAGAAAACTCGTTT |
| | | | GGCGAGAGAAAGAGAGAGAG |
| 17 | NtCYC 0.5 kb | Nucleic acid | CAATAATTTTTTATATCTATTAATGCAATTATTACTACAT |
| | promoter | | GCTTTTATAACTTGAGGCAAAAATATCTAATAGCTATAAC |
| | | | ATTTTGTTAGAAGTAATTTTTGTGGCTATAAAATTGGTAT |
| | | | TGCTACAGTAATTTCAAATGCGTGGCAAAAAAATACGATT |
| | | | AACTACGAAATTTTATTGTAGCAATAACNTATTGTAGCAA |
| | | | TAAATTTGTAGCTATTTGGGTAATATTGCTACGACAGTTA |
| | | | GCAATTATAGCAAAAATGCTAAATTAGCTTTGTTAATTTA |
| | | | ATTTTGTAGCTAAACTTTTTTATGAAATTTTAATTTTTGT |
| | | | GGCTATTGTTAGGTATTAGCTACAATTTTCATATATGTAG |
| | | | CTAAGAATTTGTAGCTATATATACATAATGTTGTAGTGGC |
| | | | AAATTCTAACATTGTACGCTTGGCTGCCCTTTTTTTTTTG |
| | | | GCTACAAAACTCTAAAGTAAAGGAACTAGAAAACTCGTTT |
| | | | GGCGAGAGAAAGAGAGAGAG |
| 18 | Geranylgeranyl | Amino acid | MAFLATISGHENMLLSNTLNNNFIFSGKPPQRHSYSFLPK |
| | diphosphate | | KIQARSVANSSKTFQVKEEEFSSKTEKFILPKFDFEEYMK |
| | synthase | | MKAIKVNKALDDAIPMQEPIKIHEAMRYSLLAGGKRVRPI |
| | (g49326) | | LCMASCEVVGGDESLAIPAACSVEMIHTMSLIHDDLPCMD |
| | | | NDDLRRGKPTSHKAFGEDTAVLTGDALLSLAFEHVASKTK |
| | | | DVTPQRVVQAVGELGSAVGSKGLVAGQIVDIASEGKQVSL |
| | | | TELEYIHNHKTGKLLEAAVVCGAIIGGGNEIEVERMRNYA |
| | | | RCLGLLFQVVDDILDVTKSSEELGKTAGKDLVTDKATYPK |
| | | | LMGLEKARELAGELVAKAMDELSYFDAAKAAPLYHFANYI |
| | | | AHRQN |
| 19 | 8-hydroxy- | Amino acid | MQVIITSSHRFFCHHLHQLKSPTSLSAQKAEFKKHGPRNW |
| | copalyl | | LFQTEGSLLYKPVRLNCATSDASYLGNVNEYLESDHSKNS |
| | diphosphate | | EEKDIQVSRTIQMKGLTEEIKHMLNSMEDGRLNVLAYDTA |
| | synthase | | WVSFIPNTTNNGNDQRPMFPSCLQWIIDNQLSDGSWGEEI |
| | (HE588139) | | VFCIYDRLLNTLVCVIALTLWNTCLHKRNKGVMFIKENLS |
| | | | KLETGEVENMTSGFELVFPTLLEKAQQLDIDIPYDAPVLK |
| | | | DIYARREVKLTRIPKDVIHTIPTTVLFSLEGLRDDLDWQR |
| | | | LLKLQMPDGSFLISPASTAFAFMETNDEKCLAYLQNVVEK |
| | | | SNGGARQYPFDLVTRLWAIDRLQRLGISYYFAEEFKELLN |
| | | | HVFRYWDEENGIFSGRNSNVSDVDDTCMAIRLLRLHGYDV |
| | | | SPDALNNFKDGDQFVCFRGEVDGSPTHMENLYRCSQVLFP |
| | | | GEKILEEAKNFTYNFLQQCLANNRCLDKWVIAKDIPGEIW |
| | | | YALEFPWYASLPRVEARYYIEQYGGADDIWIGKTLYRMPD |
| | | | VNNNVYLQAAKLDYNRCQSQHRFEWLIMQEWFEKCNFQQF |
| | | | GISKKYLLVSYFLAAASIFEVEKSRERLAWAKSRIICKMI |
| | | | TSYYNDEATTWTTRNSLLMEFKVSHDPTRKNGNETKEILV |
| | | | LKNLRQFLRQLSEETFEDLGKDIHHQLQNAWETWLVFLRE |
| | | | EKNACQEETELLVRTINLSGGYMTHDEILFDADYENLSNL |
| | | | TNKVCGKLNELQNDKVTGGSKNTNIELDMQALVKLVFGNT |
| | | | SSNINQDIKQTFFAVVKTFYYSAHVSEEIMNFHISKVLFQ |
| | | | QV |
| 20 | Cis-abienol | Amino acid | MVLGLRSKIIPLPDHKLGNIKLGSVTNAICHRPCRVRCSH |
| | synthase | | STASSMEEAKERIRETFGKIELSPSSYDTAWVAMVPSRYS |
| | ISOFORM 1 | | MNQPCFPQCLDWILENQREDGSWGLNPSHPLLVKDSLSST |
| | | | LASLLALRKWRIGDNQVQRGLGFIETHGWAVDNKDQISPL |
| | | | GFEIIFPCMINYAEKLNLDLPLDPNLVNMMLCERELTIER |
| | | | ALKNEFEGNMANVEYFAEGLGELCHWKEMMLRQRHNGSLF |
| | | | DSPATTAAALIYHQYDEKCFGYLNSILKLHDNWVPTICPT |
| | | | KIHSNLFLVDALQNLGVDRYFKTEVKRVLDEIYRLWLEKN |
| | | | EEIFSDVAHCAMAFRLLRMNNYEVSSEELEGFVDQEHFFT |
| | | | TSSGKLMNHVAILELHRASQVAIHERKDHILDKISTWTRN |
| | | | FMEQKLLDKHIPDRSKKEMEFAMRKFYGTFDRVETRRYIE |
| | | | SYKMDSFKILKAAYRSSGINNIDLLKFSEHDENLCQTRHK |
| | | | EELQQMKRWFTDCKLEQVGLSQQYLYTSYFIIAAILFEPE |
| | | | YADARLAYAKYAIIITAVDDFFDCFICKEELQNIIELVER |
| | | | WEGYSTVGFRSERVRIFFLALYKMVEEIAAKAETKQGRCV |
| | | | KDHLINLWIDMLKCMLVELDLWKIKSTTPSIEEYLSVACV |
| | | | TIGVPCFVLTSLYLLGPKLSKDVIESSEVSALCNCTAAVA |
| | | | RLINDIHSYKREQAESSTNMVSILITQSQGTISEEEAIRQ |
| | | | IKEMMESKRRELLGMVLQNKESQLPQVCKDLFWTTINAAY |
| | | | SIHTHGDGYRFPEEFKNHINDVIYKPLNQYSP |
| 21 | Cis-abienol | Amino acid | MILGLRSKIIPLPDHKLGNIKLGSVTNAICHRPCRVRCSH |
| | synthase | | STASSMEEAKERIRETFGKIELSPSSYDTAWVAMVPSRYS |
| | ISOFORM 2 | | MNQPCFPQCLDWILENQREDGSWGLNPSHPLLVKDSLSST |
| | | | LASLLALRKWRIGDNQVQRGLGFIETHGWAVDNKDQISPL |
| | | | GFEIIFPCMINYAEKLNLDLPLDPNLVNMMLCERELTIER |
| | | | ALKNEFEGNMANVEYFAEGLGELCHWKEMMLRQRHNGSLF |
| | | | DSPATTAAALIYHQYDEKCFGYLNSILKLHDNWVPTICPT |
| | | | KIHSNLFLVDALQNLGVDRYFKTEVKRVLDEIYRLWLEKN |
| | | | EEIFSDVAHCAMAFRLLRMNNYEVSSEELEGFVDQEHFFT |
| | | | TSSGKLMNHVAILELHRASQVAIHERKDHILDKISTWTRN |
| | | | FMEQKLLDKHIPDRSKKEMEFAMRKFYGTEDRVETRRYIE |
| | | | SYKMDSFKILKAAYRWEGYSTVGFRSERVRIFFLALYKMV |
| | | | EEIAAKAETKQGRCVKDHLINLWIDMLKCMLVELDLWKIK |
| | | | STTPSIEEYLSVACVTIGVPCFVLTSLYLLGPKLSKDVIE |
| | | | SSEVSALCNCTAAVARLINDIHSYKREQAESSTNMVSILI |
| | | | TQSQGTISEEEAIRQIKEMMESKRRELLGMVLQNKESQLP |
| | | | QVCKDLFWTTINAAYSIHTHGDGYRFPEEFKNHINDVIYK |
| | | | PLNQYSP |
| 22 | Cembratrienol | Amino acid | MSQSISPLICSHFAKFQSNIWRCNTSQLRVIHSSYASFGG |
| | synthase 2a | | RRKERVRRMNRAMDLSSSSRHLADFPSTIWGDHFLSYNSE |
| | (g58533) | | ITEITTQEKNEHEMLKEIVRKMLVETPDNSTQKLVLIDTI |
| | | | QRLGLAYHENDEIENSIQNIFNLSQNSEDDDEHNLYVAAL |
| | | | RFRLARQQGYYMSSDVFKQFTNHDGKFKENHTNDVQGLLS |
| | | | LYEAAHMRVHDEEILEEALIFTTTHLESVIPNLSNSLKVQ |
| | | | VTEALSHPIRKAIPRVGARKYIHIYENIGTHNDLLLKFAK |
| | | | LDENMLQKLHRKELNELTSWWKDLDRANKFPYAKDRIVEA |
| | | | YFWTVGIYFEPQYSRSRSLVTKVVKMNSIIDDTYDAYATF |
| | | | DELVLFTDAIQRWDEGAMDLLPTYLRPIYQGLLDVENEME |
| | | | EVLAKEGKADHIYYAKKEMKKVAEVYFKEAEWLNANYIPK |
| | | | CEEYMKNGLVSSTGPMYGIISLVVMEEIITKEAFEWLTNE |
| | | | PLILRAASTICRLMDDMADHEVEQQRGHVASFVECYMKEY |
| | | | GVSKQEAYVEMRKKITNAWKDINKELLRPTAVPMFILERS |
| | | | LNFSRLADTFLKDDDGYTNPKSKVKDLIASLFVESVDI |
| 23 | Levopimaradiene | Amino acid | MSCQYYLTTTTSSLRIFSFTPRRYAPNSSASQPHEFFKKQ |
| | synthetase | | VLFSSNLQCNAVSRPRAQVIKRDDNVEEVDSAEEQQEEEE |
| | (g33184) | | TQEVYRSNKIKQHIYAVRLMLQSMDDGEISISAYDTAWVA |
| | | | LVKDINGSDTPQFPSSLEWIANNQLAECSWGDKSIFLAHD |
| | | | RIINTLACVIALKSWNLHIDKRELGMSFIRENLSKIGDEN |
| | | | AVHMPIGFEVAFPSLIEIGKKIGIDIPDDSHVLREIYT |
| 24 | 2- | Amino acid | MASITINHSFSRNPNISFHPQNPLIQTQALENFKPSISKC |
| | isopropylmalate | | SPIIHCAIRRRPEYTPSHIPDPNYIRIFDTTLRDGEQSPG |
| | synthetase | | ATMTTKEKLDVARQLAKLGVDIIEAGFPASSEADLEAVKL |
| | (g36718) | | IAKEVGNGVNEEGHVPVICGLARCNKRDIDKAWEAVKYAK |
| | | | KPRIHTFIATSEIHMKFKLKMSRDEVVEKARSMVAYARSI |
| | | | GCEDVEFSPEDAGRSDPEFLYHILGEVIKAGATTINIPDT |
| | | | VGYTVPSEFGKLIADIKANTPGIGDVIISTHCQNDLGLST |
| | | | ANTLAGACAGARQVEVTINGIGERAGNASLEEVVMALKCR |
| | | | GEQVLGGLYTGINTQHILMSSKMVEEYTGLHVQPHKAIVG |
| | | | ANAFAHESGIHQDGMLKHKDTYEIISPEDIGLNRVNESGI |
| | | | VLGKLSGRHALQAKMLELGYDIEGKELEDLFWRFKSVAEK |
| | | | KKKITDDDLIALMSDEVFQPQFVWQLENVQVTCGSLGLST |
| | | | ATVKLIDADGQEHVSCSVGTGPVDAAYKAVDLIVKVPVAL |
| | | | LEYSLNAVTEGIDAIASTRVLIRGENGHTSTHALTGETVH |
| | | | RSFSGTGADMDIVISSVRAYIGALNKMLSFRKLVSKHSKP |
| | | | EGSAVV |
| 25 | 2- | Amino acid | MAILISRSRNFNHFLLSTRFRFLSRLNQTSSKTFSHHAKT |
| | oxoisovalerate | | SSFTTAAGGGFTKPTATFSGESAAVERVERFQSNKTGEQL |
| | dehydrogenase | | NTLYYEEDNHHQIIDENQVMDFPGGQLPITPQMKFIAESS |
| | subunit alpha | | EKRLPCYRVLDDDGYPIPGSIFEEVSKELAVKMYSSMVTL |
| | (g63865) | | QVMDTIFYEAQRQGRLSFYLTTAGEEGINIASAAALSVDD |
| | | | FVLPQYREVGVILWRGFPLKDIANQLFGNKFDYGKGRQMP |
| | | | CHHGSNELNYLTISSPIATQIPQAAGVAYSLKMDKKEACA |
| | | | ITYLGDGSTSEGDFHAALNFAAVLDAPVVFICRNNGWAIS |
| | | | TPVNEQFRSDGVASKGQAYGIRSIRVDGNDVLATHSAIRA |
| | | | AREMAIKEQKPILVEAMTYRVAHHSTSDDSTKYRPVEEIE |
| | | | HWKTAKSPISRFRKWIQRNGWWNDENESELRGDTRKQVLQ |
| | | | VMQAAEKVEKPPLTDLFTDVYDKVPLNLQEQHKFIRDAVK |
| | | | KSPREYPSDVPI |
| 26 | Neomenthol | Amino acid | MAEKITSHENTRYAVVTGGNKGIGYETCRQLAKEGIVVVL |
| | Dehydrogenase | | TARDERRGIEALEKLKEEYSSNKTDDDQILFHQLDVMDPA |
| | (NtNMD; | | SISSLVDFIKTKFGKLDILVNNAGIGGLMVEGDVVIIKDL |
| | g29387) | | IEGDFVTISAENGEEDGIKKSIEGIERIVTDYELTKQCLE |
| | | | TNFYGAKRMIEAFIPLLQLSNSPRIVNVASFLGKLKLLCN |
| | | | QWAIGMLSDAKSLREERVDEVLNEFIKDFKEKSIEAKGWP |
| | | | TYFSAYKVSKASLIAYTRVLATKYPNFRINSVCPGFCKTD |
| | | | VNCNTGSLTAEEGAESLVKLALVPNDGPSGLFFYRKEVTS |
| | | | F |
| 27 | Geranylgeranyl | Nucleic acid | ATGGCATTTTTGGCTACCATTTCTGGCCATGAAAATATGC |
| | diphosphate | | TTCTTTCCAATACCCTAAACAATAACTTTATTTTCAGTGG |
| | synthase | | AAAACCTCCACAGAGACATTCTTATAGTTTCCTCCCCAAG |
| | (g49326) | | AAAATCCAGGCCAGAAGTGTTGCAAACTCATCCAAAACAT |
| | | | TTCAAGTCAAAGAAGAAGAATTCTCATCTAAGACAGAGAA |
| | | | ATTCATCTTGCCTAAGTTTGACTTTGAAGAATATATGAAA |
| | | | ATGAAGGCAATTAAGGTAAACAAAGCACTAGATGATGCAA |
| | | | TACCAATGCAAGAGCCTATAAAAATTCATGAAGCCATGAG |
| | | | ATACTCACTTCTAGCTGGGGGAAAACGCGTCCGGCCGATC |
| | | | CTATGCATGGCTTCTTGTGAAGTAGTAGGAGGGGATGAAT |
| | | | CCTTAGCTATTCCTGCAGCTTGCTCGGTTGAGATGATCCA |
| | | | CACCATGTCACTCATCCACGACGATCTTCCTTGCATGGAC |
| | | | AACGATGATCTACGTCGTGGCAAGCCCACGAGCCACAAGG |
| | | | CTTTCGGGGAAGACACTGCAGTTCTAACAGGGGATGCACT |
| | | | TTTGTCTTTGGCCTTTGAACATGTAGCTTCCAAGACTAAA |
| | | | GATGTGACACCCCAAAGAGTGGTTCAAGCCGTTGGCGAAT |
| | | | TGGGTTCAGCCGTTGGCTCGAAAGGGCTTGTGGCGGGGCA |
| | | | GATTGTGGACATAGCTAGTGAGGGAAAACAAGTGAGCCTA |
| | | | ACTGAATTAGAGTACATTCACAACCATAAGACAGGGAAAC |
| | | | TATTGGAGGCTGCTGTGGTTTGTGGGGCAATAATTGGGGG |
| | | | AGGGAATGAGATTGAGGTGGAGAGAATGAGGAACTATGCT |
| | | | AGATGCCTTGGACTGTTGTTTCAAGTGGTAGATGATATTC |
| | | | TTGATGTTACTAAGTCATCAGAAGAGTTGGGAAAGACAGC |
| | | | TGGTAAAGACCTAGTGACTGATAAGGCTACATATCCTAAG |
| | | | TTGATGGGGCTAGAAAAAGCTCGGGAGCTCGCCGGAGAGC |
| | | | TGGTGGCTAAGGCCATGGATGAGCTGAGCTACTTTGATGC |
| | | | TGCCAAGGCGGCACCTCTTTATCATTTTGCTAATTATATT |
| | | | GCACATCGCCAGAATTGA |
| 28 | 8-hydroxy- | Nucleic acid | ATGCAAGTTATAATTACGTCCAGTCACAGATTTTTCTGCC |
| | copalyl | | ATCATCTTCATCAGCTCAAGAGTCCTACATCGTTGTCTGC |
| | diphosphate | | ACAGAAAGCTGAGTTTAAAAAACATGGACCCCGAAATTGG |
| | synthase | | TTGTTCCAAACTGAAGGCTCACTTCTATATAAACCAGTTC |
| | (HE588139) | | GTCTCAATTGTGCAACTAGTGATGCAAGTTATCTTGGTAA |
| | | | TGTGAATGAGTACTTAGAATCTGATCACTCAAAAAACTCC |
| | | | GAAGAAAAGGATATTCAGGTAAGCAGAACAATACAGATGA |
| | | | AAGGTTTGACAGAAGAGATCAAACACATGTTGAATTCGAT |
| | | | GGAGGATGGAAGGTTAAATGTCTTAGCCTATGACACAGCT |
| | | | TGGGTTTCCTTTATTCCAAATACTACTAATAATGGAAATG |
| | | | ATCAAAGACCTATGTTTCCATCTTGTCTTCAATGGATTAT |
| | | | AGACAATCAACTTTCTGATGGTTCATGGGGAGAGGAGATT |
| | | | GTATTCTGCATATATGATCGACTCTTGAACACACTAGTAT |
| | | | GTGTTATTGCATTGACATTATGGAACACGTGCCTTCATAA |
| | | | GAGAAACAAAGGTGTGATGTTTATCAAAGAAAACTTAAGC |
| | | | AAGCTAGAGACAGGGGAAGTTGAAAACATGACTAGTGGAT |
| | | | TTGAACTTGTGTTTCCTACTCTCCTTGAAAAAGCTCAACA |
| | | | ACTAGATATTGACATTCCGTATGATGCTCCAGTCTTAAAG |
| | | | GATATTTATGCAAGGAGAGAAGTAAAGTTAACAAGAATTC |
| | | | CTAAAGATGTTATCCATACGATTCCGACAACAGTATTGTT |
| | | | TTCATTAGAAGGATTAAGGGACGACTTAGACTGGCAAAGA |
| | | | CTTTTAAAGCTTCAAATGCCTGATGGTTCATTCTTAATAT |
| | | | CCCCTGCTTCCACTGCCTTTGCATTCATGGAAACAAATGA |
| | | | TGAAAAGTGTTTGGCATATCTTCAGAACGTTGTTGAAAAG |
| | | | AGTAATGGAGGAGCGCGACAATACCCGTTCGACTTGGTAA |
| | | | CACGACTTTGGGCAATTGATCGATTACAACGCCTTGGAAT |
| | | | CTCTTATTATTTTGCGGAAGAGTTCAAGGAACTTTTGAAT |
| | | | CATGTGTTCAGATACTGGGACGAGGAGAATGGAATTTTTA |
| | | | GTGGAAGGAATTCAAACGTTTCTGATGTTGATGATACATG |
| | | | CATGGCTATAAGGTTGCTAAGGTTACATGGGTATGATGTT |
| | | | AGTCCAGATGCGCTAAACAATTTCAAAGATGGTGATCAAT |
| | | | TCGTTTGCTTCAGAGGTGAAGTGGACGGGTCACCAACACA |
| | | | TATGTTTAATCTCTATAGATGTTCCCAAGTTTTATTCCCA |
| | | | GGAGAAAAGATTCTTGAAGAGGCAAAGAATTTTACTTATA |
| | | | ACTTCTTACAGCAATGTCTTGCAAACAACCGATGCTTAGA |
| | | | CAAATGGGTCATAGCTAAGGACATCCCCGGGGAGATATGG |
| | | | TATGCACTAGAATTTCCATGGTATGCCAGCTTACCTCGGG |
| | | | TGGAAGCTAGGTATTATATAGAACAGTATGGCGGAGCAGA |
| | | | TGATATTTGGATTGGCAAGACGTTATACAGAATGCCCGAT |
| | | | GTCAACAACAATGTTTATTTACAAGCTGCAAAATTGGATT |
| | | | ACAATAGATGCCAAAGTCAACATCGCTTTGAATGGCTGAT |
| | | | TATGCAAGAGTGGTTTGAGAAGTGCAACTTTCAACAATTT |
| | | | GGAATAAGCAAAAAGTACCTCCTAGTTTCTTATTTCCTAG |
| | | | CTGCTGCAAGTATATTTGAAGTCGAGAAGTCAAGAGAACG |
| | | | CCTTGCATGGGCTAAATCTCGTATAATATGTAAGATGATT |
| | | | ACATCTTACTACAATGATGAAGCCACAACTTGGACAACTA |
| | | | GGAATTCATTGCTAATGGAATTCAAGGTTTCTCATGATCC |
| | | | GACCAGAAAAAATGGTAATGAAACAAAAGAGATCTTAGTT |
| | | | CTCAAAAATCTTCGTCAGTTTTTGCGCCAACTATCAGAAG |
| | | | AAACTTTTGAAGACCTAGGCAAAGACATCCATCACCAACT |
| | | | ACAAAATGCTTGGGAAACGTGGTTGGTGTTCTTGAGGGAG |
| | | | GAAAAGAATGCATGTCAAGAAGAAACAGAGTTGCTGGTGC |
| | | | GCACAATTAATCTCTCGGGCGGCTATATGACACATGATGA |
| | | | GATATTATTCGATGCGGACTACGAGAATCTGTCCAACCTT |
| | | | ACCAATAAAGTTTGTGGCAAGCTTAATGAGCTCCAAAATG |
| | | | ACAAGGTGACGGGCGGCTCAAAGAACACCAATATTGAACT |
| | | | CGACATGCAAGCTCTCGTAAAGTTAGTGTTTGGTAACACC |
| | | | TCAAGCAACATCAACCAAGACATTAAGCAAACATTTTTTG |
| | | | CAGTTGTGAAGACTTTCTATTACAGTGCGCATGTTAGTGA |
| | | | GGAAATAATGAACTTTCACATATCCAAAGTGCTTTTTCAG |
| | | | CAAGTCTAG |
| 29 | Cis-abienol | Nucleic acid | ATGGTACTTGGACTGAGAAGCAAAATCATACCACTTCCTG |
| | Synthase | | ATCATAAGTTGGGAAATATCAAATTAGGTTCAGTAACCAA |
| | Isoform 1 | | TGCAATTTGCCACAGACCATGTAGAGTAAGATGCAGCCAC |
| | (NtaABS) | | AGTACTGCTTCATCAATGGAAGAGGCAAAGGAGAGAATAA |
| | | | GGGAAACATTTGGAAAAATAGAGCTATCTCCTTCTTCCTA |
| | | | TGACACAGCATGGGTAGCTATGGTCCCTTCAAGATATTCT |
| | | | ATGAACCAACCATGTTTTCCTCAGTGCTTAGATTGGATTC |
| | | | TTGAAAATCAAAGAGAAGATGGATCTTGGGGCCTAAATCC |
| | | | TAGCCATCCATTGCTTGTAAAAGACTCCCTTTCTTCCACT |
| | | | CTAGCATCTTTGCTTGCCCTTCGCAAATGGAGAATTGGAG |
| | | | ATAACCAAGTCCAAAGAGGCCTTGGCTTTATTGAAACGCA |
| | | | TGGTTGGGCAGTCGATAACAAGGATCAGATTTCACCTTTA |
| | | | GGATTTGAAATTATATTTCCCTGCATGATCAACTATGCAG |
| | | | AGAAACTTAATTTGGATCTACCTTTGGATCCTAACCTTGT |
| | | | AAATATGATGCTCTGCGAACGTGAATTAACAATTGAAAGA |
| | | | GCCTTAAAGAATGAATTCGAGGGGAATATGGCAAATGTAG |
| | | | AATATTTTGCTGAAGGGCTCGGTGAATTATGTCATTGGAA |
| | | | AGAGATGATGCTTCGTCAGAGACACAACGGGTCGCTCTTT |
| | | | GATTCACCAGCCACTACTGCAGCTGCCTTGATTTACCATC |
| | | | AGTACGATGAGAAATGCTTTGGGTACTTGAACTCAATCTT |
| | | | GAAACTGCACGATAATTGGGTCCCCACTATTTGCCCTACA |
| | | | AAGATACATTCAAATCTCTTCTTAGTTGATGCCCTTCAAA |
| | | | ATCTTGGAGTAGATCGGTATTTTAAAACAGAAGTCAAAAG |
| | | | AGTACTAGATGAAATATACAGGCTTTGGCTAGAAAAGAAT |
| | | | GAAGAAATTTTTTCAGACGTTGCTCATTGTGCCATGGCGT |
| | | | TTCGACTTTTACGGATGAATAACTATGAAGTTTCCTCAGA |
| | | | AGAACTTGAAGGATTTGTCGACCAAGAACATTTCTTTACA |
| | | | ACATCAAGTGGGAAACTTATGAATCACGTTGCAATTCTCG |
| | | | AACTTCACCGAGCTTCACAGGTGGCTATTCATGAAAGGAA |
| | | | AGATCACATTTTAGATAAAATAAGTACTTGGACAAGGAAT |
| | | | TTTATGGAGCAAAAACTCTTGGACAAGCACATCCCTGATA |
| | | | GGTCAAAGAAGGAGATGGAATTTGCTATGAGGAAATTTTA |
| | | | TGGCACATTTGATCGAGTGGAAACTAGACGTTACATCGAG |
| | | | TCATACAAAATGGACAGTTTTAAGATCTTAAAAGCGGCTT |
| | | | ACAGGTCTTCCGGTATTAACAACATAGACTTGCTAAAGTT |
| | | | CTCAGAACACGATTTTAACTTGTGCCAAACCCGACACAAA |
| | | | GAAGAACTTCAACAGATGAAAAGGTGGTTCACAGATTGCA |
| | | | AACTCGAACAAGTAGGATTATCACAACAGTACTTATACAC |
| | | | TAGTTACTTCATAATTGCTGCCATACTCTTTGAACCTGAA |
| | | | TATGCTGATGCTCGTCTAGCATATGCAAAGTACGCCATAA |
| | | | TAATAACAGCGGTGGATGATTTCTTCGATTGTTTTATTTG |
| | | | CAAAGAAGAACTGCAAAACATCATCGAATTAGTAGAGAGA |
| | | | TGGGAGGGATACTCAACCGTCGGATTCCGTTCAGAGAGGG |
| | | | TTAGAATTTTCTTTTTGGCACTTTACAAAATGGTAGAGGA |
| | | | AATTGCGGCAAAGGCGGAAACTAAGCAAGGTCGATGTGTC |
| | | | AAAGATCACCTTATTAACTTGTGGATTGATATGTTGAAGT |
| | | | GTATGCTGGTGGAATTGGACCTTTGGAAAATTAAATCAAC |
| | | | TACCCCAAGCATAGAGGAGTACTTGTCTGTTGCATGTGTA |
| | | | ACTATTGGTGTTCCATGTTTTGTTCTCACATCACTATATC |
| | | | TTCTTGGACCAAAACTGTCCAAGGATGTCATAGAAAGTTC |
| | | | TGAGGTCAGTGCCTTATGCAATTGTACAGCTGCTGTGGCC |
| | | | CGATTGATTAATGATATACACAGTTACAAGAGAGAACAAG |
| | | | CAGAAAGTTCAACAAATATGGTATCAATATTAATAACACA |
| | | | AAGTCAGGGAACTATCTCTGAAGAAGAGGCTATAAGACAG |
| | | | ATAAAGGAAATGATGGAAAGTAAGAGAAGAGAGTTGCTAG |
| | | | GGATGGTTCTACAAAATAAAGAAAGCCAATTGCCACAAGT |
| | | | GTGCAAGGATCTTTTTTGGACGACAATCAACGCAGCTTAT |
| | | | TCTATACATACACATGGCGATGGGTATCGCTTCCCAGAGG |
| | | | AATTCAAGAACCATATCAACGATGTAATTTACAAACCACT |
| | | | CAATCAATATTCCCCATAA |
| 30 | Cis-abienol | Nucleic acid | ATGATACTTGGACTGAGAAGCAAAATCATACCACTTCCTG |
| | Synthase | | ATCATAAGTTGGGAAATATCAAATTAGGTTCAGTAACCAA |
| | Isoform 2 | | TGCAATTTGCCACAGACCATGTAGAGTAAGATGCAGCCAC |
| | (NtABS) | | AGTACTGCTTCATCAATGGAAGAGGCAAAGGAGAGAATAA |
| | | | GGGAAACATTTGGAAAAATAGAGCTATCTCCTTCTTCCTA |
| | | | TGACACAGCATGGGTAGCTATGGTCCCTTCAAGATATTCT |
| | | | ATGAACCAACCATGTTTTCCTCAGTGCTTAGATTGGATTC |
| | | | TTGAAAATCAAAGAGAAGATGGATCTTGGGGCCTAAATCC |
| | | | TAGCCATCCATTGCTTGTAAAAGACTCCCTTTCTTCCACT |
| | | | CTAGCATCTTTGCTTGCCCTTCGCAAATGGAGAATTGGAG |
| | | | ATAACCAAGTCCAAAGAGGCCTTGGCTTTATTGAAACGCA |
| | | | TGGTTGGGCAGTCGATAACAAGGATCAGATTTCACCTTTA |
| | | | GGATTTGAAATTATATTTCCCTGCATGATCAACTATGCAG |
| | | | AGAAACTTAATTTGGATCTACCTTTGGATCCTAACCTTGT |
| | | | AAATATGATGCTCTGCGAACGTGAATTAACAATTGAAAGA |
| | | | GCCTTAAAGAATGAATTCGAGGGGAATATGGCAAATGTAG |
| | | | AATATTTTGCTGAAGGGCTCGGTGAATTATGTCATTGGAA |
| | | | AGAGATGATGCTTCGTCAGAGACACAACGGGTCGCTCTTT |
| | | | GATTCACCAGCCACTACTGCAGCTGCCTTGATTTACCATC |
| | | | AGTACGATGAGAAATGCTTTGGGTACTTGAACTCAATCTT |
| | | | GAAACTGCACGATAATTGGGTCCCCACTATTTGCCCTACA |
| | | | AAGATACATTCAAATCTCTTCTTAGTTGATGCCCTTCAAA |
| | | | ATCTTGGAGTAGATCGGTATTTTAAAACAGAAGTCAAAAG |
| | | | AGTACTAGATGAAATATACAGGCTTTGGCTAGAAAAGAAT |
| | | | GAAGAAATTTTTTCAGACGTTGCTCATTGTGCCATGGCGT |
| | | | TTCGACTTTTACGGATGAATAACTATGAAGTTTCCTCAGA |
| | | | AGAACTTGAAGGATTTGTCGACCAAGAACATTTCTTTACA |
| | | | ACATCAAGTGGGAAACTTATGAATCACGTTGCAATTCTCG |
| | | | AACTTCACCGAGCTTCACAGGTGGCTATTCATGAAAGGAA |
| | | | AGATCACATTTTAGATAAAATAAGTACTTGGACAAGGAAT |
| | | | TTTATGGAGCAAAAACTCTTGGACAAGCACATCCCTGATA |
| | | | GGTCAAAGAAGGAGATGGAATTTGCTATGAGGAAATTTTA |
| | | | TGGCACATTTGATCGAGTGGAAACTAGACGTTACATCGAG |
| | | | TCATACAAAATGGACAGTTTTAAGATCTTAAAAGCGGCTT |
| | | | ACAGATGGGAGGGATACTCAACCGTCGGATTCCGTTCAGA |
| | | | GAGGGTTAGAATTTTCTTTTTGGCACTTTACAAAATGGTA |
| | | | GAGGAAATTGCGGCAAAGGCGGAAACTAAGCAAGGTCGAT |
| | | | GTGTCAAAGATCACCTTATTAACTTGTGGATTGATATGTT |
| | | | GAAGTGTATGCTGGTGGAATTGGACCTTTGGAAAATTAAA |
| | | | TCAACTACCCCAAGCATAGAGGAGTACTTGTCTGTTGCAT |
| | | | GTGTAACTATTGGTGTTCCATGTTTTGTTCTCACATCACT |
| | | | ATATCTTCTTGGACCAAAACTGTCCAAGGATGTCATAGAA |
| | | | AGTTCTGAGGTCAGTGCCTTATGCAATTGTACAGCTGCTG |
| | | | TGGCCCGATTGATTAATGATATACACAGTTACAAGAGAGA |
| | | | ACAAGCAGAAAGTTCAACAAATATGGTATCAATATTAATA |
| | | | ACACAAAGTCAGGGAACTATCTCTGAAGAAGAGGCTATAA |
| | | | GACAGATAAAGGAAATGATGGAAAGTAAGAGAAGAGAGTT |
| | | | GCTAGGGATGGTTCTACAAAATAAAGAAAGCCAATTGCCA |
| | | | CAAGTGTGCAAGGATCTTTTTTGGACGACAATCAACGCAG |
| | | | CTTATTCTATACATACACATGGCGATGGGTATCGCTTCCC |
| | | | AGAGGAATTCAAGAACCATATCAACGATGTAATTTACAAA |
| | | | CCACTCAATCAATATTCCCCATAA |
| 31 | Cembratrienol | Nucleic acid | ATGAGTCAATCAATTTCTCCATTAATCTGTTCTCACTTTG |
| | synthase 2a | | CGAAATTTCAGTCGAATATTTGGAGATGCAATACTTCTCA |
| | (g58533) | | ACTCAGAGTTATACACTCATCATATGCCTCTTTTGGAGGG |
| | | | AGAAGAAAAGAGAGAGTAAGAAGAATGAATCGAGCAATGG |
| | | | ATCTTTCTTCAAGCTCTCGTCATTTGGCAGATTTTCCCTC |
| | | | AACAATTTGGGGTGACCATTTTCTCTCCTACAATTCTGAA |
| | | | ATAACAGAAATTACTACCCAAGAGAAAAATGAACATGAAA |
| | | | TGCTAAAAGAAATAGTTCGGAAAATGTTGGTAGAAACTCC |
| | | | AGATAATAGTACACAAAAACTAGTCTTGATTGACACAATT |
| | | | CAAAGATTGGGATTAGCATATCATTTCAATGATGAGATTG |
| | | | AAAACTCCATTCAAAACATCTTTAATTTGTCTCAAAATAG |
| | | | TGAAGATGACGATGAACACAACCTTTATGTTGCTGCTCTT |
| | | | CGTTTTCGACTTGCGAGGCAACAAGGATATTACATGTCTT |
| | | | CAGATGTGTTCAAGCAATTCACTAACCATGACGGAAAATT |
| | | | CAAGGAAAATCATACTAATGATGTTCAAGGATTATTGAGT |
| | | | TTGTATGAAGCAGCACATATGAGAGTGCACGACGAGGAAA |
| | | | TTCTAGAAGAAGCTCTTATCTTTACCACGACTCATCTCGA |
| | | | GTCCGTGATCCCGAATTTGAGCAACTCGCTTAAGGTACAA |
| | | | GTTACTGAAGCCTTAAGCCATCCTATTCGCAAAGCTATAC |
| | | | CAAGGGTGGGAGCAAGGAAATACATACACATATATGAAAA |
| | | | CATTGGAACACATAATGATTTACTTTTGAAATTTGCAAAG |
| | | | TTGGACTTCAACATGTTACAAAAGCTTCATCGAAAAGAGC |
| | | | TTAACGAGCTAACAAGCTGGTGGAAAGATTTGGATCGTGC |
| | | | AAACAAATTTCCATATGCAAAGGACAGATTAGTAGAAGCT |
| | | | TACTTTTGGACGGTGGGAATATATTTTGAACCTCAATATA |
| | | | GTCGTTCAAGAAGTTTGGTAACAAAAGTAGTCAAAATGAA |
| | | | CTCCATTATTGATGACACTTATGATGCTTATGCAACTTTT |
| | | | GATGAGCTTGTGCTTTTCACGGATGCGATCCAAAGATGGG |
| | | | ACGAAGGTGCCATGGATTTATTACCGACATATCTGAGACC |
| | | | TATTTATCAAGGCCTTCTCGACGTTTTCAATGAAATGGAA |
| | | | GAAGTATTGGCCAAAGAAGGTAAAGCAGATCACATCTACT |
| | | | ATGCGAAAAAAGAGATGAAAAAGGTGGCGGAAGTCTATTT |
| | | | TAAGGAAGCTGAATGGTTGAATGCTAACTACATTCCAAAA |
| | | | TGCGAGGAGTATATGAAAAATGGACTTGTAAGCTCTACCG |
| | | | GTCCGATGTATGGAATAATTTCTTTGGTTGTTATGGAGGA |
| | | | AATTATAACAAAAGAGGCTTTTGAATGGTTGACAAATGAA |
| | | | CCTTTGATTCTTCGAGCTGCATCAACAATTTGTAGATTAA |
| | | | TGGATGATATGGCTGATCATGAAGTTGAACAACAAAGAGG |
| | | | ACATGTTGCTTCATTTGTTGAGTGCTACATGAAAGAATAT |
| | | | GGAGTTTCAAAGCAAGAAGCATATGTTGAGATGCGGAAAA |
| | | | AAATCACAAATGCGTGGAAAGATATAAATAAGGAACTCTT |
| | | | GCGCCCTACTGCAGTACCAATGTTTATCCTCGAACGATCT |
| | | | TTAAATTTTTCAAGATTGGCCGATACATTTTTGAAAGATG |
| | | | ATGATGGATACACAAATCCCAAATCCAAAGTTAAAGACTT |
| | | | GATTGCTTCGTTGTTTGTCGAATCTGTCGACATATGA |
| 32 | Levopimaradiene | Nucleic acid | ATGTCTTGTCAATATTACTTAACCACGACGACCTCTTCTC |
| | synthetase | | TCAGAATATTCTCCTTCACCCCCCGCCGTTACGCACCGAA |
| | (g33184) | | TTCTTCTGCAAGTCAACCTCATGAGTTCTTTAAAAAACAA |
| | | | GTACTTTTCAGTTCCAATCTGCAATGCAATGCGGTTTCAA |
| | | | GACCTCGCGCACAAGTTATCAAGCGGGACGACAACGTGGA |
| | | | AGAAGTAGACAGTGCAGAAGAACAACAAGAAGAAGAAGAA |
| | | | ACACAAGAGGTGTACAGATCAAATAAGATAAAGCAACATA |
| | | | TTTATGCCGTCCGGTTAATGTTGCAAAGTATGGATGATGG |
| | | | AGAGATAAGTATATCAGCTTATGACACAGCTTGGGTTGCT |
| | | | CTTGTGAAAGACATTAATGGAAGCGATACTCCTCAATTCC |
| | | | CTTCAAGTCTTGAATGGATTGCCAACAATCAACTTGCTGA |
| | | | ATGTTCGTGGGGTGACAAGTCCATCTTTTTGGCTCACGAT |
| | | | CGAATCATCAACACATTGGCCTGTGTTATTGCTTTGAAAT |
| | | | CTTGGAATTTGCACATTGACAAAAGAGAACTAGGAATGTC |
| | | | GTTTATCAGAGAGAATTTAAGCAAGATTGGAGATGAAAAT |
| | | | GCTGTGCATATGCCAATAGGATTTGAAGTGGCGTTTCCTT |
| | | | CACTAATTGAGATTGGAAAAAAGATAGGCATTGATATTCC |
| | | | GGATGATTCTCATGTCTTGAGAGAGATATATACCTGA |
| 33 | 2- | Nucleic acid | ATGGCGTCTATCACCATAAACCATTCATTTTCCCGTAACC |
| | isopropylmalate | | CTAACATCTCATTCCATCCCCAAAATCCTCTCATTCAAAC |
| | synthetase | | CCAAGCTCTCTTCAATTTCAAACCATCAATCTCCAAATGT |
| | (g36718) | | TCCCCTATTATCCACTGCGCAATCCGCCGTCGACCCGAAT |
| | | | ATACCCCGAGCCACATTCCCGACCCGAACTACATTCGCAT |
| | | | CTTCGACACCACTCTCCGCGACGGCGAACAATCCCCAGGC |
| | | | GCCACAATGACCACAAAAGAAAAACTCGACGTTGCGCGTC |
| | | | AGTTAGCTAAGCTTGGTGTTGACATAATTGAAGCCGGTTT |
| | | | TCCTGCTTCTTCTGAAGCTGATCTCGAAGCTGTGAAATTA |
| | | | ATAGCGAAGGAAGTTGGAAATGGTGTGAATGAAGAGGGAC |
| | | | ATGTTCCGGTAATTTGTGGACTTGCGAGGTGTAATAAGAG |
| | | | GGATATTGATAAGGCTTGGGAGGCTGTGAAGTATGCGAAA |
| | | | AAACCGAGGATTCATACGTTTATTGCGACTAGTGAGATAC |
| | | | ATATGAAGTTTAAGTTGAAGATGAGTAGAGATGAAGTTGT |
| | | | GGAGAAAGCTAGGAGTATGGTTGCTTATGCTAGGAGTATT |
| | | | GGTTGTGAGGATGTTGAATTTAGCCCAGAAGATGCTGGAA |
| | | | GATCCGATCCAGAGTTCCTCTATCATATCCTTGGAGAGGT |
| | | | CATCAAAGCTGGGGCAACAACCCTTAACATCCCTGATACT |
| | | | GTTGGATACACTGTTCCCAGCGAATTTGGAAAATTGATTG |
| | | | CTGATATAAAGGCCAATACCCCAGGAATTGGAGATGTGAT |
| | | | CATCTCAACACACTGCCAGAACGATCTTGGGCTTTCTACT |
| | | | GCCAACACCTTAGCTGGAGCATGCGCAGGTGCAAGACAAG |
| | | | TAGAAGTGACCATCAACGGAATCGGTGAAAGAGCTGGAAA |
| | | | TGCTTCTTTGGAGGAGGTTGTAATGGCCTTAAAATGTCGT |
| | | | GGAGAGCAAGTACTAGGTGGCCTGTATACAGGAATTAATA |
| | | | CACAACATATACTCATGTCAAGCAAGATGGTAGAGGAGTA |
| | | | CACCGGGCTTCATGTGCAGCCACACAAGGCCATTGTTGGA |
| | | | GCTAATGCGTTTGCTCATGAAAGTGGCATCCATCAGGATG |
| | | | GAATGTTAAAACACAAAGATACATATGAGATTATATCTCC |
| | | | TGAAGATATTGGGCTTAACCGAGTTAATGAATCTGGCATC |
| | | | GTCCTTGGGAAACTCAGTGGGCGTCATGCTTTGCAAGCCA |
| | | | AAATGCTCGAGCTTGGATACGATATTGAGGGAAAAGAACT |
| | | | TGAGGACCTCTTTTGGCGATTCAAATCTGTGGCCGAGAAG |
| | | | AAAAAGAAAATTACAGATGATGACCTGATAGCATTAATGT |
| | | | CAGATGAAGTTTTCCAGCCTCAATTTGTTTGGCAACTTGA |
| | | | AAATGTACAGGTTACATGTGGAAGTCTTGGCCTTTCTACG |
| | | | GCAACTGTTAAGCTCATTGACGCTGATGGTCAAGAGCATG |
| | | | TTTCTTGTTCTGTTGGAACGGGGCCAGTTGATGCGGCTTA |
| | | | TAAGGCAGTTGATCTCATTGTAAAGGTACCTGTAGCACTC |
| | | | CTTGAATATTCCTTGAATGCAGTCACGGAAGGTATAGATG |
| | | | CCATAGCTTCAACCAGAGTTTTAATTCGTGGGGAGAATGG |
| | | | CCATACATCAACCCATGCTTTAACTGGAGAGACTGTACAC |
| | | | CGTTCTTTTAGTGGAACCGGAGCAGATATGGATATTGTTA |
| | | | TCTCCAGTGTCCGAGCCTATATTGGTGCATTGAATAAGAT |
| | | | GTTGAGTTTCAGAAAGCTGGTATCGAAACACAGCAAACCT |
| | | | GAAGGCAGTGCAGTCGTATAG |
| 34 | 2- | Nucleic acid | ATGGCGATTTTGATATCAAGATCAAGAAACTTTAACCATT |
| | oxoisovalerate | | TTCTTCTAAGCACAAGGTTTCGTTTCTTATCACGCCTAAA |
| | dehydrogenase | | CCAAACAAGTTCAAAAACATTTTCCCACCATGCCAAAACT |
| | (g63865) | | TCATCATTTACAACTGCAGCCGGCGGTGGTTTTACAAAGC |
| | | | CGACGGCGACATTTTCCGGCGAGTCTGCCGCCGTTTTCCG |
| | | | GGTAGAACGTTTCCAGTCCAATAAAACTGGAGAGCAACTA |
| | | | AATACACTCTACTATGAAGAAGATAACCATCACCAAATTA |
| | | | TTGATGAAAATCAGGTCATGGATTTTCCTGGAGGGCAGCT |
| | | | TCCAATTACTCCTCAAATGAAATTTATTGCAGAGTCATCT |
| | | | GAAAAGAGGTTACCTTGTTATAGAGTCCTTGATGATGATG |
| | | | GCTATCCAATTCCAGGCAGCATTTTTGAGGAGGTGAGCAA |
| | | | AGAATTGGCTGTAAAAATGTATAGTTCAATGGTGACACTT |
| | | | CAAGTTATGGATACCATATTTTATGAAGCACAAAGGCAGG |
| | | | GGAGGTTATCTTTCTATCTCACTACTGCTGGAGAAGAAGG |
| | | | TATCAACATAGCATCTGCTGCTGCTCTCTCCGTCGATGAC |
| | | | TTCGTCTTGCCTCAGTATAGGGAAGTAGGGGTTATCTTAT |
| | | | GGCGGGGCTTCCCCCTGAAAGATATTGCCAATCAATTGTT |
| | | | CGGAAACAAGTTTGATTATGGAAAAGGAAGGCAAATGCCA |
| | | | TGCCACCATGGTTCTAATGAGCTCAATTACTTAACTATTT |
| | | | CTTCGCCAATAGCGACACAGATTCCTCAGGCCGCGGGCGT |
| | | | TGCTTACTCTCTGAAAATGGATAAAAAGGAGGCTTGTGCT |
| | | | ATTACTTATCTTGGAGATGGTAGCACCAGTGAGGGTGATT |
| | | | TTCATGCTGCTTTAAACTTTGCAGCGGTTTTGGACGCTCC |
| | | | TGTTGTCTTTATATGCCGCAACAATGGATGGGCCATTAGC |
| | | | ACTCCTGTAAACGAACAATTTCGAAGTGATGGAGTTGCCT |
| | | | CAAAGGGTCAAGCCTATGGAATTAGAAGCATTCGTGTAGA |
| | | | TGGCAATGATGTCTTGGCAACTCATAGTGCTATTCGTGCA |
| | | | GCTCGCGAAATGGCAATTAAGGAACAAAAGCCAATATTAG |
| | | | TAGAGGCCATGACTTATAGAGTAGCCCACCATTCAACATC |
| | | | TGATGATTCAACCAAGTATCGACCCGTCGAAGAAATAGAG |
| | | | CACTGGAAAACAGCAAAAAGTCCAATATCCAGATTCAGAA |
| | | | AATGGATTCAGAGAAATGGTTGGTGGAATGATGAAAATGA |
| | | | ATCTGAACTTCGCGGAGACACCAGAAAACAGGTATTGCAA |
| | | | GTAATGCAAGCAGCAGAGAAGGTGGAGAAACCTCCATTGA |
| | | | CAGATTTGTTTACGGATGTTTATGACAAAGTGCCATTAAA |
| | | | TCTTCAAGAGCAACACAAGTTTATTAGGGATGCTGTAAAG |
| | | | AAATCTCCAAGAGAGTATCCTTCTGATGTTCCTATATAA |
| 35 | Neomenthol | Nucleic acid | ATGGCAGAAAAAATCACCAGCCACGAGAACACAAGGTATG |
| | Dehydrogenase | | CAGTGGTGACAGGGGGAAATAAAGGAATAGGATATGAAAC |
| | (NtNMD; | | ATGCAGGCAACTAGCAAAGGAAGGAATAGTGGTAGTGTTG |
| | g29387) | | ACAGCAAGGGATGAAAGGAGAGGAATTGAAGCTCTCGAAA |
| | | | AGCTCAAGGAAGAGTACTCAAGCAATAAAACTGATGATGA |
| | | | TCAGATTTTATTTCATCAACTTGATGTTATGGATCCAGCT |
| | | | AGTATTTCTTCTCTTGTGGACTTCATCAAAACTAAATTTG |
| | | | GAAAGCTCGATATTCTGGTTAACAACGCAGGGATTGGTGG |
| | | | ATTAATGGTAGAAGGAGATGTTGTTATAATAAAAGATTTA |
| | | | ATAGAAGGAGACTTCGTAACCATTTCTGCTGAAAATGGGG |
| | | | AAGAGGATGGTATTAAGAAATCAATTGAAGGTATTGAGCG |
| | | | TATTGTTACAGATTATGAGTTGACAAAACAATGCCTGGAG |
| | | | ACAAACTTCTATGGTGCAAAAAGAATGATTGAAGCATTTA |
| | | | TTCCCCTCCTTCAGCTCTCTAACTCCCCAAGAATTGTTAA |
| | | | TGTCGCTTCTTTCTTGGGGAAGTTAAAGCTATTGTGCAAC |
| | | | CAATGGGCTATAGGAATGCTAAGTGATGCTAAAAGCCTGA |
| | | | GAGAAGAAAGGGTGGATGAAGTGTTGAATGAATTTATAAA |
| | | | AGATTTTAAAGAGAAATCAATAGAAGCCAAAGGATGGCCA |
| | | | ACTTATTTCTCAGCTTACAAAGTCTCGAAAGCATCCCTGA |
| | | | TTGCTTACACAAGGGTTTTAGCTACGAAATATCCAAATTT |
| | | | TCGGATAAATTCTGTGTGTCCTGGATTTTGCAAAACAGAC |
| | | | GTGAACTGCAATACTGGGAGCTTAACTGCTGAAGAAGGTG |
| | | | CTGAAAGCTTGGTGAAGCTTGCTTTGGTGCCAAATGATGG |
| | | | ACCCTCTGGTCTCTTCTTTTATAGAAAGGAGGTCACCTCT |
| | | | TTTTGA |
| | Regulatory | Nucleic acid | TATA |
| | element | | |
| | (TATA-box) | | |
| | Regulatory | Nucleic acid | CGTGG |
| | element | | |
| | (Unnamed_1) | | |
| | Regulatory | Nucleic acid | TATATAAA |
| | element (AT- | | |
| | TATA-box) | | |
| | Regulatory | Nucleic acid | CATTTG |
| | element (MYC) | | |
| | Regulatory | Nucleic acid | CAAAT/CAAT |
| | element | | |
| | (CAAT-box) | | |
| 41 | Regulatory | Nucleic acid | ATGTACAAAATTTC |
| | element | | |
| | (Unnamed) | | |
| 42 | Regulatory | Nucleic acid | CATGTCAAACGTTA |
| | element | | |
| | (Unnamed) | | |
| | Regulatory | Nucleic acid | CTCC |
| | element | | |
| | (Unnamed_4) | | |
| | Regulatory | Nucleic acid | TAACCA/CAACAG |
| | element (MYB) | | |
| 45 | Cannabis | Nucleic acid | TCTTAAACCCTTATGTGTTTATTTCATTGTTTTAGAA |
| | Cyclase | | TTCATATTAGGATGTTAATGAAACATACTTGTTAGTA |
| | promoter | | AATCTAGATCCTCGTAAAATATTTCCAAGAAATATAA |
| | | | CAGCTACTCAGATTATACACAAAAATCCCAATAGACA |
| | | | AAAACACTGTAAAAGTCATACTAAAACTCAAAAAAAA |
| | | | AAAAAAATACAACCAAACCAAATCAATAAAAACTAAA |
| | | | AAATCATCAATGGGCAGGCAAGACTTCAATTGTAGTT |
| | | | TTGACTTGCCTGAGTGAATCGATTCCGGAAAAGAATT |
| | | | GAAATTGTGGTCGTCGTTCAAGCTCTTGGCTGAATCT |
| | | | CCTGATTGTTCAAGCCCTCTCATCAGAATTATCTCCA |
| | | | TTGTTCAAGCCCTCTCATCGGAAGCTCCTGGTTTTTC |
| | | | CATCAATTTTTGGCCATGGTCACTATGGAGGTAAGTA |
| | | | GATTGAAGAAGACGAATGTGAGAGAGAAGGCAAAGGG |
| | | | TCGAGGTGGGCGTGGTGCAGTTTTTGAGGTGGGAGTT |
| | | | GTTTCTCGGCCGCAACAATCCACCATGTTTCTTAGTT |
| | | | TTTTTTTCTTTTTGTTCTGGGAATCGATTGTTTTTGG |
| | | | GTGTGGATTGTTGGTTGATAGGTTTTGGGGGTGGTTT |
| | | | CTGGGCAGTGAGAAGGTGAGAAGAAGAAGAAGAAGAA |
| | | | GAAAGTTATGGTTTGAAGAAGAAGAAGAAGGAAAATC |
| | | | AGGTGGGTGGGTGGGGTTTCGTGTGAAGCAGAAAAAA |
| | | | GAAAAAAAAAACAAGTTATTTTATGATTTGAAAATAT |
| | | | TATTTTTAGTTTTTTTATATTATTTTAAATTTTTTTT |
| | | | ATTAAAATTATAATTTGGACCAGTAATACTTCAAGAC |
| | | | GTGGCATTTTAAAAATTAATAAACGGACAGTTATCTT |
| | | | AGGGACCAACGGACTCACAGAAAATGTGACCTTGGGG |
| | | | ACTATTACCGCCAATTTTTGAGGTTTGGGAATAATCT |
| | | | CCATCAACCCTTAATTTTTGGGGACTTTTACCGCAAT |
| | | | TATCCCTATTATACATAATATTTTATAACAAGTTTTT |
| | | | TTTTATTATTTTTTATTATTTGATGAAGTAATTAAAG |
| | | | AGGAGTTTTCAATTTGTTAATTTTTCAGATTAGCTTG |
| | | | AAAAAAGGATTAGCTTGAAAATACAATTCTATAATCA |
| | | | TACAATTTCAAAGCATAGAAAAAAAATTGTTCAAAAA |
| | | | ATAAGAAAAGAAAATTAAGCATAATTCTATATTGGCT |
| | | | GCTACATTGCAATATACGTACGTACAGTCATAAAAAT |
| | | | ATGCACATGGATGAACTATTACAATTAGAACAGAAGA |
| | | | AAAGTAAATGATAAAGCTTCTTACTCTTGACTAACTC |
| | | | TTATTAAGTACTGTTGATTAATTTGAAATTTTCAAAT |
| | | | CAAAATACACTATAAATAGCTGAGTACATGAAACGAG |
| | | | TTTTCCATCAATTAAAGCTAACTCCATCTCGTAATTT |
| | | | ATATAATTATATAGTGATCGTTTATATACATATATAT |
| | | | CACCAAATCGTGATTTCAAA |
| 46 | Cannabis PSO | Nucleic acid | AATGTTTACAATTTTAACATGTCGATTATTTTCAATT |
| | promoter | | ACTAATTTTTTGAAAAAAAAAATGTGTAAAGTTAAAT |
| | | | ATAAAAATACATAAGTCAATATTTATATTTTGATATA |
| | | | TTATTAAAATAATATATTTACTAAAATTTAATACAAA |
| | | | ATTATATATATACAAACATATTTAAAAATTTGTTTAT |
| | | | ATTTTAAATAAACAACATATATGCATGTTTATTAAAT |
| | | | ATTTAATTATTATTAATGTTTATAATACTAGTATTGA |
| | | | TCTAATATGTTTATTATGATAATATTTTAATAGAATC |
| | | | AATATAATTTGTTGATTAAAAGTTCATAAATAAGTTT |
| | | | AAAAATAAAACATTTATGTTTATTTTGTTTTATGAAA |
| | | | CAAAGCTATAAACAAATGTGAAATTATGTATAAACAT |
| | | | TATATTTATATTTTATATTGATAATGTTTATAAATTA |
| | | | TTAATCTAATATATTTAATATATAGTTTTATAAATAA |
| | | | AGTATGTATATTTATTTTATTTTACTTTTTTTTGTTG |
| | | | TAAGAAAAAATATGTATGTTTATTGTTTATTTATATA |
| | | | AGACTTAGGAAAAATTAATTACCTCATATAGATTGTA |
| | | | CAGAAAATTAAAATTATGTGTCTCTAATATAGAATGT |
| | | | ATAATTAAATGTAAATAAATTATTTTGTATTATATAG |
| | | | TATAATAAAATTATATAATGTATACAAAATTATAAAT |
| | | | TTCTCATATCTTTTCTTCTAATTTTTATTTATTTATT |
| | | | TTTTTAATGTCAATATGTATAAAGTGTTGGACTCCAA |
| | | | TATAAACACTTAAAGTTAAACTTAAATTGAGTTTAAT |
| | | | TAATTCAAATTTTTTGTCAAGTTGTATTTTTTAAGTA |
| | | | GTTTGTTTGTGCTTGTGGCTTGTTATAAAACATGCTA |
| | | | TGCCCTAATTTTGTATACATAACACTAAGGTGGCATT |
| | | | TGGTTGAAAGAAATGAAAACATATATAAAAATAAAAA |
| | | | TGAAATAAAATAAAATTTAAAATGTATAAAAAAATCA |
| | | | TTAGTTTCTTTTAATGTTATATTAACATGATTTTTTA |
| | | | TTCTATTTTAAAATAGAATAGTCATTCCACTAAAATA |
| | | | GTGTAAAGGACATTCCATTAGAATGATATTCTAATAC |
| | | | TTTAAAATGTAACCAAATAAATAAATAAAATAAAAAT |
| | | | AATTTCTTTTTCATTCTATTCCATTTCATTACTCATT |
| | | | TTCATTCCATTCTATTTCATTTAACAAAAATGATAAA |
| | | | TGAACATTTTTGTTATCTTGTAGTAGTACAAACTACA |
| | | | AATAATAGAAAAATGAAACTTTAATATAAGGAGGAGA |
| | | | CAAAGTCTATTATTTACCTTGGTGATCAAGTTTTATA |
| | | | TACAACTTTGAAATAAGTACTTTCTTTCTCTCTCTTA |
| | | | TTAACATAATCCTACTTGAGTTCCCACTTGCATTTAC |
| | | | CTTAATATAATACTATGTCAACCATGTGTATAATTAG |
| | | | ATCTAACATCTACTTCAAATCTAAAGAACTTATTTAC |
| | | | CAACTAATCAAAATCAAAAT |
| 47 | Cannabis | Nucleic acid | GTAGTACATAAATTTATAGCTGATGATAAGTCTCATT |
| | Ribulose | | CTCAGTCGAGGAATATCTACTGTGTTTTGGATCAACT |
| | bisphosphate | | GAAAAGGGAGCTTAAAAGTTCATCCATAGCTCAATTT |
| | carboxylase | | TCAACCATGTATTTTTAGTTTATAATAGTAAAGATTA |
| | small chain | | TTCAGTCCCGTATAGAGAGAAAACAAATGACAAATCA |
| | promoter | | TATGATTCAGCAGCAAAAAATGGACTTTTTACAACTC |
| | | | AAAATAAAACAGAAATATCTTACTATAATATAATACA |
| | | | TAGGGATAATGTACGTTGATCGGTTTATGTTAAGTAC |
| | | | AGTTCGTCTAATTAAAAAAAAAAAAAAACAGAAACCA |
| | | | ATCCAATAAAATTAGTCGAGTTAGTCAGGTTAATCCG |
| | | | TCCAAATCGATCATTAATATATTTTTTTTACATTCAA |
| | | | TTGCTATATTTTAATTAGATGTAATAACGAGACAAAT |
| | | | AGATATATATTTATAAATTACACTTACAGTTCAATTC |
| | | | AACACATAAAAGAAACATATTAATTTTAACTTTATAT |
| | | | GTTTTAAAAATTCATATAAAGTATATGATATATATAA |
| | | | ATATGTATATACATAAAAACCCTTTTGATTGTGTTGG |
| | | | TTGGATTAGTAAGAGTTGAATAGTGGGGTTGATATTA |
| | | | TTTTCAATCCACCCAATATAAAGATCAGACAGATTAT |
| | | | ATTTTTATCGAATTTTTCGATTTGTATTTTTTGTCAG |
| | | | CTTATCTCAATTTAATTTAAGCAAGTTATTCAGATTA |
| | | | GGCGGTTTAAAAAAATGAAATAATAGCATGAAAGTTT |
| | | | TCAGTCATAACATTAAAAAAAAAAATGTCATTTTGTT |
| | | | ATGATGAGTGGCTATGAACTGCAGCCAGTTTTTGATG |
| | | | GTATTCATATGCAATTTTAATGGCTTTCTTCAGCTTA |
| | | | ATGATTTAATTAGTAGATAAGAGATTCTTGAATCTAG |
| | | | TACTCATGATGTATTTGATATCCATAGTGACCATTGA |
| | | | AGATTGAAACAGTTTTAACTTGGCTTCTCTCTCATAT |
| | | | TGTTCAAGCATTATTTTTGGAACATGACGAAAGTTGT |
| | | | TCAGGAACCCATGTTGTCCACATCCTGAATTTCCAAG |
| | | | CTTAAGAGAACAAAAAATCCAAATAGGCATTCATCAC |
| | | | TAACTTCACAGACATATGTCACATATCTCTACATTTA |
| | | | ATCAAAACTGTTGTATGCTGATGTCTTCAATTCACAA |
| | | | GTGGCAAAAATGTAAGGTGGGACCCCAAAAAATTTCC |
| | | | ACTAGAGCCAAACTCTAAATAGGGAAAACTCTACACC |
| | | | ACCTCGTAGTACCCACCGAAATCCCATACCACTCATC |
| | | | TTTTAGAAAAGAAAAAAACCACACTTGATCTCACAAC |
| | | | AAATATAAAGCTGACTTCTTTTGTTGTTTTTAGAAAC |
| | | | CATTTCCACCATTTTCCTAATAAACTACTTGATACTA |
| | | | AAATAATCAAAATAAGAATAAAGGAAAGGAAAGGAAA |
| | | | TAATACTACAACACTGCAATTATTTTCTTTCTCCTGT |
| | | | TAACCAAGTTTGAGGTTAGG |
| 48 | geranylgeranyl | Nucleic acid | ATGAGCACTGTAAATCTCACATGGGTTCAAACCTGTT |
| | pyrophosphate | | CCATGTTCAACCAAGGAGGTAGATCCAGATCCTTATC |
| | synthase. | | AACTTTCAATCTCAATCTCTACCACCCTTTGAAAAAA |
| | chloroplastic | | ACACCCTTTTCAATCCAAACCCCAAAACAAAAACGAC |
| | [Cannabis sativa] | | CCACTTCACCATTTTCATCAATCTCAGCTGTTCTAAC |
| | | | CGAGCAAGAAGCCGTTAAAGAAGGCGATGAAGAAAAA |
| | | | TCCATCTTCAATTTCAAGTCTTACATGGTCCAAAAAG |
| | | | CCAACTCAGTCAACCAAGCTTTAGACTCAGCCGTTTT |
| | | | GCTCAGAGATCCCATTATGATACACGAGTCCATGCGT |
| | | | TACTCACTCCTCGCCGGAGGAAAACGAGTCAGACCCA |
| | | | TGCTCTGTCTCTCAGCCTGTGAACTCGTAGGCGGAAA |
| | | | AGAATCCGTAGCCATGCCGGCTGCCTGCGCCGTCGAA |
| | | | ATGATCCACACCATGTCTCTAATCCACGACGACCTCC |
| | | | CTTGTATGGACAACGATGACCTCCGCCGTGGAAAGCC |
| | | | CACAAACCACAAAGTCTTCGGAGAAGACGTGGCCGTT |
| | | | TTAGCCGGCGATGCACTTTTAGCCTTTGCTTTTGAGC |
| | | | ACATGGCGGTCTCTACCGTTGGTGTTCCGGCAGCCAA |
| | | | GATTGTCAGGGCGATTGGTGAGCTTGCTAAGTCAATT |
| | | | GGGTCAGAAGGATTAGTGGCTGGTCAAGTGGTTGATA |
| | | | TTGATTCAGAGGGTTTGGCTAATGTTGGGCTTGAACA |
| | | | ACTTGAGTTCATTCATCTCCATAAGACTGGGGCTCTT |
| | | | CTAGAAGCTTCTGTTGTTTTGGGGGCTATTCTTGGTG |
| | | | GTGGTACAGATGAAGAAGTTGAAAAACTTAGGAGCTT |
| | | | TGCTAGGTGTATTGGCTTGCTTTTTCAGGTTGTTGAT |
| | | | GACATTCTTGATGTGACTAAATCTTCTCAAGAATTGG |
| | | | GTAAAACTGCTGGGAAAGATTTGGTGGCTGATAAGGT |
| | | | TACTTATCCAAGGCTAATGGGTATTGACAAATCAAGA |
| | | | GAATTTGCTGAGCAATTGAACACAGAAGCCAAACAGC |
| | | | ATCTTTCTGGTTTTGATCCCATAAAGGCTGCTCCTTT |
| | | | AATTGCTTTGGCTAATTATATTGCTTATAGGCAAAAT |
| | | | TGA |
| 49 | (−)-kolavenyl | Nucleic acid | ATGCCTTCTCTCTTCTCCCAATCACTACTTCTCCCTT |
| | diphosphate | | TCTCTCAAAACACTAATACTCTCTCCCTTTTCCATCA |
| | synthase TPS28. | | ACCAAAACTTCTTCCTCCAGGTGCTTCGCTATTGGAA |
| | chloroplastic | | GCTAAAGACAAACAAGTTAACTTTGATCGTGATATTC |
| | [Cannabis sativa] | | GCTCAAAATGCAGCGCTATATCAAAACCCCGCACTCA |
| | | | CGACGTGTTTCAAAGTGGTGGTCTGCCAGTTATAAAG |
| | | | TGGCACGAGATTGTGGAGGATGACATAGATGGAGAAG |
| | | | AAGAAGATACTAAGTGGACAAGATCGAATGAGATCGA |
| | | | GGAACGTGTCGCTTCAATCAAATCAATGTTGGAGAGT |
| | | | ATGGATGAGGGAGAGATAAGCATTTCAGCGTACGACA |
| | | | CAGCATGGGTAGCCCTTGTGGAAGATATTCATGGGAG |
| | | | TGGCTTACCTCAATTCCCATCGAGTCTCCAATGGATC |
| | | | GCCACACATCAGCTCTCCGACGGTTCTTGGGGCGATG |
| | | | CTGACATTTTCTCCGCACACGATCGCCTCATCAACAC |
| | | | TTTGGCTTGTGTTGTTGCTTTGAAATCTTGGAACCTT |
| | | | TATCCCGAAAAATGTCAAAAAGGTATGGCCTTTTTCA |
| | | | ATGCAAATATAAGTAAGCTTGAGAGGGAGAATCCGGA |
| | | | ACACATGCCTATTGGTTTCGAAGTGGCTTTCCCTTCT |
| | | | TTACTTGAAATAGCTCGAAAATTAAACCTTGAAGTGC |
| | | | CTGAGGATTCTCCTGTGTTAAAAGTCATATATGCTAG |
| | | | GAGAGATTTCAAGCTCACAAGGATTCCGAGGGACATA |
| | | | ATGCACACAGTGCCCACGACGCTACTCCATAGCTTGG |
| | | | AAGGAATGGTAGGTCTGGACTGGGAAAAGCTTTTGAA |
| | | | ACTGCAGTCCCAAGATGGGTCATTCTTGTTCTCACCA |
| | | | TCCTCAACTGCTTTTGCACTCATGGAGACCAAAGACC |
| | | | GAAATTGCTTGCAATATTTAACTAAAGCGGTCCAAAG |
| | | | GTTCAACGGGGGTGTCCCAAATGTTTACCCGGTTGAC |
| | | | TTGTTCGAGCACCTTTGGGTTGCGGATCGGTTGCAGC |
| | | | GCTTGGGAATATCAAGATTCTTTGAGCCACAAATTGA |
| | | | GGAATGTATCGATTATGTATTCAGAAATTGGACTGAG |
| | | | AAAGGAATTGGCTGGGCAAGAAATTCCAAGGTTGAAG |
| | | | ATATTGACGATACCGCAATGGGTTTCAGACTACTAAG |
| | | | ATTGCATGGTCACAAAGTTTCTGCCGATGTGTTCCAA |
| | | | CACTTTAAGAAAGGTGACGATTTTTTCTGCTTTCGGG |
| | | | GCCAGTCAACTCAAGCAGTGACTGGGATGTATAACCT |
| | | | TTATAGAGCTTCTCAGTTGGTTTTCCCTGGAGAAAAA |
| | | | ATTCTTGAAGATGCCATGGAATTCTCATCGAAATTTC |
| | | | TCAGAAAAAAACAGGCGTCCAATGAATTGCTAGATAA |
| | | | ATGGATCATAACAAAGGACTTACCTGGTGAGGTGGGT |
| | | | TTCGCATTGGAGGTTCCATGGAATGCAAACTTACCTC |
| | | | GAGTAGAGACCAGATTCTACATTGAACAGTATGGTGG |
| | | | ACAAAATGATGTTTGGATTGGCAAGACACTCTACAGA |
| | | | ATGCGAAAAGTTAACAATGACGAATATCTGGAGTTAG |
| | | | CAAAACTTGATTACAACATTTGCCAAGCTTTGCATTC |
| | | | GATTGAGTGGCACAATTTGCTAAAATGGTACCGAGAT |
| | | | TGTAAGTTGGAAAATTATGGAGTGAGCAGAAGGAACC |
| | | | TCCTCTTGGCCTATTTTCTTGCTGCGGCCAGTATTTT |
| | | | CGAACCGGATAGGGCCGATGAGCGGCTTGCATGGGCT |
| | | | AAAACGGCAGCACTGATGCAGGCCATCCAATCTCATT |
| | | | TCGATGACCAGAAAGCTTCTTCGGAGCATCGTATAGC |
| | | | TTTTGTCTCTGCTTTTAAAAGGAGTTGTAACATGCCA |
| | | | TCGTATTTGATTACAAGGGTGTCGAACATAAGTGATA |
| | | | CAGATCATGGCCTTCTTAGAACGTTGATGACGACTCT |
| | | | CAGCCACCTCTCTTTGGACACAATGATGCTGTATGGT |
| | | | CGGGACATCACCCACCATTTACGTCAAGCTTGGGAAA |
| | | | AGTGGCTGGTGAAGTGGCAAGAGGGTGGTGATGGACA |
| | | | TTACGAAGAAGAAGCAGAATTATTGATCCAAACAATA |
| | | | AACCTTAGCTCAGGCCGTACACTTGTGAAGGCCCTCT |
| | | | TGCTGTCAAATCCTCACTATGAAAAACTCTTCAGTAC |
| | | | CACAAACAAAGTTTGCTGCAAAATTCGTCACTTTCAA |
| | | | AGACAAAGGCATAGGGCCAAGGCAAATCAAAATGGAG |
| | | | AATTTAACAGAAACATCTTAACACCAGAAATAGAGTC |
| | | | AGATATGCAAGAGGTTGTGCAATTGGTGCTACAAAAA |
| | | | TCTTCAGATGACATGATCAACACAAAAATTAAGCAGA |
| | | | CATTTCTACTGGTGGCCAAGTCTTTTTATTATGCTGC |
| | | | CTACTGTGATTCTAAGACCATCAATTTCCACATTGGC |
| | | | AAAGTAATATTTGAGACTGTGGACTGA |
| 50 | solanesyl | Nucleic acid | ATGCTATTCTCAAGGGGATTTCGTCGGATTCCGACCA |
| | diphosphate | | CCACCTTCAATGGGTTTTCCCGTTGGTTCGTCTCTCA |
| | synthase 3. | | CCGACCCGGGTACTCACAGTCACAGACCACTACTCAT |
| | chloroplastic/ | | TGTTCTAGAGATTCAACCCACAAGATTTTTGGCGGTT |
| | mitochondrial | | TCGAAGAAAGTCGTTTCTGGGGTTTTGCTGGCTCTAG |
| | [Cannabis sativa] | | ATACCAAATTCATCACCAGAGTAGCTCCTTAGTTGAG |
| | | | GAAGAATTAGACCCTTTTTCTCTTGTTGCGGATGAAC |
| | | | TCTCACTTGTTGCTAATAGGTTAAGGGATATGGTAGT |
| | | | TGCTGAGGTGCCCAAGCTTGCTTCTGCTGCTGAGTAC |
| | | | TTCTTTAAAATGGGTGTGGAGGGGAAGAGATTTCGTC |
| | | | CAACGGTATTATTGTTAATGGCAACAGCATTAAATGT |
| | | | TAAGGTTCCCGAGCCTGCTAAAGCGTTAGCAGATACT |
| | | | TTAACACCCGAGTTACGTACAAGGCAGCAATCTGTTG |
| | | | CAGAAATAACAGAGATGATCCATGTGGCAAGCCTACT |
| | | | ACATGATGATGTCTTAGATGATGCAGAAACTAGACGC |
| | | | GGTGTTGGTTCGTTGAATTGCATTATGGGAAACAAGG |
| | | | TATCGGTATTAGCAGGAGATTTTCTGCTTTCTCGAGC |
| | | | TTGTGTTGCCCTCGCAGCTTTAAAGAATACCGAGGTA |
| | | | GTTACGCTACTAGCCACGGTTCTAGAACAGCTCGTGA |
| | | | CAGGTGAAACCATGCAAATGACATGTACATCTGAACA |
| | | | ACGTTGTAGTATGGAATATTATATGCAAAAGACATAT |
| | | | TACAAAACTGCATCGTTAATTTCGAACAGTTGCAAGT |
| | | | CGGTTGCAGTCATTGCTGGACAAACTACAGAAGTTGC |
| | | | AATGCTAGCATTCGAGTACGGCAAAAATTTGGGTTTG |
| | | | GCTTATCAATTGATAGATGATATTCTTGATTTCACCG |
| | | | GCACGTCAGCTTCCCTCGGAAAGGGTTCACTATCTGA |
| | | | CATCCGTCATGGTATTGTTACAGCTCCATTATTGTAC |
| | | | GCCATGGAAGAGTTTCCTCAGTTGCGCGCCGTGGTTG |
| | | | AGCAGGGATTTGAAAACCCCAAAAATGTTGACATTGC |
| | | | ACTCGACTACCTTGGAAAGAGTCGCGGGATCCAAAAG |
| | | | GCGAGGGAACTTGCAATAAAGCATGCAAACCTCGCTG |
| | | | CTGAGGCAATCGAGTCACTTCCAGAGAGCGATGATGA |
| | | | AGATGTAAAAAGATCGAGACGAGCACTAGTAGATCTC |
| | | | ACCCAAAGAGTCGTTACAAGAACAAAGTGA |
| 51 | terpene synthase | Nucleic acid | ATGTCTACCAATAATAATAATAATATTAATAATATTA |
| | 10-like isoform | | TTTCTCGAAGATCAGCAAACTATCAACCTTCACTTTG |
| | X1 [Cannabis | | GCATTTTGATTATGTACAATCACTTTCTACCCCTTTC |
| | sativa] | | AAGGAAGGAGCATATGCCAAAAGAGTTGAGAAAGTAA |
| | | | AGGAAGAGGTAAGAGTAATGGTGAAGAGAGCAAAAGA |
| | | | GGAGGAGAAGCCTTTATCTCAACTTGAGCTTATTGAT |
| | | | GTAATGCAAAGACTTGGAATCTCTTACCACTTTGAGA |
| | | | ATGAAATTAATGATACATTGAAAGATATATATAACAA |
| | | | CAATAATGTGTACAACACCAACAATAATGTGTATGCC |
| | | | AATTCTCTTGAATTTAGACTCCTAAGACAACATGGTT |
| | | | ATCCGGTGTCTCAAGAAATATTTAGTACGTGCAAAGA |
| | | | TGAAAGAGGCAATTTTATGGTGTCTTCCAATGATATC |
| | | | AAAGGAATGTTATCTTTATATGAAGCTTCATTCTATT |
| | | | TGGTAGAAAATGAAGATGGTATTTTGGAAGAGACAAG |
| | | | AGAAAAAACAAAGAAATATCTTGAGGAATACATAATC |
| | | | ATGATCATGGAAAAACAACAATCATTATTAGATCAAA |
| | | | ATAATAATAATGATTATGATTATGATTATGAACTAGT |
| | | | GAGCCATGCATTGGAACTTCCACTTCATTGGAGAATG |
| | | | TTAAGATTGGAGAGTAGGTGGTTTATTGATGTGTATG |
| | | | AGAAGAGACTAGACATGAACCCTACTCTACTTACCTT |
| | | | AGCTAAACTAGATTTCAACATTGTCCAATCAATATAC |
| | | | CAAGATGATCTTAAACATGTCTTCAGCTGGTGGGAAA |
| | | | GCACTAATATGGGAAAGAAGTTGGAATTTGCAAGAGA |
| | | | TAGAACAATGGTGAATTTCTTATGGACAGTAGGAGTT |
| | | | GCATTTGAGCCACATTTCAAAAGTTTTAGAAGAATGA |
| | | | TTACAAAAGTAAATGCTTTAATAACAGTAATAGATGA |
| | | | CATATATGATGTTTATGGTGCACTAGATGAATTGGAG |
| | | | CTCTTCACTAACGCAGTTGAGAGATGGGATATTAGTG |
| | | | CTATGGATGGGCTCCCTGAGTATATGAAGACATGTTT |
| | | | TCTTGCTTTATACAATTTCATAAATGATCTTCCATTT |
| | | | GATGTGTTAAAAGGGGAAGAAGGCCTCCATGTAATAA |
| | | | AATTCCTTCAGAAATCGTGGGCAGATCTTTGCAAATC |
| | | | TTATTTAAGAGAAGCAAGATGGTATTATAATGGATAC |
| | | | ACACCAAGATTTGAAGAGTACATTGAGAATGCATGGA |
| | | | TATCAATATCAGGACCTGTTATACTATCACATTTATA |
| | | | CTTTTTTGTAGTGAATCCAAACATGGAAGATGCCTTA |
| | | | TTAAGTACTTGCTTTAATGGATACCCTACCATAATAC |
| | | | GACATTCATCGATGATTTTACGTCTTACAGATGATCT |
| | | | TGCAACTTCAACGGATGAATTGAAAAGAGGCGATGTT |
| | | | CCCAAATCAATCCAATGCAAAATGTACGAAGATGGTA |
| | | | TATCTGAAGAGGAAGCTCGTCAACGTATTAAGTTATT |
| | | | AATAAGTGAAACATGGAAGCTTATTAATAAAGATTAC |
| | | | ATAAATTTGGATGATGATGATGATGATGGTGATGACT |
| | | | ACTCTCCAATGTTCTATGAGTCTAATAATATTAATAA |
| | | | GGCTTTCATTGAAATGTGTTTAAACCTTGGTAGAATG |
| | | | GCACATTGCATTTATCAATATGGAGATGGACATGGAA |
| | | | TTCAAGATCGCCAAACAAAAGATCATGTACTATCATT |
| | | | ACTTATTCACCCTATTCCTCTTACCCAATAG |
| 52 | (−)-limonene | Nucleic acid | ATGCAGTGCATAGCTTTTCACCAATTTGCTTCATCAT |
| | synthase. | | CATCCCTCCCTATTTGGAGTAGTATTGATAATCGTTT |
| | chloroplastic | | TACACCAAAAACTTCTATTACTTCTATTTCAAAACCA |
| | isoform X1 | | AAACCAAAACTAAAATCAAAATCAAACTTGAAATCGA |
| | [Cannabis | | GATCGAGATCAAGTACTTGCTACCCCATACAATGTAC |
| | sativa] | | TGTGGTCGATAACCCTAGTTCTACGATTACTAATAAT |
| | | | AGTGATCGAAGATCAGCCAACTATGGACCTCCCATTT |
| | | | GGTCTTTTGATTTTGTTCAATCTCTTCCAATCCAATA |
| | | | TAAGGGTGAATCTTATACAAGTCGATTAAATAAGTTG |
| | | | GAGAAAGATGTGAAAAGGATGCTAATTGGAGTGGAAA |
| | | | ACTCTTTAGCCCAACTTGAACTAATTGATACAATACA |
| | | | AAGACTTGGAATATCTTATCGTTTTGAAAATGAAATC |
| | | | ATTTCTATTTTGAAAGAAAAATTCACCAATAATAATA |
| | | | ACAACCCTAATCCTATTAATTATGATTTATATGCTAC |
| | | | TGCTCTCCAATTTAGGCTTCTACGCCAATATGGATTT |
| | | | GAAGTACCTCAAGAAATTTTCAATAATTTTAAAAATC |
| | | | ACAAGACAGGAGAGTTCAAGGCAAATATAAGTAATGA |
| | | | TATTATGGGAGCATTGGGCTTATATGAAGCTTCATTC |
| | | | CATGGGAAAAAGGGTGAAAGTATTTTGGAAGAAGCAA |
| | | | GAATTTTCACAACAAAATGTCTCAAAAAATACAAATT |
| | | | AATGTCAAGTAGTAATAATAATAATATGACATTAATA |
| | | | TCATTATTAGTGAATCATGCTTTGGAGATGCCACTTC |
| | | | AATGGAGAATCACAAGATCAGAAGCTAAATGGTTTAT |
| | | | TGAAGAAATATATGAAAGAAAACAAGACATGAATCCA |
| | | | ACTTTACTTGAGTTTGCCAAATTGGATTTCAATATGC |
| | | | TGCAATCAACATATCAAGAGGAGCTCAAAGTACTCTC |
| | | | TAGGTGGTGGAAGGATTCTAAACTTGGAGAGAAATTG |
| | | | CCTTTCGTTAGAGATAGATTGGTGGAGTGTTTCTTAT |
| | | | GGCAAGTTGGAGTAAGATTTGAGCCACAATTCAGTTA |
| | | | CTTTAGAATAATGGATACAAAACTCTATGTTCTATTA |
| | | | ACAATAATTGATGATATGCATGACATTTATGGAACAT |
| | | | TGGAGGAACTACAACTTTTCACTAATGCTCTTCAAAG |
| | | | ATGGGATTTGAAAGAATTAGATAAGTTACCAGATTAT |
| | | | ATGAAGACAGCTTTCTACTTTACATACAATTTCACAA |
| | | | ATGAATTGGCATTTGATGTATTACAAGAACATGGTTT |
| | | | TGTTCACATTGAATACTTCAAGAAACTGATGGTAGAG |
| | | | TTGTGTAAACATCATTTGCAAGAGGCAAAATGGTTTT |
| | | | ATAGTGGATACAAACCAACATTGCAAGAATATGTTGA |
| | | | GAATGGATGGTTGTCTGTGGGAGGACAAGTTATTCTT |
| | | | ATGCATGCATATTTCGCTTTTACAAATCCTGTTACCA |
| | | | AAGAGGCATTGGAATGTCTAAAAGACGGTCATCCTAA |
| | | | CATAGTTCGCCATGCATCGATAATATTACGACTTGCA |
| | | | GATGATCTAGGAACATTGTCGGATGAACTGAAAAGAG |
| | | | GCGATGTTCCTAAATCAATTCAATGTTATATGCACGA |
| | | | TACTGGTGCTTCTGAAGATGAAGCTCGTGAGCACATA |
| | | | AAATATTTAATAAGTGAATCATGGAAGGAGATGAATA |
| | | | ATGAAGATGGAAATATTAACTCTTTTTTCTCAAATGA |
| | | | ATTTGTTCAAGTTTGCAAAAATCTTGGTAGAGCGTCA |
| | | | CAATTCATGTATCAGTATGGCGATGGACATGCTTCTC |
| | | | AGAATAATCTATCGAAAGAGCGCGTTTTAGGGTTGAT |
| | | | TATTACTCCTATCCCCATGTAA |
| 53 | cytochrome | Nucleic acid | ATGGAGTTCCAACAAATAATACCCTCTTTCCAAGTCC |
| | P450 71D11 | | TTCTCTTTTCATTTTTTATGATTATTGTGGTGAGCAT |
| | [Cannabis | | ACTCTTGAAGAGAGCTCAAACAAGCAATAGGTCAGCT |
| | sativa] | | TCAAAACTACCCCCAGGTCCATGGAGACTACCCTTCC |
| | | | TGGGAAACTTGCACCAACTTTTGGGCCCTTTGCCTCA |
| | | | TCACACATTCAGAGACTTAGCCAAAAAACATGGACCA |
| | | | TTTATGTACCTCAAAATTGGACAAGTTCCAACCATAG |
| | | | TAGTTTCATCACCAGAGTATGCTAAAGAGGTCATGAA |
| | | | AGTCCATGATAATGCTCTTGCATCAAGACCCAATAAT |
| | | | CTTGTTACGCAAATCTTGGCATATAATGGTACGGACA |
| | | | TTATATTTGCTCCATATGGTCAGTATTGGAAAGAGGT |
| | | | AAAAAAGATTTGTGTGCAAGAGCTTCTAACCCTATCT |
| | | | AGGGTTAAAACTTTTCAACCCATTAGAGAAGAAGAGT |
| | | | CGTTTAATATTGTAAAAAATATAGCTTCAAAAGTTGG |
| | | | CTCACCTATCAATCTTACTCAAATGTTGAAAAGTTTG |
| | | | TCTTATAGCATCATAGCTAGGGCTGCCTTTGGCGAGA |
| | | | AAAGAAGTGATCATGATGACTTCGTATATATTATGAA |
| | | | GGAAACTGTGAAATTGTCTGCAGGGTTTGCATTTGGA |
| | | | GATGTGTTTCCATCTTTGAGTTTTCTTGATTGGTATA |
| | | | CTATAAGTAAATTCAAAGATTTGAAACTAAGGTCTTC |
| | | | AAGAATAGTGGACAGAATTATCAAGTTACATATAGAT |
| | | | GATCAAGACAAGGACAATCTTAAGAAAAGTGGAGAAG |
| | | | AGGAAGACTTGGTTGATGTTCTTTTGAGGTTTCATAA |
| | | | AAGTGAAGATCCTAATAACTTCACATTAACAAAGGAC |
| | | | AATCTTAAAGCAATTATTGCTAGCATATTTGGAGCTG |
| | | | GGAGTGATACATCATCAGTAACTATGGAGTTGGCTAT |
| | | | GGCAGAAATGATGAGAAATCCAAGAGTGATGAAAAAA |
| | | | GCTCAAGACGAGGTTAGAGAAGTCTTTGGCAAAAAAG |
| | | | GGTTTTTGGATGAATCTTCAATCAATGAGATGACATA |
| | | | CTTAAAATCAGTTGTGAAAGAAACTTTAAGGTTGCAT |
| | | | CCCCCAGCTGCCATGTTATTTCCAAGAGAAAGTAGAG |
| | | | AAAAGTGTGATATTAATGGTTATGAAATTCCTATGAA |
| | | | AACTAAGATACTTGTAAATGCATGGGCAATTGGAAGA |
| | | | GATCCTAAATATTGGATTGAACCTGAGAGTTTTATGC |
| | | | CAGAGAGGTTTCTTGAAAGCTCTATTGATTTTAAGGG |
| | | | AAATAATTTTGAGTTTATCCCATTCGGCGCTGGAAGG |
| | | | AGAATATGTCCCGGAATATCATTTGCTCTCACCAGTA |
| | | | TTGAGCTTCCTCTAGCATTTTTGCTATATCATTTCGA |
| | | | TTGGAAACTTCCCAATGGAATGAAACCTGAAGAGTTG |
| | | | GACATGACAGAGAGATTTGGTATTACAGTCTGTAGAA |
| | | | AGAAGGATTTGTACTTGATTCCTTCTAAATATGAACC |
| | | | ACCTTCTATGGCCAAAGCAATGAATATAAAGTAG |
| 54 | cytochrome | Nucleic acid | ATGGATCTTCAACTACTTTCATTCCCAATAATCCTCA |
| | P450 71D9 | | TTACTTTATTTTTTATGTTTATGGTAGTGAAAATAGT |
| | [Cannabis | | TTTGAAAATAGCTCATCATCAAACAAAGAACTCAGTT |
| | sativa] | | TCAAAGCTACCCCCAGGACCATGGAAATTACCATTGG |
| | | | TGGGAAATATACACCAAATCTTTGGCTCTTCACCCCA |
| | | | TGTTTCATTCAGAGACTTAGCCAAGAAATATGGGCCA |
| | | | TTCATGTACCTCAAAATTGGACAAATTCCAACTCTAA |
| | | | TAGTTTCATCACCAGAGTATGTTAAAGAGATCATGAG |
| | | | AACCCATGATGTTGTTTTTGCATCTAGGCCTCAAACT |
| | | | CTTGCTGCTCAGATCATGGCATATAATTGTACTGACA |
| | | | TTATATTTTCTTCATATGGTGAACATTGGAGACAACT |
| | | | CAGAAAGATTTCTATGCAAGAGCTTCTAAGCCCGGGA |
| | | | AGAGTTCAAACTTTTCGACCGGTTAGAGAAGAAGAGT |
| | | | TGTGTAATCTTGTTGAAGGGATCATGACATCTTCAAA |
| | | | AGATGGGTCACCTATCAATGTTACTAAAATGGTTACA |
| | | | AAATGTTCTTATGGCATCACATCTAGGGCTGCCTTTG |
| | | | GCAAGAAAAGCAGTGATCACGACGAGTTCATTTCGAT |
| | | | TGTTGAGGAAGCTATCGAGGCAGCTGGAGGCTTTGAA |
| | | | TTTGCAGAAGTGTTTCCTGCTTTGAGATTTCTTGATT |
| | | | GGAAAAGTCGTCCTATTTTTGAGAGCATCAAACTAAG |
| | | | ATCTTCAAGAATAATGGAAAATATCATCAAGGAGCAT |
| | | | ATAAAAGAGAAGGAAATTTCATTTGAGAAAATTGGAA |
| | | | AGGATGAAGATTTGGTTGATGTTCTTTTGAAGTTTCA |
| | | | TAAGAATGGAGATGATCTTGGGCGGTTCACCCTAACA |
| | | | AAAGACAATATTAAAGCAGTAATCTTTGATATCTTTG |
| | | | TAGCTGGAAGTGAAACAACATCTTTATCTGTAGATTG |
| | | | GGCTATGGTAGAAATGATGAGATATCCAAAAGTGATG |
| | | | AAAAAGGCTCAAGAAGAGGTGAGAAAAATTATTGGTA |
| | | | CAAAAGGGTCAGTGAATGAATCATCAATCAATGAGAT |
| | | | GAAATACTTAAAATTAGTTGTTAAAGAAACTCTAAGG |
| | | | TTGCATCCTCCAGCTCCTTTGTTACTTCCAAGGGAAA |
| | | | GTACAGAAAAATGTGACATTGATGGTTATGAGATACC |
| | | | TAAGAAAACAAGAGTAATAGTAAATGCTTGGGCAATT |
| | | | GGAAGAGATCCAAAGTATTGGATTGAACCTGAGAATT |
| | | | TTATGCCAGAGAGGTTTATGGAAAGCTCTATTGATTT |
| | | | TAAGGGCAACAATTTTGAGTACATTCCATTTGGTGGT |
| | | | GGAAGGAGAATATGTCCAGGCATGTTATTTGGTGTTA |
| | | | TTAATATTGAGCTTTCACTAGCATATTTGTTATACCA |
| | | | TTTTGATTGGAAACTTCCTAATGGAATGAACCATGAA |
| | | | AATTTGGATATGACAGAATTATTTGGTCTTACAATGA |
| | | | GAAGAAAAGATGATTTGTATTTGATTCCTACTATTTA |
| | | | TGACCATTCTTCTATAGCAAAATCATGA |
| 55 | cytochrome | Nucleic acid | ATGGATCTCCAACTACCCTCTTTCCCAGTCCTCTTGT |
| | P450 71D11 | | CCCTTCTTTTTTCTCTCCTTATGGCAGTTACCATACT |
| | [Cannabis | | CATGAAAAGAGCTCGAAACTCAAAACTACCTCCAGGG |
| | sativa] | | CCATGGAGACTTCCCTTAGTGGGAAATCTCCACCAGC |
| | | | TTTTATTGGGGTATTCATCATCATCCTCATCTTACGA |
| | | | GGTCTTCAGTGACTTAGCCAAAAAACATGGACCCTTC |
| | | | ATGTACCTCGAAATCGGACAAGTTCCAACCGTAATAG |
| | | | TGTCATCACCAGAGTACGCTAAAGAGATCATGAGAAC |
| | | | CCACGACGTCGTTTTTGCGTCTAGGCCACGGACTCTT |
| | | | GCCGCTCAAATCATTGGATACGATTGTACGGACATCG |
| | | | CATTTGCTCCCTATGGTGATTACTGGAGACAGCTCAG |
| | | | AAAGATTTGTATGCAAGCTCTTTTTAGTCCCAAAAGA |
| | | | GTTCAATCTTTGGAACCCATTAGAGAGAAAGAGGTGT |
| | | | TTAATACGTTACAACATATCATTGCTAATTCCAATAA |
| | | | ACTCAATTTTACTCAAATGGTCACAAATTTGTCTTAT |
| | | | AGCATCGTATCTCGAGCAGCTTTTGGGGAAAAAAGCA |
| | | | GTGATCATGATGAGTTCATATCGATTGTGGAGGAAGA |
| | | | TATAAAGGTAGCTGGAGGGTTTGAATTTGGGGAGTTG |
| | | | TTTCCTTCTTTGAGATTTCTTGATTGGACCAGTAGGC |
| | | | CTAAATATGAAAGCCTCAAACAGAGGTCTTCTAGAAT |
| | | | ATTGGAAAAGATCATCAAACAACATATGATTAATCAG |
| | | | AATAATGAGAAAAGTGAAGAAGAGCAAGACTTGGTTG |
| | | | ATGTTCTTCTCAAGTATCATAACAAGGCAAATCTTGG |
| | | | GTTAACCCTTGACAATATCAAAGGAGTAATCTGGGAC |
| | | | ATTTTTGAAGCTGGAAGTGAAACAACAGCTGTAACAG |
| | | | AGGATTGGGCTATGGTAGAATTGATGAGAAATCCAAT |
| | | | AATGATGAAAAAGGCTCAAGATGAGGTTAGGGAAGTT |
| | | | TTTGGAAGAAAAGGATTAGTTGATAAAACATCAATCC |
| | | | ATGAGATGAAATACTTAAAATTAATTATTAAAGAAAC |
| | | | TCTAAGGTTGCATCCTCCTGCTCCTTTTTTACTTCCA |
| | | | AGGGAAAATAGTGAAAAATGTGAAATTAATGGTTATG |
| | | | AGATACCTAATGGAACAAGAGTATTGGTAAATGTTTG |
| | | | GGGAATTGGAAGACATGCTAAGTATTGGAATGAACCT |
| | | | GAAAGTTTTATACCAGAACGGTTTGATGATAGCTCTA |
| | | | TTGACTTCAAAGGTAATAATTTTGAGTATATTCCATT |
| | | | TGGTGCTGGAAGGAGAATATGTCCTGGCATAACATTT |
| | | | GGTGTTGTTAGTCTTGAGTATTCTCTTGCTTTAATGT |
| | | | TATACCACTTTGATTGGAAACTTCCTAATGGAATGAA |
| | | | ACCTCAAGATTTAGACATGAGTGAGTTATTTGGCATT |
| | | | GCAGTAAGGAGAAAAGATGATTTGTACTTAATTCCTA |
| | | | CAATTTATCATCAGTCACCTCTTGCAAATTAA |
| 56 | (−)- | Nucleic acid | ATGACGGAACACTCACCACTAACCCCCAAAACCAAGC |
| | isopiperitenol/ | | TTCATGGCAAGGTGGCAGTCGTCACCGGCGGAGCCAG |
| | (−)-carveol | | CGGCATCGGAGAAGCCACGGCTCGAAAGTTCGCTGCT |
| | dehydrogenase. | | GATGGAGCACGCGCCGTCGTGATTGCAGATATCCAAG |
| | mitochondrial- | | ACGAGAAAGGCCAAAACGTGGCCGCATCAATCGGCCT |
| | like [Cannabis | | GGAACGCTCCACCTACGTCCACTGCGACGTGACCGAC |
| | sativa] | | GAGGCTCAAGTCGCAGCCCTCATCGACTCAACGGTCC |
| | | | AAAAGTACGGTCAAGTCGACGTGATGTTCAGCAACGC |
| | | | CGGGGTGCCTTGCGAGTCGGATCAGACGATTCTGGAT |
| | | | TTGGATCTGGTGGCGTACGATAAGGTGTTTGCGGTGA |
| | | | ACGCGAGGGGGATGGCGGCGTGTGTGAAGCACGCGGC |
| | | | GAGGGCGATGGTGGAGGGTGGAGTGAGGGGGAGCGTG |
| | | | ATATGCACGGCGAGCAATCTGGCGAGTATAGGAACTG |
| | | | AGAAGTACACGGACTACACTATGTCGAAGCACGCCGT |
| | | | TTTGGGGCTGGTGAGGTCAGCGAGTCTTCAGCTGGGG |
| | | | GCGCGTGGAATTCGCGTGAACGCGGTTTCGCCGGGGC |
| | | | CGTTGAGGACGCCTTTGCTGAAAGCGTTTATAGATAG |
| | | | GAGTGAGGAGGAGCAGGATAAGATGATTGAGGCTTCG |
| | | | TTATCGCTGAAGAATGGAAGGACTCCGTCGGTGGAGA |
| | | | ACGTGGCTGATGCGGTGTCGTTTTTGGCTTCAGATGA |
| | | | GTCTGAGTTTATCACTGGCCATAATCTTGCCGTCGAC |
| | | | GGTGGTTATATTCATCATCCACCCTAA |
| 57 | 2-alkenal | Nucleic acid | ATGGAAGAAGTGAGCAATAAACAACTGCTGCTGAAGA |
| | reductase | | ACTATGTTTCCGGTTATCCGAAAGAGTCGGATATGGT |
| | (NADP(+)- | | CTTAGCCACTTCCACCATCAAGCTTAAGCTTCCAGAA |
| | dependent) | | GGCTCCAATGGTGTTCTAGTAAAGAACCTTTATTTGT |
| | [Cannabis | | CATGCGATCCTTACATGGGACCCCGAATGAAGAACCT |
| | sativa] | | CAACAATGGCTTTTTCATAGAGCCCTTCAAACTTGGT |
| | | | TCTCCTATCACTGGGAATGGAATTTGTAAGGTACTAA |
| | | | AATCTGGAAATCCAAACTTCAAGGAAGGAGATTTGGT |
| | | | CTCTGGAGTGACAGGGTGGGAGCAATACAGTGTTATT |
| | | | GAGTCCACGAAATATATGTCTAAAATTCAAAACACTG |
| | | | ACGTGCCTCTGTCTTACTATACTGGATTACTAGGAAT |
| | | | GCCTGGTATGACTGCTTATGCTGGCTTCTTCGAGGTT |
| | | | TGCTCTCCGAAAAAGGGGGAATATGTGTTCATTTCTG |
| | | | CAGCATCAGGAGCAGTTGGTCAGCTTGTTGGGCAGTT |
| | | | CGCAAAGCTCTTGGGTTGTTATGTTGTTGGAAGTGCT |
| | | | GGAAGGAACGAAAAGGTTGATTTGTTGAAGAACAAAT |
| | | | TTGGCTTTGATGAGGCTTTCAACTACAAAGAAGAGTC |
| | | | TGACTTTGATGTAGCTTTAAAAAGGTATTTTCCAGAA |
| | | | GGTATAGATATATACTTTGATAATGTTGGGGGAAAGC |
| | | | TGCTAGATGCTGTTCTACAAAACATGAGAAGCCATGC |
| | | | TCGAATTTCTGTTTGTGGAATGATCTCACAGTACAAC |
| | | | CTTGAACAAACTGAAGGTGTACATAATCTGACGAATC |
| | | | TTGTGTGGAAAAGTGCTCGAATGGTTGGATTTCTGGT |
| | | | TGCTGACTATTACCACCTATACCCAAAATTTCTCGAA |
| | | | TATGTTATGCCTTACTTAAAAGAAGGAAAAATTGTGT |
| | | | ATGTTGAAGATATAGCTGAAGGGCTAAAGAGTGCCCC |
| | | | AAAAGCTTTGGTAGGGCTCTTCAATGGATGCAATGTG |
| | | | GGAAAACAGGTGGTTCTAGTCTCTTCGGAATGA |
| 58 | uncharacterized | Nucleic acid | ATGGAACAAGAGTACTCCTCTTCATTTCTCTCAACAA |
| | protein | | AGAGATATGCTGTTGTTACAGGTGGAAACAAGGGGAT |
| | LOC115704491 | | CGGGTTTGAAATATGCAGACAATTAGCTTCAAATGGC |
| | [Cannabis | | ATCAAGGTCGTGTTAACTGCCAGAGATGAAAAGAGGG |
| | sativa] | | GTGTTGAAGCTGTTGAGAAACTGATCAAAGAATCAAA |
| | | | TTTCACTAGTGAAGACAATGTCGTTTTTCACAGGCTT |
| | | | GATGTCGTTGACCCTGACACCATTGCTTCTTTGGCAG |
| | | | ATTACATCAAATCCCACTTCGGGAAGCTTGACATTTT |
| | | | GATAAATAATGCAGGAATTGCTGGGGGTACACTTGAT |
| | | | TCTTATGGTTATGCACAAGCCACTGAGCTTGCTGGTG |
| | | | GTAATTGGCCAGAGAACGGCAATTGGAATGAGATAAT |
| | | | GACCCAGAACTATGAATCGGCTGAAGAATGCCTGAAA |
| | | | ACAAATTATTATGGAGCCAAAGCAACGATTGAAGCAC |
| | | | TTGTTCCACTCCTCCAATTGTCTGATTCACCAAGAAT |
| | | | CGTCAATGTTTCATCCTCTCTTGGTCTCTTACAATAC |
| | | | ATACCGAATGAATGGGCCAAAAACATGCTGAGCGATG |
| | | | TTGATAAGCTAAGAGAAGAGCAAATAGATGAGGTAGT |
| | | | GAGTGAGTTTCTGAATGATTTCAAACAAGGTAAGTTA |
| | | | GAAGCCAAGAAGTGGCCTACAGAGATTTCGGGGTACA |
| | | | AAGTTTCGAAAGCTTCGCTGAACGCGTACACAAGGAT |
| | | | TTTGGCGAAGAAATACCCTCAAATGTGCGTTAACTGT |
| | | | GTGTGCCCTGGCTATGTCAAAACTGATATCACTTGCA |
| | | | ATACTGGGCAGTTGGTTGCTGCCGAAGGTGCTGAAAG |
| | | | TCCCGTGATGCTAGCCTTGTTGCCCCTCGCCAAGCCT |
| | | | TCCGGCTTCTTCTTCTCCAGGAAGCAACTCTCCCCTT |
| | | | TCTGCCATTCATTCATCACTAAACGAACCAAAACATT |
| | | | TTTATTACAAACCCAAGTTTCATCAACAATGTCTGGA |
| | | | GCTTCAGAAAGATATGCAATTGTAACAGGGGCAAATA |
| | | | AGGGGATTGGACTAGAGATAGTGAGACAATTGGCCTT |
| | | | GAATGGAGTCAATGTGGTCTTAACAGCAAGAGATGAG |
| | | | AAAAGGGGTCTTGAAGCTTTGGAGAAACTCAAAGAGA |
| | | | AAGAGAAAAACCTCTCTCACAAAGTGCTGTTTCACCA |
| | | | GCTCGACGTGGCTGATCCAGCTAGCATTGTTGCTATG |
| | | | GTTGATTTCATTAAAACACATTTCGGCAAACTTGATA |
| | | | TTTTGGTGAACAATGCTGGCGTTGGTGGAACAGAAGA |
| | | | AGACATGCATGCAATTGTAGCTTCTTTAAATGCTAAG |
| | | | ACCCCAAAAGAAGGTGATATTAAGAAAACTACTCAAA |
| | | | CTTATGAGTCAGCCAAAGAATGCATGCAAATAAACTA |
| | | | TTATGGTGCTAAAAAAACTGCTGAAGAGCTTATTCCC |
| | | | CTTCTCCAGTTATCTGATTCACCACGAATTGTTAATG |
| | | | TTTCTTCTACCATGGGAAAGCTACAGAATATATCAAA |
| | | | TGACTGGGCTAAAGGTGTTCTTAGTGATGCTGAGAGT |
| | | | CTCACAGAGGATAAAATTGATGAAGTGATAAGAGAGT |
| | | | TTCTGAAAGATTTCAAAGAAGGTTCATTGGAAACCAA |
| | | | AGGCTGGCCTAGTTTTTTGTCCCCATATACTGTCTCA |
| | | | AAAGCAGCCCTTACTGCCTTCACAAGGGTACTAGCAA |
| | | | AGAAGCACCCCAACTTTATCATCAACTGTGTATGCCC |
| | | | TGGATTTGTGAAGACAGAGATAAACTTCAACACCGGT |
| | | | ATTTTACAGCCTGAAGAAGGCGCTGCGAGTCCAGTAA |
| | | | GGTTAGCATTGCTTCCAAATGATGCACCTTCAGGCCT |
| | | | CTTCTTTGATCGGTCACAGGTTTCGTCTTTTTGA |
| 59 | cytochrome | Nucleic acid | ATGTTATTATTATTCCATTACAATATGTCCTTCTTAG |
| | P450 71A22- | | ACTTAACCAGTAATAATCTCTCTTCCTTTACCATTTT |
| | like [Cannabis | | ATTAGCAACCTTATTATTCTTTGTGCTGCTATATAAA |
| | sativa] | | TCATGGTTCTCTATTAAAACAAATTCTCCACCATCGC |
| | | | CTCCAAAGCTTCCAATAATTGGAAACCTCCACCAGCT |
| | | | CGGGTTGTACCCTCACCGAACGCTGCAGGCCTGGAGC |
| | | | AGGCGCTATGGCCCCGTCATGCAGCTCCGGCTAGGCA |
| | | | GCGTGCCGGTTCTCGTCATCTCCTCTGCCACCGCCGC |
| | | | TCGTGAGATTATGAAGACTCACGACATTGCCTTTTCC |
| | | | AACCGACCCAAGTCCTGTGCCCTCGAGAAGCTCCTCT |
| | | | ACAACTACAGAGACATTGCTTCGGCACCTTATGGCGA |
| | | | GTACTGGAGGCAGATAAAGAGCGTTTCCGTGCTTCAT |
| | | | CTGTTGAATAATAAAAGGGTTCAGTCCTATAGAGCTG |
| | | | TAAGGGAAGAGGAGACCAAGCTCATGGTTGAGAAGAT |
| | | | TCGAAAGTCTTGTGGGACTGGGGTGAATTTGAGCGAG |
| | | | TTGTTTGTAAGGCTAACCAACGACGTCGTTTGTAGGG |
| | | | TGGCCTTGGGGAGAAAGTACGGTGAAGAAAGTGGTGG |
| | | | GAAGAGGTTTAAGGAGCTTCTGGGGGAGTTTACGGAG |
| | | | CTACTTGGGGGTTTCTATGTAAGAGACTATTTTCCTA |
| | | | AGCTTGGTTGGTTGAGTCGTGTGAGTGGTTTGGATGG |
| | | | TAGAATGGATAAAGTGGCTAAGGAGTTCGATGAGTTT |
| | | | CTGGAAGGTGTTCTTCATGACCATATGAATACAAATA |
| | | | AGAATGTTGATGATGAACAGAAAGATTTTGTGGATAT |
| | | | TTTGCTTTGGATTCAGAGGGAAAACTCGCTTGGATTT |
| | | | TCTATTGATAGGACTTCCATAAAGGCTCTCATATTGG |
| | | | ACACATTTGCAGCGGGAACAGACACAACCTATACAGT |
| | | | CCTAGAATGGGCAATGACTGAGCTCATAAGACATCCA |
| | | | AACGCCATGAAAAAGCTTCAAAACGAGATCAGAACAA |
| | | | CAATCCTTAATAAGAAGATAACTAACATTGCCATGCC |
| | | | AGAAGAATATATTAATAGTGTTACAGAAGACGACCTA |
| | | | GAAAAAATGCCATACTTGAAGGCAGTTTTCAAAGAAA |
| | | | CTCTCCGTCTGCATCCACCAATCCCTTTAATCGTTCC |
| | | | TCGACTCACAATACAAGACATGAAAATAAGTGGATAC |
| | | | GACGTCGCTTCAGGCACCCAAGTATTCATCAATGCAT |
| | | | GGGCAATCGGGAGAGATCCGACCTTGTGGGAAGAGGA |
| | | | ACCAGACAAGTTTGAACCTGAGAGGTTTTTGCTGAAG |
| | | | AACGCTGCAATTGATTACAAAGGACATGACTTCGAGT |
| | | | TGATCCCTTTCGGGGCCGGGAGGCGAGGCTGCCCTGG |
| | | | GATTGTATTTGCCATGGCTGTTAACGAGCTTGCTTTG |
| | | | GCTAGTGTGGTCTATAAGTTTGATTGGGCGTTGTTGA |
| | | | GTAGTGGAGAAGAGGATTTGGATTATTATCATATGAC |
| | | | TGAAACCACAGGTTTGACTACGCATAGAAAGTTTCCT |
| | | | CTTATGGCTGTGCCAACTGAATATTATCAATGA |
| 60 | (−)-germacrene | Nucleic acid | ATGTCTCCTTGCGAAGCTACAATTGATGAAAAACGCC |
| | D synthase-like | | CTAATATGCCAAAGTTTACTCCAACCATTTGGGGTGA |
| | [Cannabis | | TTATTTCATGTCTCATGCTTCAAGTCATCACTCATCT |
| | sativa] | | CTTATGGAAACTATGGAGAATAATAACAAAGAGAGTT |
| | | | ATGAGAAGATTATTGAGATGAAGGAACAAGTGAAGAA |
| | | | TAAATTACTTCATGGTCTTCATCCTTTGGAAAACCCT |
| | | | TTGGAGACACTTGAATATATTGATGATATTCAACGAT |
| | | | TGGGGTTGTCTTATTATTTTGAAAATGAAATTGAACA |
| | | | AAATTTGGAGCAATTTCATAATAATTATCAAAATCTA |
| | | | ATTGATTTTGGTGATAATAACCTTTATGCTGATGCTC |
| | | | TTTGCTTTCGGTTGCTTAGGCAACAAGGTTATAATAT |
| | | | TGCATGTGACATATTCGACAAGTACAAGAATGAAAAT |
| | | | GAAAAATTTAAAGAATCAATTTCGAGTGACATTCGAG |
| | | | GAATGTTGAACTTGTATGAAGCTGCACAAATGAGAGT |
| | | | TCATGGAGAGAAAATACTAGACGAAGCACTTATCTTT |
| | | | ACAACTACTCATCTTGAATCCTCAGTTAAAACATGTC |
| | | | AATTGAGCTCTCCTTATCTAGACCTAGTGAAACATGC |
| | | | CCTAATGCACCCTATTCGAAAGAGCTTACAAAGAAGA |
| | | | GAGGCAAGACTTTACATATCACTTTATCATCAACTAC |
| | | | CTTCTCATGAGGAGATTCTTTTAATACTTGCTAAACT |
| | | | AGATTTCAACCTGCTTCAAAAACTACATCAAAAGGAA |
| | | | CTAAGTTACATAACGAGGTGGTGGAAGGAGTTTGATT |
| | | | ACAAAAGTAAGCATTCATTTATAAAAGACAGAATAGT |
| | | | GGAGTGCTATTTCTGGGTTTATGGAGTGTTTTTTGAG |
| | | | GCAGAAACTTCCCAAATCAGACTAATAATCACCAAAT |
| | | | TAATTGCTATTCTCACAATAATTGATGATGCTTATGA |
| | | | TAGCTTTGGTACACTTGAAGAACTAGAGCCTTTTACT |
| | | | CAAGCAATAGAAAGGTGGGATATATGTGCCATAGATA |
| | | | CTCTGCCTGAGTACATGAAAATATTTTACATGAAACT |
| | | | TTTGGAGATCTACAATGAAATTGAACAATTTTCTAAG |
| | | | GAAAGATCATACTGCCCTAGCTATGCTAAGAAAGGGG |
| | | | TGCAATCTCTAATTAGAGCTTATTTTAAGGAAGCCAA |
| | | | ATGGTTACACACAAAATATATACCAACATTAGAAGAA |
| | | | TATATGCCAGTTGGGATTGATAGTGCAGGATCTTTTA |
| | | | TGCTCATTTCAATGGTTTTTATTGGAATGGGAGATAT |
| | | | TGTTACAAAACATTCTATGGATTGGATATTTTCTAAT |
| | | | CCTCAACCTAAAATTATACAAACTATGGCAATAGTTG |
| | | | GAAGAGTTATGAATGACATTGGCTACCATAAGTCGGA |
| | | | GCGAAAGAAATCATCAGGAGAAATTGTGGCTTCAACT |
| | | | GTGGAGTGTTACATGAAACAATATGGCGTGACTGGTG |
| | | | AAGAAGCTATAGAGAAACTTAGCCAACAAGTTAAAGA |
| | | | TTCATGGAAAGATCTCAATGAAGATCTTCTCAATCCA |
| | | | ATCACTATCCCTAGGCCACTCTTAATGCAAGTTCTAA |
| | | | AGCTTGTACGAGTGAACCATGAGATTTATAGAGAAGG |
| | | | AGATGGCTTTACACAACCCACTTTGCTCAAGAATTTG |
| | | | ATTCATTCTCTCATCATCAATCCAATTGACTTTTGA |
| 61 | (−)-kolavenyl | Nucleic acid | ATGCCTTCTCTCTTCTCCCAATCACTACTTCTCCCTT |
| | diphosphate | | TCTCTCAAAACACTAATACTCTCTCCCTTTTCCATCA |
| | synthase | | ACCAAAACTTCTTCCTCCAGGTGCTTCGCTATTGGAA |
| | TPS28. | | GCTAAAGACAAACAAGTTAACTTTGATCGTGATATTC |
| | chloroplastic | | GCTCAAAATGCAGCGCTATATCAAAACCCCGCACTCA |
| | [Cannabis | | CGACGTGTTTCAAAGTGGTGGTCTGCCAGTTATAAAG |
| | sativa] | | TGGCACGAGATTGTGGAGGATGACATAGATGGAGAAG |
| | | | AAGAAGATACTAAGTGGACAAGATCGAATGAGATCGA |
| | | | GGAACGTGTCGCTTCAATCAAATCAATGTTGGAGAGT |
| | | | ATGGATGAGGGAGAGATAAGCATTTCAGCGTACGACA |
| | | | CAGCATGGGTAGCCCTTGTGGAAGATATTCATGGGAG |
| | | | TGGCTTACCTCAATTCCCATCGAGTCTCCAATGGATC |
| | | | GCCACACATCAGCTCTCCGACGGTTCTTGGGGCGATG |
| | | | CTGACATTTTCTCCGCACACGATCGCCTCATCAACAC |
| | | | TTTGGCTTGTGTTGTTGCTTTGAAATCTTGGAACCTT |
| | | | TATCCCGAAAAATGTCAAAAAGGTATGGCCTTTTTCA |
| | | | ATGCAAATATAAGTAAGCTTGAGAGGGAGAATCCGGA |
| | | | ACACATGCCTATTGGTTTCGAAGTGGCTTTCCCTTCT |
| | | | TTACTTGAAATAGCTCGAAAATTAAACCTTGAAGTGC |
| | | | CTGAGGATTCTCCTGTGTTAAAAGTCATATATGCTAG |
| | | | GAGAGATTTCAAGCTCACAAGGATTCCGAGGGACATA |
| | | | ATGCACACAGTGCCCACGACGCTACTCCATAGCTTGG |
| | | | AAGGAATGGTAGGTCTGGACTGGGAAAAGCTTTTGAA |
| | | | ACTGCAGTCCCAAGATGGGTCATTCTTGTTCTCACCA |
| | | | TCCTCAACTGCTTTTGCACTCATGGAGACCAAAGACC |
| | | | GAAATTGCTTGCAATATTTAACTAAAGCGGTCCAAAG |
| | | | GTTCAACGGGGGTGTCCCAAATGTTTACCCGGTTGAC |
| | | | TTGTTCGAGCACCTTTGGGTTGCGGATCGGTTGCAGC |
| | | | GCTTGGGAATATCAAGATTCTTTGAGCCACAAATTGA |
| | | | GGAATGTATCGATTATGTATTCAGAAATTGGACTGAG |
| | | | AAAGGAATTGGCTGGGCAAGAAATTCCAAGGTTGAAG |
| | | | ATATTGACGATACCGCAATGGGTTTCAGACTACTAAG |
| | | | ATTGCATGGTCACAAAGTTTCTGCCGATGTGTTCCAA |
| | | | CACTTTAAGAAAGGTGACGATTTTTTCTGCTTTCGGG |
| | | | GCCAGTCAACTCAAGCAGTGACTGGGATGTATAACCT |
| | | | TTATAGAGCTTCTCAGTTGGTTTTCCCTGGAGAAAAA |
| | | | ATTCTTGAAGATGCCATGGAATTCTCATCGAAATTTC |
| | | | TCAGAAAAAAACAGGCGTCCAATGAATTGCTAGATAA |
| | | | ATGGATCATAACAAAGGACTTACCTGGTGAGGTGGGT |
| | | | TTCGCATTGGAGGTTCCATGGAATGCAAACTTACCTC |
| | | | GAGTAGAGACCAGATTCTACATTGAACAGTATGGTGG |
| | | | ACAAAATGATGTTTGGATTGGCAAGACACTCTACAGA |
| | | | ATGCGAAAAGTTAACAATGACGAATATCTGGAGTTAG |
| | | | CAAAACTTGATTACAACATTTGCCAAGCTTTGCATTC |
| | | | GATTGAGTGGCACAATTTGCTAAAATGGTACCGAGAT |
| | | | TGTAAGTTGGAAAATTATGGAGTGAGCAGAAGGAACC |
| | | | TCCTCTTGGCCTATTTTCTTGCTGCGGCCAGTATTTT |
| | | | CGAACCGGATAGGGCCGATGAGCGGCTTGCATGGGCT |
| | | | AAAACGGCAGCACTGATGCAGGCCATCCAATCTCATT |
| | | | TCGATGACCAGAAAGCTTCTTCGGAGCATCGTATAGC |
| | | | TTTTGTCTCTGCTTTTAAAAGGAGTTGTAACATGCCA |
| | | | TCGTATTTGATTACAAGGGTGTCGAACATAAGTGATA |
| | | | CAGATCATGGCCTTCTTAGAACGTTGATGACGACTCT |
| | | | CAGCCACCTCTCTTTGGACACAATGATGCTGTATGGT |
| | | | CGGGACATCACCCACCATTTACGTCAAGCTTGGGAAA |
| | | | AGTGGCTGGTGAAGTGGCAAGAGGGTGGTGATGGACA |
| | | | TTACGAAGAAGAAGCAGAATTATTGATCCAAACAATA |
| | | | AACCTTAGCTCAGGCCGTACACTTGTGAAGGCCCTCT |
| | | | TGCTGTCAAATCCTCACTATGAAAAACTCTTCAGTAC |
| | | | CACAAACAAAGTTTGCTGCAAAATTCGTCACTTTCAA |
| | | | AGACAAAGGCATAGGGCCAAGGCAAATCAAAATGGAG |
| | | | AATTTAACAGAAACATCTTAACACCAGAAATAGAGTC |
| | | | AGATATGCAAGAGGTTGTGCAATTGGTGCTACAAAAA |
| | | | TCTTCAGATGACATGATCAACACAAAAATTAAGCAGA |
| | | | CATTTCTACTGGTGGCCAAGTCTTTTTATTATGCTGC |
| | | | CTACTGTGATTCTAAGACCATCAATTTCCACATTGGC |
| | | | AAAGTAATATTTGAGACTGTGGACTGA |
| 62 | 2- | Nucleic acid | ATGGCGACCGCTATTTTCTCCAACCCCAAGTTCTCCC |
| | isopropylmalate | | CCACAATCACCACCACCTCCTCCAAAAACCATTACCA |
| | synthase 2. | | CTACCAACGCCGTACACATTTACTACTCCACGACAAA |
| | chloroplastic | | GTCCAAGCTTTTCAAGCCGCCTCTCTTAAACTCAACC |
| | [Cannabis | | CAAATCCTCATTACAAGAAACCCCAGATCGTTTCCTG |
| | sativa] | | CCAGAGCTCCGGTAGCGAGTCACCGGACACAGAGAAG |
| | | | GTGCGGCGGCCGGAGTACATTCCGAACCGTATCTCCG |
| | | | ATCCAAACTATGTACGTATCTTCGACACTACTCTCCG |
| | | | AGACGGTGAGCAGTCCCCTGGGGCCGCCCTGACGTCA |
| | | | AAGGAGAAGCTGGACATTGCCAGACAGCTTTCCAAGC |
| | | | TCGGCGTTGACATAATCGAGGCTGGATTCCCCGCCGC |
| | | | CTCGAAAGATGACTTCGAGGCCGTCAAGATTATTGCC |
| | | | AAAGAGGTCGGTAACGCCGTTGACGCCGACGGCTATG |
| | | | TTCCCGTTATCTGTGGTCTGTCGAGGTGTAATGAGAA |
| | | | CGATATTAGGAGGGCTTGGGAGGCGGTCAAGTACGCC |
| | | | AAAAGGCCTAGGATTCATACTTTCATTGCTACAAGTC |
| | | | CAATTCACATGGAGTACAAGTTGAGAAAGAGTAAGGA |
| | | | GCAGGTGATTGAGATCGCTAGGAACATGGTGAAGTTT |
| | | | GCTCGGAGTTTGGGGTGTGATGATGTTGAGTTTAGCC |
| | | | CTGAAGATGCTGGCAGGTCTGAGAGGGAATTCTTGTA |
| | | | TCAGATTTTGGGAGAAGTTATAAAGGCTGGAGCAACA |
| | | | ACTCTAAACATACCTGACACTGTTGGTTACAACGTGC |
| | | | CAAAAGAATTTGGAGAATTGATTGCTGACATTAAAGC |
| | | | CAATACCCCTGGAATTGAGAATGTTGTCATTTCTACA |
| | | | CACTGTCAAAATGATCTTGGACTTTCTACTGCAAACA |
| | | | CGATATCGGGGGCATGCGCAGGTGCTAGACAATTAGA |
| | | | AGTAACAATCAACGGCATTGGTGAAAGAGCCGGGAAT |
| | | | GCATCTCTGGAGGAGGTTGTAATGGCCATAAAATGCC |
| | | | GTGGAGATCAACAACTGGGAGGACTTTATACTGGAAT |
| | | | CAACACAAGACACATCTCAATGACAAGCATAATGGTT |
| | | | GAGGAATACACAGGGTTGCAAGTACAGCCACATAAGG |
| | | | CTATTGTTGGAGCCAATGCTTTTGCACATGAAAGCGG |
| | | | TATCCATCAGGATGGAATGCTTAAGCACAAAGGTACA |
| | | | TATGAAATCATATCCCCAGAAGATATAGGGCTTGAAC |
| | | | GAAGCAATGAAGCTGGTATAGTCCTTGGAAAACTCAG |
| | | | TGGTCGCCATGCATTGAAACAACAACTTGAGGAGCTT |
| | | | GGTTATGAGCTTGAGGATGAGCAACTTGAGAGTATAT |
| | | | TCTGGCGCTTCAAATCTGTGGCTGAACTTAAGAAGAG |
| | | | GATAACTGATGCTGACCTCAGAGCACTAGTTTCGGAT |
| | | | GAAGTTTTTCAACCAGAAGTCATCTGGAAGTTCGTTG |
| | | | ATTTGCAGGTTACATGTGGAACTCTTGGTCTTTCAAC |
| | | | TGCAACCGTCAAACTTATTGGTTCAGATGGGAAAGAG |
| | | | CATGTTGCTTGTTCAGTAGGAACTGGTCCAGTGGACT |
| | | | CGGCTTACAAAGCTGTTGATCTGATTGTGAAGGAACC |
| | | | AGTAGCACTCCTGGAGTATTCAATGAATGCTGTTACT |
| | | | GAAGGTATAGATGCAATTGCAACCACCCGTGTGCTAA |
| | | | TCCGAGAGGAAACGAGCGACTTGTCAGGTCATGGTTC |
| | | | AACTGTTGAACGAGTTACTCGGACATTTAGTGGGAAC |
| | | | GGGTCAGGAATGGATATTGTGGTTTCAAGTGTAAAGG |
| | | | CTTACATTGGTGCGATAAACAAGATGTTAGGTTTCAA |
| | | | AGATAGGACCGTTGTGAATTCTTCTGAAGAGAGAATA |
| | | | CCCATATCTGCATAA |
| 63 | transcription | Nucleic acid | ATGGGGAGGCACTCTTGTTGTTACAAGCAGAAACTGA |
| | factor MYB61 | | GAAAAGGGTTGTGGTCACCAGAAGAAGATGAGAAACT |
| | isoform X1 | | TCTTAATTATATAACCAAGCATGGACATGGCTGCTGG |
| | [Cannabis | | AGCTCTGTCCCTAAGCTAGCTGGTCTTCAGAGATGTG |
| | sativa] | | GAAAAAGTTGCAGGCTAAGGTGGATAAATTATTTGAG |
| | | | GCCTGATTTGAAAAGAGGCCCATTTTCACAACAAGAG |
| | | | GAGAATTTGATAATTGAACTTCATGCAGTTCTTGGCA |
| | | | ACAGATGGTCACAGATTGCAGCTCAGTTACCAGGAAG |
| | | | AACAGATAATGAGATTAAAAACTTATGGAATTCTTGC |
| | | | ATTAAGAAGAAACTGAGGCAAAAAGGGATTGACCCAA |
| | | | ATACTCATAAGCCATTATCTGAGGTAGAAAATGACAT |
| | | | TGGTAATAAATTGGAGAACAAGGGTAACAAAGCTGCA |
| | | | ACCAATAACAACAACAATGAGAATATTAATAATTCTA |
| | | | CTGTTAGAGCTTCTTCATTAGGAAACTTATCCAATGA |
| | | | TCATCATCATCATCATCATCATCATCTGAATCTAGCT |
| | | | GACCAGTCACAACCATCAATGGCGGCCATCAATCGTT |
| | | | ACCCACTATTGGAAGTCTCATCCTCAACTCCGCCGAC |
| | | | ACAAGAATTCTTCATAGAAAAATCAACAGATACCAGA |
| | | | TCATCACCATCAATATCATCATCATCACCTTGTGATT |
| | | | TTTCTACCTACTTCTCTTTCCACTCAAACAATTACAA |
| | | | TACGACGTCGTCCGCTGCTGCAGCTGCTGCTGTTTCT |
| | | | CATCATCAAGATCAAAACAACAACAACAACATGGCCA |
| | | | GTTTCTGCTTCAACATTAATCAAAATTCAACTAGACC |
| | | | TCCACAACACCATCATCATAATCAGATGATTAGTAAT |
| | | | CTCATCCAGCCACTACAACAACAAGTATCACCTTCAT |
| | | | CAACAACAACAGCATCATCATCATCACCACCCTCCAA |
| | | | TATTCCACGTGTAAAGCCCTCCATAAGTCTCCCCTTA |
| | | | TTATCTGATCACCAAAACAACAGTAATAGCACTACTA |
| | | | CTACTACTACAACAACTACTGGAGCCGTACAAAATTG |
| | | | GGAAACTAGTACTTTCAGCAACAACGGAAGTAGTAGT |
| | | | AGTAGCTGCAATATCGAATTACAAGGTAATAATAATA |
| | | | ACAACAACAACAACTTCTTTGATCACAACACTAATTC |
| | | | CACCGCCGCGGCCGCCGCCGCCGCCGCTCCTAATAAC |
| | | | TTCTCGTGGGGATTAGTCAATGAAAGTACTGTTGGTA |
| | | | GCATAAAATCTGATGACCCAGAAGACATAAAATGGTC |
| | | | TGAATATCTCCATAGCCCTTTTCTTCTTGGTGGAGGA |
| | | | ATTAGTAATACTAATAATCAAAATTCTTCTTCTTCTT |
| | | | CACATCTTCAACCCATTTTGTACAGTAACATTGTGAA |
| | | | ACCAGAATCACACTTTAGTAATACTACTACTGCTACA |
| | | | GGATCAAACCCCACGTGGCATCATCAGAACGATCATC |
| | | | ATCAGCTACAAGCGGCTTCATCAGAAATAATGTACAC |
| | | | TAATAAAGATCTACAGAGACTTGCTGTAGCTTTTGGA |
| | | | CAGACCCTTTAG |
| 64 | geranylgeranyl | Amino acid | MSTVNLTWVQTCSMFNQGGRSRSLSTENLNLYHPLKK |
| | pyrophosphate | | TPFSIQTPKQKRPTSPESSISAVLTEQEAVKEGDEEK |
| | synthase. | | SIFNFKSYMVQKANSVNQALDSAVLLRDPIMIHESMR |
| | chloroplastic | | YSLLAGGKRVRPMLCLSACELVGGKESVAMPAACAVE |
| | [Cannabis sativa] | | MIHTMSLIHDDLPCMDNDDLRRGKPTNHKVFGEDVAV |
| | | | LAGDALLAFAFEHMAVSTVGVPAAKIVRAIGELAKSI |
| | | | GSEGLVAGQVVDIDSEGLANVGLEQLEFIHLHKTGAL |
| | | | LEASVVLGAILGGGTDEEVEKLRSFARCIGLLFQVVD |
| | | | DILDVTKSSQELGKTAGKDLVADKVTYPRLMGIDKSR |
| | | | EFAEQLNTEAKQHLSGEDPIKAAPLIALANYIAYRQN |
| 65 | (−)-kolavenyl | Amino acid | MPSLFSQSLLLPFSQNTNTLSLFHQPKLLPPGASLLE |
| | diphosphate | | AKDKQVNFDRDIRSKCSAISKPRTHDVFQSGGLPVIK |
| | synthase TPS28. | | WHEIVEDDIDGEEEDTKWTRSNEIEERVASIKSMLES |
| | chloroplastic | | MDEGEISISAYDTAWVALVEDIHGSGLPQFPSSLQWI |
| | [Cannabis sativa] | | ATHQLSDGSWGDADIFSAHDRLINTLACVVALKSWNL |
| | | | YPEKCQKGMAFFNANISKLERENPEHMPIGFEVAFPS |
| | | | LLEIARKLNLEVPEDSPVLKVIYARRDEKLTRIPRDI |
| | | | MHTVPTTLLHSLEGMVGLDWEKLLKLQSQDGSFLESP |
| | | | SSTAFALMETKDRNCLQYLTKAVQRENGGVPNVYPVD |
| | | | LFEHLWVADRLQRLGISRFFEPQIEECIDYVERNWTE |
| | | | KGIGWARNSKVEDIDDTAMGFRLLRLHGHKVSADVFQ |
| | | | HFKKGDDFFCFRGQSTQAVTGMYNLYRASQLVFPGEK |
| | | | ILEDAMEFSSKELRKKQASNELLDKWIITKDLPGEVG |
| | | | FALEVPWNANLPRVETRFYIEQYGGQNDVWIGKTLYR |
| | | | MRKVNNDEYLELAKLDYNICQALHSIEWHNLLKWYRD |
| | | | CKLENYGVSRRNLLLAYFLAAASIFEPDRADERLAWA |
| | | | KTAALMQAIQSHEDDQKASSEHRIAFVSAFKRSCNMP |
| | | | SYLITRVSNISDTDHGLLRTLMTTLSHLSLDTMMLYG |
| | | | RDITHHLRQAWEKWLVKWQEGGDGHYEEEAELLIQTI |
| | | | NLSSGRTLVKALLLSNPHYEKLESTTNKVCCKIRHFQ |
| | | | RQRHRAKANQNGEENRNILTPEIESDMQEVVQLVLQK |
| | | | SSDDMINTKIKQTFLLVAKSFYYAAYCDSKTINFHIG |
| | | | KVIFETVD |
| 66 | solanesyl | Amino acid | MLFSRGERRIPTTTENGFSRWFVSHRPGYSQSQTTTH |
| | diphosphate | | CSRDSTHKIFGGFEESREWGFAGSRYQIHHQSSSLVE |
| | synthase 3. | | EELDPFSLVADELSLVANRLRDMVVAEVPKLASAAEY |
| | chloroplastic/ | | FFKMGVEGKRFRPTVLLLMATALNVKVPEPAKALADT |
| | mitochondrial | | LTPELRTRQQSVAEITEMIHVASLLHDDVLDDAETRR |
| | [Cannabis sativa] | | GVGSLNCIMGNKVSVLAGDELLSRACVALAALKNTEV |
| | | | VTLLATVLEQLVTGETMQMTCTSEQRCSMEYYMQKTY |
| | | | YKTASLISNSCKSVAVIAGQTTEVAMLAFEYGKNLGL |
| | | | AYQLIDDILDFTGTSASLGKGSLSDIRHGIVTAPLLY |
| | | | AMEEFPQLRAVVEQGFENPKNVDIALDYLGKSRGIQK |
| | | | ARELAIKHANLAAEAIESLPESDDEDVKRSRRALVDL |
| | | | TQRVVTRTK |
| 67 | terpene synthase | Amino acid | MSTNNNNNINNIISRRSANYQPSLWHFDYVQSLSTPF |
| | 10-like isoform | | KEGAYAKRVEKVKEEVRVMVKRAKEEEKPLSQLELID |
| | X1 [Cannabis | | VMQRLGISYHFENEINDTLKDIYNNNNVYNTNNNVYA |
| | sativa] | | NSLEFRLLRQHGYPVSQEIFSTCKDERGNFMVSSNDI |
| | | | KGMLSLYEASFYLVENEDGILEETREKTKKYLEEYII |
| | | | MIMEKQQSLLDQNNNNDYDYDYELVSHALELPLHWRM |
| | | | LRLESRWFIDVYEKRLDMNPTLLTLAKLDENIVQSIY |
| | | | QDDLKHVESWWESTNMGKKLEFARDRTMVNFLWTVGV |
| | | | AFEPHFKSFRRMITKVNALITVIDDIYDVYGALDELE |
| | | | LFTNAVERWDISAMDGLPEYMKTCFLALYNFINDLPF |
| | | | DVLKGEEGLHVIKFLQKSWADLCKSYLREARWYYNGY |
| | | | TPRFEEYIENAWISISGPVILSHLYFFVVNPNMEDAL |
| | | | LSTCENGYPTIIRHSSMILRLTDDLATSTDELKRGDV |
| | | | PKSIQCKMYEDGISEEEARQRIKLLISETWKLINKDY |
| | | | INLDDDDDDGDDYSPMFYESNNINKAFIEMCLNLGRM |
| | | | AHCIYQYGDGHGIQDRQTKDHVLSLLIHPIPLTQ |
| 68 | (−)-limonene | Amino acid | MQCIAFHQFASSSSLPIWSSIDNRFTPKTSITSISKP |
| | synthase. | | KPKLKSKSNLKSRSRSSTCYPIQCTVVDNPSSTITNN |
| | chloroplastic | | SDRRSANYGPPIWSFDFVQSLPIQYKGESYTSRLNKL |
| | isoform X1 | | EKDVKRMLIGVENSLAQLELIDTIQRLGISYRFENEI |
| | [Cannabis | | ISILKEKFTNNNNNPNPINYDLYATALQFRLLRQYGE |
| | sativa] | | EVPQEIFNNFKNHKTGEFKANISNDIMGALGLYEASE |
| | | | HGKKGESILEEARIFTTKCLKKYKLMSSSNNNNMTLI |
| | | | SLLVNHALEMPLQWRITRSEAKWFIEEIYERKQDMNP |
| | | | TLLEFAKLDENMLQSTYQEELKVLSRWWKDSKLGEKL |
| | | | PFVRDRLVECFLWQVGVRFEPQFSYFRIMDTKLYVLL |
| | | | TIIDDMHDIYGTLEELQLFTNALQRWDLKELDKLPDY |
| | | | MKTAFYFTYNFTNELAFDVLQEHGFVHIEYFKKLMVE |
| | | | LCKHHLQEAKWFYSGYKPTLQEYVENGWLSVGGQVIL |
| | | | MHAYFAFTNPVTKEALECLKDGHPNIVRHASIILRLA |
| | | | DDLGTLSDELKRGDVPKSIQCYMHDTGASEDEAREHI |
| | | | KYLISESWKEMNNEDGNINSFFSNEFVQVCKNLGRAS |
| | | | QFMYQYGDGHASQNNLSKERVLGLIITPIPM |
| 69 | cytochrome | Amino acid | MEFQQIIPSFQVLLFSFFMIIVVSILLKRAQTSNRSA |
| | P450 71D11 | | SKLPPGPWRLPFLGNLHQLLGPLPHHTERDLAKKHGP |
| | [Cannabis | | FMYLKIGQVPTIVVSSPEYAKEVMKVHDNALASRPNN |
| | sativa] | | LVTQILAYNGTDIIFAPYGQYWKEVKKICVQELLTLS |
| | | | RVKTFQPIREEESFNIVKNIASKVGSPINLTQMLKSL |
| | | | SYSIIARAAFGEKRSDHDDFVYIMKETVKLSAGFAFG |
| | | | DVFPSLSFLDWYTISKFKDLKLRSSRIVDRIIKLHID |
| | | | DQDKDNLKKSGEEEDLVDVLLRFHKSEDPNNFTLTKD |
| | | | NLKAIIASIFGAGSDTSSVTMELAMAEMMRNPRVMKK |
| | | | AQDEVREVFGKKGELDESSINEMTYLKSVVKETLRLH |
| | | | PPAAMLFPRESREKCDINGYEIPMKTKILVNAWAIGR |
| | | | DPKYWIEPESEMPERFLESSIDFKGNNFEFIPFGAGR |
| | | | RICPGISFALTSIELPLAFLLYHEDWKLPNGMKPEEL |
| | | | DMTERFGITVCRKKDLYLIPSKYEPPSMAKAMNIK |
| 70 | cytochrome | Amino acid | MDLQLLSFPIILITLFFMEMVVKIVLKIAHHQTKNSV |
| | P450 71D9 | | SKLPPGPWKLPLVGNIHQIFGSSPHVSERDLAKKYGP |
| | [Cannabis | | FMYLKIGQIPTLIVSSPEYVKEIMRTHDVVFASRPQT |
| | sativa] | | LAAQIMAYNCTDIIFSSYGEHWRQLRKISMQELLSPG |
| | | | RVQTFRPVREEELCNLVEGIMTSSKDGSPINVTKMVT |
| | | | KCSYGITSRAAFGKKSSDHDEFISIVEEAIEAAGGFE |
| | | | FAEVFPALRELDWKSRPIFESIKLRSSRIMENIIKEH |
| | | | IKEKEISFEKIGKDEDLVDVLLKFHKNGDDLGRETLT |
| | | | KDNIKAVIFDIFVAGSETTSLSVDWAMVEMMRYPKVM |
| | | | KKAQEEVRKIIGTKGSVNESSINEMKYLKLVVKETLR |
| | | | LHPPAPLLLPRESTEKCDIDGYEIPKKTRVIVNAWAI |
| | | | GRDPKYWIEPENEMPERFMESSIDFKGNNFEYIPFGG |
| | | | GRRICPGMLFGVINIELSLAYLLYHEDWKLPNGMNHE |
| | | | NLDMTELFGLTMRRKDDLYLIPTIYDHSSIAKS |
| 71 | cytochrome | Amino acid | MDLQLPSFPVLLSLLESLLMAVTILMKRARNSKLPPG |
| | P450 71D11 | | PWRLPLVGNLHQLLLGYSSSSSSYEVESDLAKKHGPF |
| | [Cannabis | | MYLEIGQVPTVIVSSPEYAKEIMRTHDVVFASRPRTL |
| | sativa] | | AAQIIGYDCTDIAFAPYGDYWRQLRKICMQALFSPKR |
| | | | VQSLEPIREKEVENTLQHIIANSNKLNFTQMVTNLSY |
| | | | SIVSRAAFGEKSSDHDEFISIVEEDIKVAGGFEFGEL |
| | | | FPSLRFLDWTSRPKYESLKQRSSRILEKIIKQHMINQ |
| | | | NNEKSEEEQDLVDVLLKYHNKANLGLTLDNIKGVIWD |
| | | | IFEAGSETTAVTEDWAMVELMRNPIMMKKAQDEVREV |
| | | | FGRKGLVDKTSIHEMKYLKLIIKETLRLHPPAPELLP |
| | | | RENSEKCEINGYEIPNGTRVLVNVWGIGRHAKYWNEP |
| | | | ESFIPERFDDSSIDEKGNNFEYIPFGAGRRICPGITE |
| | | | GVVSLEYSLALMLYHFDWKLPNGMKPQDLDMSELFGI |
| | | | AVRRKDDLYLIPTIYHQSPLAN |
| 72 | (−)- | Amino acid | MTEHSPLTPKTKLHGKVAVVTGGASGIGEATARKFAA |
| | isopiperitenol/ | | DGARAVVIADIQDEKGQNVAASIGLERSTYVHCDVTD |
| | (−)-carveol | | EAQVAALIDSTVQKYGQVDVMFSNAGVPCESDQTILD |
| | dehydrogenase. | | LDLVAYDKVFAVNARGMAACVKHAARAMVEGGVRGSV |
| | mitochondrial- | | ICTASNLASIGTEKYTDYTMSKHAVLGLVRSASLQLG |
| | like [Cannabis | | ARGIRVNAVSPGPLRTPLLKAFIDRSEEEQDKMIEAS |
| | sativa] | | LSLKNGRTPSVENVADAVSFLASDESEFITGHNLAVD |
| | | | GGYIHHPP |
| 73 | 2-alkenal | Amino acid | MEEVSNKQLLLKNYVSGYPKESDMVLATSTIKLKLPE |
| | reductase | | GSNGVLVKNLYLSCDPYMGPRMKNLNNGFFIEPFKLG |
| | (NADP(+)- | | SPITGNGICKVLKSGNPNFKEGDLVSGVTGWEQYSVI |
| | dependent) | | ESTKYMSKIQNTDVPLSYYTGLLGMPGMTAYAGFFEV |
| | [Cannabis | | CSPKKGEYVFISAASGAVGQLVGQFAKLLGCYVVGSA |
| | sativa] | | GRNEKVDLLKNKFGFDEAFNYKEESDEDVALKRYFPE |
| | | | GIDIYFDNVGGKLLDAVLQNMRSHARISVCGMISQYN |
| | | | LEQTEGVHNLTNLVWKSARMVGELVADYYHLYPKFLE |
| | | | YVMPYLKEGKIVYVEDIAEGLKSAPKALVGLENGCNV |
| | | | GKQVVLVSSE |
| 74 | uncharacterized | Amino acid | MEQEYSSSFLSTKRYAVVTGGNKGIGFEICRQLASNG |
| | protein | | IKVVLTARDEKRGVEAVEKLIKESNFTSEDNVVEHRL |
| | LOC115704491 | | DVVDPDTIASLADYIKSHFGKLDILINNAGIAGGTLD |
| | [Cannabis | | SYGYAQATELAGGNWPENGNWNEIMTQNYESAEECLK |
| | sativa] | | TNYYGAKATIEALVPLLQLSDSPRIVNVSSSLGLLQY |
| | | | IPNEWAKNMLSDVDKLREEQIDEVVSEFLNDEKQGKL |
| | | | EAKKWPTEISGYKVSKASLNAYTRILAKKYPQMCVNC |
| | | | VCPGYVKTDITCNTGQLVAAEGAESPVMLALLPLAKP |
| | | | SGFFFSRKQLSPFCHSFITKRTKTFLLQTQVSSTMSG |
| | | | ASERYAIVTGANKGIGLEIVRQLALNGVNVVLTARDE |
| | | | KRGLEALEKLKEKEKNLSHKVLFHQLDVADPASIVAM |
| | | | VDFIKTHEGKLDILVNNAGVGGTEEDMHAIVASLNAK |
| | | | TPKEGDIKKTTQTYESAKECMQINYYGAKKTAEELIP |
| | | | LLQLSDSPRIVNVSSTMGKLQNISNDWAKGVLSDAES |
| | | | LTEDKIDEVIREFLKDFKEGSLETKGWPSFLSPYTVS |
| | | | KAALTAFTRVLAKKHPNFIINCVCPGFVKTEINENTG |
| | | | ILQPEEGAASPVRLALLPNDAPSGLFFDRSQVSSF |
| 75 | cytochrome | Amino acid | MLLLFHYNMSFLDLTSNNLSSFTILLATLLFFVLLYK |
| | P450 71A22- | | SWFSIKTNSPPSPPKLPIIGNLHQLGLYPHRTLQAWS |
| | like [Cannabis | | RRYGPVMQLRLGSVPVLVISSATAAREIMKTHDIAFS |
| | sativa] | | NRPKSCALEKLLYNYRDIASAPYGEYWRQIKSVSVLH |
| | | | LLNNKRVQSYRAVREEETKLMVEKIRKSCGTGVNLSE |
| | | | LFVRLTNDVVCRVALGRKYGEESGGKRFKELLGEFTE |
| | | | LLGGFYVRDYFPKLGWLSRVSGLDGRMDKVAKEFDEF |
| | | | LEGVLHDHMNTNKNVDDEQKDFVDILLWIQRENSLGE |
| | | | SIDRTSIKALILDTFAAGTDTTYTVLEWAMTELIRHP |
| | | | NAMKKLQNEIRTTILNKKITNIAMPEEYINSVTEDDL |
| | | | EKMPYLKAVEKETLRLHPPIPLIVPRLTIQDMKISGY |
| | | | DVASGTQVFINAWAIGRDPTLWEEEPDKFEPERFLLK |
| | | | NAAIDYKGHDFELIPFGAGRRGCPGIVFAMAVNELAL |
| | | | ASVVYKFDWALLSSGEEDLDYYHMTETTGLTTHRKEP |
| | | | LMAVPTEYYQ |
| 76 | (−)-germacrene | Amino acid | MSPCEATIDEKRPNMPKFTPTIWGDYFMSHASSHHSS |
| | D synthase-like | | LMETMENNNKESYEKIIEMKEQVKNKLLHGLHPLENP |
| | [Cannabis | | LETLEYIDDIQRLGLSYYFENEIEQNLEQFHNNYQNL |
| | sativa] | | IDFGDNNLYADALCFRLLRQQGYNIACDIEDKYKNEN |
| | | | EKFKESISSDIRGMLNLYEAAQMRVHGEKILDEALIF |
| | | | TTTHLESSVKTCQLSSPYLDLVKHALMHPIRKSLQRR |
| | | | EARLYISLYHQLPSHEEILLILAKLDENLLQKLHQKE |
| | | | LSYITRWWKEFDYKSKHSFIKDRIVECYFWVYGVFFE |
| | | | AETSQIRLIITKLIAILTIIDDAYDSFGTLEELEPFT |
| | | | QAIERWDICAIDTLPEYMKIFYMKLLEIYNEIEQFSK |
| | | | ERSYCPSYAKKGVQSLIRAYFKEAKWLHTKYIPTLEE |
| | | | YMPVGIDSAGSFMLISMVFIGMGDIVTKHSMDWIESN |
| | | | PQPKIIQTMAIVGRVMNDIGYHKSERKKSSGEIVAST |
| | | | VECYMKQYGVTGEEAIEKLSQQVKDSWKDLNEDLLNP |
| | | | ITIPRPLLMQVLKLVRVNHEIYREGDGFTQPTLLKNL |
| | | | IHSLIINPIDE |
| 77 | (−)-kolavenyl | Amino acid | MPSLFSQSLLLPFSQNTNTLSLFHQPKLLPPGASLLE |
| | diphosphate | | AKDKQVNFDRDIRSKCSAISKPRTHDVFQSGGLPVIK |
| | synthase | | WHEIVEDDIDGEEEDTKWTRSNEIEERVASIKSMLES |
| | TPS28. | | MDEGEISISAYDTAWVALVEDIHGSGLPQFPSSLQWI |
| | chloroplastic | | ATHQLSDGSWGDADIFSAHDRLINTLACVVALKSWNL |
| | [Cannabis | | YPEKCQKGMAFFNANISKLERENPEHMPIGFEVAFPS |
| | sativa] | | LLEIARKLNLEVPEDSPVLKVIYARRDEKLTRIPRDI |
| | | | MHTVPTTLLHSLEGMVGLDWEKLLKLQSQDGSFLESP |
| | | | SSTAFALMETKDRNCLQYLTKAVQRENGGVPNVYPVD |
| | | | LFEHLWVADRLQRLGISRFFEPQIEECIDYVERNWTE |
| | | | KGIGWARNSKVEDIDDTAMGERLLRLHGHKVSADVFQ |
| | | | HFKKGDDFFCFRGQSTQAVTGMYNLYRASQLVFPGEK |
| | | | ILEDAMEFSSKFLRKKQASNELLDKWIITKDLPGEVG |
| | | | FALEVPWNANLPRVETRFYIEQYGGQNDVWIGKTLYR |
| | | | MRKVNNDEYLELAKLDYNICQALHSIEWHNLLKWYRD |
| | | | CKLENYGVSRRNLLLAYFLAAASIFEPDRADERLAWA |
| | | | KTAALMQAIQSHEDDQKASSEHRIAFVSAFKRSCNMP |
| | | | SYLITRVSNISDTDHGLLRTLMTTLSHLSLDTMMLYG |
| | | | RDITHHLRQAWEKWLVKWQEGGDGHYEEEAELLIQTI |
| | | | NLSSGRTLVKALLLSNPHYEKLFSTINKVCCKIRHFQ |
| | | | RQRHRAKANQNGEFNRNILTPEIESDMQEVVQLVLQK |
| | | | SSDDMINTKIKQTFLLVAKSFYYAAYCDSKTINFHIG |
| | | | KVIFETVD |
| 78 | 2- | Amino acid | MATAIFSNPKESPTITTTSSKNHYHYQRRTHLLLHDK |
| | isopropylmalate | | VQAFQAASLKLNPNPHYKKPQIVSCQSSGSESPDTEK |
| | synthase 2. | | VRRPEYIPNRISDPNYVRIFDTTLRDGEQSPGAALTS |
| | chloroplastic | | KEKLDIARQLSKLGVDIIEAGFPAASKDDFEAVKIIA |
| | [Cannabis | | KEVGNAVDADGYVPVICGLSRCNENDIRRAWEAVKYA |
| | sativa] | | KRPRIHTFIATSPIHMEYKLRKSKEQVIEIARNMVKF |
| | | | ARSLGCDDVEFSPEDAGRSEREFLYQILGEVIKAGAT |
| | | | TLNIPDTVGYNVPKEFGELIADIKANTPGIENVVIST |
| | | | HCQNDLGLSTANTISGACAGARQLEVTINGIGERAGN |
| | | | ASLEEVVMAIKCRGDQQLGGLYTGINTRHISMTSIMV |
| | | | EEYTGLQVQPHKAIVGANAFAHESGIHQDGMLKHKGT |
| | | | YEIISPEDIGLERSNEAGIVLGKLSGRHALKQQLEEL |
| | | | GYELEDEQLESIFWRFKSVAELKKRITDADLRALVSD |
| | | | EVFQPEVIWKFVDLQVTCGTLGLSTATVKLIGSDGKE |
| | | | HVACSVGTGPVDSAYKAVDLIVKEPVALLEYSMNAVT |
| | | | EGIDAIATTRVLIREETSDLSGHGSTVERVTRTESGN |
| | | | GSGMDIVVSSVKAYIGAINKMLGFKDRTVVNSSEERI |
| | | | PISA |
| 79 | transcription | Amino acid | MGRHSCCYKQKLRKGLWSPEEDEKLLNYITKHGHGCW |
| | factor MYB61 | | SSVPKLAGLQRCGKSCRLRWINYLRPDLKRGPFSQQE |
| | isoform X1 | | ENLIIELHAVLGNRWSQIAAQLPGRTDNEIKNLWNSC |
| | [Cannabis | | IKKKLRQKGIDPNTHKPLSEVENDIGNKLENKGNKAA |
| | sativa] | | TNNNNNENINNSTVRASSIGNLSNDHHHHHHHHLNLA |
| | | | DQSQPSMAAINRYPLLEVSSSTPPTQEFFIEKSTDTR |
| | | | SSPSISSSSPCDESTYFSFHSNNYNTTSSAAAAAAVS |
| | | | HHQDQNNNNNMASFCFNINQNSTRPPQHHHHNQMISN |
| | | | LIQPLQQQVSPSSTTTASSSSPPSNIPRVKPSISLPL |
| | | | LSDHQNNSNSTTTTTTTTTGAVQNWETSTESNNGSSS |
| | | | SSCNIELQGNNNNNNNNFFDHNTNSTAAAAAAAAPNN |
| | | | FSWGLVNESTVGSIKSDDPEDIKWSEYLHSPELLGGG |
| | | | ISNTNNQNSSSSSHLQPILYSNIVKPESHFSNTTTAT |
| | | | GSNPTWHHQNDHHQLQAASSEIMYTNKDLQRLAVAFG |
| | | | QTL |
| 80 | MALD1 | Nucleic acid | ACTAAATTTTGATTACTTTAAAACTGTGGCTATTCTT |
| | promoter | | TAATTATCAGTTGTAAATCTGGCTATTTTTAAAATTA |
| | | | ACTAGTTTTACGCTTGCCAATTTACCACAACTCCTCC |
| | | | TTTGTTAGCATGTAATAATATGAGGGAGCTCACACAG |
| | | | ACCGAGTAAATTCCCTATGAGCAGTGGTGGATCTAGA |
| | | | GGGCAATGTGCGAGTTCCTGGATTGCACAGTCCTTGT |
| | | | ATATATATATTAAAAAATTTATTAAATATCTATAAAT |
| | | | ATTTAATTGTCAACCCAATTACTAACTTGAGCCCGAT |
| | | | CATTATAGAAATTCATAAATTTCAAAGTCTGGAATCG |
| | | | CCTAAGTTGGTCAACTAAAATCTCCTCTTGAGCTACT |
| | | | AATCACTTCTAGAAGAGAGTACACAGCAGTCTCATTT |
| | | | AATGCAACATCAACCTACTGAAAAGGGAAATGATCAG |
| | | | GACCAAAGTTTACTTTAAAGAGTCAAAACTTCATAAA |
| | | | CCGTATAGATAGATAATATAGCTTGACAAAATAGACC |
| | | | CCGTGGTAAAATCATCATTTCAATAGAAATTAGGAAA |
| | | | AGCAATTTGTTTTTTCCTTATCAGATGTCGAGAGTCT |
| | | | ATCGGAAACAATCTTTATATCTTTTCAAGGTAAAGTA |
| | | | AAGCTGCGTACACCGTACCCTCTCCAAACCCCACTTA |
| | | | TGAGATTACGTTGAATTATTATTGTTGTTGTTGTTGT |
| | | | TGTTGATGTCAAGATTAACTTGCTTGTTATCTTTACT |
| | | | ATTTGTTAGATACCTACTATTTTTCATCGACACAAGT |
| | | | AATAAATAATTTTGTCGTAGATTATCCACAAGATAGC |
| | | | TACCTCCTATAAATATTATATGTACCGAATAATTCTC |
| | | | CCCATCATATTTAAGCATATGAAAAAATCACCTATCT |
| | | | TTTTAAATGTTTCATAATTTTCATCTTACTTTATTAA |
| | | | CCGTTAGACCACGCTTTAATCCGTAACTTCACTTGTA |
| | | | TTTAAGAGATTTCAACTCAATAAAAAAGATGACAGGT |
| | | | GGAAAAGGAAAATGTCGGCATGTTTAGTTGAAAAAAA |
| | | | ACTTGTACTGTTATTTTCTGTGGCACATTCAAGATCG |
| | | | TATTCATAGTAATAAAAAGTTTGGTCCTGGCTAGCTG |
| | | | AAAAATTTGGTTAGTAGGGAATGCATGCATGTAAACG |
| | | | CCGCCCTTCATTAAAAGATTGTATGCTACTATATTTT |
| | | | TTGGGATCTTTACGTAAAATAGCTGATCATATTTACT |
| | | | GTTTACTTTTTCTAGCCATATACATAGATCATACATT |
| | | | GATTATATATGATTATGCACATATATTATATAAATCA |
| | | | TGTATATATTATACCTCAACCGGATATTTTTAGTTTT |
| | | | GAGTGGTTAAGTGAGCGGCTATTTGGATTAATTATTC |
| | | | ATATTTTTTTTGGTTTCCCACTTTGTGTCCGGTATCC |
| | | | TCTTTGGAGTTTGATTAATTTGGATTCGTGGTGGAAA |
| | | | ATTCAATTTTGGAGTGTAAAGCTCTCCCTACCAAACA |
| | | | AGGGCGGATTTAGGCCGTATGGTGCACATGTACCCAT |
| | | | GGTTTTTCCGCCAAATAGGTATTTTATGTATATATTT |
| | | | CATAAAATTGATCTAATATTATCTGTTGAGCCCCATG |
| | | | CTCCAAAAAAGTTGAATAGTGCACCTGGTTGAATTCT |
| | | | AAGTTATTCATCCAAAGGAACATGGATAGATTCTAGC |
| | | | TTAGCACTTCTTTTTTCTGGCTTTTTCAATGGTGCAC |
| | | | CTATGATCTAGAAATCCTAGATTCGCCTCTGCTACCA |
| | | | TAGGCGACTCCATACCCAAGGCTCGAAACTGAAATCT |
| | | | CTGATTAAAGATAGAGGAGAGTACTTACCGCTCCAAT |
| | | | ACAACCTTGGTGCCTCGTGGTAGGTTGTATAGTACAC |
| | | | TTTAATAAACAAGAAAGCTTAAGCGTTAAAATTAACT |
| | | | TCATTTCTCAAGCTATAAATACCATCATGAAACAACA |
| | | | CAATTTATACTACAATACACTCCAAGTTTTTTTAGAG |
| | | | GAAAAAAAAAATG |
| 81 | MALD1 | Nucleic acid | TATTAACCGTTAGACCACGCTTTAATCCGTAACTTCA |
| | promoter | | CTTGTATTTAAGAGATTTCAACTCAATAAAAAAGATG |
| | (truncated) | | ACAGGTGGAAAAGGAAAATGTCGGCATGTTTAGTTGA |
| | | | AAAAAAACTTGTACTGTTATTTTCTGTGGCACATTCA |
| | | | AGATCGTATTCATAGTAATAAAAAGTTTGGTCCTGGC |
| | | | TAGCTGAAAAATTTGGTTAGTAGGGAATGCATGCATG |
| | | | TAAACGCCGCCCTTCATTAAAAGATTGTATGCTACTA |
| | | | TATTTTTTGGGATCTTTACGTAAAATAGCTGATCATA |
| | | | TTTACTGTTTACTTTTTCTAGCCATATACATAGATCA |
| | | | TACATTGATTATATATGATTATGCACATATATTATAT |
| | | | AAATCATGTATATATTATACCTCAACCGGATATTTTT |
| | | | AGTTTTGAGTGGTTAAGTGAGCGGCTATTTGGATTAA |
| | | | TTATTCATATTTTTTTTGGTTTCCCACTTTGTGTCCG |
| | | | GTATCCTCTTTGGAGTTTGATTAATTTGGATTCGTGG |
| | | | TGGAAAATTCAATTTTGGAGTGTAAAGCTCTCCCTAC |
| | | | CAAACAAGGGCGGATTTAGGCCGTATGGTGCACATGT |
| | | | ACCCATGGTTTTTCCGCCAAATAGGTATTTTATGTAT |
| | | | ATATTTCATAAAATTGATCTAATATTATCTGTTGAGC |
| | | | CCCATGCTCCAAAAAAGTTGAATAGTGCACCTGGTTG |
| | | | AATTCTAAGTTATTCATCCAAAGGAACATGGATAGAT |
| | | | TCTAGCTTAGCACTTCTTTTTTCTGGCTTTTTCAATG |
| | | | GTGCACCTATGATCTAGAAATCCTTGATTCGCCTCTG |
| | | | CTACCATAGGCGACTCCATACCCAAGGCTCGAAACTG |
| | | | AAATCTCTGATTAAAGATAGAGGAGAGTACTTACCGC |
| | | | TCCAATACAACCTTGGTGCCTCGTGGTAGGTTGTATA |
| | | | GTACACTTTAATAAACAAGAAAGCTTAAGCGTTAAAA |
| | | | TTAACTTCATTTCTCAAGCTATAAATACCATCATGAA |
| | | | ACAACACAATTTATACTACAATACACTCCAAGTTTTT |
| | | | TTAGAGGAAAAAAAAAATG |
| 82 | MALD1 | Nucleic acid | ATGGTGCACATGTACCCATGGTTTTTCCGCCAAATAG |
| | promoter | | GTATTTTATGTATATATTTCATAAAATTGATCTAATA |
| | (truncated) | | TTATCTGTTGAGCCCCATGCTCCAAAAAAGTTGAATA |
| | | | GTGCACCTGGTTGAATTCTAAGTTATTCATCCAAAGG |
| | | | AACATGGATAGATTCTAGCTTAGCACTTCTTTTTTCT |
| | | | GGCTTTTTCAATGGTGCACCTATGATCTAGAAATCCT |
| | | | TGATTCGCCTCTGCTACCATAGGCGACTCCATACCCA |
| | | | AGGCTCGAAACTGAAATCTCTGATTAAAGATAGAGGA |
| | | | GAGTACTTACCGCTCCAATACAACCTTGGTGCCTCGT |
| | | | GGTAGGTTGTATAGTACACTTTAATAAACAAGAAAGC |
| | | | TTAAGCGTTAAAATTAACTTCATTTCTCAAGCTATAA |
| | | | ATACCATCATGAAACAACACAATTTATACTACAATAC |
| | | | ACTCCAAGTTTTTTTAGAGGAAAAAAAAAATG |
| 83 | B3; ARF | Nucleic acid | ATGTCGGCAT |
| | transcription | | |
| | factor binding | | |
| | site | | |
| 84 | bZIP; | Nucleic acid | GAATTATTAT |
| | Homeodomain; | | |
| | HD-ZIP | | |
| | transcription | | |
| | factor binding | | |
| | site | | |
| 85 | Homeodomain; | Nucleic acid | GTTGAATTATTATT |
| | bZIP; HD-ZIP | | |
| | transcription | | |
| | factor binding | | |
| | site | | |
| 86 | Homeodomain; | Nucleic acid | TTTAATTATC |
| | bZIP; HD-ZIP; | | |
| | WOX | | |
| | transcription | | |
| | factor binding | | |
| | site | | |
| 87 | Homeodomain; | Nucleic acid | CATTTAATGC |
| | bZIP; HD-ZIP | | |
| | transcription | | |
| | factor binding | | |
| | site | | |
| 88 | Homeodomain; | Nucleic acid | GATGACAGGT |
| | TALE | | |
| | transcription | | |
| | factor binding | | |
| | site | | |
| 89 | MYB-related | Nucleic acid | TAAATATCTA |
| | transcription | | |
| | factor binding | | |
| | site | | |
| 90 | Sox; YABBY | Nucleic acid | CTTTAATTAT |
| | transcription | | |
| | factor binding | | |
| | site | | |
| 91 | AP2 | Nucleic acid | CACATGTACCCATG |
| | transcription | | |
| | factor binding | | |
| | site | | |
| 92 | Myb/SANT; | Nucleic acid | TAGATTCTAG |
| | G2-like | | |
| | transcription | | |
| | factor binding | | |
| | site | | |
| 93 | MYB; G2-like | Nucleic acid | ATAGATTCTA |
| | transcription | | |
| | factor binding | | |
| | site | | |
| 94 | Circadian motif | Nucleic acid | CAAAGATATC |
| 95 | Unnamed_1 | Nucleic acid | GGATTTTACAGT |
| | motif | | |
| 96 | Unnamed_6 | Nucleic acid | TATAAATATCT |
| | motif | | |
| 97 | TATA-box | Nucleic acid | CCTATAAAAA |
| | motif | | |
| 98 | Circadian motif | Nucleic acid | CAAAGATATC |
| 99 | AT-rich | Nucleic acid | ATAGAAATCAA |
| | element motif | | |
| 100 | TC-rich repeats | Nucleic acid | ATTCTCTAAC |
| | motif | | |
| 101 | GATA-motif | Nucleic acid | AAGGATAAGG |
| 102 | TATA-box | Nucleic acid | TATATAAATC |
| | motif | | |

### BACKGROUND

Glandular trichomes are epidermal outgrowths in plants that are the site of metabolic compound synthesis and storage. Their presence on stem and leaf tissues provides protection for plants against various biotic and abiotic stresses. Glandular trichomes also play a role in the biosynthesis and storage of specialized metabolites.

Metabolites produced and secreted by glandular trichomes are often hydrophobic (e.g., fatty acid derivatives, flavonoids, terpenoids). Terpenoids constitute the largest and most diverse class of plant metabolites. The olefinic backbone of terpenoids is made of multiples of the five-carbon (C) isoprene unit, with the major groups being monoterpenes (10C), sesquiterpenes (15C), and diterpenes (20C). These terpenoids are produced through the condensation of five-carbon isoprene units (dimethylallyl diphosphate [DMAPP] and isopentenyl diphosphate [IPP]) most often by the sequential head-to-tail addition of DMAPP to IPP.

Due to the important role of glandular trichomes in the biosynthesis and secretion of terpenoids, there is a need for the identification of trichome-preferred, or trichome-specific, promoters and associated cis-regulatory elements.

### SUMMARY

In one aspect, this disclosure provides a modified plant, seed, or plant part, comprising a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof.

In one aspect, this disclosure provides a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof.

In one aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 80% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b).

In one aspect, this disclosure provides a method comprising preparing a tobacco product using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof.

In one aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof.

In one aspect, this disclosure provides a method comprising transforming a plant cell with a recombinant nucleic acid molecule, where the recombinant nucleic acid molecule comprises a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof.

In one aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof; and (b) selecting for at least one progeny seed produced in step (a), or a plant germinated therefrom, where the at least one progeny seed or plant germinated therefrom comprises the recombinant nucleic acid molecule.

### BRIEF DESCRIPTION OF THE DRAWINGS

FIG. 1 depicts a tobacco glandular trichome.

FIG. 2 depicts a vector construct used for promoter analysis in tobacco. Each promoter is operably linked to a sequence encoding GREEN FLUORESCENT PROTEIN (G3GFP).

FIG. 3 depicts the phylogenetic analysis of the Rubisco gene family in plants. The figure depicts the split between trichome specific (T-clade) and mesophyll specific (M-clade) Rubsico groups.

FIG. 4 depicts the expression of GREEN FLUORESCENT PROTEIN (G3GFP) driven by the Rubisco RbT 1.2 kb promoter (SEQ ID NO: 10) in a tobacco glandular trichome.

FIG. 5 depicts the expression of GREEN FLUORESCENT PROTEIN (G3GFP) driven by the Rubisco RbT 0.4 kb promoter (SEQ ID NO: 11) in a tobacco glandular trichome.

FIG. 6 depicts the expression of GREEN FLUORESCENT PROTEIN (G3GFP) driven by the NtPSO 1.0 kb promoter (SEQ ID NO: 13) in a tobacco glandular trichome.

FIG. 7 depicts the expression of GREEN FLUORESCENT PROTEIN (G3GFP) driven by the NtPHY 0.5 kb promoter (SEQ ID NO: 15) in a tobacco glandular trichome.

FIG. 8 depicts the expression of GREEN FLUORESCENT PROTEIN (G3GFP) driven by the NtCYC 0.5 kb promoter (SEQ ID NO: 17) in a tobacco glandular trichome.

FIG. 9 depicts a menthol biosynthesis pathway.

FIG. 10 depicts a cis-abienol biosynthesis pathway.

FIG. 11 depicts a cembratrienediol biosynthesis pathway.

FIG. 12 depicts a levopimaric acid biosynthesis pathway.

FIG. 13 depicts a L-leucine biosynthesis pathway.

FIG. 14 depicts a Venn diagram showing which trichome promoters share different promoter motifs.

FIG. 15 comprises FIG. 15A, FIG. 15B, FIG. 15C, and FIG. 15D. FIG. 15A depicts trichomes in young leaf and inflorescence tissue of the wildtype tobacco line Izmir Ego.

FIG. 15B depicts the expression of GREEN FLUORESCENT PROTEIN (G3GFP) driven by the 1971 nucleotide long MALD1 promoter (SEQ ID NO: 80) in young leaf tissue. White arrows point to G3GFP accumulation. FIG. 15C depicts the expression of GREEN FLUORESCENT PROTEIN (G3GFP) driven by the 1055 nucleotide long MALD1 promoter (SEQ ID NO: 81) in young leaf and inflorescence tissue. White arrows point to G3GFP accumulation. FIG. 15D depicts the expression of GREEN FLUORESCENT PROTEIN (G3GFP) driven by the 476 nucleotide long MALD1 promoter (SEQ ID NO: 82). White arrow points to G3GFP accumulation.

FIG. 16 depicts the frequency of cis-elements identified in SEQ ID NO: 80.

FIG. 17 depicts the frequency of cis-elements identified between positions −1055 and −476 of SEQ ID NO: 80, where the final nucleotide position (e.g., the most 3′) of SEQ ID NO: 80 has the position −1. These cis-elements can be found in SEQ ID NO: 81, but not in SEQ ID NO: 82.

FIG. 18 depicts the frequency of cis-elements identified in SEQ ID NO: 82.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

As used herein, the singular form “a,” “an,” and “the” include plural references unless the context clearly dictates otherwise. For example, the term “a compound” or “at least one compound” may include a plurality of compounds, including mixtures thereof.

Any tobacco plant, or part thereof, provided herein is specifically envisioned for use with any method provided herein. Similarly, any modified tobacco plant, or part thereof, is specifically envisioned for use with any method provided herein. Any nucleic acid sequence, amino acid sequence, or other composition provided herein is specifically envisioned for use with any method provided herein.

Any cannabis plant, or part thereof, provided herein is specifically envisioned for use with any method provided herein. Similarly, any modified cannabis plant, or part thereof, is specifically envisioned for use with any method provided herein. Any nucleic acid sequence, amino acid sequence, or other composition provided herein is specifically envisioned for use with any method provided herein.

Trichomes, in general, are hair-like epidermal outgrowths covering most aerial plant tissues. Trichomes tend to be multicellular, but unicellular trichomes are known as well. Multiple types of trichomes can be found on an individual plant, and trichomes vary in shape, size, and cellular organization. An individual trichome can be classified as a glandular trichome or a non-glandular trichome.

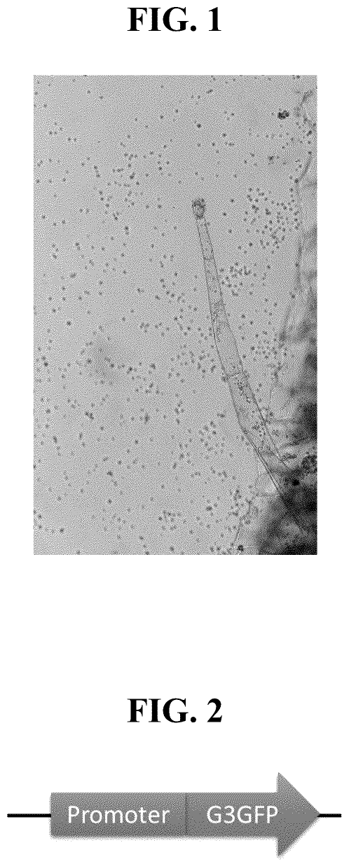

Glandular trichomes (see FIG. 1) are characterized by the presence of a head made of cells that can secrete or store large quantities of specialized metabolites (e.g., terpenes). Within the group of glandular trichomes, a trichome can be further characterized as being peltate or capitate. A capitate glandular trichome typically possesses a stalk with a length that is more than twice the height of the head, and the number of cells in the trichome is highly variable. A peltate trichome is a short-stalked trichome with a large head made of between four and eighteen cells arranged in one or two concentric circles.

In an aspect, a trichome is a glandular trichome. In an aspect, a glandular trichome is a capitate glandular trichome. In an aspect, a glandular trichome is a peltate glandular trichome. In an aspect, a glandular trichome is selected from the group consisting of a capitate glandular trichome and a peltate glandular trichome.

In an aspect, this disclosure provides a modified plant, seed, or plant part comprising a recombinant nucleic acid molecule comprising a trichome-specific promoter operably linked to a heterologous polynucleotide. In another aspect, this disclosure provides a modified plant, seed, or plant part comprising a recombinant nucleic acid molecule comprising a glandular trichome-specific promoter operably linked to a heterologous polynucleotide. In an aspect, this disclosure provides a modified plant, seed, or plant part comprising a recombinant nucleic acid molecule comprising a capitate glandular trichome-specific promoter operably linked to a heterologous polynucleotide. In an aspect, this disclosure provides a modified plant, seed, or plant part comprising a recombinant nucleic acid molecule comprising a peltate glandular trichome-specific promoter operably linked to a heterologous polynucleotide.

In an aspect, this disclosure provides a modified plant, seed, or plant part comprising a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 85% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 90% identical to a sequence selected from the group consisting of SEQ ID NOS: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 92.5% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 95% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 96% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 97% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 98% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 99% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence at least 99.5% identical to a sequence selected from the group consisting of SEQ ID NOs: 10-17, 45-47, and 80-82, or a functional fragment thereof. In an aspect, this disclosure provides a modified plant, seed, or plant part comprising a recombinant nucleic acid molecule comprising a promoter operably linked to a heterologous polynucleotide, where the promoter comprises a nucleic acid sequence 100% identical to a sequence selected from the group consisting of SEQ ID NOs: 10-17, 45-47, and 80-82, or a functional fragment thereof.

As commonly understood in the art, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

### Promoters

Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. As a non-limiting example, a trichome tissue-preferred promoter exhibits higher activity in trichomes, but may also exhibit activity, albeit at lower levels, in additional tissues such as stem, leaves, and floral tissues. A “tissue-specific” promoter causes expression only in a specific tissue. As a non-limiting example, a trichome tissue-specific promoter drives expression only in trichomes. In an aspect, a tissue-specific promoter is a trichome tissue-specific promoter. In another aspect, a tissue-preferred promoter is a trichome tissue-preferred promoter. In an aspect, a trichome tissue-specific promoter is selected from the group consisting of SEQ ID NOs: 10-17, 45-47, and 80-82, or a functional fragment thereof. In an aspect, a trichome tissue-preferred promoter is selected from the group consisting of SEQ ID NOs: 10-17, 45-47, and 80-82, or a functional fragment thereof.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. In an aspect, a promoter provided herein is operably linked to a heterologous nucleic acid molecule.

In an aspect, a promoter comprises a sequence at least 70% identical to a sequence selected from the group consisting of SEQ ID NOs: 10-17, 45-47, and 80-82. In an aspect, a promoter comprises a sequence at least 75% identical to a sequence selected from the group consisting of SEQ ID NOs: 10-17, 45-47, and 80-82. In an aspect, a promoter comprises a sequence at least 80% identical to a sequence selected from the group consisting of SEQ ID NOs: 10-17, 45-47, and 80-82. In an aspect, a promoter comprises a sequence at least 85% identical to a sequence selected from the group consisting of SEQ ID NOs: 10-17, 45-47, and 80-82. In an aspect, a promoter comprises a sequence at least 90% identical to a sequence selected from the group consisting of SEQ ID NOs: 10-17, 45-47, and 80-82. In an aspect, a promoter comprises a sequence at least 92.5% identical to a sequence selected from the group consisting of SEQ ID NOs: 10-17, 45-47, and 80-82. In an aspect, a promoter comprises a sequence at least 95% identical to a sequence selected from the group consisting of SEQ ID NOs: 10-17, 45-47, and 80-82. In an aspect, a promoter comprises a sequence at least 96% identical to a sequence selected from the group consisting of SEQ ID NOs: 10-17, 45-47, and 80-82. In an aspect, a promoter comprises a sequence at least 97% identical to a sequence selected from the group consisting of SEQ ID NOs: 10-17, 45-47, and 80-82. In an aspect, a promoter comprises a sequence at least 98% identical to a sequence selected from the group consisting of SEQ ID NOs: 10-17, 45-47, and 80-82. In an aspect, a promoter comprises a sequence at least 99% identical to a sequence selected from the group consisting of SEQ ID NOs: 10-17, 45-47, and 80-82. In an aspect, a promoter comprises a sequence 100% identical to a sequence selected from the group consisting of SEQ ID NOs: 10-17, 45-47, and 80-82.

It is appreciated in the art that a fragment of a promoter sequence can function to drive transcription of an operably linked nucleic acid molecule. For example, without being limiting, if a 1000 bp promoter is truncated to 500 bp, and the 500 bp fragment is capable of driving transcription, the 500 bp fragment is referred to as a “functional fragment.” As non-limiting examples, SEQ ID NO: 11 can be considered a functional fragment of SEQ ID NO: 10; SEQ ID NO: 13 can be considered a functional fragment of SEQ ID NO: 12; SEQ ID NO: 15 can be considered a functional fragment of SEQ ID NO: 14; and SEQ ID NO: 17 can be considered a functional fragment of SEQ ID NO: 16.

In an aspect, a trichome-preferred promoter comprises at least one regulatory element motif selected from the group consisting of TATA, CGTGG, TATATAAA, CATTTG, CAAAT, CAAT, CTCC, TAACCA, CAACAG, and SEQ ID NOs: 41 and 42. In an aspect, a trichome-preferred promoter comprises at least two different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-preferred promoter comprises at least three different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-preferred promoter comprises at least four different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-preferred promoter comprises at least five different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-preferred promoter comprises at least six different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-preferred promoter comprises at least seven different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-preferred promoter comprises at least eight different regulatory element motifs selected from the group consisting of TATA, CGTGG, TATATAAA, CATTTG, CAAAT, CAAT, CTCC, TAACCA, CAACAG, and SEQ ID NOs: 41 and 42.

In an aspect, a trichome-specific promoter comprises at least one regulatory element motif selected from the group consisting of TATA, CGTGG, TATATAAA, CATTTG, CAAAT, CAAT, CTCC, TAACCA, CAACAG, and SEQ ID NOs: 41 and 42. In an aspect, a trichome-specific promoter comprises at least two different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-specific promoter comprises at least three different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-specific promoter comprises at least four different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-specific promoter comprises at least five different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-specific promoter comprises at least six different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-specific promoter comprises at least seven different regulatory element motifs selected from the group consisting of TATA, CGTGG, TATATAAA, CATTTG, CAAAT, CAAT, CTCC, TAACCA, CAACAG, and SEQ ID NOs: 41 and 42. In an aspect, a trichome-specific promoter comprises at least eight different regulatory element motifs selected from the group consisting of TATA, CGTGG, TATATAAA, CATTTG, CAAAT, CAAT, CTCC, TAACCA, CAACAG, and SEQ ID NOs: 41 and 42.

### Plants

In an aspect, a plant provided herein is a modified plant. In an aspect, a seed provided herein is a modified seed. In an aspect, a plant part provided herein is a modified plant part. As used herein, “modified,” in the context of a plant, seed, or plant part, refers to a plant, seed, or plant part, comprising a genetic alteration introduced for certain purposes and beyond natural polymorphisms. Without being limiting, a modified plant, seed, or plant part comprises a recombinant nucleic acid molecule. In another aspect, a modified plant, seed, or plant part comprises a genetic modification. In an aspect, a modified plant, seed, or plant part is a transgenic plant, seed, or plant part.

In an aspect, a plant is a tobacco plant. In an aspect, a plant is a Nicotiana plant. In an aspect, a tobacco plant is a Nicotiana tabacum plant.

In an aspect, a Nicotiana plant, seed, or plant part is selected from the group consisting of Nicotiana tabacum, Nicotiana amplexicaulis PI 271989; Nicotiana benthamiana PI 555478; Nicotiana bigelovii PI 555485; Nicotiana debneyi; Nicotiana excelsior PI 224063; Nicotiana glutinosa PI 555507; Nicotiana goodspeedii PI 241012; Nicotiana gossei PI 230953; Nicotiana hesperis PI 271991; Nicotiana knightiana PI 555527; Nicotiana maritima PI 555535; Nicotiana megalosiphon PI 555536; Nicotiana nudicaulis PI 555540; Nicotiana paniculata PI 555545; Nicotiana plumbaginifolia PI 555548; Nicotiana repanda PI 555552; Nicotiana rustica; Nicotiana suaveolens PI 230960; Nicotiana sylvestris PI 555569; Nicotiana tomentosa PI 266379; Nicotiana tomentosiformis; and Nicotiana trigonophylla PI 555572.

In an aspect, a seed is a tobacco seed. In an aspect, a seed is a Nicotiana seed. In an aspect, a tobacco seed is a Nicotiana tabacum seed.

In an aspect, a plant part is a tobacco plant part. In an aspect, a plant part is a Nicotiana plant part. In an aspect, a tobacco plant part is a Nicotiana tabacum plant part.

In an aspect, a plant is a cannabis plant. In an aspect, a plant is a Cannabis plant. In an aspect, a cannabis plant is a Cannabis sativa plant. In an aspect, a cannabis plant is a Cannabis indica plant. In an aspect, a cannabis plant is a Cannabis ruderalis plant. In an aspect, a cannabis plant is selected from the group consisting of Cannabis sativa, Cannabis indica, and Cannabis ruderalis.

In an aspect, a seed is a cannabis seed. In an aspect, a seed is a Cannabis seed. In an aspect, a cannabis seed is a Cannabis sativa seed. In an aspect, a cannabis seed is a Cannabis indica seed. In an aspect, a cannabis seed is a Cannabis ruderalis seed. In an aspect, a cannabis seed is selected from the group consisting of Cannabis sativa, Cannabis indica, and Cannabis ruderalis.

In an aspect, a plant part is a cannabis plant part. In an aspect, a plant part is a Cannabis plant part. In an aspect, a cannabis plant part is a Cannabis sativa plant part. In an aspect, a cannabis plant part is a Cannabis indica plant part. In an aspect, a cannabis plant part is a Cannabis ruderalis plant part. In an aspect, a cannabis plant part is selected from the group consisting of Cannabis sativa, Cannabis indica, and Cannabis ruderalis.

In an aspect, a plant part provided includes, but is not limited to, a leaf, a stem, a root, a trichome, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In an aspect, a plant part does not include a seed. In an aspect, this disclosure provides plant cells, tissues, and organs that are not reproductive material and do not mediate the natural reproduction of the plant. In another aspect, this disclosure also provides plant cells, tissues, and organs that are reproductive material and mediate the natural reproduction of the plant. In another aspect, this disclosure provides plant cells, tissues, and organs that cannot maintain themselves via photosynthesis. In another aspect, this disclosure provides somatic plant cells. Somatic cells, contrary to germline cells, do not mediate plant reproduction.

Cells, tissues and organs can be from seed, fruit, leaf, cotyledon, hypocotyl, meristem, embryos, endosperm, root, shoot, stem, trichome, pod, flower, inflorescence, stalk, pedicel, style, stigma, receptacle, petal, sepal, pollen, anther, filament, ovary, ovule, pericarp, phloem, vascular tissue. In another aspect, this disclosure provides a plant chloroplast. In a further aspect, this disclosure provides epidermal cells, stomata cell, leaf or root hairs, a storage root, or a tuber. In another aspect, this disclosure provides a tobacco protoplast.

Skilled artisans understand that tobacco and cannabis plants naturally reproduce via seeds, not via asexual reproduction or vegetative propagation. In an aspect, this disclosure provides plant endosperm.

This disclosure provides cells from plants provided herein.

As used herein, a “progeny plant” or “progeny seed” can be from any filial generation, e.g., F₁, F₂, F₃, F₄, F₅, F₆, F₇, etc.

In an aspect, a tobacco plant, seed, or plant part, is of a tobacco variety selected from the group consisting of a flue-cured variety, a bright variety, a Burley variety, a Virginia variety, a Maryland variety, a dark variety, a Galpão variety, an Oriental variety, and a Turkish variety.

In an aspect, a tobacco cell is of a tobacco variety selected from the group consisting of a flue cured variety, a bright variety, a Burley variety, a Virginia variety, a Maryland variety, a dark variety, a Galpão variety, an Oriental variety, and a Turkish variety.

In an aspect, a tobacco leaf is of a tobacco variety selected from the group consisting of a flue cured variety, a bright variety, a Burley variety, a Virginia variety, a Maryland variety, a dark variety, a Galpão variety, an Oriental variety, and a Turkish variety.

In an aspect, a cured tobacco leaf or plant part is of a tobacco variety selected from the group consisting of a flue cured variety, a bright variety, a Burley variety, a Virginia variety, a Maryland variety, a dark variety, a Galpão variety, an Oriental variety, and a Turkish variety. Skilled artisans further understand that cured tobacco does not constitute a living organism and is not capable of growth or reproduction

Flue-cured tobaccos (also called “Virginia” or “bright” tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the United States of America. In one aspect, tobacco plants, seeds, or plant parts provided herein are of a flue-cured tobacco variety selected from the group consisting of the varieties listed in Table 2, and any variety essentially derived from any one of the foregoing varieties. See WO 2004/041006 A1. In a further aspect, tobacco plants, seeds, or plant parts provided herein are in a flue-cured variety selected from the group consisting of K326, K346, and NC196.

TABLE 2
Flue-cured Tobacco Varieties

400 (TC 225)
401 (TC 226)
401 Cherry Red (TC 227)
401 Cherry Red Free (TC 228)
Cash (TC 250)
Cash (TI 278)
CC 101
CC 1063
CC 13
CC 143
CC 200
CC 27
CC 301
CC 33
CC 35
CC 37
CC 400
CC 500
CC 600
CC 65
CC 67
CC 700
CC 800
CC 900
Coker 139 (TC 259)
Coker 139 yb1, yb2
Coker 140 (TC 260)
Coker 176 (TC 262)
Coker 187 (TC 263)
Coker 187-Hicks (TC 265)
Coker 209 (TC 267)
Coker 258 (TC 270)
Coker 298 (TC 272)
Coker 316 (TC 273)
Coker 319 (TC 274)
Coker 347 (TC 275)
Coker 371-Gold (TC 276)
Coker 411 (TC 277)
Coker 48 (TC 253)
Coker 51 (TC 254)
Coker 86 (TC 256)
CU 263 (TC 619)
CU 561
DH95-1562-1
Dixie Bright 101 (TC 290)
Dixie Bright 102 (TC 291)
Dixie Bright 244 (TC 292)
Dixie Bright 27 (TC 288)
Dixie Bright 28 (TC 289)
GF 157
GF 318
GL 26H
GL 338
GL 350
GL 368
GL 395
GL 600
GL 737
GL 939
GL 939 (TC 628)
Hicks (TC 310)
Hicks Broadleaf (TC 311)
K 149 (TC 568)
K 317
K 326
K 326 (TC 319)
K 340 (TC 320)
K 346
K 346 (TC 569)
K 358
K 394 (TC 321)
K 399
K 399 (TC 322)
K 730
Lonibow (TI 1573)
Lonibow (TI 1613)
McNair 10 (TC 330)
McNair 135 (TC 337)
McNair 30 (TC 334)
McNair 373 (TC 338)
McNair 944 (TC 339)
MK94 (TI 1512)
MS K 326
MS NC 71
MS NC 72
NC 100
NC 102
NC 1071 (TC 364)
NC 1125-2
NC 12 (TC 346)
NC 1226
NC 196
NC 2326 (TC 365)
NC 27 NF (TC 349)
NC 291
NC 297
NC 299
NC 37 NF (TC 350)
NC 471
NC 55
NC 567 (TC 362)
NC 60 (TC 352)
NC 606
NC 6140
NC 71
NC 72
NC 729 (TC 557)
NC 810 (TC 659)
NC 82 (TC 356)
NC 8640
NC 89 (TC 359)
NC 92
NC 925
NC 95 (TC 360)
NC 98 (TC 361)
NC EX 24
NC PY 10 (TC 367)
NC TG 61
Oxford 1 (TC 369)
Oxford 1-181 (TC 370)
Oxford 2 (TC 371)
Oxford 207 (TC 632)
Oxford 26 (TC 373)
Oxford 3 (TC 372)
Oxford 414 NF
PD 611 (TC 387)
PVH 03
PVH 09
PVH 1118
PVH 1452
PVH 1600
PVH 2110
PVH 2275
R 83 (Line 256-1) (TI 1400)
Reams 134
Reams 158
Reams 713
Reams 744
Reams M1
RG 11 (TC 600)
RG 13 (TC 601)
RG 17 (TC 627)
RG 22 (TC 584)
RG 8 (TC 585)
RG 81 (TC 618)
RG H51
RG4H 217
RGH 12
RGH 4
RGH 51
RGH 61
SC 58 (TC 400)
SC 72 (TC 403)
Sp. G-168
SPEIGHT 168
Speight 168 (TC 633)
Speight 172 (TC 634)
Speight 178
Speight 179
Speight 190
Speight 196
SPEIGHT 220
SPEIGHT 225
SPEIGHT 227
SPEIGHT 236
Speight G-10 (TC 416)
Speight G-102
Speight G-108
Speight G-111
Speight G-117
Speight G-126
Speight G-15 (TC 418)
Speight G-23
Speight G-28 (TC 420)
Speight G-33
Speight G-41
Speight G-5
Speight G-52
Speight G-58
Speight G-70
Speight G-70 (TC 426)
Speight G-80 (TC 427)
Speight NF3 (TC 629)
STNCB
VA 182
VA 45 (TC 559)
Vesta 30 (TC 439)
Vesta 33 (TC 440)
Vesta 5 (TC 438)
Vesta 62 (TC 441)
Virginia (TI 220)
Virginia (TI 273)
Virginia (TI 877)
Virginia 115 (TC 444)
Virginia 21 (TC 443)
Virginia Bright (TI 964)
Virginia Bright Leaf (TC 446)
Virginia Gold (TC 447)
White Stem Orinoco (TC 451)

Air-cured tobaccos include “Burley,” “Maryland,” and “dark” tobaccos. The common factor linking air-cured tobaccos is that curing occurs primarily without artificial sources of heat and humidity. Burley tobaccos are light to dark brown in color, high in oil, and low in sugar. Burley tobaccos are typically air-cured in barns. Major Burley growing countries include Argentina, Brazil, Italy, Malawi, and the United States of America.

Maryland tobaccos are extremely fluffy, have good burning properties, low nicotine and a neutral aroma. Major Maryland growing countries include the United States of America and Italy.

In one aspect, tobacco plants, seeds, or plant parts provided herein are of a Burley tobacco variety selected from the group consisting of the tobacco varieties listed in Table 3, and any variety essentially derived from any one of the foregoing varieties. In a further aspect, tobacco plants, seeds, or plant parts provided herein are in a Burley variety selected from the group consisting of TN 90, KT 209, KT 206, KT212, and HB 4488.

TABLE 3
Burley Tobacco Varieties

4407 LC
AA-37-1
Burley 21 (TC 7)
Burley 49 (TC 10)
Burley 64 (TC 11)
Burley Mammoth KY 16 (TC 12)
Clay 402
Clay 403
Clay 502
Clays 403
GR 10 (TC 19)
GR 10 (TC 19)
GR 10A (TC 20)
GR 13 (TC 21)
GR 14 (TC 22)
GR 149 LC
GR 153
GR 17 (TC 23)
GR 17B (TC 24)
GR 18 (TC 25)
GR 19 (TC 26)
GR 2 (TC 15)
GR 24 (TC 27)
GR 36 (TC 28)
GR 38 (TC 29)
GR 38A (TC 30)
GR 40 (TC 31)
GR 42 (TC 32)
GR 42C (TC 33)
GR 43 (TC 34)
GR 44 (TC 35)
GR 45 (TC 36)
GR 46 (TC 37)
GR 48 (TC 38)
GR 5 (TC 16)
GR 53 (TC 39)
GR 6 (TC 17)
GR 9 (TC 18)
GR139 NS
GR139 S
HB 04P
HB 04P LC
HB 3307P LC
HB 4108P
HB 4151P
HB 4192P
HB 4194P
HB 4196
HB 4488
HB 4488P
HB04P
HB 4488 LC
HIB 21
HPB 21
HY 403
Hybrid 403 LC
Hybrid 404 LC
Hybrid 501 LC
KDH-959 (TC 576)
KDH-960 (TC 577)
KT 200 LC
KT 204 LC
KT 206 LC
KT 209 LC
KT 210 LC
KT 212 LC
KT 215 LC
KY 1 (TC 52)
KY 10 (TC 55)
KY 12 (TC 56)
KY 14 (TC 57)
KY 14 × L8 LC
KY 15 (TC 58)
KY 16 (TC 59)
KY 17 (TC 60)
KY 19 (TC 61)
KY 21 (TC 62)
KY 22 (TC 63)
KY 24 (TC 64)
KY 26 (TC 65)
KY 33 (TC 66)
KY 34 (TC 67)
KY 35 (TC 68)
KY 41A (TC 69)
KY 5 (TC 53)
KY 52 (TC 70)
KY 54 (TC 71)
KY 56 (TC 72)
KY 56 (TC 72)
KY 57 (TC 73)
KY 58 (TC 74)
KY 8654 (TC 77)
KY 8959
KY 9 (TC 54)
KY 907 LC
KY 908 (TC 630)
NBH 98 (Screened)
NC 1206
NC 129
NC 2000 LC
NC 2002 LC
NC 3 LC
NC 5 LC
NC 6 LC
NC 7 LC
NC BH 129 LC
NC03-42-2
Newton 98
R 610 LC
R 630 LC
R 7-11
R 7-12 LC
RG 17
TKF 1801 LC
TKF 2002 LC
TKF 4024 LC
TKF 4028 LC
TKF 6400 LC
TKF 7002 LC
TKS 2002 LC
TN 86 (TC 82)
TN 90 LC
TN 97 Hybrid LC
TN 97 LC
VA 116
VA 119
Virgin A Mutante (TI 1406)
Virginia 509 (TC 84)

In another aspect, tobacco plants, seeds, or plant parts provided herein are of a Maryland tobacco variety selected from the group consisting of the tobacco varieties listed in Table 4, and any variety essentially derived from any one of the foregoing varieties.

TABLE 4
Maryland Tobacco Varieties

Maryland 10 (TC 498)
Maryland 14 D2 (TC 499)
Maryland 201 (TC 503)
Maryland 21 (TC 500)
Maryland 341 (TC 504)
Maryland 40
Maryland 402
Maryland 59 (TC 501)
Maryland 601
Maryland 609 (TC 505)
Maryland 64 (TC 502)
Maryland 872 (TC 506)
Maryland Mammoth (TC 507)

Dark air-cured tobaccos are distinguished from other tobacco types primarily by its curing process, which gives dark air-cured tobacco its medium-brown to dark-brown color and a distinct aroma. Dark air-cured tobaccos are mainly used in the production of chewing tobacco and snuff. In one aspect, tobacco plants, seeds, or plant parts provided herein are of a dark air-cured tobacco variety selected from the group consisting of Sumatra, Jatim, Dominican Cubano, Besuki, One sucker, Green River, Virginia sun-cured, and Paraguan Passado, and any variety essentially derived from any one of the foregoing varieties.

Dark fire-cured tobaccos are generally cured with low-burning wood fires on the floors of closed curing barns. Dark fire-cured tobaccos are typically used for making pipe blends, cigarettes, chewing tobacco, snuff, and strong-tasting cigars. Major growing regions for dark fire-cured tobaccos are Tennessee, Kentucky, and Virginia in the United States of America. In one aspect, tobacco plants, seeds, or plant parts provided herein are of a dark fire-cured tobacco variety selected from the group consisting of the tobacco varieties listed in Table 5, and any variety essentially derived from any one of the foregoing varieties.

TABLE 5
Dark Fire-Cured Tobacco Varieties

Black Mammoth (TC 461)
Black Mammoth Small Stalk (TC 641)
Certified Madole (TC 463)
D-534-A-1 (TC 464)
DAC ULT 302
DAC ULT 303
DAC ULT 306
DAC ULT 308
DAC ULT 312
DF 300 (TC 465)
DF 485 (TC 466)
DF 516 (TC 467)
DF 911 (TC 468)
DT 508
DT 518 (Screened)
DT 538 LC
DT 592
Improved Madole (TC 471)
Jernigan's Madole (TC 472)
KT 14LC
KT D17LC
KT D4 LC
KT D6 LC
KT D8 LC
KY 153 (TC 216)
KY 157 (TC 217)
KY 160
KY 160 (TC 218)
KY 163 (TC 219)
KY 165 (TC 220)
KY 170 (TC 474)
KY 171 (PhPh)
KY 171 (TC 475)
KY 171 LC
KY 171 NS
KY 180 (TC 573)
KY 190 (TC 574)
Little Crittenden
Little Crittenden (TC 476)
Little Crittenden LC (certified)
Little Crittenden PhPh
Lizard Tail Turtle Foot
Madole (TC 478)
Madole (TC 479)
MS KY 171
MS NL Madole LC
MS TN D950 LC
Nance (TC 616)
Narrow Leaf Madole LC (certified)
Neal Smith Madole (TC 646)
Newtons VH Madole
NL Madole
NL Madole (PhPh)
NL Madole (TC 484)
NL Madole LC
NL Madole LC (PhPh)
NL Madole NS
One Sucker (TC 224)
OS 400
PD 302H
PD 312H
PD 318H
PD 7302 LC
PD 7305
PD 7309 LC
PD 7312 LC
PD 7318 LC
PD 7319 LC
Petico M PG04
PY KY 160 (TC 612)
PY KY 171 (TC 613)
Shirey
TI 1372
TN D94
TN D94 (TC 621)
TN D950
TN D950 (PhPh)
TN D950
TN D950 (TC 622)
TR Madole (TC 486)
VA 309
VA 309 (TC 560)
VA 309 LC (certified)
VA 310 (TC 487)
VA 331 (TC 592)
VA 355 (TC 638)
VA 359
VA 359 (Screened)
VA 359 (TC 639)
VA 359 LC (certified)
VA 403 (TC 580)
VA 405 (TC 581)
VA 409 (TC 562)
VA 510 (TC 572)

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant size, small leaf size, and unique aroma properties of Oriental tobacco varieties are a result of their adaptation to the poor soil and stressful climatic conditions in which they have been developed. In one aspect, tobacco plants, seeds, or plant parts provided herein are of an Oriental tobacco variety selected from the group consisting of the tobacco varieties listed in Table 6, and any variety essentially derived from any one of the foregoing varieties.

TABLE 6
Oriental Tobacco Varieties

Bafra (TI 1641)
Bahce (TI 1730)
Bahia (TI 1416)
Bahia (TI 1455)
Baiano (TI 128)
Basma
Basma (TI 1666)
Basma Drama
Basma Hybrid (PhPh)
Basma Zihna I
Bitlis (TI 1667)
Bitlis (TI 1725)
Bubalovac (TI 1282)
Bursa (TI 1650)
Bursa (TI 1668)
Canik (TI 1644)
Djebel 174 (TI 1492)
Djebel 359 (TI 1493)
Djebel 81
Dubec 566 (TI 1409)
Dubec 7 (TI 1410)
Dubek 566 (TI 1567)
Duzce (TI 1670)
Edirne (TI 1671)
Ege (TI 1642)
Ege-64 (TI 1672)
Izmir (Akhisar) (TI 1729)
Izmir (Gavurkoy) (TI 1727)
Izmir Ege 64
Izmir-Incekara (TI 1674)
Izmir-Ozbas (TI 1675)
Jaka Dzebel (TI 1326)
Kaba-Kulak
Kagoshima Maruba (TI 158)
Katerini
Katerini S53
Krumovgrad 58
MS Basma
MS Katerini S53
Nevrokop 1146
Ozbas (TI 1645)
Perustitza (TI 980)
Prilep (TI 1291)
Prilep (TI 1325)
Prilep 12-2/1
Prilep 23
Samsun (TC 536)
Samsun 959 (TI 1570)
Samsun Evkaf (TI 1723)
Samsun Holmes NN (TC 540)
Samsun Maden (TI 1647)
Samsun NO 15 (TC 541)
Samsun-BLK SHK Tol (TC 542)
Samsun-Canik (TI 1678)
Samsun-Maden (TI 1679)
Saribaptar 407 - Izmir Region
Smyrna (TC 543)
Smyrna No. 23 (TC 545)
Smyrna No. 9 (TC 544)
Smyrna-Blk Shk Tol (TC 546)
Trabzon (TI 1649)
Trabzon (TI 1682)
Trapezund 161 (TI 1407)
Turkish (TC 548)
Turkish Angshit (TI 90)
Turkish Samsum (TI 92)
Turkish Tropizoid (TI 93)
Turkish Varotic (TI 89)
Xanthi (TI 1662)

In an aspect, tobacco plants, seeds, or plant parts provided herein are of a cigar tobacco variety selected from the group consisting of the tobacco varieties listed in Table 7, and any variety essentially derived from any one of the foregoing varieties.

TABLE 7
Cigar Tobacco Varieties

Bahai (TI 62)
Beinhart 1000
Beinhart 1000 (TI 1562)
Beinhart 1000-1 (TI 1561)
Bergerac C
Bergerac C (TI 1529)
Big Cuban (TI 1565)
Castillo Negro, Blanco, Pina (TI 448)
Castillo Negro, Blanco, Pina (TI 448A)
Castillo Negro, Blanco, Pina (TI 449)
Caujaro (TI 893)
Chocoa (TI 289)
Chocoa (TI 313)
Connecticut 15 (TC 183)
Connecticut Broadleaf
Connecticut Broadleaf (TC 186)
Connecticut Shade (TC 188)
Criollo, Colorado (TI 1093)
Enshu (TI 1586)
Florida 301
Florida 301 (TC 195)
PA Broadleaf (TC 119)
Pennsylvania Broadleaf
Pennsylvania Broadleaf (TC 119)
Petite Havana SR1
Petite Havana SR1 (TC 105)

In an aspect, tobacco plants, seeds, or plant parts provided herein are of a tobacco variety selected from the group consisting of the tobacco varieties listed in Table 8, and any variety essentially derived from any one of the foregoing varieties.

TABLE 8
Other Tobacco Varieties

Chocoa (TI 319)
Hoja Parada (TI 1089)
Hoja Parado (Galpoa) (TI 1068)
Perique (St. James Parrish)
Perique (TC 556)
Perique (TI 1374)
Sylvestris (TI 984)
TI 179

In an aspect, a tobacco plant or plant part is from a variety selected from the group consisting of the tobacco varieties listed in Table 2, Table 3, Table 4, Table 5, Table 6, Table 7, and Table 8. In another aspect, a tobacco plant or plant part is from a variety listed in Table 2. In another aspect, a tobacco plant or plant part is from a variety listed in Table 3. In another aspect, a tobacco plant or plant part is from a variety listed in Table 4. In another aspect, a tobacco plant or plant part is from a variety listed in Table 5. In another aspect, a tobacco plant or plant part is from a variety listed in Table 6. In another aspect, a tobacco plant or plant part is from a variety listed in Table 7. In another aspect, a tobacco plant or plant part is from a variety listed in Table 8.

In an aspect, a tobacco seed is from a variety selected from the group consisting of the tobacco varieties listed in Table 2, Table 3, Table 4, Table 5, Table 6, Table 7, and Table 8. In another aspect, a tobacco seed is from a variety listed in Table 2. In another aspect, a tobacco seed is from a variety listed in Table 3. In another aspect, a tobacco seed is from a variety listed in Table 4. In another aspect, a tobacco seed is from a variety listed in Table 5. In another aspect, a tobacco seed is from a variety listed in Table 6. In another aspect, a tobacco seed is from a variety listed in Table 7. In another aspect, a tobacco seed is from a variety listed in Table 8.

In an aspect, a tobacco cell is from a variety selected from the group consisting of the tobacco varieties listed in Table 2, Table 3, Table 4, Table 5, Table 6, Table 7, and Table 8. In another aspect, a tobacco cell is from a variety listed in Table 2. In another aspect, a tobacco cell is from a variety listed in Table 3. In another aspect, a tobacco cell is from a variety listed in Table 4. In another aspect, a tobacco cell is from a variety listed in Table 5. In another aspect, a tobacco cell is from a variety listed in Table 6. In another aspect, a tobacco cell is from a variety listed in Table 7. In another aspect, a tobacco cell is from a variety listed in Table 8.

All foregoing mentioned specific varieties of flue-cured, dark air-cured, Burley, Maryland, dark fire-cured, cigar, or Oriental type are listed only for exemplary purposes. Any additional flue-cured, dark air-cured, Burley, Maryland, dark fire-cured, cigar, or Oriental varieties are also contemplated in the present application.

In an aspect, a plant or variety provided herein is an inbred plant or variety. As used herein, an “inbred” variety is a variety that has been bred for genetic homogeneity.

As used herein, a “hybrid” is created by crossing two plants from different varieties or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an E×F hybrid. The first and second hybrids can be further crossed to create the higher order hybrid (C×D)×(E×F) comprising genetic information from all four parent varieties. In an aspect, a plant or seed provided herein is a hybrid plant or seed.

In an aspect, a tobacco plant provided herein is an inbred tobacco plant. In an aspect, a tobacco seed provided herein is an inbred tobacco seed. In an aspect, a tobacco plant provided herein is a hybrid tobacco plant. In another aspect, a tobacco seed provided herein is a hybrid tobacco seed.

In an aspect, a cannabis plant provided herein is an inbred cannabis plant. In an aspect, a cannabis seed provided herein is an inbred cannabis seed. In an aspect, a cannabis plant provided herein is a hybrid cannabis plant. In an aspect, a cannabis seed provided herein is a hybrid cannabis seed.

Unless specified otherwise, all comparisons to control plants require similar growth conditions or comparable growth conditions for the two plants being compared. As used herein, “grown under comparable conditions,” “similar growth conditions” or “comparable growth conditions” refer to similar environmental conditions and/or agronomic practices for growing and making meaningful comparisons between two or more plant genotypes so that neither environmental conditions nor agronomic practices would contribute to or explain any difference observed between the two or more plant genotypes. Environmental conditions include, for example, light, temperature, water (humidity), and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp 70-103. As used herein, a “control plant” refers to a plant of identical, or nearly identical, genetic makeup as the modified plant being compared, except for the non-natural mutation or recombinant DNA construct provided herein that was introduced to the modified plant.

In an aspect, a plant or variety provided herein is male sterile. In another aspect, a plant or variety provided herein is cytoplasmic male sterile (CMS). Male sterile plants can be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fehr (ed.), MacMillan Publishing Co., Inc., New York, N.Y. 761 pp.

In another aspect, a plant or variety provided herein is female sterile. As a non-limiting example, female sterile plants can be made by mutating the STIG1 gene. See, for example, Goldman et al. 1994, EMBO Journal 13:2976-2984.

In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 85% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 90% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 92.5% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 95% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 96% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 97% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 98% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 99% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence at least 99.9% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous polynucleotide, where the promoter comprises a nucleic acid sequence 100% identical to a sequence selected from the group consisting of SEQ ID NOs: 10-17, 45-47, and 80-82, or a functional fragment thereof; and (b) selecting at least one progeny seed produced in step (a), or a plant germinated therefrom, where the at least one progeny seed or plant germinated therefrom comprises the recombinant nucleic acid molecule.

In an aspect, a first plant variety and a second plant variety are the same variety. In an aspect, a first plant variety and a second plant variety are two different varieties. In an aspect, a second plant variety comprises a recombinant nucleic acid molecule.

In an aspect, a first plant variety is heterozygous for a recombinant nucleic acid molecule. In an aspect, a first plant variety is hemizygous for a recombinant nucleic acid molecule. In an aspect, a first plant variety is homozygous for a recombinant nucleic acid molecule. In an aspect, a second plant variety is heterozygous for a recombinant nucleic acid molecule. In an aspect, a second plant variety is hemizygous for a recombinant nucleic acid molecule. In an aspect, a second plant variety is homozygous for a recombinant nucleic acid molecule. In an aspect, a progeny seed, or a plant germinated therefrom, is heterozygous for a recombinant nucleic acid molecule. In an aspect, a progeny seed, or a plant germinated therefrom, is hemizygous for a recombinant nucleic acid molecule. In an aspect, a progeny seed, or a plant germinated therefrom, is homozygous for a recombinant nucleic acid molecule.

In an aspect, a first plant variety is a tobacco plant variety. In an aspect, a second plant variety is a tobacco plant variety. In an aspect, a first plant variety is a cannabis plant variety. In an aspect, a second plant variety is a cannabis plant variety.

As used herein, the term “crossing” refers to the deliberate mating of two plants. In an aspect, crossing comprises pollination and/or fertilization of a first plant by a second plant. The two plants being crossed can be distantly related, closely related, or identical. In an aspect, the two plants being crossed are both modified plants. In an aspect, the two plants being crossed are of the same variety. In an aspect, the two plants being crossed are of two different varieties. In an aspect, one of the two plants being crossed is male sterile. In an aspect, one of the two plants being crossed is female sterile. In an aspect, at least one of the two plants being crossed is a hybrid tobacco plant. In an aspect, at least one of the two plants being crossed is a modified plant.

In an aspect, a plant of a first variety is the male parent in a crossing step. In an aspect, a plant of a first variety is the female parent in a crossing step. In an aspect, a plant of a second variety is the male parent in a crossing step. In an aspect, a plant of a second variety is the female parent in a crossing step.

### Nucleic Acids and Amino Acids

As used herein, “heterologous” refers to a sequence (nucleic acid or amino acid) that originates from a foreign species, or, if from the same species, is substantially modified from its native form in composition and/or genomic locus by deliberate human intervention. The term also is applicable to nucleic acid constructs, also referred to herein as “polynucleotide constructs.” In this manner, a “heterologous” nucleic acid construct is intended to mean a construct that originates from a foreign species, or, if from the same species, is substantially modified from its native form in composition and/or genomic locus by deliberate human intervention. Heterologous nucleic acid constructs include, but are not limited to, recombinant nucleotide constructs that have been introduced into a plant or plant part thereof, for example, via transformation methods or subsequent breeding of a transgenic plant with another plant of interest. It will be appreciated that an endogenous promoter can be considered heterologous to an operably linked endogenous gene if the endogenous promoter and endogenous gene are not naturally operably linked (e.g., human intervention is required to put them in operable linkage). As used herein, an “endogenous” nucleic acid sequence refers to a nucleic acid sequence that occurs naturally in the genome of an organism.

In an aspect, a heterologous polynucleotide comprises a gene. In an aspect, a heterologous polynucleotide encodes a small RNA molecule or a precursor thereof. In an aspect, a heterologous polynucleotide encodes a polypeptide.

As used herein, a “gene” refers to a polynucleotide that can produce a functional unit (e.g., without being limiting, for example, a polypeptide, or a small RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a small RNA molecule or a precursor thereof. In another aspect, a gene encodes a polypeptide.

In an aspect, a gene encodes a polypeptide that has anti-herbivore properties. In an aspect, a gene encodes a polypeptide that has anti-insect properties. In an aspect, a gene encodes a polypeptide that has anti-fungal properties. In an aspect, a gene encodes a polypeptide that has anti-microbial properties.

In an aspect, a gene encodes a premnaspirodiene oxygenase-like gene. In an aspect, a gene encodes a polypeptide involved in cembratrieneol biosynthesis.

In an aspect, a gene comprises a nucleic acid sequence at least 80% identical to a nucleic acid sequence selected from the group consisting of SEQ ID NOs: 27-35 and 48-63. In an aspect, a gene comprises a nucleic acid sequence at least 85% identical to a nucleic acid sequence selected from the group consisting of SEQ ID NOs: 27-35 and 48-63. In an aspect, a gene comprises a nucleic acid sequence at least 90% identical to a nucleic acid sequence selected from the group consisting of SEQ ID NOs: 27-35 and 48-63. In an aspect, a gene comprises a nucleic acid sequence at least 92.5% identical to a nucleic acid sequence selected from the group consisting of SEQ ID NOs: 27-35 and 48-63. In an aspect, a gene comprises a nucleic acid sequence at least 95% identical to a nucleic acid sequence selected from the group consisting of SEQ ID NOs: 27-35 and 48-63. In an aspect, a gene comprises a nucleic acid sequence at least 96% identical to a nucleic acid sequence selected from the group consisting of SEQ ID NOs: 27-35 and 48-63. In an aspect, a gene comprises a nucleic acid sequence at least 97% identical to a nucleic acid sequence selected from the group consisting of SEQ ID NOs: 27-35 and 48-63. In an aspect, a gene comprises a nucleic acid sequence at least 98% identical to a nucleic acid sequence selected from the group consisting of SEQ ID NOs: 27-35 and 48-63. In an aspect, a gene comprises a nucleic acid sequence at least 99% identical to a nucleic acid sequence selected from the group consisting of SEQ ID NOs: 27-35 and 48-63. In an aspect, a gene comprises a nucleic acid sequence at least 99.5% identical to a nucleic acid sequence selected from the group consisting of SEQ ID NOs: 27-35 and 48-63. In an aspect, a gene comprises a nucleic acid sequence 100% identical to a nucleic acid sequence selected from the group consisting of SEQ ID NOs: 27-35 and 48-63.

In an aspect, a gene comprises a nucleic acid sequence at least 80% identical to a nucleic acid sequence selected from the group consisting of SEQ ID NOs: 29 and 30. In an aspect, a gene comprises a nucleic acid sequence at least 85% identical to a nucleic acid sequence selected from the group consisting of SEQ ID NOs: 29 and 30. In an aspect, a gene comprises a nucleic acid sequence at least 90% identical to a nucleic acid sequence selected from the group consisting of SEQ ID NOs: 29 and 30. In an aspect, a gene comprises a nucleic acid sequence at least 92.5% identical to a nucleic acid sequence selected from the group consisting of SEQ ID NOs: 29 and 30. In an aspect, a gene comprises a nucleic acid sequence at least 95% identical to a nucleic acid sequence selected from the group consisting of SEQ ID NOs: 29 and 30. In an aspect, a gene comprises a nucleic acid sequence at least 96% identical to a nucleic acid sequence selected from the group consisting of SEQ ID NOs: 29 and 30. In an aspect, a gene comprises a nucleic acid sequence at least 97% identical to a nucleic acid sequence selected from the group consisting of SEQ ID NOs: 29 and 30. In an aspect, a gene comprises a nucleic acid sequence at least 98% identical to a nucleic acid sequence selected from the group consisting of SEQ ID NOs: 29 and 30. In an aspect, a gene comprises a nucleic acid sequence at least 99% identical to a nucleic acid sequence selected from the group consisting of SEQ ID NOs: 29 and 30. In an aspect, a gene comprises a nucleic acid sequence at least 99.5% identical to a nucleic acid sequence selected from the group consisting of SEQ ID NOs: 29 and 30. In an aspect, a gene comprises a nucleic acid sequence 100% identical to a nucleic acid sequence selected from the group consisting of SEQ ID NOs: 29 and 30.

In an aspect, a gene comprises a nucleic acid sequence encoding an amino acid sequence at least 80% identical or similar to an amino acid sequence selected from the group consisting of SEQ ID NOs: 18-26 and 64-79. In an aspect, a gene comprises a nucleic acid sequence encoding an amino acid sequence at least 85% identical or similar to an amino acid sequence selected from the group consisting of SEQ ID NOs: 18-26 and 64-79. In an aspect, a gene comprises a nucleic acid sequence encoding an amino acid sequence at least 90% identical or similar to an amino acid sequence selected from the group consisting of SEQ ID NOs: 18-26 and 64-79. In an aspect, a gene comprises a nucleic acid sequence encoding an amino acid sequence at least 92.5% identical or similar to an amino acid sequence selected from the group consisting of SEQ ID NOs: 18-26 and 64-79. In an aspect, a gene comprises a nucleic acid sequence encoding an amino acid sequence at least 95% identical or similar to an amino acid sequence selected from the group consisting of SEQ ID NOs: 18-26 and 64-79. In an aspect, a gene comprises a nucleic acid sequence encoding an amino acid sequence at least 96% identical or similar to an amino acid sequence selected from the group consisting of SEQ ID NOs: 18-26 and 64-79. In an aspect, a gene comprises a nucleic acid sequence encoding an amino acid sequence at least 97% identical or similar to an amino acid sequence selected from the group consisting of SEQ ID NOs: 18-26 and 64-79. In an aspect, a gene comprises a nucleic acid sequence encoding an amino acid sequence at least 98% identical or similar to an amino acid sequence selected from the group consisting of SEQ ID NOs: 18-26 and 64-79. In an aspect, a gene comprises a nucleic acid sequence encoding an amino acid sequence at least 99% identical or similar to an amino acid sequence selected from the group consisting of SEQ ID NOs: 18-26 and 64-79. In an aspect, a gene comprises a nucleic acid sequence encoding an amino acid sequence at least 99.9% identical or similar to an amino acid sequence selected from the group consisting of SEQ ID NOs: 18-26 and 64-79. In an aspect, a gene comprises a nucleic acid sequence encoding an amino acid sequence 100% identical or similar to an amino acid sequence selected from the group consisting of SEQ ID NOs: 18-26 and 64-79.

In an aspect, a gene comprises a nucleic acid sequence encoding an amino acid sequence at least 80% identical or similar to an amino acid sequence selected from the group consisting of SEQ ID NOs: 20 and 21. In an aspect, a gene comprises a nucleic acid sequence encoding an amino acid sequence at least 85% identical or similar to an amino acid sequence selected from the group consisting of SEQ ID NOs: 20 and 21. In an aspect, a gene comprises a nucleic acid sequence encoding an amino acid sequence at least 90% identical or similar to an amino acid sequence selected from the group consisting of SEQ ID NOs: 20 and 21. In an aspect, a gene comprises a nucleic acid sequence encoding an amino acid sequence at least 92.5% identical or similar to an amino acid sequence selected from the group consisting of SEQ ID NOs: 20 and 21. In an aspect, a gene comprises a nucleic acid sequence encoding an amino acid sequence at least 95% identical or similar to an amino acid sequence selected from the group consisting of SEQ ID NOs: 20 and 21. In an aspect, a gene comprises a nucleic acid sequence encoding an amino acid sequence at least 96% identical or similar to an amino acid sequence selected from the group consisting of SEQ ID NOs: 20 and 21. In an aspect, a gene comprises a nucleic acid sequence encoding an amino acid sequence at least 97% identical or similar to an amino acid sequence selected from the group consisting of SEQ ID NOs: 20 and 21. In an aspect, a gene comprises a nucleic acid sequence encoding an amino acid sequence at least 98% identical or similar to an amino acid sequence selected from the group consisting of SEQ ID NOs: 20 and 21. In an aspect, a gene comprises a nucleic acid sequence encoding an amino acid sequence at least 99% identical or similar to an amino acid sequence selected from the group consisting of SEQ ID NOs: 20 and 21. In an aspect, a gene comprises a nucleic acid sequence encoding an amino acid sequence at least 99.9% identical or similar to an amino acid sequence selected from the group consisting of SEQ ID NOs: 20 and 21. In an aspect, a gene comprises a nucleic acid sequence encoding an amino acid sequence 100% identical or similar to an amino acid sequence selected from the group consisting of SEQ ID NOs: 20 and 21.

In an aspect, a polypeptide comprises an amino acid sequence at least 80% identical or similar to an amino acid sequence selected from the group consisting of SEQ ID NOs: 18-26 and 64-79. In an aspect, a polypeptide comprises an amino acid sequence at least 85% identical or similar to an amino acid sequence selected from the group consisting of SEQ ID NOs: 18-26 and 64-79. In an aspect, a polypeptide comprises an amino acid sequence at least 90% identical or similar to an amino acid sequence selected from the group consisting of SEQ ID NOs: 18-26 and 64-79. In an aspect, a polypeptide comprises an amino acid sequence at least 92.5% identical or similar to an amino acid sequence selected from the group consisting of SEQ ID NOs: 18-26 and 64-79. In an aspect, a polypeptide comprises an amino acid sequence at least 95% identical or similar to an amino acid sequence selected from the group consisting of SEQ ID NOs: 18-26 and 64-79. In an aspect, a polypeptide comprises an amino acid sequence at least 96% identical or similar to an amino acid sequence selected from the group consisting of SEQ ID NOs: 18-26 and 64-79. In an aspect, a polypeptide comprises an amino acid sequence at least 97% identical or similar to an amino acid sequence selected from the group consisting of SEQ ID NOs: 18-26 and 64-79. In an aspect, a polypeptide comprises an amino acid sequence at least 98% identical or similar to an amino acid sequence selected from the group consisting of SEQ ID NOs: 18-26 and 64-79. In an aspect, a polypeptide comprises an amino acid sequence at least 99% identical or similar to an amino acid sequence selected from the group consisting of SEQ ID NOs: 18-26 and 64-79. In an aspect, a polypeptide comprises an amino acid sequence at least 99.9% identical or similar to an amino acid sequence selected from the group consisting of SEQ ID NOs: 18-26 and 64-79. In an aspect, a polypeptide comprises an amino acid sequence 100% identical or similar to an amino acid sequence selected from the group consisting of SEQ ID NOs: 18-26 and 64-79.

In an aspect, a polypeptide comprises an amino acid sequence at least 80% identical or similar to an amino acid sequence selected from the group consisting of SEQ ID NOs: 20 and 21. In an aspect, a polypeptide comprises an amino acid sequence at least 85% identical or similar to an amino acid sequence selected from the group consisting of SEQ ID NOs: 20 and 21. In an aspect, a polypeptide comprises an amino acid sequence at least 90% identical or similar to an amino acid sequence selected from the group consisting of SEQ ID NOs: 20 and 21. In an aspect, a polypeptide comprises an amino acid sequence at least 92.5% identical or similar to an amino acid sequence selected from the group consisting of SEQ ID NOs: 20 and 21. In an aspect, a polypeptide comprises an amino acid sequence at least 95% identical or similar to an amino acid sequence selected from the group consisting of SEQ ID NOs: 20 and 21. In an aspect, a polypeptide comprises an amino acid sequence at least 96% identical or similar to an amino acid sequence selected from the group consisting of SEQ ID NOs: 20 and 21. In an aspect, a polypeptide comprises an amino acid sequence at least 97% identical or similar to an amino acid sequence selected from the group consisting of SEQ ID NOs: 20 and 21. In an aspect, a polypeptide comprises an amino acid sequence at least 98% identical or similar to an amino acid sequence selected from the group consisting of SEQ ID NOs: 20 and 21. In an aspect, a polypeptide comprises an amino acid sequence at least 99% identical or similar to an amino acid sequence selected from the group consisting of SEQ ID NOs: 20 and 21. In an aspect, a polypeptide comprises an amino acid sequence at least 99.9% identical or similar to an amino acid sequence selected from the group consisting of SEQ ID NOs: 20 and 21. In an aspect, a polypeptide comprises an amino acid sequence 100% identical or similar to an amino acid sequence selected from the group consisting of SEQ ID NOs: 20 and 21.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31:3497-3500 (2003); Thompson et al., “Clustal W: Improving the sensitivity of progressive multiple sequence alignment through sequence weighting, position-specific gap penalties and weight matrix choice,” Nucleic Acids Research 22:4673-4680 (1994); Larkin M A et al., “Clustal W and Clustal X version 2.0,” Bioinformatics 23:2947-48 (2007); and Altschul et al. “Basic local alignment search tool.” J. Mol. Biol. 215:403-410 (1990), the entire contents and disclosures of which are incorporated herein by reference.

The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded. A nucleic acid molecule can encode a polypeptide or a small RNA.

As used herein, a “recombinant nucleic acid molecule” refers to a nucleic acid molecule formed by laboratory methods of genetic recombination, such as, without being limiting, molecular cloning. Similarly, a “recombinant DNA construct” refers to a DNA molecule formed by laboratory methods of genetic recombination.

In an aspect, this disclosure provides a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 85% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 90% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 92.5% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 95% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 96% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 97% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 98% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 99% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence at least 99.9% identical to a sequence selected from the group consisting of SEQ ID NOs: 10-17, 45-47, and 80-82, or a functional fragment thereof. In an aspect, this disclosure provides a recombinant nucleic acid molecule comprising a promoter operably linked to a heterologous polynucleotide, where the promoter comprises a nucleic acid sequence 100% identical to a sequence selected from the group consisting of SEQ ID NOs: 10-17, 45-47, and 80-82, or a functional fragment thereof.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

In one aspect, this disclosure provides methods of detecting recombinant nucleic acids and polypeptides in plant cells. Without being limiting, nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Detection (e.g., of an amplification product, of a hybridization complex, of a polypeptide) can be accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

In an aspect, this disclosure provides a small RNA molecule, or a precursor thereof. As used herein, a “small RNA molecule” refers to a non-coding RNA molecule of between 16 nucleotides and 50 nucleotides in length. In an aspect, a small RNA molecule comprises between 16 nucleotides and 40 nucleotides. In another aspect, a small RNA molecule comprises between 16 nucleotides and 30 nucleotides. In another aspect, a small RNA molecule comprises between 18 nucleotides and 50 nucleotides. In another aspect, a small RNA molecule comprises between 18 nucleotides and 40 nucleotides. In another aspect, a small RNA molecule comprises between 18 nucleotides and 30 nucleotides. In another aspect, a small RNA molecule comprises between 18 nucleotides and 25 nucleotides. In another aspect, a small RNA molecule comprises between 20 nucleotides and 28 nucleotides. In another aspect, a small RNA molecule comprises between 20 nucleotides and 24 nucleotides. In another aspect, a small RNA molecule comprises between 21 nucleotides and 23 nucleotides. In another aspect, a small RNA molecule comprises 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In an aspect, a small RNA molecule is selected from the group consisting of a double-stranded RNA, a small interfering RNA (siRNA), a trans-acting siRNA, and a microRNA (miRNA).

miRNAs are generally of between about 19 to about 25 nucleotides (commonly about 20-24 nucleotides in plants), that guide cleavage in trans of target transcripts, negatively regulating the expression of genes involved in various regulation and development pathways. In some cases, miRNAs serve to guide in-phase processing of siRNA primary transcripts.

Many microRNA genes (MIR genes) have been identified and made publicly available in a database (“miRBase”, available online at microrna[dot]sanger[dot]ac[dot]uk/sequences; also see Griffiths-Jones et al. (2003) Nucleic Acids Res., 31:439-441). MIR genes have been reported to occur in intergenic regions, both isolated and in clusters in the genome, but can also be located entirely or partially within introns of other genes (both protein-coding and non-protein-coding). For a review of miRNA biogenesis, see Kim (2005) Nature Rev. Mol. Cell. Biol., 6:376-385. Transcription of MIR genes can be, at least in some cases, under promotional control of a MIR gene's own promoter. The primary transcript, termed a “pri-miRNA”, can be quite large (several kilobases) and can be polycistronic, containing one or more pre-miRNAs (fold-back structures containing a stem-loop arrangement that is processed to the mature miRNA) as well as the usual 5′ “cap” and polyadenylated tail of an mRNA.

Maturation of a mature miRNA from its corresponding precursors (pri-miRNAs and pre-miRNAs) differs significantly between animals and plants. For example, in plant cells, microRNA precursor molecules are believed to be largely processed to the mature miRNA entirely in the nucleus, whereas in animal cells, the pri-miRNA transcript is processed in the nucleus by the animal-specific enzyme Drosha, followed by export of the pre-miRNA to the cytoplasm where it is further processed to the mature miRNA. Mature miRNAs in plants are typically 21 nucleotides in length.

Transgenic expression of miRNAs (whether a naturally occurring sequence or an artificial sequence) can be employed to regulate expression of the miRNA's target gene or genes. Inclusion of a miRNA recognition site in a transgenically expressed transcript is also useful in regulating expression of the transcript. Recognition sites of miRNAs have been validated in all regions of an mRNA, including the 5′ untranslated region, coding region, and 3′ untranslated region, indicating that the position of the miRNA target site relative to the coding sequence may not necessarily affect suppression. Because miRNAs are important regulatory elements in eukaryotes, transgenic suppression of miRNAs is useful for manipulating biological pathways and responses. Finally, promoters of MIR genes can have very specific expression patterns (e.g., cell-specific, tissue-specific, temporally specific, or inducible), and thus are useful in recombinant constructs to induce such specific transcription of a DNA sequence to which they are operably linked. Various utilities of miRNAs, their precursors, their recognition sites, and their promoters are described in detail in U.S. Patent Application Publication 2006/0200878 A1, incorporated by reference herein. Non-limiting examples of these utilities include: (1) the expression of a native miRNA or miRNA precursor sequence to suppress a target gene; (2) the expression of an artificial miRNA or miRNA precursor sequence to suppress a target gene; (3) expression of a transgene with a miRNA recognition site, where the transgene is suppressed when the mature miRNA is expressed; (4) expression of a transgene driven by a miRNA promoter.

Designing an artificial miRNA sequence can be as simple as substituting sequence that is complementary to the intended target for nucleotides in the miRNA stem region of the miRNA precursor, as demonstrated by Zeng et al. (2002) Mol. Cell, 9:1327-1333. One non-limiting example of a general method for determining nucleotide changes in the native miRNA sequence to produce the engineered miRNA precursor includes the following steps: (a) Selecting a unique target sequence of at least 18 nucleotides specific to the target gene, e.g., by using sequence alignment tools such as BLAST (see, for example, Altschul et al. (1990) J. Mol. Biol., 215:403-410; Altschul et al. (1997) Nucleic Acids Res., 25:3389-3402), for example, of both tobacco cDNA and genomic DNA databases, to identify target transcript orthologues and any potential matches to unrelated genes, thereby avoiding unintentional silencing of non-target sequences; (b) Analyzing the target gene for undesirable sequences (e.g., matches to sequences from non-target species), and score each potential 19-mer segment for GC content, Reynolds score (see Reynolds et al. (2004) Nature Biotechnol., 22:326-330), and functional asymmetry characterized by a negative difference in free energy (“.DELTA..DELTA.G” or “ΔΔG”) (see Khvorova et al. (2003) Cell, 115:209-216). Preferably 19-mers are selected that have all or most of the following characteristics: (1) a Reynolds score>4, (2) a GC content between about 40% to about 60%, (3) a negative ΔΔG, (4) a terminal adenosine, (5) lack of a consecutive run of 4 or more of the same nucleotide; (6) a location near the 3′ terminus of the target gene; (7) minimal differences from the miRNA precursor transcript. Positions at every third nucleotide in an siRNA have been reported to be especially important in influencing RNAi efficacy and an algorithm, “siExplorer” is publicly available at rna[dot]chem[dot]t[dot]u-tokyo[dot]ac[dot]jp/siexplorer.htm (see Katoh and Suzuki (2007) Nucleic Acids Res., 10.1093/nar/gk11120); (c) Determining the reverse complement of the selected 19-mers to use in making a modified mature miRNA. The additional nucleotide at position 20 is preferably matched to the selected target sequence, and the nucleotide at position 21 is preferably chosen to either be unpaired to prevent spreading of silencing on the target transcript or paired to the target sequence to promote spreading of silencing on the target transcript; and (d) transforming the artificial miRNA into a plant.

### Terpenes

Terpenes are a class of aromatic organic compound produced by plants and some insects. Terpenes are hydrocarbon molecules that are often used by plants to either directly deter herbivory or to attract predators or parasites of plant herbivores. Non-limiting examples of terpenes include citral, menthol, camphor, salvinorin A, cannabinoids, and curcuminoids.

As used herein, a “terpene” refers to a volatile unsaturated hydrocarbon found in the essential oils of plants based on a cyclic molecule having the formula C₁₀H₁₆, as well as related structures and simple derivatives. As a non-limiting example, a sesquiterpene having the formula C₁₅H₂₄ is a terpene.

In an aspect, a terpene is a terpenoid. Terpenoids (also referred to as isoprenoids) are modified terpenes that contain additional functional groups, which often include oxygen. Terpenoids, which can be cyclic or acyclic, vary in size from five-carbon hemiterpenes to long complex molecules containing thousands of isoprene units. Terpenoids are produced through the condensation of five-carbon isoprene units (e.g., dimethylallyl diphosphate (DMAPP) and isopentenyl diphosphate (IPP)), most often by the sequential head-to-tail addition of DMAPP to IPP. The initial cyclization processes are catalyzed by different terpene synthases and enzyme variation leads to variation in monoterpene structure.

Terpenoids are classified according to the number of isoprene units that comprise the parent terpene. A hemiterpenoid comprises one isoprene unit. A monoterpenoid comprises two isoprene units. A sesquiterpenoid comprises three isoprene units. A diterpenoid comprises four isoprene units. A sesterterpenoid comprises five isoprene units. A triterpenoid comprises six isoprene units. A tetraterpenoid comprises eight isoprene units. A polyterpenoid comprises more than eight isoprene units.

In an aspect, a terpene is a hemiterpene. In an aspect, a terpene is a monoterpene. In an aspect, a terpene is a sesquiterpene. In an aspect, a terpene is a diterpene. In an aspect, a terpene is a sesterterpene. In an aspect, a terpene is a triterpene. In an aspect, a terpene is a tetraterpene. In an aspect, a terpene is a polyterpene.

In an aspect, a polypeptide is involved in the biosynthesis of at least one terpene. In an aspect, a polypeptide is involved in the biosynthesis of at least one terpenoid. In an aspect, a polypeptide is involved in the biosynthesis of at least one terpenoid selected from the group consisting of a hemiterpenoid, a monoterpenoid, a sesquiterpenoid, a diterpenoid, a sesterterpenoid, a triterpenoid, a tetraterpenoid, and a polyterpenoid.

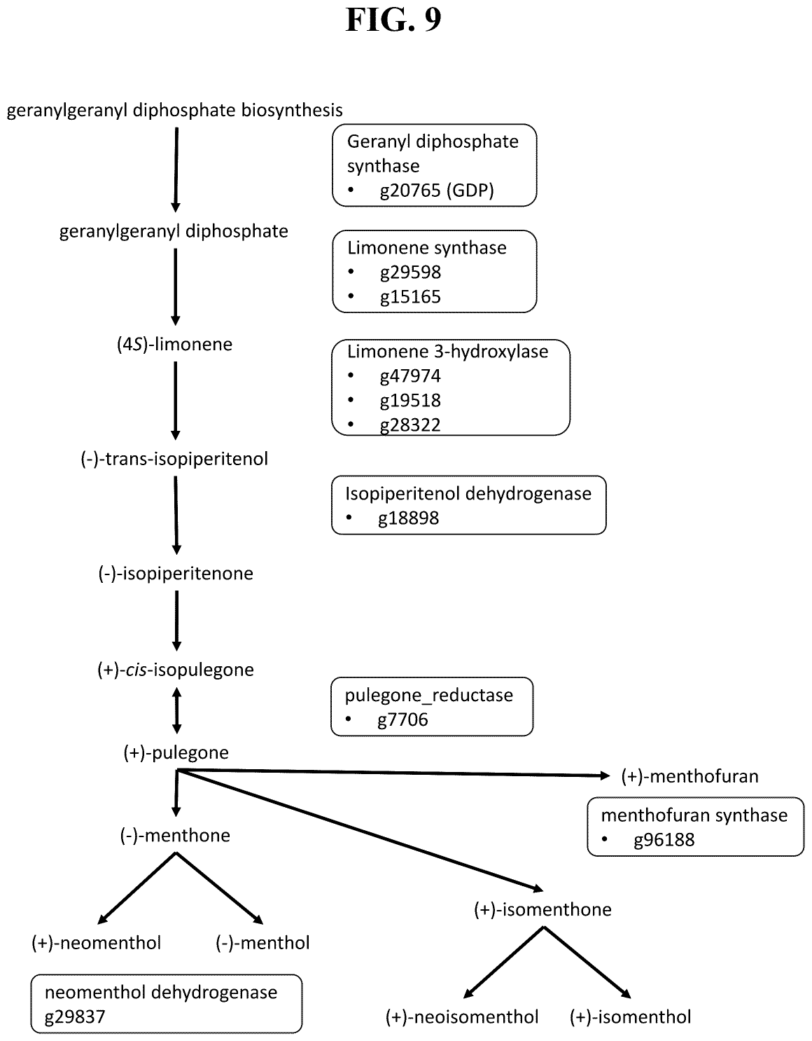

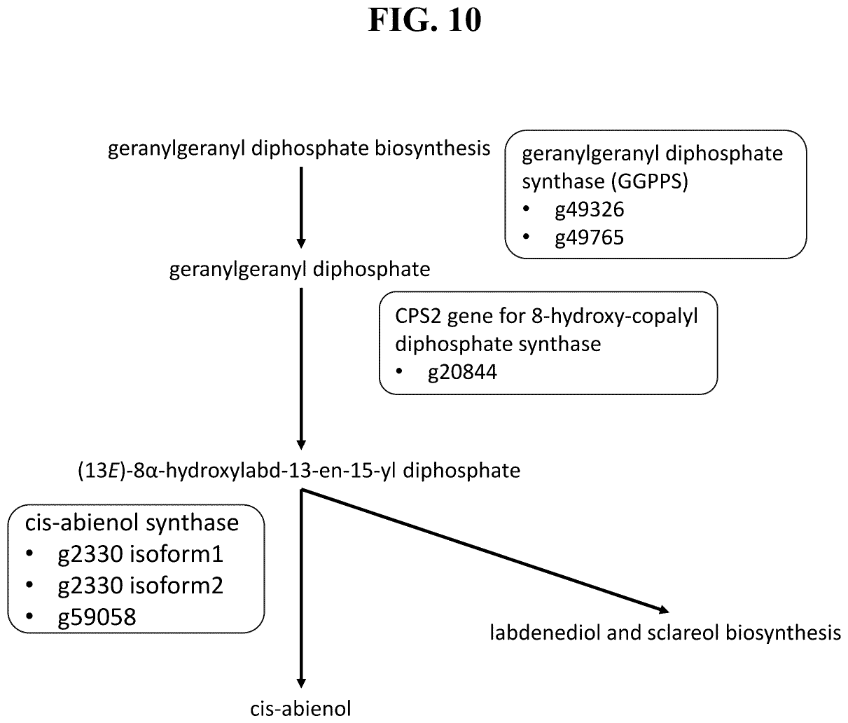

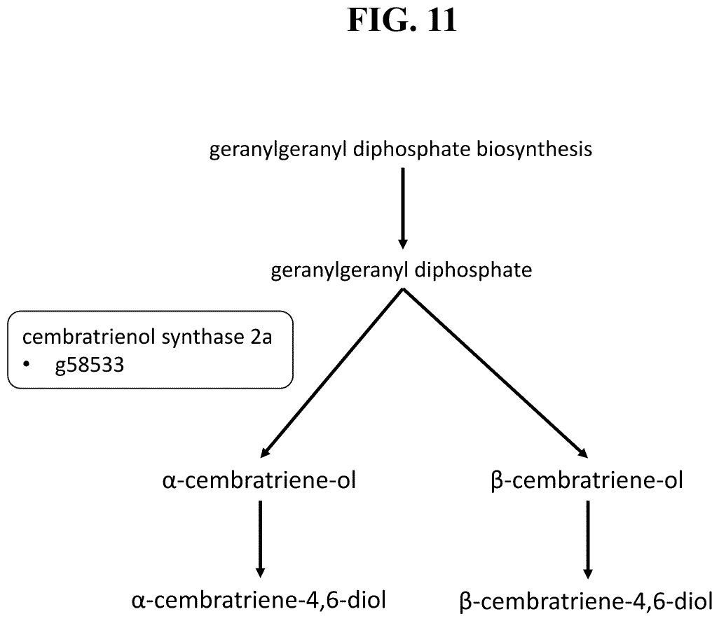

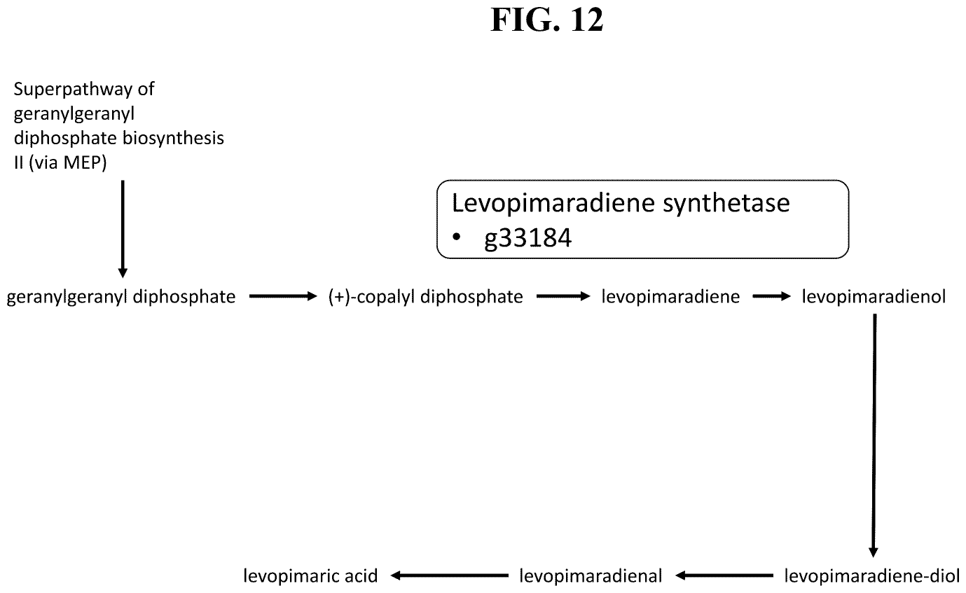

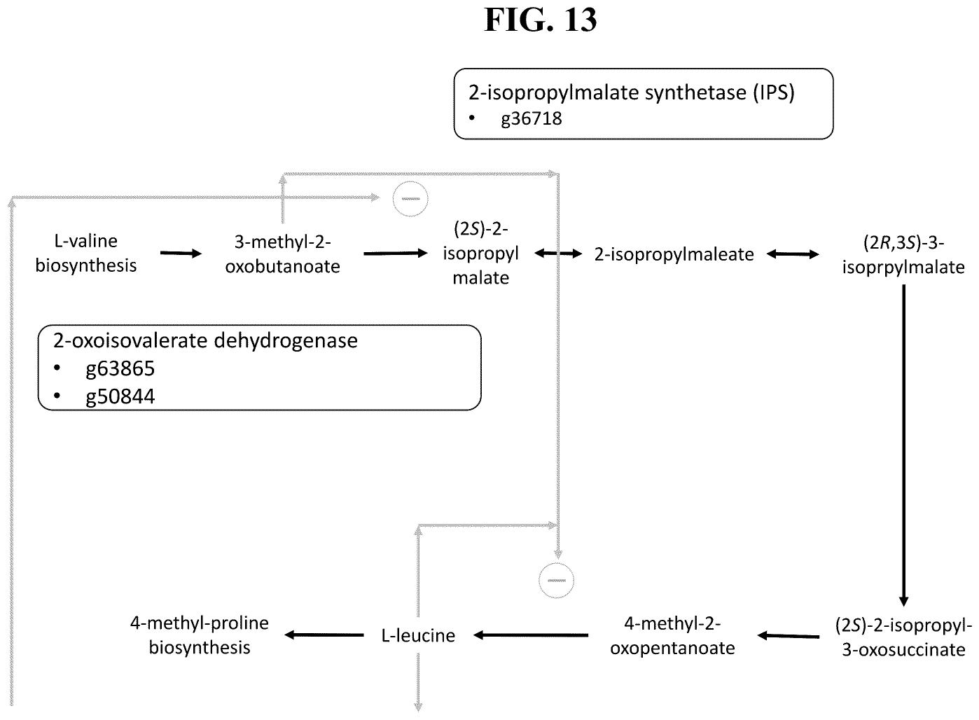

As used herein, the term “biosynthesis” refers to the production of a complex molecule (e.g., without being limiting, a terpene or terpenoid) within a plant or plant cell. To be “involved” with the biosynthesis of a compound, a polypeptide can directly interact with a substrate during the biosynthesis of the compound, or the polypeptide can affect the expression (positively or negatively) of a polypeptide that directly interacts with a substrate (e.g., a transcription factor that promotes the expression of an enzyme that converts a substrate to a new form or a repressor that inhibits expression of an enzyme that converts a substrate to a new form). Examples of biosynthetic pathways can be found in FIGS. 9-13.

In an aspect, a polypeptide is involved in the biosynthesis of a hemiterpene. In an aspect, a polypeptide is involved in the biosynthesis of a hemiterpenoid. In an aspect, a polypeptide is involved in the biosynthesis of a monoterpene. In an aspect, a polypeptide is involved in the biosynthesis of a monoterpenoid. In an aspect, a polypeptide is involved in the biosynthesis of a sesquiterpene. In an aspect, a polypeptide is involved in the biosynthesis of a sesquiterpenoid. In an aspect, a polypeptide is involved in the biosynthesis of a diterpene. In an aspect, a polypeptide is involved in the biosynthesis of a diterpenoid. In an aspect, a polypeptide is involved in the biosynthesis of a sesterterpene. In an aspect, a polypeptide is involved in the biosynthesis of a sesterterpenoid. In an aspect, a polypeptide is involved in the biosynthesis of a triterpene. In an aspect, a polypeptide is involved in the biosynthesis of a triterpenoid. In an aspect, a polypeptide is involved in the biosynthesis of a tetraterpene. In an aspect, a polypeptide is involved in the biosynthesis of a polyterpenoid. In an aspect, a polypeptide is involved in the biosynthesis of a monoterpene. In an aspect, a polypeptide is involved in the biosynthesis of a polyterpenoid.

Terpene synthase (TPS) genes can be grouped into seven clades: TPS-a, TPS-b, TPS-c, TPS-d, TPS-e/f, TPS-g, and TPS-h. TPS-a, TPS-b, and TPS-g are restricted to angiosperms, and TPS-d and TPS-h are specific to gymnosperms and the lycopod Selaginalla moellendorffii. The TPS-a clade comprises mostly sesquiterpene synthases and diterpene synthases, while the TPS-b and TPS-g clades comprise mostly monoterpene synthases.

In an aspect, a polypeptide involved in the biosynthesis of at least one terpenoid is a TPS-a clade member. In an aspect, a polypeptide involved in the biosynthesis of at least one terpenoid is a TPS-b clade member. In an aspect, a polypeptide involved in the biosynthesis of at least one terpenoid is a TPS-c clade member. In an aspect, a polypeptide involved in the biosynthesis of at least one terpenoid is a TPS-e/f clade member. In an aspect, a polypeptide involved in the biosynthesis of at least one terpenoid is a TPS-g clade member. In an aspect, a polypeptide involved in the biosynthesis of at least one terpenoid is a member of a clade selected from the group consisting of TPS-a, TPS-b, TPS-c, TPS-e/f, and TPS-g.

In an aspect, a terpene is menthol. In an aspect, a terpene is menthol or a related compound. In an aspect, a terpene is a labdanoid. In an aspect, a terpene is cembratrienediol. In an aspect, a terpene is levopimaric acid. In an aspect, a terpene is L-leucine. In an aspect, a terpene is neophytadiene. In an aspect, a labdanoid is cis-abienol. In an aspect, a terpene is selected from the group consisting of menthol or a related compound, a labdanoid, cembratrienediol, levopimaric acid, and L-leucine. In an aspect, a terpene is selected from the group consisting of menthol or a related compound, a labdanoid, cembratrienediol, levopimaric acid, L-leucine, and neophytadiene. In an aspect, a terpene is selected from the group consisting of menthol, a labdanoid, cembratrienediol, levopimaric acid, and L-leucine. In an aspect, a terpene is selected from the group consisting of menthol, a labdanoid, cembratrienediol, levopimaric acid, L-leucine, and neophytadiene.

As used herein, “menthol” refers to the organic compound having a chemical formula of C₁₀H₂₀O and the International Union of Pure and Applied Chemistry (IUPAC) name 5-Methyl-2-(propan-2-yl)cyclohexan-1-ol. Menthol is also referred to as “(−)-Menthol.” Related compounds of menthol include, but are not limited to, (+)-Menthol, (+)-Isomenthol, (+)-Neomenthol, (+)-Neoisomenthol, (−)-Isomenthol, (−)-Neomethol, and (−)-Neoisomenthol. In an aspect, a related compound of menthol is selected from the group consisting of (+)-Menthol, (+)-Isomenthol, (+)-Neomenthol, (+)-Neoisomenthol, (−)-Isomenthol, (−)-Neomethol, and (−)-Neoisomenthol.

As used herein, “neophytadiene” refers to the organic compound having a chemical formula of C₂₀H₃₈ and the IUPAC name of 7,11,15-trimethyl-3-methylidenehexadec-1-ene.

As used herein, “cembratrienediol” refers to the organic compound having a chemical formula of C₂₀H₃₄O₂ and the IUPAC name (1R,3R,4Z,8Z,12S,13Z)-1,5,9-trimethyl-12-propan-2-ylcyclotetradeca-4,8,13-triene-1,3-diol. Cembratrienediol is also referred to as “beta-Cembrenediol.”

As used herein, “levopimaric acid” refers to the organic compound having a chemical formula of C₂₀H₃₀O₂ and the IUPAC name (1R,4aR,4bS,10aR)-1,4a-dimethyl-7-propan-2-yl-2,3,4,4b,5,9,10,10a-octahydrophenanthrene-1-carboxylic acid. Levopimaric acid is also referred to as “L-Pimaric acid.”

As used herein, “L-leucine” refers to the amino acid having the chemical formula C₆H₁₂NO₂ and the IUPAC name (2S)-2-amino-4-methylpentanoic acid.

As used herein, a “labdanoid” refers to a terpenoid derivative of the fundamental parent labdane, a diterpene. A labdane has the chemical formula C₂₀H₃₈ and the IUPAC name (1S,2S,4aS,8aR)-2,5,5,8a-tetramethyl-1-[(3R)-3-methylpentyl]-1,2,3,4,4a,6,7,8-octahydronaphthalene.

A non-limiting example of a labdanoid is cis-abienol. As used herein, “cis-abienol” refers to the organic compound having a chemical formula of C₂₀H₃₄O and the IUPAC name (1R,2R,4aS,8aS)-2,5,5,8a-tetramethyl-1-[(2Z)-3-methylpenta-2,4,-dienyl]-3,4,4a,6,7,8-hexahydro-1H-naphthalen-2-ol.

In an aspect, a polypeptide is geranylgeranyl diphosphate synthase. In an aspect, a polypeptide is 8-hydroxy-copalyl diphosphate synthase. In an aspect, a polypeptide is cis-abienol synthase. In an aspect, a polypeptide is cembratrienol synthase 2a. In an aspect, a polypeptide is levopimardiene synthase. In an aspect, a polypeptide is 2-isopropylmalate synthetase. In an aspect, a polypeptide is 2-oxoisovalerate dehydrogenase. In an aspect, a polypeptide is neomenthol dehydrogenase. In an aspect, a polypeptide is selected from the group consisting of geranylgeranyl diphosphate synthase, 8-hydroxy-copalyl diphosphate synthase, cis-abienol synthase, cembratrienol synthase 2a, levopimaradiene synthetase, 2-isopropylmalate synthetase, 2-oxoisovalerate dehydrogenase, and neomenthol dehydrogenase.

As a non-limiting example, SEQ ID NOs: 18 and 27 are representative examples of amino acid and nucleic acid sequences, respectively, for geranylgeranyl diphosphate synthase. As a non-limiting example, SEQ ID NOs: 19 and 28 are representative examples of amino acid and nucleic acid sequences, respectively, for 8-hydroxy-copalyl diphosphate synthase. As a non-limiting example, SEQ ID NOs: 22 and 31 are representative examples of amino acid and nucleic acid sequences, respectively, for cembratrienol synthase 2a. As a non-limiting example, SEQ ID NOs: 23 and 32 are representative examples of amino acid and nucleic acid sequences, respectively, for levopimaradiene synthetase. As a non-limiting example, SEQ ID NOs: 24 and 33 are representative examples of amino acid and nucleic acid sequences, respectively, for 2-isopropylmalate synthetase. As a non-limiting example, SEQ ID NOs: 25 and 34 are representative examples of amino acid and nucleic acid sequences, respectively, for 2-oxoisovalerate dehydrogenase. As a non-limiting example, SEQ ID NOs: 26 and 35 are representative examples of amino acid and nucleic acid sequences, respectively, for neomenthol dehydrogenase.

In an aspect, a cis-abienol synthase is selected from the group consisting of cis-abienol synthase ISOFORM1 and cis-abienol synthase ISOFORM 2. In an aspect, a cis-abienol synthase is cis-abienol synthase ISOFORM 1. In an aspect, a cis-abienol synthase is cis-abienol synthase ISOFORM 2. As a non-limiting example, SEQ ID NOs: 20 and 29 are representative examples of amino acid and nucleic acid sequences, respectively, for cis-abienol synthase ISOFORM 1. As a non-limiting example, SEQ ID NOs: 21 and 30 are representative examples of amino acid and nucleic acid sequences, respectively, for cis-abienol synthase ISOFORM 2.

In an aspect, a modified plant, seed, or plant part comprising a recombinant nucleic acid provided herein comprises an increased amount of at least one terpene as compared to a control plant, seed, or plant part lacking the recombinant nucleic acid molecule when grown under comparable conditions. In an aspect, a modified tobacco plant, tobacco seed, or tobacco plant part comprising a recombinant nucleic acid provided herein comprises an increased amount of at least one terpene as compared to a control tobacco plant, tobacco seed, or tobacco plant part lacking the recombinant nucleic acid molecule when grown under comparable conditions. In an aspect, a modified cannabis plant, cannabis seed, or cannabis plant part comprising a recombinant nucleic acid provided herein comprises an increased amount of at least one terpene as compared to a control cannabis plant, cannabis seed, or cannabis plant part lacking the recombinant nucleic acid molecule when grown under comparable conditions.

In an aspect, an increased amount of at least one terpene comprises an increase of at least 0.5%. In an aspect, an increased amount of at least one terpene comprises an increase of at least 1%. In an aspect, an increased amount of at least one terpene comprises an increase of at least 2%. In an aspect, an increased amount of at least one terpene comprises an increase of at least 3%. In an aspect, an increased amount of at least one terpene comprises an increase of at least 4%. In an aspect, an increased amount of at least one terpene comprises an increase of at least 5%. In an aspect, an increased amount of at least one terpene comprises an increase of at least 10%. In an aspect, an increased amount of at least one terpene comprises an increase of at least 12.5%. In an aspect, an increased amount of at least one terpene comprises an increase of at least 15%. In an aspect, an increased amount of at least one terpene comprises an increase of at least 17.5%. In an aspect, an increased amount of at least one terpene comprises an increase of at least 20%. In an aspect, an increased amount of at least one terpene comprises an increase of at least 25%. In an aspect, an increased amount of at least one terpene comprises an increase of at least 30%. In an aspect, an increased amount of at least one terpene comprises an increase of at least 40%. In an aspect, an increased amount of at least one terpene comprises an increase of at least 50%. In an aspect, an increased amount of at least one terpene comprises an increase of at least 60%. In an aspect, an increased amount of at least one terpene comprises an increase of at least 70%. In an aspect, an increased amount of at least one terpene comprises an increase of at least 80%. In an aspect, an increased amount of at least one terpene comprises an increase of at least 90%. In an aspect, an increased amount of at least one terpene comprises an increase of at least 100%. In an aspect, an increased amount of at least one terpene comprises an increase of at least 150%. In an aspect, an increased amount of at least one terpene comprises an increase of at least 200%. In an aspect, an increased amount of at least one terpene comprises an increase of at least 250%. In an aspect, an increased amount of at least one terpene comprises an increase of at least 500%.

In an aspect, an increased amount of at least one terpene comprises an increase of between 0.5% and 500%. In an aspect, an increased amount of at least one terpene comprises an increase of between 0.5% and 250%. In an aspect, an increased amount of at least one terpene comprises an increase of between 0.5% and 100%. In an aspect, an increased amount of at least one terpene comprises an increase of between 0.5% and 75%. In an aspect, an increased amount of at least one terpene comprises an increase of between 0.5% and 50%. In an aspect, an increased amount of at least one terpene comprises an increase of between 0.5% and 25%. In an aspect, an increased amount of at least one terpene comprises an increase of between 0.5% and 10%. In an aspect, an increased amount of at least one terpene comprises an increase of between 0.5% and 5%. In an aspect, an increased amount of at least one terpene comprises an increase of between 0.5% and 500%. In an aspect, an increased amount of at least one terpene comprises an increase of between 5% and 250%. In an aspect, an increased amount of at least one terpene comprises an increase of between 5% and 100%. In an aspect, an increased amount of at least one terpene comprises an increase of between 5% and 50%. In an aspect, an increased amount of at least one terpene comprises an increase of between 25% and 500%. In an aspect, an increased amount of at least one terpene comprises an increase of between 25% and 250%. In an aspect, an increased amount of at least one terpene comprises an increase of between 50% and 100%. In an aspect, an increased amount of at least one terpene comprises an increase of between 100% and 500%.

The amount of terpenes in a plant can be measured using any method known in the art, including, without being limiting, gas chromatography mass spectrometry (GC-MS), Nuclear Magnetic Resonance Spectroscopy, and liquid chromatography-linked mass spectrometry. See The Handbook of Plant Metabolomics, edited by Weckwerth and Kahl, (Wiley-Blackwell) (May 28, 2013). In an aspect, an amount of at least one terpene refers to the concentration of the at least one terpene in the tissue sampled.

Cannabinoids are chemicals found in Cannabis plants. Many cannabinoids are concentrated in a resin produced in glandular trichomes, and at least 113 cannabinoids are known.

In an aspect, a heterologous polynucleotide is involved in the biosynthesis of a cannabinoid. In an aspect, a cannabinoid is selected from the group consisting of a cannabigerol-type (CBG) cannabinoid, a cannabichromene-type (CBC) cannabinoid, a cannabidiol-type (CBD) cannabinoid, a tetrahydrocannabinol-type (THC) cannabinoid, a cannabinol-type (CBN) cannabinoid, a cannabielsoin-type (CBE) cannabinoid, an iso-tetrahydrocannabinol-type (iso-THC) cannabinoid, a cannabicyclol-type (CBL) cannabinoid, and a cannabicitrain-type (CBT) cannaboinoid.

In an aspect, a cannabinoid is selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran.

### Products

In an aspect, this disclosure provides cured plant material from any plant or plant part provided herein. In an aspect, this disclosure provides cured tobacco material from any tobacco plant or tobacco plant part provided herein.

In an aspect, cured plant material is made by a curing process selected from the group consisting of flue curing, air curing, fire curing, and sun curing. In an aspect, cured tobacco material is made by a curing process selected from the group consisting of flue curing, air curing, fire curing, and sun curing. In an aspect, cured tobacco material is selected from the group consisting of flue cured tobacco material, air cured tobacco material, fire cured tobacco material, and sun cured tobacco material.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In one aspect, tobacco plants or plant components provided herein can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation.

Information regarding the harvesting of burley and dark tobacco varieties can be found in the 2019-2020 Burley and Dark Tobacco Production Guide (December 2018) published by the University of Kentucky, The University of Tennessee, Virginia Tech, and North Carolina State University, which is incorporated herein by reference in its entirety.

In an aspect, cured tobacco material comprises tobacco material selected from the group selected from cured leaf material, cured stem material, cured bud material, cured flower material, and cured root material. In an aspect, cured tobacco material comprises cured leaf material, cured stem material, or both. In an aspect, cured tobacco material comprises cured leaf material. In an aspect, cured tobacco material comprises cured stem material.

In an aspect, cured tobacco material comprises flue-cured tobacco material. In an aspect, cured tobacco material comprises air-cured tobacco material. In an aspect, cured tobacco material comprises fire-cured tobacco material. In an aspect, cured tobacco material comprises sun-cured tobacco material. In an aspect, cured tobacco material provided herein is selected from the group consisting of air-cured tobacco material, fire-cured tobacco material, sun-cured tobacco material, and flue-cured tobacco material. In an aspect, cured tobacco material is from a tobacco variety selected from the group consisting of a flue-cured variety, a bright variety, a Burley variety, a Virginia variety, a Maryland variety, a dark variety, an Oriental variety, and a Turkish variety.

In an aspect, cured tobacco leaf provided herein is selected from the group consisting of air-cured tobacco leaf, fire-cured tobacco leaf, sun-cured tobacco leaf, and flue-cured tobacco leaf. In an aspect, cured tobacco leaf is from a tobacco variety selected from the group consisting of a flue-cured variety, a bright variety, a Burley variety, a Virginia variety, a Maryland variety, a dark variety, an Oriental variety, and a Turkish variety.

Fermentation typically is characterized by high initial moisture content, heat generation, and a 10 to 20% loss of dry weight. See, for example, U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cured, aged, and fermented tobacco can be further processed (e.g., cut, shredded, expanded, or blended). See, for example, U.S. Pat. Nos. 4,528,993; 4,660,577; and 4,987,907. In an aspect, this disclosure provides fermented tobacco material from any tobacco plant, or part thereof, provided herein. In another aspect, this disclosure provides fermented tobacco material from any modified tobacco plant, or part thereof, provided herein.

Tobacco material obtained from the tobacco lines, varieties or hybrids of the present disclosure can be used to make tobacco products. As used herein, “tobacco product” is defined as any product made or derived from tobacco that is intended for human use or consumption. In an aspect, this disclosure provides a tobacco product comprising plant material from a tobacco plant provided herein. In another aspect, this disclosure provides a tobacco product comprising plant material from a modified tobacco plant provided herein. In another aspect, this disclosure provides a tobacco product comprising cured tobacco material. In another aspect, this disclosure provides a tobacco product comprising fermented tobacco material. In another aspect, this disclosure provides a tobacco product comprising a tobacco blend.

Tobacco products include, without limitation, cigarette products (e.g., cigarettes and bidi cigarettes), cigar products (e.g., cigar wrapping tobacco and cigarillos), pipe tobacco products, products derived from tobacco, tobacco-derived nicotine products, smokeless tobacco products (e.g., moist snuff, dry snuff, and chewing tobacco), films, chewables, tabs, shaped parts, gels, consumable units, insoluble matrices, hollow shapes, reconstituted tobacco, expanded tobacco, and the like. See, e.g., U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

In an aspect, a tobacco product comprises reconstituted tobacco. In another aspect, this disclosure provides reconstituted tobacco comprising cured tobacco material. As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

In an aspect, a tobacco product comprises expanded tobacco. As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokable material includes cured tobacco within a tobacco blend. In an aspect, a tobacco product of the present disclosure is selected from the group consisting of a kretek, a bidi cigarette, a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco.

In an aspect, a tobacco product of the present disclosure is selected from the group consisting of a cigarette, a heated tobacco product, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.

In an aspect, a tobacco product of the present disclosure is a smokeless tobacco product. In an aspect, a smokeless tobacco product is selected from the group consisting of loose leaf chewing tobacco, plug chewing tobacco, moist snuff, nasal snuff, dry snuff, and snus.

Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat-treated smokeless tobacco. Dry snuff is finely ground tobacco that is placed in the mouth or used nasally.

In yet another aspect, a tobacco product of the present disclosure is selected from the group consisting of an electronically heated cigarette, an e-cigarette, an electronic vaporing device.

In an aspect, a tobacco product of the present disclosure can be a blended tobacco product.

In another aspect, this disclosure provides a tobacco blend comprising cured tobacco material. A tobacco blend can comprise any combination of cured tobacco, uncured tobacco, fermented tobacco, unfermented tobacco, expanded tobacco, and reconstituted tobacco.

In an aspect, a tobacco blend comprises at least 5% cured tobacco by weight. In an aspect, a tobacco blend comprises at least 10% cured tobacco by weight. In an aspect, a tobacco blend comprises at least 15% cured tobacco by weight. In an aspect, a tobacco blend comprises at least 20% cured tobacco by weight. In an aspect, a tobacco blend comprises at least 25% cured tobacco by weight. In an aspect, a tobacco blend comprises at least 30% cured tobacco by weight. In an aspect, a tobacco blend comprises at least 35% cured tobacco by weight. In an aspect, a tobacco blend comprises at least 40% cured tobacco by weight. In an aspect, a tobacco blend comprises at least 45% cured tobacco by weight. In an aspect, a tobacco blend comprises at least 50% cured tobacco by weight. In an aspect, a tobacco blend comprises at least 55% cured tobacco by weight. In an aspect, a tobacco blend comprises at least 60% cured tobacco by weight. In an aspect, a tobacco blend comprises at least 65% cured tobacco by weight. In an aspect, a tobacco blend comprises at least 70% cured tobacco by weight. In an aspect, a tobacco blend comprises at least 75% cured tobacco by weight. In an aspect, a tobacco blend comprises at least 80% cured tobacco by weight. In an aspect, a tobacco blend comprises at least 85% cured tobacco by weight. In an aspect, a tobacco blend comprises at least 90% cured tobacco by weight. In an aspect, a tobacco blend comprises at least 95% cured tobacco by weight.

In an aspect, a tobacco blend comprises at least 5% cured tobacco by volume. In an aspect, a tobacco blend comprises at least 10% cured tobacco by volume. In an aspect, a tobacco blend comprises at least 15% cured tobacco by volume. In an aspect, a tobacco blend comprises at least 20% cured tobacco by volume. In an aspect, a tobacco blend comprises at least 25% cured tobacco by volume. In an aspect, a tobacco blend comprises at least 30% cured tobacco by volume. In an aspect, a tobacco blend comprises at least 35% cured tobacco by volume. In an aspect, a tobacco blend comprises at least 40% cured tobacco by volume. In an aspect, a tobacco blend comprises at least 45% cured tobacco by volume. In an aspect, a tobacco blend comprises at least 50% cured tobacco by volume. In an aspect, a tobacco blend comprises at least 55% cured tobacco by volume. In an aspect, a tobacco blend comprises at least 60% cured tobacco by volume. In an aspect, a tobacco blend comprises at least 65% cured tobacco by volume. In an aspect, a tobacco blend comprises at least 70% cured tobacco by volume. In an aspect, a tobacco blend comprises at least 75% cured tobacco by volume. In an aspect, a tobacco blend comprises at least 80% cured tobacco by volume. In an aspect, a tobacco blend comprises at least 85% cured tobacco by volume. In an aspect, a tobacco blend comprises at least 90% cured tobacco by volume. In an aspect, a tobacco blend comprises at least 95% cured tobacco by volume.

In an aspect, this disclosure provides a cannabis product comprising material from a cannabis plant, cannabis seed, or cannabis plant part provided herein. In an aspect, a cannabis product is a smokeless product. In an aspect, a cannabis product is an edible product. In an aspect, a cannabis product is a smokable product. In a further aspect, a smokeless cannabis product is a fiber based product. In an aspect, a cannabis product is derived from cannabis biomass. In an aspect, a cannabis product is a distillate derived from cannabis biomass.

In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 85% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 90% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 92.5% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 95% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 96% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 97% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 98% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 99% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 99.9% identical to a sequence selected from the group consisting of SEQ ID NOs: 10-17, 45-47, and 80-82, or a functional fragment thereof. In an aspect, this disclosure provides a method comprising preparing a tobacco producing using cured tobacco material from a modified tobacco plant or part therefrom, where the modified tobacco plant or part therefrom comprises a recombinant nucleic acid molecule comprising a promoter operably linked to a heterologous polynucleotide, where the promoter comprises a nucleic acid sequence at least 100% identical to a sequence selected from the group consisting of SEQ ID NOs: 10-17, 45-47, and 80-82, or a functional fragment thereof.

In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 85% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 90% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 92.5% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 95% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 96% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 97% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 98% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 99% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence at least 99.9% identical to a sequence selected from the group consisting of SEQ ID NOs: 10-17, 45-47, and 80-82, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where the promoter comprises a nucleic acid sequence 100% identical to a sequence selected from the group consisting of SEQ ID NOs: 10-17, 45-47, and 80-82, or a functional fragment thereof.

### Transformation

In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 80% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 85% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 90% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 92.5% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 95% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 96% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 97% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 98% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 99% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence at least 99.9% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous polynucleotide to at least one plant cell, where the promoter comprises a nucleic acid sequence 100% identical to a sequence selected from the group consisting of SEQ ID NOs: 10-17, 45-47, and 80-82, or a functional fragment thereof; (b) selecting at least one plant cell from step (a), where the at least one plant cell comprises the recombinant nucleic acid molecule; and (c) regenerating a modified plant from the at least one plant cell selected in step (b).

In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 80% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 85% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 90% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 92.5% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 95% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 96% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 97% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 98% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 99% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence at least 99.9% identical to a sequence selected from the group consisting of SEQ ID NOs: 10-17, 45-47, and 80-82, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where the promoter comprises a nucleic acid sequence 100% identical to a sequence selected from the group consisting of SEQ ID NOs: 10-17, 45-47, and 80-82, or a functional fragment thereof.

Numerous methods for “introducing” a recombinant nucleic acid molecule to a plant cell are known in the art, which can be used according to methods of the present application to produce a modified plant cell, plant, seed, or plant part. As used herein, the terms “introducing” and “transforming” can be used interchangeably. Any suitable method or technique for transformation of a plant cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, polyethylene glycol (PEG)-mediated transformation, etc., are also known in the art. Modified plants produced by these transformation methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell (e.g., tobacco cell, cannabis cell) with any of the nucleic acid molecules provided herein.

In an aspect, a method of introducing a recombinant nucleic acid molecule to a plant cell comprises Agrobacterium-mediated transformation. In another aspect, a method of introducing a recombinant nucleic acid molecule to a plant cell comprises PEG-mediated transformation. In another aspect, a method of introducing a recombinant nucleic acid molecule to a plant cell comprises biolistic transformation. In another aspect, a method of introducing a recombinant nucleic acid molecule to a plant cell comprises liposome-mediated transfection (lipofection). In another aspect, a method of introducing a recombinant nucleic acid molecule to a plant cell comprises lentiviral transfection.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of WO 91/17424 and WO 91/16024. Delivery can be to cells (e.g. in vitro or ex vivo administration) or target tissues (e.g. in vivo administration).

Any plant cell from which a fertile plant can be regenerated is contemplated as a useful recipient cell for practice of this disclosure.

In an aspect, a recombinant nucleic acid molecule is introduced to a tobacco cell. In an aspect, a recombinant nucleic acid molecule is introduced to a tobacco protoplast cell. In another aspect, a recombinant nucleic acid molecule is introduced to a tobacco callus cell. In an aspect, a recombinant nucleic acid molecule is introduced to a tobacco cell selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

In an aspect, a recombinant nucleic acid molecule is introduced to a cannabis cell. In an aspect, a recombinant nucleic acid molecule is introduced to a cannabis protoplast cell. In another aspect, a recombinant nucleic acid molecule is introduced to a cannabis callus cell. In an aspect, a recombinant nucleic acid molecule is introduced to a cannabis cell selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference.

### EMBODIMENTS

The following examples of non-limiting embodiments are envisioned:
1. A modified plant, seed, or plant part, comprising a recombinant nucleic acid molecule comprising a promoter operably linked to a heterologous polynucleotide, wherein the promoter comprises a nucleic acid sequence at least 80% identical to a sequence selected from the group consisting of SEQ ID NOs: 10-17, 45-47, and 80-82, or a functional fragment thereof.
2. The modified plant, seed, or plant part, of embodiment 1, wherein the nucleic acid sequence is at least 90% identical to a sequence selected from the group consisting of SEQ ID NOS: 10-17, 45-47, and 80-82, or a functional fragment thereof.
3. The modified plant, seed, or plant part, of embodiment 1, wherein the nucleic acid sequence is 100% identical to a sequence selected from the group consisting of SEQ ID NOs: 10-17, 45-47, and 80-82, or a functional fragment thereof.
4. The modified plant, seed, or part thereof of any one of embodiments 1-3, wherein the modified plant, seed, or plant part is a tobacco plant, tobacco seed, or tobacco plant part.
5. The modified plant, seed, or part thereof of any one of embodiments 1-3, wherein the modified plant, seed, or plant part is a cannabis plant, cannabis seed, or cannabis plant part.
6. The modified plant, seed, or plant part, of one of embodiments 1-5, wherein the heterologous polynucleotide encodes a gene.
7. The modified plant, seed, or plant part of embodiment 6, wherein the gene comprises a nucleic acid sequence encoding an amino acid sequence at least 80% identical or similar to an amino acid sequence selected from the group consisting of SEQ ID NOs: 18-26 and 64-79.
8. The modified plant, seed, or plant part of embodiment 6, wherein the gene comprises a nucleic acid sequence at least 80% identical to a nucleic acid sequence selected from the group consisting of SEQ ID NOs: 27-35 and 48-63.
9. The modified plant, seed, or plant part, of embodiment 6, wherein the gene encodes a small RNA molecule, or a precursor thereof.
10. The modified plant, seed, or plant part, of embodiment 6, wherein the gene encodes a polypeptide.
11. The modified plant, seed, or plant part, of embodiment 10, wherein the polypeptide is involved in the biosynthesis of at least one terpene.
12. The modified plant, seed, or plant part, of embodiment 11, wherein the at least one terpene is selected from the group consisting of menthol or a related compound, a labdanoid, cembratrienediol, levopimaric acid, and L-leucine.
13. The modified plant, seed, or plant part of embodiment 12, wherein the labdanoid is cis-abienol.
14. The modified plant, seed, or plant part of embodiment 11, wherein the at least one terpene is neophytadiene.
15. The modified plant, seed, or plant part of embodiment 10, wherein the polypeptide is selected from the group consisting of geranylgeranyl diphosphate synthase, 8-hydroxy-copalyl diphosphate synthase, cis-abienol synthase, cembratrienol synthase 2a, levopimaradiene synthetase, 2-isopropylmalate synthetase, 2-oxoisovalerate dehydrogenase, and neomenthol dehydrogenase.
16. The modified plant, seed, or plant part of embodiment 15, wherein the polypeptide comprises an amino acid sequence at least 80% identical or similar to a sequence selected from the group consisting of SEQ ID NOs: 18-26 and 64-79.
17. The modified plant, seed, or plant part of embodiment 15, wherein the cis-abienol synthase is selected from the group consisting of cis-abienol synthase ISOFORM1 and cis-abienol synthase ISOFORM2.
18. The modified plant, seed, or plant part of embodiment 17, wherein the cis-abienol synthase comprises an amino acid sequence at least 80% identical or similar to a sequence selected from the group consisting of SEQ ID NOs: 20 and 21.
19. The modified plant, seed, or plant part of any one of embodiments 11-18, wherein the modified plant comprises an increased amount of the at least one terpene as compared to a control plant lacking the recombinant nucleic acid molecule when grown under comparable conditions.
20. The modified plant, seed, or plant part of embodiment 19, wherein the increased amount of the at least one terpene comprises an increase of at least 5% as compared to the control plant.
21. The modified plant, seed, or plant part of embodiment 4, wherein the tobacco plant, tobacco seed, or plant part is of a tobacco variety selected from the group consisting of a flue-cured variety, a bright variety, a Burley variety, a Virginia variety, a Maryland variety, a dark variety, a Galpão variety, an Oriental variety, and a Turkish variety.
22. The modified plant, seed, or plant part of any one of embodiments 1-21, wherein the plant is male sterile or cytoplasmically male sterile.
23. Cured tobacco material from the modified tobacco plant or tobacco plant part of any one of embodiments 4, 21, or 22.
24. The cured tobacco material of embodiment 23, wherein the cured tobacco material is made by a curing process selected from the group consisting of flue curing, air curing, fire curing, and sun curing.
25. A tobacco product comprising the cured tobacco material of embodiment 23 or 24, wherein the tobacco product is selected from the group consisting of a kretek, a bidi cigarette, a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco.
26. The tobacco product of embodiment 25, wherein the tobacco product is a smokeless tobacco product.
27. The tobacco product of embodiment 26, wherein the smokeless tobacco product is selected from the group consisting of loose leaf chewing tobacco, plug chewing tobacco, moist snuff, nasal snuff, dry snuff, and snus.
28. A reconstituted tobacco comprising the cured tobacco material of embodiment 23 or 24.
29. A cannabis product comprising material from the modified cannabis plant, cannabis seed, or cannabis plant part of embodiment 5.
30. A recombinant nucleic acid molecule comprising a promoter operably linked to a heterologous polynucleotide, wherein the promoter comprises a nucleic acid sequence at least 80% identical to a sequence selected from the group consisting of SEQ ID NOs: 10-17, 45-47, and 80-82, or a functional fragment thereof.
31. A method of generating a modified plant, the method comprising:
   (a) introducing a recombinant nucleic acid molecule comprising a promoter operably linked to a heterologous polynucleotide to at least one plant cell, wherein the promoter comprises a nucleic acid sequence at least 80% identical to a sequence selected from the group consisting of SEQ ID NOs: 10-17, 45-47, and 80-82, or a functional fragment thereof;
   (b) selecting at least one plant cell from step (a), wherein the at least one plant cell comprises the recombinant nucleic acid molecule; and
   (c) regenerating a modified plant from the at least one plant cell selected in step (b).
32. A method comprising preparing a tobacco product using cured tobacco material from a modified tobacco plant or part therefrom, wherein the modified tobacco plant or part therefrom comprises a recombinant nucleic acid molecule comprising a promoter operably linked to a heterologous polynucleotide, wherein the promoter comprises a nucleic acid sequence at least 80% identical to a sequence selected from the group consisting of SEQ ID NOs: 10-17, 45-47, and 80-82, or a functional fragment thereof.
33. A method comprising preparing a cannabis product using material from a modified cannabis plant or part therefrom, wherein the modified cannabis plant or part therefrom comprises a recombinant nucleic acid molecule comprising a promoter operably linked to a heterologous polynucleotide, wherein the promoter comprises a nucleic acid sequence at least 80% identical to a sequence selected from the group consisting of SEQ ID NOs: 10-17, 45-47, and 80-82, or a functional fragment thereof.
34. A method comprising transforming a plant cell with a recombinant nucleic acid molecule, wherein the recombinant nucleic acid molecule comprises a promoter operably linked to a heterologous polynucleotide, wherein the promoter comprises a nucleic acid sequence at least 80% identical to a sequence selected from the group consisting of SEQ ID NOs: 10-17, 45-47, and 80-82, or a functional fragment thereof.
35. A method for producing a plant, the method comprising:
   (a) crossing at least one plant of a first variety with at least one plant of a second variety to produce at least one progeny seed, wherein the at least one plant of the first variety comprises a recombinant nucleic acid molecule comprising a promoter operably linked to a heterologous polynucleotide, wherein the promoter comprises a nucleic acid sequence at least 80% identical to a sequence selected from the group consisting of SEQ ID NOs: 10-17, 45-47, and 80-82, or a functional fragment thereof; and
   (b) selecting at least one progeny seed produced in step (a), or a plant germinated therefrom, wherein the at least one progeny seed or plant germinated therefrom comprises the recombinant nucleic acid molecule.
36. The method of embodiment 35, wherein the first variety and the second variety are the same variety.
37. The method of any one of embodiments 35-36, wherein the at least one plant of a second variety comprises the recombinant nucleic acid molecule.
38. The method of embodiment 35, wherein the at least one progeny seed, or the plant germinated therefrom, is heterozygous for the recombinant nucleic acid molecule.
39. The method of embodiment 35 or 37, wherein the at least one progeny seed, or the plant germinated therefrom, is homozygous for the recombinant nucleic acid molecule.

Having now generally described the disclosure, the same will be more readily understood through reference to the following examples that are provided by way of illustration, and are not intended to be limiting of the present disclosure, unless specified.

### EXAMPLES

### Example 1. Isolation of Trichome-Specific Promoter Sequences

Specific oligonucleotide primers (SEQ ID NOs: 1-9) are used to amplify and isolate the promoter regions from several tobacco genes, including ribulose-1,5-bisphosphate carboxylase/oxygenase (RUBISCO) small subunit (NtRbcST; SEQ ID NOs: 10 and 11), premnaspirodiene oxygenase (NtPSO; SEQ ID NOs: 12 and 13), phylloplanin (NtPHY; SEQ ID NOs: 14 and 15), and cyclase (NtCYC; SEQ ID NOs: 16 and 17), using PCR.

### Example 2. NtRbcST is a Trichome-Specific RUBISCO Subunit

NtRbcST is a subunit of RUBISCO, which is the most abundant enzyme found in plants and is primarily involved on carbon dioxide fixation during photosynthesis. RUBISCO is assembled from eight large subunits (RbcL) encoded by a single chloroplast gene, and eight small subunits (RbcS) encoded by a nuclear gene family. In C3 plants, RUBISCO is mostly found in the chloroplasts of mesophyll cells, while RUBISCO is mostly found in the bundle-sheath and guard cells of C4 plants. However, RUBISCO can also be found in specialized cells like trichomes.

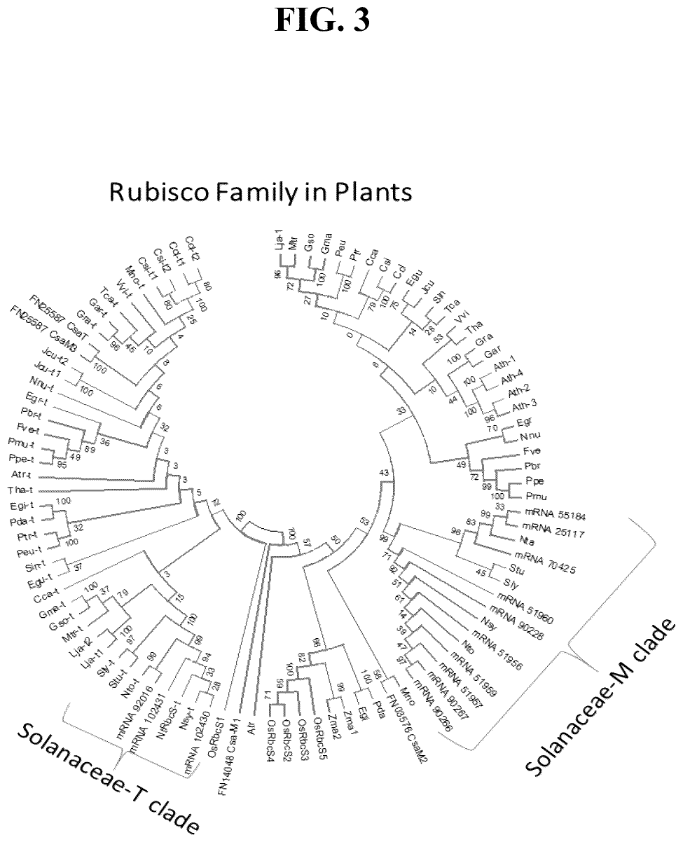

Phylogenetic analysis of RUBISCO small subunits in plants found that there are two distinct clades of RUBISCO small subunits: a mesophyll-specific clade and a trichome-specific clade. See FIG. 3.

### Example 3. Construction of Vectors

The PCR products obtained in Example 1 are cloned using the GATEWAY cloning system (ThermoFisher Scientific). The cloned promoters are subsequently subcloned into a GATEWAY expression vector, where the subcloned promoter drives the expression of GREEN FLUORESCENCE PROTEIN (G3GFP). See FIG. 2.

Various promoter lengths are used in different vector constructs. For example, an approximately 1.2 kilobase (kb) (SEQ ID NO: 10) and an approximately 0.4 kb (SEQ ID NO: 11) are tested for NtRbcST. Similarly, an approximately 1.0 kb promoter is tested for NtPSO (SEQ ID NO: 13); an approximately 0.5 kb promoter is tested for NtPHY (SEQ ID NO: 15); and an approximately 0.5 kb promoter is tested for NtCYC (SEQ ID NO: 17).

Additional constructs comprising SEQ ID NO: 80 operably linked to G3GFP, SEQ ID NO: 81 operably linked to G3GFP, and SEQ ID NO: 82 operably linked to G3GFP are also produced. See Example 9.

### Example 4. Transformation and Regeneration of Modified Tobacco Plants

Each of the vector constructs generated in Example 3 is separately transformed into tobacco cells in separate experiments. Briefly, the vectors are introduced into tobacco leaf discs via Agrobacterium transformation. See, for example, Mayo et al., Nat. Protoc., 1:1105-1111 (2006); and Horsch et al., Science, 227:1229-1231 (1985).

Tobacco plants (e.g., TN and K326 varieties; Nicotiana benthamiana) are grown in Magenta™ GA-7 boxes and leaf discs are cut and placed into Petri plates. Agrobacterium tumefaciens cells comprising a transformation vector are collected by centrifuging a 20 mL cell suspension in a 50 mL centrifuge tube at 3500 RPM for 10 minutes. The supernatant is removed, and the Agrobacterium tumefaciens cell pellet is re-suspended in 40 mL liquid re-suspension medium. Tobacco leaves, avoiding the midrib, are cut into eight 0.6 cm discs with a #15 razor blade and placed upside down in a Petri plate. A thin layer of Murashige & Skoog (MS) with B5 vitamin liquid re-suspension medium is added to the Petri plate and the leaf discs are poked uniformly with a fine point needle. About 25 mL of the Agrobacterium tumefaciens suspension is added to the Petri plate and the leaf discs are incubated in the suspension for 10 minutes.

Leaf discs are transferred to co-cultivation Petri plates (½ MS medium) and discs are placed upside down in contact with filter paper overlaid on the co-cultivation TOM medium (MS medium with 30 g/L sucrose; 0.1 mg/L 1-napthaleneacetic acid (NAA); and 1 mg/L 6-benzyl aminopurine (BAP)). The Petri plate is sealed with parafilm and incubated in the dark for two days.

After incubation, leaf discs are transferred to regeneration/selection TOM-Hyg medium Petri plates (TOM medium plus 200 mg/L cefotaxime and 50 mg/L hygromycin). Calli formed from leaf discs are sub-cultured bi-weekly to fresh TOM-Hyg medium in dim light (between 60 and 80 mE/ms) with photoperiods of 18 hours light, 6 hours dark, at 24° C. until shoots (plantlets) become excisable. Plantlets formed from calli are removed with forceps and subculture into MS rooting medium (MS medium with 3 g/L sucrose; 7 g/L dextrose; 200 mg/L cefotaxime; 50 mg/L hygromycin). Shoots on MS basal medium with 50 mg/L hygromycin are incubated with the dim light with photoperiods of 18 hours light, 6 hours dark, at 24° C. to induce rooting.

When plantlets comprising both shoots and roots grow large enough (e.g., over half the height of a Magenta™ GA-7 box), they are transferred Jiffy peat pellets for acclimatization in the growth room. Once established, seedlings are transferred to a greenhouse for further growth, breeding, and analysis.

### Example 5. Expression Pattern of Tested Promoters in Tobacco

The expression pattern of the trichome-specific promoters (see Example 3) is examined in the modified plants produced in Example 4. First generation (e.g., To) modified plants are sampled during the vegetative stage. Sections of young leaves are mounted onto glass slides with water, covered with a glass cover slip, and sealed with clear nail polish. Slides are viewed with a confocal laser scanning microscope under brightfield conditions and under conditions allowing the visualization of G3GFP (excitation/emission wavelengths are 488 nm/500-550 nm) expressed by the trichome-specific promoters.

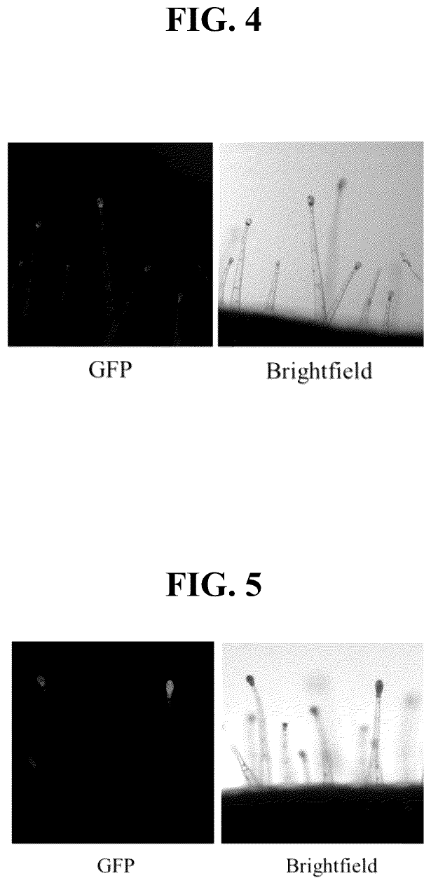

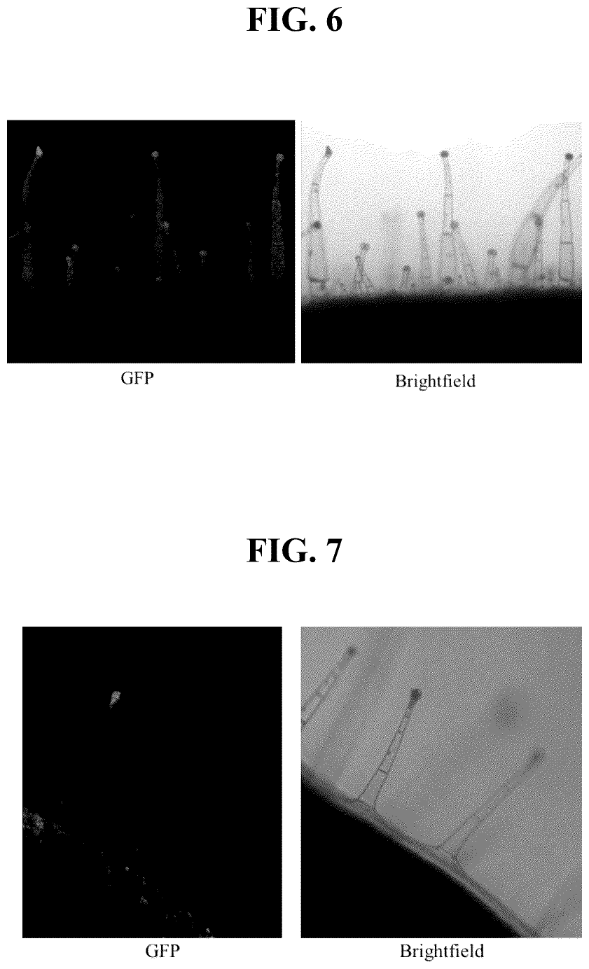

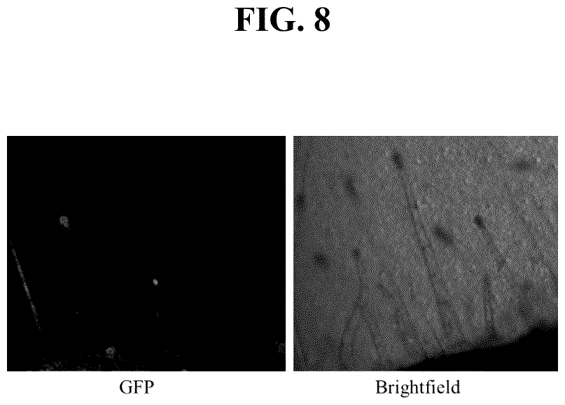

FIGS. 4-8 depict glandular trichomes as visualized under brightfield conditions and under conditions that allow the visualization of G3GFP. The 1.2 kb and 0.4 kb fragments (SEQ ID NOs: 10 and 11) of the NtRbcST promoter both drive expression in tobacco trichomes. Similarly, the 1.0 kb NtPSO promoter (SEQ ID NO: 13), the 0.5 kb NtPHY promoter (SEQ ID NO: 15), and the 0.5 kb NtCYC promoter (SEQ ID NO: 17) also drive expression in tobacco trichomes.

### Example 6. Expressing Terpenoid Biosynthesis Genes with Trichome-Specific Promoters

Each of the trichome-specific promoters tested in Examples 3-5 is also used to drive the expression of genes involved in terpenoid biosynthesis. Fifteen constructs are produced as described in Example 3, with each trichome-specific promoter (e.g., SEQ ID NOs: 10-17, 45-47, and 80-82) driving the expression of NEOMENTHOL DEHYDROGENASE (NtNMD; SEQ ID NO: 35) or one of two isoforms of CIS-ABIENOL SYNTHASE (NtaABS (Isoform 1; SEQ ID NO: 29) and NtABS (Isoform 2; SEQ ID NO: 30) in separate constructs.

Each of the fifteen constructs are separately transformed into tobacco cells and modified tobacco plants are regenerated as described in Example 4.

### Example 7. Confirming Expression of Terpene Biosynthesis Genes in Modified Tobacco Plants

During the vegetative stage of growth, RNA is extracted from young leaves from modified tobacco plants produced in Example 6, and from control tobacco plants lacking the recombinant nucleic acid constructs grown under comparable conditions. The extracted RNA is used to generate cDNA. Gene expression of NtNMD, NtaABS, and NtABS is quantified using quantitative real-time PCR (qRT-PCR). To confirm the constructs are functional, expression of NtNMD, NtaABS, and NtABS in the modified plants is compared to control tobacco plants.

### Example 8. Measuring Terpene Levels in Modified Tobacco Plants

During the vegetative stage of growth, young leaves are harvested from the modified tobacco plants from Example 6, and from control tobacco plants lacking the recombinant nucleic acid constructs grown under comparable conditions, for use in a qualitative metabolic profile analysis. Leaf samples are ground in liquid nitrogen, and then the samples are mixed with 60:40 hexane:ethyl acetate (v/v), supplemented with heptadecanol (an internal standard) and incubated overnight with shaking.

The solvent extracts are concentrated in a refrigerated SpeedVac™ (ThermoFisher Scientific) and placed into a silica column. The column is washed with hexane and allowed to flow through into collection tubes. Samples are aliquoted from the collection tubes and used for gas chromatography-mass spectrometry (GC-MS) analysis of metabolites.

### Example 9. Identification of Trichome-Specific Promoter Motifs

Trichome-specific promoter sequences (e.g., SEQ ID NOs: 10-12, 14, and 16) are scanned for regulatory element motifs using the publicly available online database PLANTCARE (bioinformatics[dot]psb[dot]ugent[dot]be/webtools/plantcare/html) using default settings. Tables 9-13 detail the regulatory element motifs that were identified for each of the trichome-specific promoters.

TABLE 9
Regulatory elements in RbT (1243 nt) promoter (SEQ ID NO: 10).

| | | Nucleotide | | |
|---|---|---|---|---|
| Motif | Sequence | Position | Strand | Function |
| ABRE | AACCCGG | 11 | − | cis-acting element involved in the abscisic |
| | | | | acid responsiveness |
| ARE | AAACCA | 210 | + | cis-acting regulatory element essential for |
| | | | | the anaerobic induction |
| as-1 | TGACG | 104 | − | |
| as-1 | TGACG | 964 | + | |
| AT~TATA-box | TATATAAA | 571 | − | |
| AT~TATA-box | TATATA | 573 | + | |
| ATC-motif | AGTAATCT | 1128 | − | part of a conserved DNA module involved |
| | | | | in light responsiveness |
| Box 4 | ATTAAT | 1063 | − | part of a conserved DNA module involved |
| | | | | in light responsiveness |
| CAAT-box | CAAT | 20 | + | |
| CAAT-box | CCAAT | 22 | − | common cis-acting element in promoter and |
| | | | | enhancer regions |
| CAAT-box | CAAT | 33 | − | |
| CAAT-box | CAAAT | 69 | + | common cis-acting element in promoter and |
| | | | | enhancer regions |
| CAAT-box | CAAT | 274 | + | |
| CAAT-box | CAAAT | 281 | − | common cis-acting element in promoter and |
| | | | | enhancer regions |
| CAAT-box | CAAT | 325 | + | |
| CAAT-box | CAAAT | 338 | + | common cis-acting element in promoter and |
| | | | | enhancer regions |
| CAAT-box | CAAAT | 346 | + | common cis-acting element in promoter and |
| | | | | enhancer regions |
| CAAT-box | CAAT | 377 | − | |
| CAAT-box | CAAAT | 468 | + | common cis-acting element in promoter and |
| | | | | enhancer regions |
| CAAT-box | CAAT | 488 | + | |
| CAAT-box | CAAT | 518 | − | |
| CAAT-box | TGCAAATCT | 666 | − | common cis-acting element in promoter and |
| | | | | enhancer regions |
| CAAT-box | CAAAT | 668 | − | common cis-acting element in promoter and |
| | | | | enhancer regions |
| CAAT-box | CAAT | 691 | − | |
| CAAT-box | CCAAT | 782 | + | common cis-acting element in promoter and |
| | | | | enhancer regions |
| CAAT-box | CAAT | 783 | + | |
| CAAT-box | CAAT | 796 | − | |
| CAAT-box | CAAT | 818 | + | |
| CGTCA-motif | CGTCA | 104 | + | cis-acting regulatory element involved in the |
| | | | | MeJA-responsiveness |
| CGTCA-motif | CGTCA | 964 | − | cis-acting regulatory element involved in the |
| | | | | MeJA-responsiveness |
| chs-CMA2a | TCACTTGA | 113 | − | part of a light responsive element |
| GT1-motif | GGTTAA | 833 | − | light responsive element |
| LAMP-element | CTTTATCA | 1202 | + | part of a light responsive element |
| MYB | TAACCA | 393 | + | |
| MYB | CAACCA | 429 | − | |
| MYB-like | TAACCA | 393 | + | |
| sequence | | | | |
| MYC | CAATTG | 20 | + | |
| MYC | CATTTG | 280 | + | |
| MYC | CATTTG | 468 | − | |
| MYC | CATGTG | 542 | + | |
| MYC | CATGTG | 718 | + | |
| STRE | AGGGG | 1028 | − | |
| STRE | AGGGG | 1043 | − | |
| TATA-box | TATTTAAA | 166 | − | core promoter element around −30 of |
| | | | | transcription start |
| TATA-box | taTATAAAtc | 526 | − | core promoter element around −30 of |
| | (SEQ ID | | | transcription start |
| | NO: 102) | | | |
| TATA-box | TACAAAA | 549 | + | core promoter element around −30 of |
| | | | | transcription start |
| TATA-box | taTATAAAg | 570 | − | core promoter element around −30 of |
| | | | | transcription start |
| TATA-box | TATAAA | 571 | − | core promoter element around −30 of |
| | | | | transcription start |
| TATA-box | TATATAA | 572 | − | core promoter element around −30 of |
| | | | | transcription start |
| TATA-box | TATATA | 573 | + | core promoter element around −30 of |
| | | | | transcription start |
| TATA-box | TATA | 575 | + | core promoter element around −30 of |
| | | | | transcription start |
| TATA-box | TATA | 599 | + | core promoter element around −30 of |
| | | | | transcription start |
| TATA-box | ATTATA | 842 | + | core promoter element around −30 of |
| | | | | transcription start |
| TATA-box | TATAA | 843 | − | core promoter element around −30 of |
| | | | | transcription start |
| TATA-box | TATA | 844 | − | core promoter element around −30 of |
| | | | | transcription start |
| TATA-box | TATACA | 854 | − | core promoter element around −30 of |
| | | | | transcription start |
| TATA-box | TATA | 856 | − | core promoter element around −30 of |
| | | | | transcription start |
| TATA-box | TACAAAA | 1115 | + | core promoter element around −30 of |
| | | | | transcription start |
| TATA-box | ATATAA | 1161 | + | core promoter element around −30 of |
| | | | | transcription start |
| TATA-box | TATA | 1162 | − | core promoter element around −30 of |
| | | | | transcription start |
| TATA-box | TATAAA | 1189 | − | core promoter element around −30 of |
| | | | | transcription start |
| TATA-box | TATAA | 1190 | − | core promoter element around −30 of |
| | | | | transcription start |
| TATA-box | TATA | 1191 | − | core promoter element around −30 of |
| | | | | transcription start |
| TCCC-motif | TCTCCCT | 1151 | + | part of a light responsive element |
| TGACG-motif | TGACG | 104 | − | cis-acting regulatory element involved in the |
| | | | | MeJA-responsiveness |
| TGACG-motif | TGACG | 964 | + | cis-acting regulatory element involved in the |
| | | | | MeJA-responsiveness |
| Unnamed_1 | CGTGG | 234 | − | |
| Unnamed_2 | CCCCGG | 769 | − | |
| Unnamed_4 | CTCC | 46 | − | |
| Unnamed_4 | CTCC | 197 | − | |
| Unnamed_4 | CTCC | 581 | − | |
| Unnamed_4 | CTCC | 740 | + | |
| Unnamed 4 | CTCC | 824 | − | |
| Unnamed 4 | CTCC | 893 | − | |
| Unnamed_4 | CTCC | 1152 | + | |
| W box | TTGACC | 432 | + | |
| WUN-motif | CAATTACAT | 274 | + | |

TABLE 10
Regulatory elements in RbT (436 nt) promoter (SEQ ID NO: 11).

| | | Nucleotide | | |
|---|---|---|---|---|
| Motif | Sequence | Position | Strand | Function |
| as-1 | TGACG | 157 | + | |
| ATC-motif | AGTAATCT | 321 | − | part of a conserved DNA module involved in |
| | | | | light responsiveness |
| Box 4 | ATTAAT | 256 | − | part of a conserved DNA module involved in |
| | | | | light responsiveness |
| CAAT-box | CAAT | 11 | + | |
| CGTCA-motif | CGTCA | 157 | − | cis-acting regulatory element involved in the |
| | | | | MeJA-responsiveness |
| GT1-motif | GGTTAA | 26 | − | light responsive element |
| LAMP-element | CTTTATCA | 395 | + | part of a light responsive element |
| STRE | AGGGG | 221 | − | |
| STRE | AGGGG | 236 | − | |
| TATA-box | ATTATA | 35 | + | core promoter element around −30 of |
| | | | | transcription start |
| TATA-box | TATAA | 36 | − | core promoter element around −30 of |
| | | | | transcription start |
| TATA-box | TATA | 37 | + | core promoter element around −30 of |
| | | | | transcription start |
| TATA-box | TATACA | 47 | − | core promoter element around −30 of |
| | | | | transcription start |
| TATA-box | TATA | 49 | + | core promoter element around −30 of |
| | | | | transcription start |
| TATA-box | TACAAAA | 308 | + | core promoter element around −30 of |
| | | | | transcription start |
| TATA-box | ATATAA | 354 | + | core promoter element around −30 of |
| | | | | transcription start |
| TATA-box | TATA | 355 | − | core promoter element around −30 of |
| | | | | transcription start |
| TATA-box | TATAAA | 382 | − | core promoter element around −30 of |
| | | | | transcription start |
| TATA-box | TATAA | 383 | − | core promoter element around −30 of |
| | | | | transcription start |
| TATA-box | TATA | 384 | − | core promoter element around −30 of |
| | | | | transcription start |
| TCCC-motif | TCTCCCT | 344 | + | part of a light responsive element |
| TGACG-motif | TGACG | 157 | + | cis-acting regulatory element involved in the |
| | | | | MeJA-responsiveness |
| Unnamed_4 | CTCC | 17 | | |
| Unnamed_4 | CTCC | 86 | − | |
| Unnamed_4 | CTCC | 345 | + | |

TABLE 11
Regulatory elements in PSO (1.5 kb) promoter (SEQ ID NO: 12).

| | | Nucleotide | | |
|---|---|---|---|---|
| Motif | Sequence | Position | Strand | Function |
| AAGAA-motif | GAAAGAA | 9 | + | |
| ARE | AAACCA | 181 | + | cis-acting regulatory element essential for |
| | | | | the anaerobic induction |
| ARE | AAACCA | 755 | + | cis-acting regulatory element essential for |
| | | | | the anaerobic induction |
| as-1 | TGACG | 337 | − | |
| AT~TATA-box | TATATA | 701 | + | |
| AT~TATA-box | TATATA | 703 | + | |
| AT~TATA-box | TATATA | 858 | − | |
| AT~TATA-box | TATATA | 860 | − | |
| AT~TATA-box | TATATAAA | 938 | − | |
| AT~TATA-box | TATATA | 940 | − | |
| AT-rich sequence | TAAAATACT | 542 | − | element for maximal elicitor-mediated |
| | | | | activation (2 copies) |
| Box 4 | ATTAAT | 718 | + | part of a conserved DNA module |
| | | | | involved in light responsiveness |
| CAAT-box | CAAAT | 5 | − | common cis-acting element in promoter |
| | | | | and enhancer regions |
| CAAT-box | CAAT | 19 | − | |
| CAAT-box | CAAT | 66 | + | |
| CAAT-box | CAAT | 160 | + | |
| CAAT-box | CAAAT | 194 | + | common cis-acting element in promoter |
| | | | | and enhancer regions |
| CAAT-box | CAAT | 243 | + | |
| CAAT-box | CAAT | 281 | − | |
| CAAT-box | CAAT | 348 | + | |
| CAAT-box | CAAAT | 370 | − | common cis-acting element in promoter |
| | | | | and enhancer regions |
| CAAT-box | CAAAT | 392 | + | common cis-acting element in promoter |
| | | | | and enhancer regions |
| CAAT-box | CAAAT | 453 | − | common cis-acting element in promoter |
| | | | | and enhancer regions |
| CAAT-box | CAAT | 470 | + | |
| CAAT-box | CAAAT | 479 | + | common cis-acting element in promoter |
| | | | | and enhancer regions |
| CAAT-box | CAAT | 535 | + | |
| CAAT-box | CAAT | 555 | + | |
| CAAT-box | CAAT | 600 | + | |
| CAAT-box | CAAT | 627 | + | |
| CAAT-box | CAAAT | 760 | − | common cis-acting element in promoter |
| | | | | and enhancer regions |
| CAAT-box | CAAAT | 795 | − | common cis-acting element in promoter |
| | | | | and enhancer regions |
| CAAT-box | CAAT | 829 | − | |
| CAAT-box | CAAAT | 865 | − | common cis-acting element in promoter |
| | | | | and enhancer regions |
| CAAT-box | CAAAT | 894 | − | common cis-acting element in promoter |
| | | | | and enhancer regions |
| CAAT-box | CAAT | 1002 | − | |
| CAAT-box | CAAT | 1050 | − | |
| CAAT-box | CCAAT | 1115 | − | common cis-acting element in promoter |
| | | | | and enhancer regions |
| CAAT-box | CAAAT | 1289 | − | common cis-acting element in promoter |
| | | | | and enhancer regions |
| CAAT-box | CAAT | 1311 | + | |
| CAAT-box | CAAT | 1336 | − | |
| CAAT-box | CAAAT | 1436 | − | common cis-acting element in promoter |
| | | | | and enhancer regions |
| CAAT-box | CAAAT | 1469 | + | common cis-acting element in promoter |
| | | | | and enhancer regions |
| CGTCA-motif | CGTCA | 337 | + | cis-acting regulatory element involved in |
| | | | | the MeJA-responsiveness |
| chs-CMA2a | TCACTTGA | 1318 | − | part of a light responsive element |
| ERE | ATTTCATA | 575 | + | |
| ERE | ATTTTAAA | 878 | − | |
| ERE | ATTTCATA | 1068 | + | |
| ERE | ATTTTAAA | 1128 | − | |
| ERE | ATTTCATA | 1282 | + | |
| GATA-motif | AAGGATAAGG | 1204 | + | part of a light responsive element |
| | (SEQ ID | | | |
| | NO: 101) | | | |
| G-box | CACGAC | 1194 | + | cis-acting regulatory element involved in |
| | | | | light responsiveness |
| GCN4_motif | TGAGTCA | 1314 | + | cis-regulatory element involved in |
| | | | | endosperm expression |
| GT1-motif | GGTTAA | 73 | + | light responsive element |
| GT1-motif | GGTTAA | 170 | − | light responsive element |
| GT1-motif | GGTTAA | 548 | − | light responsive element |
| I-box | ccttatcct | 1205 | − | part of a light responsive element |
| Myb | TAACTG | 654 | + | |
| Myb | TAACTG | 944 | + | |
| Myb | TAACTG | 1089 | + | |
| MYB | TAACCA | 171 | + | |
| MYB | TAACCA | 268 | + | |
| MYB | TAACCA | 549 | + | |
| MYB | TAACCA | 948 | − | |
| MYB | CAACAG | 1444 | + | |
| Myb-binding site | CAACAG | 1444 | + | |
| MYB-like | TAACCA | 171 | + | |
| sequence | | | | |
| MYB-like | TAACCA | 268 | + | |
| sequence | | | | |
| MYB-like | TAACCA | 549 | + | |
| sequence | | | | |
| MYB-like | TAACCA | 948 | − | |
| sequence | | | | |
| MYC | CATGTG | 85 | − | |
| MYC | CATTTG | 759 | + | |
| MYC | CATTTG | 893 | + | |
| STRE | AGGGG | 1479 | − | |
| TATA | TATAAAAT | 230 | + | |
| TATA-box | TATA | 69 | + | core promoter element around −30 of |
| | | | | transcription start |
| TATA-box | TACAAAA | 109 | − | core promoter element around −30 of |
| | | | | transcription start |
| TATA-box | ATATAA | 151 | + | core promoter element around −30 of |
| | | | | transcription start |
| TATA-box | TATA | 152 | + | core promoter element around −30 of |
| | | | | transcription start |
| TATA-box | TATA | 230 | + | core promoter element around −30 of |
| | | | | transcription start |
| TATA-box | ATATAA | 265 | + | core promoter element around −30 of |
| | | | | transcription start |
| TATA-box | TATA | 266 | + | core promoter element around −30 of |
| | | | | transcription start |
| TATA-box | ATATAT | 277 | + | core promoter element around −30 of |
| | | | | transcription start |
| TATA-box | TATA | 278 | + | core promoter element around −30 of |
| | | | | transcription start |
| TATA-box | TACAAAA | 383 | − | core promoter element around −30 of |
| | | | | transcription start |
| TATA-box | TATA | 494 | + | core promoter element around −30 of |
| | | | | transcription start |
| TATA-box | TATA | 513 | + | core promoter element around −30 of |
| | | | | transcription start |
| TATA-box | TATAA | 562 | − | core promoter element around −30 of |
| | | | | transcription start |
| TATA-box | TATA | 563 | + | core promoter element around −30 of |
| | | | | transcription start |
| TATA-box | TACAAAA | 569 | + | core promoter element around −30 of |
| | | | | transcription start |
| TATA-box | TACAAAA | 638 | + | core promoter element around −30 of |
| | | | | transcription start |
| TATA-box | TATAA | 666 | − | core promoter element around −30 of |
| | | | | transcription start |
| TATA-box | TATA | 667 | + | core promoter element around −30 of |
| | | | | transcription start |
| TATA-box | TATA | 692 | + | core promoter element around −30 of |
| | | | | transcription start |
| TATA-box | ATTATA | 699 | + | core promoter element around −30 of |
| | | | | transcription start |
| TATA-box | TATATAA | 700 | − | core promoter element around −30 of |
| | | | | transcription start |
| TATA-box | TATATA | 701 | + | core promoter element around −30 of |
| | | | | transcription start |
| TATA-box | ATATAT | 702 | + | core promoter element around −30 of |
| | | | | transcription start |
| TATA-box | TATATA | 703 | + | core promoter element around −30 of |
| | | | | transcription start |
| TATA-box | TATA | 705 | + | core promoter element around −30 of |
| | | | | transcription start |
| TATA-box | TATTTAAA | 728 | − | core promoter element around −30 of |
| | | | | transcription start |
| TATA-box | ATTATA | 856 | + | core promoter element around −30 of |
| | | | | transcription start |
| TATA-box | TATATAA | 857 | − | core promoter element around −30 of |
| | | | | transcription start |
| TATA-box | TATATA | 858 | − | core promoter element around −30 of |
| | | | | transcription start |
| TATA-box | ATATAT | 859 | − | core promoter element around −30 of |
| | | | | transcription start |
| TATA-box | TATATA | 860 | − | core promoter element around −30 of |
| | | | | transcription start |
| TATA-box | ATATAT | 861 | − | core promoter element around −30 of |
| | | | | transcription start |
| TATA-box | TATA | 862 | − | core promoter element around −30 of |
| | | | | transcription start |
| TATA-box | ATATAT | 884 | − | core promoter element around −30 of |
| | | | | transcription start |
| TATA-box | TATA | 885 | − | core promoter element around −30 of |
| | | | | transcription start |
| TATA-box | TACATAAA | 906 | − | core promoter element around −30 of |
| | | | | transcription start |
| TATA-box | TATAAAA | 937 | − | core promoter element around −30 of |
| | | | | transcription start |
| TATA-box | TATAAA | 938 | − | core promoter element around −30 of |
| | | | | transcription start |
| TATA-box | TATATAA | 939 | − | core promoter element around −30 of |
| | | | | transcription start |
| TATA-box | TATATA | 940 | − | core promoter element around −30 of |
| | | | | transcription start |
| TATA-box | ATATAA | 941 | + | core promoter element around −30 of |
| | | | | transcription start |
| TATA-box | TATA | 942 | − | core promoter element around −30 of |
| | | | | transcription start |
| TATA-box | TATAAGAA | 961 | − | core promoter element around −30 of |
| | | | | transcription start |
| TATA-box | TATAA | 964 | − | core promoter element around −30 of |
| | | | | transcription start |
| TATA-box | TATA | 965 | − | core promoter element around −30 of |
| | | | | transcription start |
| TATA-box | TATTTAAA | 983 | − | core promoter element around −30 of |
| | | | | transcription start |
| TATA-box | ATATAA | 1110 | + | core promoter element around −30 of |
| | | | | transcription start |
| TATA-box | TATA | 1111 | − | core promoter element around −30 of |
| | | | | transcription start |
| TATA-box | ccTATAAAaa | 1419 | + | core promoter element around −30 of |
| | (SEQ ID | | | transcription start |
| | NO: 97) | | | |
| TATA-box | TATA | 1421 | − | core promoter element around −30 of |
| | | | | transcription start |
| TATA-box | ATATAT | 1432 | − | core promoter element around −30 of |
| | | | | transcription start |
| TATA-box | TATA | 1433 | − | core promoter element around −30 of |
| | | | | transcription start |
| TC-rich repeats | ATTCTCTAAC | 294 | + | cis-acting element involved in defense |
| | (SEQ ID | | | and stress responsiveness |
| | NO: 100_ | | | |
| TC-rich repeats | ATTCTCTAAC | 433 | − | cis-acting element involved in defense |
| | (SEQ ID | | | and stress responsiveness |
| | NO: 100) | | | |
| TCT-motif | TCTTAC | 1119 | − | part of a light responsive element |
| TGACG-motif | TGACG | 337 | − | cis-acting regulatory element involved in |
| | | | | the MeJA-responsiveness |
| Unnamed_1 | CGTGG | 134 | − | |
| Unnamed_1 | CGTGG | 271 | − | |
| Unnamed_4 | CTCC | 900 | − | |
| Unnamed_4 | CTCC | 1139 | − | |
| Unnamed_4 | CTCC | 1199 | + | |
| Unnamed_4 | CTCC | 1269 | + | |
| Unnamed_4 | CTCC | 1295 | + | |
| Unnamed_4 | CTCC | 1492 | + | |
| WUN-motif | AAATTACT | 955 | − | |
| WUN-motif | AAATTACTA | 1242 | + | |

TABLE 12
Regulatory elements in PHY (1.5 kb) promoter (SEQ ID NO: 14).

| | | Nucleotide | | |
|---|---|---|---|---|
| Motif | Sequence | Position | Strand | Function |
| ABRE | ACGTG | 390 | + | cis-acting element involved in the |
| | | | | abscisic acid responsiveness |
| ABRE | ACGTG | 654 | + | cis-acting element involved in the |
| | | | | abscisic acid responsiveness |
| ABRE | ACGTG | 827 | − | cis-acting element involved in the |
| | | | | abscisic acid responsiveness |
| ABRE | ACGTG | 1346 | − | cis-acting element involved in the |
| | | | | abscisic acid responsiveness |
| ABRE3a | TACGTG | 827 | − | |
| ABRE3a | TACGTG | 1346 | − | |
| ABRE4 | CACGTA | 827 | + | |
| ABRE4 | CACGTA | 1346 | + | |
| AE-box | AGAAACAA | 497 | + | part of a module for light response |
| ARE | AAACCA | 1389 | − | cis-acting regulatory element essential |
| | | | | for the anaerobic induction |
| AT~TATA-box | TATATA | 447 | + | |
| AT~TATA-box | TATATA | 1413 | − | |
| AT-rich element | ATAGAAATCAA | 44 | + | binding site of AT-rich DNA binding |
| | (SEQ ID | | | protein (ATBP-1) |
| | NO: 99) | | | |
| AT-rich sequence | TAAAATACT | 1174 | + | element for maximal elicitor-mediated |
| | | | | activation (2copies) |
| CAAT-box | CAAAT | 41 | + | common cis-acting element in |
| | | | | promoter and enhancer regions |
| CAAT-box | CAAT | 52 | + | |
| CAAT-box | CCAAT | 77 | + | common cis-acting element in |
| | | | | promoter and enhancer regions |
| CAAT-box | CAAT | 78 | + | |
| CAAT-box | CAAT | 115 | + | |
| CAAT-box | CAAAT | 206 | + | common cis-acting element in |
| | | | | promoter and enhancer regions |
| CAAT-box | CAAT | 225 | + | |
| CAAT-box | CAAAT | 294 | + | common cis-acting element in |
| | | | | promoter and enhancer regions |
| CAAT-box | CAAT | 321 | + | |
| CAAT-box | CAAT | 356 | | |
| CAAT-box | CAAAT | 376 | + | common cis-acting element in |
| | | | | promoter and enhancer regions |
| CAAT-box | CAAT | 502 | + | |
| CAAT-box | CAAT | 578 | + | |
| CAAT-box | CCAAT | 584 | + | common cis-acting element in |
| | | | | promoter and enhancer regions |
| CAAT-box | CAAT | 585 | + | |
| CAAT-box | CAAT | 594 | + | |
| CAAT-box | CAAAT | 621 | + | common cis-acting element in |
| | | | | promoter and enhancer regions |
| CAAT-box | CAAT | 626 | + | |
| CAAT-box | CAAT | 838 | − | |
| CAAT-box | CCAAT | 847 | − | common cis-acting element in |
| | | | | promoter and enhancer regions |
| CAAT-box | CAAAT | 888 | − | common cis-acting element in |
| | | | | promoter and enhancer regions |
| CAAT-box | CAAAT | 961 | + | common cis-acting element in |
| | | | | promoter and enhancer regions |
| CAAT-box | CAAT | 982 | + | |
| CAAT-box | CAAT | 1090 | + | |
| CAAT-box | CAAAT | 1092 | − | common cis-acting element in |
| | | | | promoter and enhancer regions |
| CAAT-box | CAAAT | 1102 | + | common cis-acting element in |
| | | | | promoter and enhancer regions |
| CAAT-box | CAAAT | 1119 | − | common cis-acting element in |
| | | | | promoter and enhancer regions |
| CAAT-box | CAAT | 1231 | − | |
| CAAT-box | CAAT | 1245 | + | |
| CAAT-box | CAAT | 1247 | − | |
| CAAT-box | CAAT | 1292 | + | |
| CAAT-box | CAAT | 1325 | + | |
| CAAT-box | CAAAT | 1338 | + | common cis-acting element in |
| | | | | promoter and enhancer regions |
| CAAT-box | CCAAT | 1406 | + | common cis-acting element in |
| | | | | promoter and enhancer regions |
| CAAT-box | CAAT | 1407 | + | |
| CAAT-box | CAAT | 1425 | − | |
| CAAT-box | CAAT | 1446 | − | |
| CAAT-box | CAAT | 1457 | + | |
| CAAT-box | CAAT | 1470 | + | |
| CAAT-box | CAAT | 1489 | + | |
| CAT-box | GCCACT | 1364 | + | cis-acting regulatory element related to |
| | | | | meristem expression |
| circadian | CAAAGATATC | 1059 | + | cis-acting regulatory element involved |
| | (SEQ ID | | | in circadian control |
| | NO: 98) | | | |
| ERE | ATTTCATA | 84 | − | |
| ERE | ATTTTAAA | 1314 | + | |
| GATA-motif | GATAGGA | 239 | − | part of a light responsive element |
| G-Box | CACGTT | 389 | − | cis-acting regulatory element involved |
| | | | | in light responsiveness |
| G-Box | CACGTT | 653 | − | cis-acting regulatory element involved |
| | | | | in light responsiveness |
| G-box | TACGTG | 827 | − | cis-acting regulatory element involved |
| | | | | in light responsiveness |
| G-box | TACGTG | 1346 | − | cis-acting regulatory element involved |
| | | | | in light responsiveness |
| MRE | AACCTAA | 803 | − | MYB binding site involved in light |
| | | | | responsiveness |
| MYB | CAACAG | 1374 | + | |
| MYB | CAACCA | 1403 | + | |
| Myb | TAACTG | 1492 | + | |
| Myb-binding site | CAACAG | 1374 | + | |
| MYC | CATTTG | 961 | − | |
| MYC | CAATTG | 1245 | − | |
| STRE | AGGGG | 811 | − | |
| TATA-box | ATTATA | 427 | + | core promoter element around −30 of |
| | | | | transcription start |
| TATA-box | TATAA | 428 | − | core promoter element around −30 of |
| | | | | transcription start |
| TATA-box | TATA | 429 | + | core promoter element around −30 of |
| | | | | transcription start |
| TATA-box | TATATA | 447 | + | core promoter element around −30 of |
| | | | | transcription start |
| TATA-box | TATA | 449 | + | core promoter element around −30 of |
| | | | | transcription start |
| TATA-box | ATATAA | 545 | + | core promoter element around −30 of |
| | | | | transcription start |
| TATA-box | TATA | 546 | + | core promoter element around −30 of |
| | | | | transcription start |
| TATA-box | ATTATA | 863 | + | core promoter element around −30 of |
| | | | | transcription start |
| TATA-box | TATAA | 864 | − | core promoter element around −30 of |
| | | | | transcription start |
| TATA-box | TATA | 865 | − | core promoter element around −30 of |
| | | | | transcription start |
| TATA-box | ATATAA | 998 | + | core promoter element around −30 of |
| | | | | transcription start |
| TATA-box | TATA | 999 | − | core promoter element around −30 of |
| | | | | transcription start |
| TATA-box | ccTATAAAaa | 1007 | + | core promoter element around −30 of |
| | (SEQ ID | | | transcription start |
| | NO: 97) | | | |
| TATA-box | ATTATA | 1025 | + | core promoter element around −30 of |
| | | | | transcription start |
| TATA-box | TATAA | 1026 | − | core promoter element around −30 of |
| | | | | transcription start |
| TATA-box | TATA | 1027 | − | core promoter element around −30 of |
| | | | | transcription start |
| TATA-box | TATAA | 1258 | − | core promoter element around −30 of |
| | | | | transcription start |
| TATA-box | TATA | 1259 | − | core promoter element around −30 of |
| | | | | transcription start |
| TATA-box | TATA | 1287 | − | core promoter element around −30 of |
| | | | | transcription start |
| TATA-box | TATA | 1342 | − | core promoter element around −30 of |
| | | | | transcription start |
| TATA-box | ATTATA | 1357 | + | core promoter element around −30 of |
| | | | | transcription start |
| TATA-box | TATAA | 1358 | − | core promoter element around −30 of |
| | | | | transcription start |
| TATA-box | TATA | 1359 | − | core promoter element around −30 of |
| | | | | transcription start |
| TATA-box | ATATAT | 1412 | − | core promoter element around −30 of |
| | | | | transcription start |
| TATA-box | TATATA | 1413 | − | core promoter element around −30 of |
| | | | | transcription start |
| TATA-box | TATA | 1415 | − | core promoter element around −30 of |
| | | | | transcription start |
| TATA-box | TATA | 1420 | − | core promoter element around −30 of |
| | | | | transcription start |
| TATA-box | ATATAA | 1483 | + | core promoter element around −30 of |
| | | | | transcription start |
| TATA-box | TATA | 1484 | − | core promoter element around −30 of |
| | | | | transcription start |
| TCT-motif | TCTTAC | 489 | − | part of a light responsive element |
| TCT-motif | TCTTAC | 1462 | + | part of a light responsive element |
| TGA-element | AACGAC | 419 | − | auxin-responsive element |
| Unnamed_1 | CGTGG | 529 | − | |
| Unnamed_4 | CTCC | 695 | + | |
| Unnamed_4 | CTCC | 1056 | + | |
| Unnamed_4 | CTCC | 1143 | + | |
| Unnamed_6 | taTAAATATct | 999 | + | |
| | (SEQ ID | | | |
| | NO: 96) | | | |
| Unnamed_1 | GGATTTTACAGT | 230 | − | cis-acting element involved in |
| | (SEQ ID | | | phytochrome down-regulation |
| | NO: 95) | | | expression |
| W box | TTGACC | 222 | − | |
| WUN-motif | TAATTACTC | 869 | − | |
| WUN-motif | TTATTACAT | 1072 | + | |

TABLE 13
Regulatory elements in CYC (1.5 kb) promoter (SEQ ID NO: 16).

| | | Nucleotide | | |
|---|---|---|---|---|
| Motif | Sequence | Position | Strand | Function |
| ABRE | ACGTG | 956 | + | cis-acting element involved in the abscisic |
| | | | | acid responsiveness |
| AT~TATA-box | TATATA | 1375 | − | |
| AT~TATA-box | TATATA | 1377 | − | |
| Box 4 | ATTAAT | 931 | − | part of a conserved DNA module involved |
| | | | | in light responsiveness |
| Box 4 | ATTAAT | 1018 | − | part of a conserved DNA module involved |
| | | | | in light responsiveness |
| CAAT-box | CAAT | 19 | − | |
| CAAT-box | CAAT | 23 | + | |
| CAAT-box | CAAT | 27 | − | |
| CAAT-box | CAAT | 361 | − | |
| CAAT-box | CAAT | 371 | − | |
| CAAT-box | CAAAT | 379 | + | common cis-acting element in promoter |
| | | | | and enhancer regions |
| CAAT-box | CAAT | 404 | − | |
| CAAT-box | CAAT | 480 | − | |
| CAAT-box | CAAT | 558 | − | |
| CAAT-box | CAAT | 613 | − | |
| CAAT-box | CAAAT | 646 | + | common cis-acting element in promoter |
| | | | | and enhancer regions |
| CAAT-box | CAAAT | 692 | − | common cis-acting element in promoter |
| | | | | and enhancer regions |
| CAAT-box | CAAAT | 732 | + | common cis-acting element in promoter |
| | | | | and enhancer regions |
| CAAT-box | CAAT | 741 | − | |
| CAAT-box | CAAAT | 772 | − | common cis-acting element in promoter |
| | | | | and enhancer regions |
| CAAT-box | CAAT | 783 | + | |
| CAAT-box | CAAT | 811 | − | |
| CAAT-box | CAAAT | 855 | + | common cis-acting element in promoter |
| | | | | and enhancer regions |
| CAAT-box | CAAT | 867 | − | |
| CAAT-box | CAAT | 937 | − | |
| CAAT-box | CAAT | 990 | − | |
| CAAT-box | CAAT | 1000 | + | |
| CAAT-box | CAAT | 1025 | + | |
| CAAT-box | CCAAT | 1112 | − | common cis-acting element in promoter |
| | | | | and enhancer regions |
| CAAT-box | CAAT | 1118 | − | |
| CAAT-box | CAAAT | 1134 | + | common cis-acting element in promoter |
| | | | | and enhancer regions |
| CAAT-box | CAAT | 1174 | − | |
| CAAT-box | CAAT | 1181 | + | |
| CAAT-box | CAAT | 1190 | − | |
| CAAT-box | CAAT | 1197 | + | |
| CAAT-box | CAAAT | 1203 | − | common cis-acting element in promoter |
| | | | | and enhancer regions |
| CAAT-box | CAAAT | 1213 | − | common cis-acting element in promoter |
| | | | | and enhancer regions |
| CAAT-box | CAAT | 1224 | − | |
| CAAT-box | CAAT | 1241 | + | |
| CAAT-box | CAAT | 1324 | − | |
| CAAT-box | CAAT | 1342 | + | |
| CAAT-box | CAAAT | 1366 | − | common cis-acting element in promoter |
| | | | | and enhancer regions |
| CAAT-box | CAAAT | 1399 | + | common cis-acting element in promoter |
| | | | | and enhancer regions |
| CAAT-box | CAAT | 1410 | − | |
| CAT-box | GCCACT | 1394 | − | cis-acting regulatory element related to |
| | | | | meristem expression |
| circadian | CAAAGATATC | 1057 | + | cis-acting regulatory element involved in |
| | (SEQ ID | | | circadian control |
| | NO: 94) | | | |
| DRE1 | ACCGAGA | 677 | − | |
| ERE | ATTTCATA | 156 | − | |
| ERE | ATTTTAAA | 179 | − | |
| ERE | ATTTTAAA | 250 | + | |
| ERE | ATTTTAAA | 252 | − | |
| ERE | ATTTTAAA | 297 | − | |
| ERE | ATTTTAAA | 430 | − | |
| ERE | ATTTTAAA | 698 | + | |
| ERE | ATTTTAAA | 700 | − | |
| ERE | ATTTCATA | 1300 | − | |
| G-box | CACGTC | 955 | − | cis-acting regulatory element involved in |
| | | | | light responsiveness |
| GT1-motif | GGTTAAT | 54 | − | light responsive element |
| GT1-motif | GGTTAA | 55 | − | light responsive element |
| MYB | TAACCA | 56 | + | |
| Myb | TAACTG | 598 | + | |
| Myb | TAACTG | 1234 | − | |
| MYB-like | TAACCA | 56 | + | |
| sequence | | | | |
| MYC | CATTTG | 379 | − | |
| MYC | CATGTG | 578 | − | |
| MYC | CATTTG | 1134 | − | |
| TATA | TATAAAAT | 1106 | + | |
| TATA-box | TACAAAA | 9 | − | core promoter element around −30 of |
| | | | | transcription start |
| TATA-box | TATAA | 64 | − | core promoter element around −30 of |
| | | | | transcription start |
| TATA-box | TATA | 65 | + | core promoter element around −30 of |
| | | | | transcription start |
| TATA-box | TATAAA | 77 | − | core promoter element around −30 of |
| | | | | transcription start |
| TATA-box | TATAA | 78 | − | core promoter element around −30 of |
| | | | | transcription start |
| TATA-box | TATA | 79 | + | core promoter element around −30 of |
| | | | | transcription start |
| TATA-box | ATATAT | 153 | + | core promoter element around −30 of |
| | | | | transcription start |
| TATA-box | TATA | 154 | + | core promoter element around −30 of |
| | | | | transcription start |
| TATA-box | TATTTAAA | 177 | + | core promoter element around −30 of |
| | | | | transcription start |
| TATA-box | TATA | 237 | + | core promoter element around −30 of |
| | | | | transcription start |
| TATA-box | ATATAA | 270 | + | core promoter element around −30 of |
| | | | | transcription start |
| TATA-box | TATA | 27 | + | core promoter element around −30 of |
| | | | | transcription start |
| TATA-box | TATTTAAA | 295 | + | core promoter element around −30 of |
| | | | | transcription start |
| TATA-box | TATA | 401 | + | core promoter element around −30 of |
| | | | | transcription start |
| TATA-box | TATAAAA | 423 | − | core promoter element around −30 of |
| | | | | transcription start |
| TATA-box | TATAAA | 424 | − | core promoter element around −30 of |
| | | | | transcription start |
| TATA-box | TATAA | 425 | − | core promoter element around −30 of |
| | | | | transcription start |
| TATA-box | TATA | 426 | + | core promoter element around −30 of |
| | | | | transcription start |
| TATA-box | TATTTAAA | 428 | + | core promoter element around −30 of |
| | | | | transcription start |
| TATA-box | TACAAAA | 442 | − | core promoter element around −30 of |
| | | | | transcription start |
| TATA-box | TATAA | 476 | − | core promoter element around −30 of |
| | | | | transcription start |
| TATA-box | TATA | 477 | + | core promoter element around −30 of |
| | | | | transcription start |
| TATA-box | ATTATA | 590 | + | core promoter element around −30 of |
| | | | | transcription start |
| TATA-box | TATAA | 591 | − | core promoter element around −30 of |
| | | | | transcription start |
| TATA-box | TATA | 592 | + | core promoter element around −30 of |
| | | | | transcription start |
| TATA-box | TATA | 636 | + | core promoter element around −30 of |
| | | | | transcription start |
| TATA-box | ATATAA | 687 | + | core promoter element around −30 of |
| | | | | transcription start |
| TATA-box | TATA | 688 | + | core promoter element around −30 of |
| | | | | transcription start |
| TATA-box | TATAA | 756 | − | core promoter element around −30 of |
| | | | | transcription start |
| TATA-box | TATA | 757 | − | core promoter element around −30 of |
| | | | | transcription start |
| TATA-box | TATAA | 789 | − | core promoter element around −30 of |
| | | | | transcription start |
| TATA-box | TATA | 790 | − | core promoter element around −30 of |
| | | | | transcription start |
| TATA-box | TACAAAA | 800 | + | core promoter element around −30 of |
| | | | | transcription start |
| TATA-box | TATAAAA | 860 | − | core promoter element around −30 of |
| | | | | transcription start |
| TATA-box | TATAAA | 861 | − | core promoter element around −30 of |
| | | | | transcription start |
| TATA-box | TATAA | 862 | − | core promoter element around −30 of |
| | | | | transcription start |
| TATA-box | TATA | 863 | − | core promoter element around −30 of |
| | | | | transcription start |
| TATA-box | TACAAAA | 899 | − | core promoter element around −30 of |
| | | | | transcription start |
| TATA-box | TACAAAA | 982 | + | core promoter element around −30 of |
| | | | | transcription start |
| TATA-box | TATAAAA | 1008 | − | core promoter element around −30 of |
| | | | | transcription start |
| TATA-box | TATAAA | 1009 | − | core promoter element around −30 of |
| | | | | transcription start |
| TATA-box | TATAA | 1010 | − | core promoter element around −30 of |
| | | | | transcription start |
| TATA-box | TATA | 1011 | − | core promoter element around −30 of |
| | | | | transcription start |
| TATA-box | TATAAAA | 1042 | − | core promoter element around −30 of |
| | | | | transcription start |
| TATA-box | TATAAA | 1043 | − | core promoter element around −30 of |
| | | | | transcription start |
| TATA-box | TATAA | 1044 | − | core promoter element around −30 of |
| | | | | transcription start |
| TATA-box | TATA | 1045 | − | core promoter element around −30 of |
| | | | | transcription start |
| TATA-box | TATA | 1074 | − | core promoter element around −30 of |
| | | | | transcription start |
| TATA-box | TATA | 1106 | − | core promoter element around −30 of |
| | | | | transcription start |
| TATA-box | ATTATA | 1243 | + | core promoter element around −30 of |
| | | | | transcription start |
| TATA-box | TATAA | 1244 | − | core promoter element around −30 of |
| | | | | transcription start |
| TATA-box | TATA | 1245 | − | core promoter element around −30 of |
| | | | | transcription start |
| TATA-box | TACAAAA | 1281 | − | core promoter element around −30 of |
| | | | | transcription start |
| TATA-box | ATATAT | 1350 | − | core promoter element around −30 of |
| | | | | transcription start |
| TATA-box | TATA | 1351 | − | core promoter element around −30 of |
| | | | | transcription start |
| TATA-box | TATATA | 1375 | − | core promoter element around −30 of |
| | | | | transcription start |
| TATA-box | ATATAT | 1376 | − | core promoter element around −30 of |
| | | | | transcription start |
| TATA-box | TATATA | 1377 | − | core promoter element around −30 of |
| | | | | transcription start |
| TATA-box | TATA | 1379 | − | core promoter element around −30 of |
| | | | | transcription start |
| TATA-box | TACAAAA | 1442 | + | core promoter element around −30 of |
| | | | | transcription start |
| TCT-motif | TCTTAC | 13 | − | part of a light responsive element |
| Unnamed_1 | CGTGG | 1140 | + | |
| Unnamed_4 | CTCC | 776 | − | |
| WUN-motif | AAATTACT | 160 | + | |
| WUN-motif | AAATTTCTT | 287 | + | |
| WUN-motif | AAATTACT | 945 | − | |
| WUN-motif | AAATTACT | 1091 | − | |
| WUN-motif | AAATTACT | 1126 | − | |

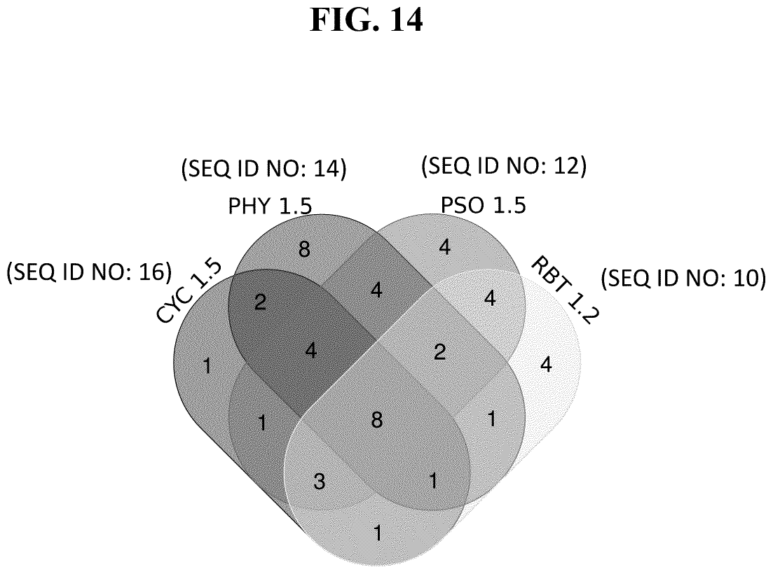

Several regulatory element motifs (e.g., TATA, CGTGG, TATATAAA, CATTTG, CAAAT, CAAT, CTCC, TAACCA, CAACAG, and SEQ ID NOs: 41 and 42) were identified as common elements. FIG. 14 depicts which promoters contain each of the common regulatory element motifs. Table 14 provides the regulatory element motifs used to generate FIG. 14.

TABLE 14
List of regulatory elements used to
create FIG. 14.

| CYC 1.5 | PHY 1.5 | PSO 1.5 | RBT 1.2 |
|---|---|---|---|
| (SEQ ID | (SEQ ID | (SEQ ID | (SEQ ID |
| NO: 16) | NO: 14) | NO: 12) | NO: 10) |
| Unnamed | Unnamed | Unnamed | Unnamed |
| ABRE | ABRE | AAGAA- | ABRE |
| | | MOTIF | |
| AT-TATA- | ABRE3a | ARE | ARE |
| BOX | | | |
| BOX4 | ABRE4 | AT-RICH | ATC- |
| | | SEQ | MOTIF |
| CAAT-BOX | AE-BOX | AT-TATA- | AT-TATA- |
| | | BOX | BOX |
| CAT-BOX | ARE | BOX4 | BOX4 |
| DRE1 | AT-RICH | CAAT- | CAAT- |
| | ELEMENT | BOX | BOX |
| ERE | AT-RICH | CGTCA- | CGTCA- |
| | SEQ | MOTIF | MOTIF |
| G-BOX | AT-TATA- | ERE | GT1- |
| | BOX | | MOTIF |
| GT1- | CAAT-BOX | G-BOX | LAMP- |
| MOTIF | | | ELEMENT |
| MYB | CAT-BOX | GATA- | MYB |
| | | MOTIF | |
| MYB-LIKE | ERE | GCN4 | MYB-LIKE |
| SEQ | | MOTIF | SEQ |
| MYC | G-BOX | GT1- | MYC |
| | | MOTIF | |
| Myb | G-BOX | I-BOX | STRE |
| TATA | GATA- | MYB | TATA-BOX |
| | MOTIF | | |
| TATA-BOX | MRE | MYB-LIKE | TCCC- |
| | | SEQ | MOTIF |
| TCT- | MYB | MYC | TGACG- |
| MOTIF | | | MOTIF |
| Un- | MYC | Myb | Un- |
| named_1 | | | named_1 |
| Un- | Myb | Myb- | Un- |
| named_4 | | BINDING | named_2 |
| | | SITE | |
| WUN- | Myb- | STRE | Un- |
| MOTIF | BINDING | | named_4 |
| | SITE | | |
| CIRCADIAN | STRE | TATA | W-BOX |
| | TATA-BOX | TATA-BOX | WUN-MOTIF |
| | TCT- | TC-RICH | AS-1 |
| | MOTIF | REPEATS | |
| | TGA- | TCT-MOTIF | CHS-CMA2a |
| | ELEMENT | | |
| | Unnamed_1 | TGACG- | |
| | | MOTIF | |
| | Unnamed_1 | Unnamed_1 | |
| | Unnamed_4 | Unnamed_4 | |
| | Unnamed-6 | WUN MOTIF | |
| | W-BOX | AS-1 | |
| | WUN MOTIF | CHS-CMA2a | |
| | CIRCADIAN | | |

### Example 10. MALD1 Promoter is a Trichome-Specific Promoter

The expression pattern of trichome-specific promoters (SEQ ID NOs: 80-82; see Example 3) is examined in the modified plants as produced in Example 4. First generation (e.g., To) modified plants are sampled during the vegetative stage. Young leaves, inflorescences, or both are examined for G3GFP accumulation under conditions allowing the visualization of G3GFP (excitation/emission wavelengths are 488 nm/500-550 nm) expressed by the trichome-specific promoters.

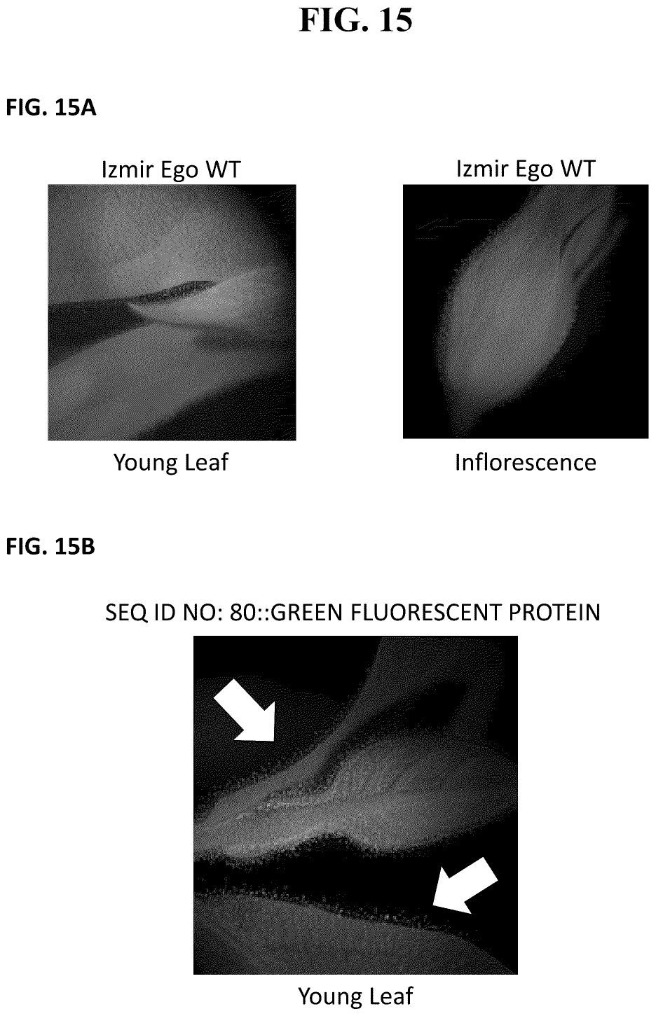

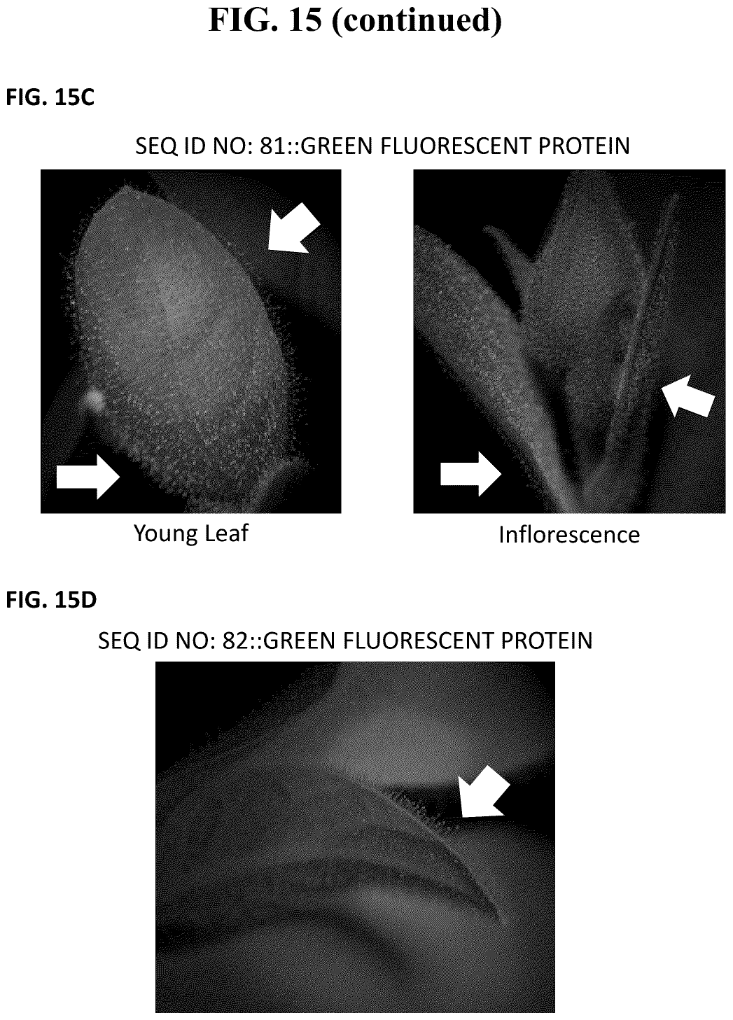

FIG. 15 depicts the accumulation of G3GFP in tobacco trichomes. Each of SEQ ID NOs: 80-82 drive the expression/accumulation of G3GFP in trichomes in tobacco. See FIG. 15B, FIG. 15C, and FIG. 15D.

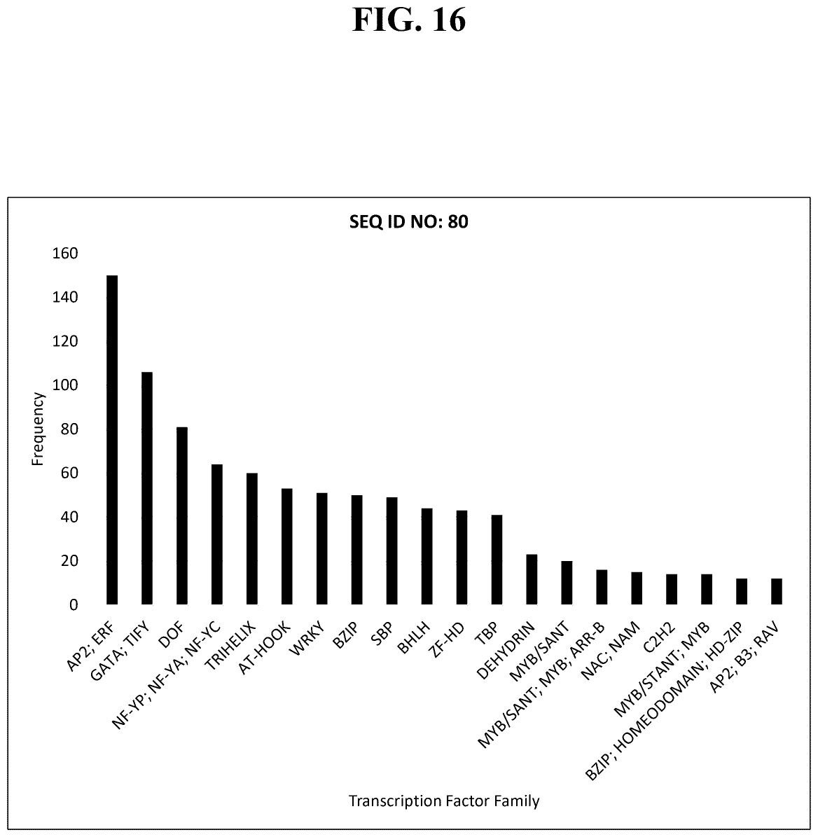

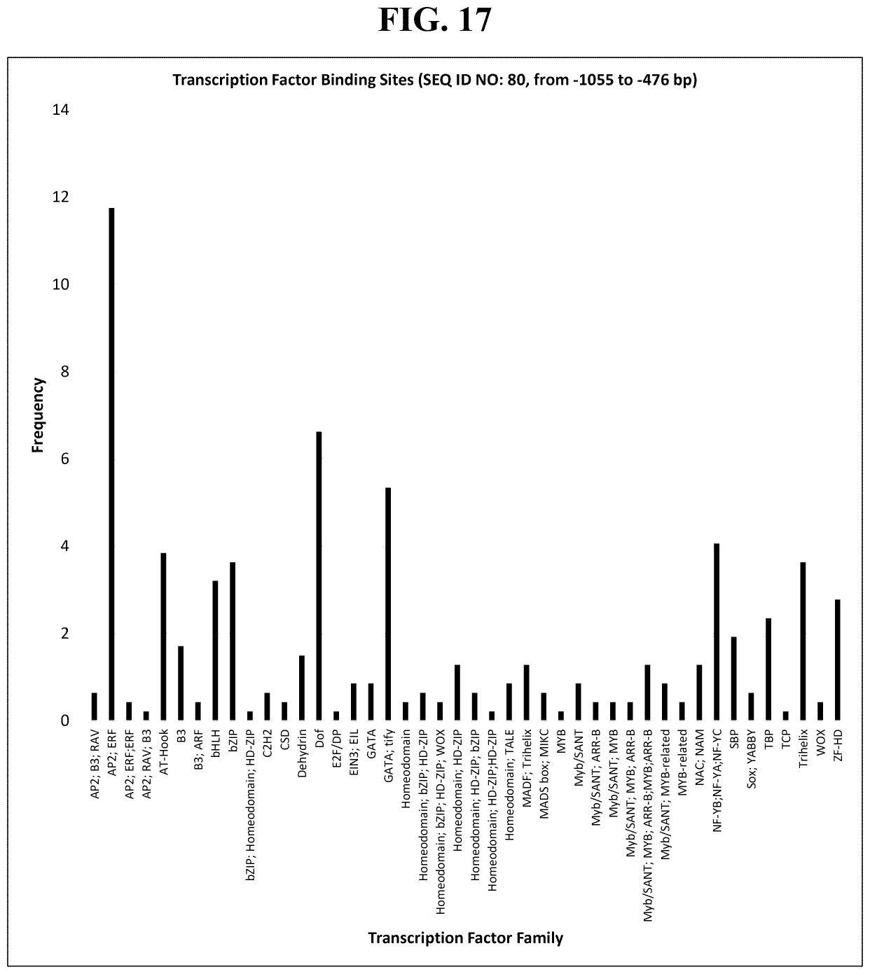

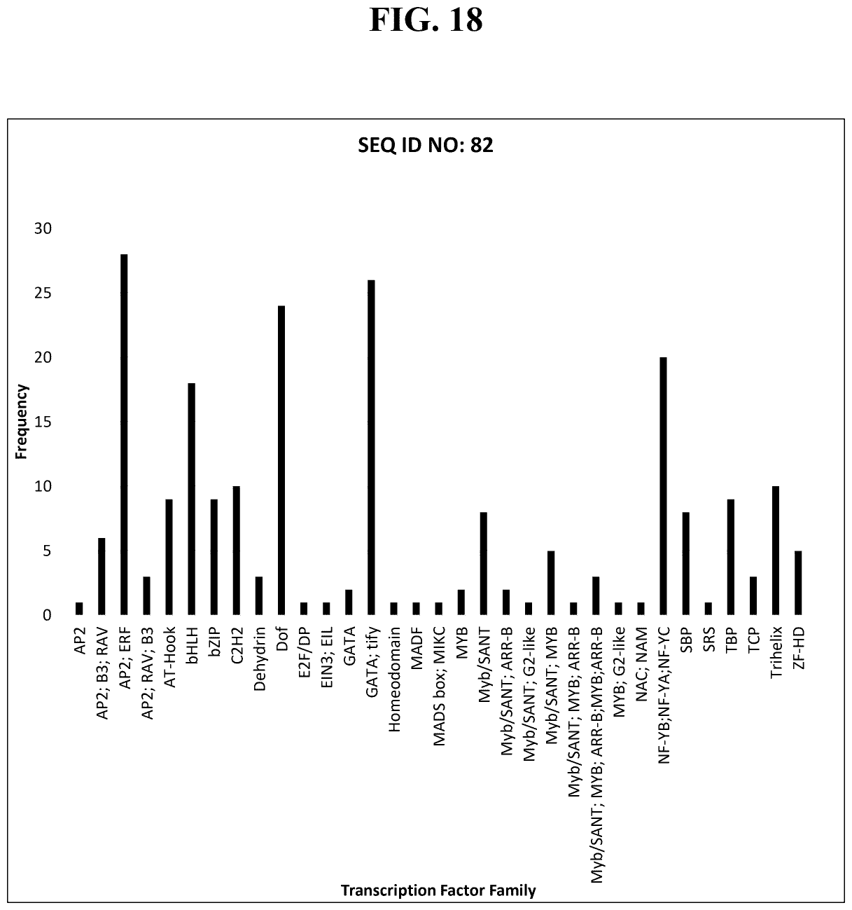

Trichome-specific promoter sequences (e.g., SEQ ID NOs: 80-82) are scanned for cis-regulatory element motifs using the publicly available online database PlantPAN3.0 (plantpan[dot]itps[dot]ncku[dot]edu[dot]tw/index[dot]html) using default settings. Graphs depicting the frequency of the cis-elements are shown in FIGS. 16-18. Further, Tables 15 and 16 detail the cis-regulatory element motifs that were identified that are unique to SEQ ID NO: 81 when compared to SEQ ID NO: 82, and the cis-elements that are unique to SEQ ID NO: 82 when compared to SEQ ID NO: 81, respectively.

TABLE 15
Cis-acting regulatory elements that are unique
to SEQ ID NO: 81 when compared to SEQ ID NO: 82.
Uppercase nucleotides are considered invariable
essential core sequences for the motifs.

| Transcription | | |
|---|---|---|
| Factor | | |
| Family (e.g., | | |
| cis-acting | | |
| regulatory | Binding | |
| element motif) | Sequence | Frequency |
| AP2; ERF; ERF | tGTCGGca | 2 |
| B3 | atGCATGc | 8 |
| B3; ARF | atGTCGGcat | 2 |
| | (SEQ ID NO: 83) | |
| bZIP; | gaATTATtat | 1 |
| Homeodomain; | (SEQ ID NO: 84) | |
| HD-ZIP | | |
| CSD | aATAAAaa | 2 |
| Homeodomain; | gttgaATTATtatt | 3 |
| bZIP; HD-ZIP | (SEQ ID NO: 85) | |
| Homeodomain; | ttTAATTatc | 2 |
| bZIP; HD-ZIP; | (SEQ ID NO: 86 | |
| WOX | | |
| Homeodomain; | catTTAATgc | 6 |
| HD-ZIP | (SEQ ID NO: 87) | |
| Homeodomain; | gaATTATt | 3 |
| HD-ZIP; bZIP | | |
| Homeodomain; | TAAATgtt | 1 |
| HD-ZIP; HD-ZIP | | |
| Homeodomain; | gaTGACAggt | 4 |
| TALE | (SEQ ID NO: 88) | |
| MADF; Trihelix | tGGTTAag | 6 |
| Myb/SANT; | aaATATCta | 4 |
| MYB-related | | |
| MYB-related | taaATATCta | 2 |
| | (SEQ ID NO: 89) | |
| Sox; YABBY | cttTAATTat | 3 |
| | (SEQ ID NO: 90) | |
| WOX | TTAATgc | 2 |

TABLE 16
Cis-acting regulatory elements that are unique
to SEQ ID NO: 82 when compared to SEQ ID NO: 81.
Uppercase nucleotides are considered invariable
essential core sequences for the motifs.

| Transcription | | |
|---|---|---|
| Factor | | |
| Family (e.g., | | |
| cis-acting | | |
| regulatory | | |
| element motif) | Binding Sequence | Frequency |
| AP2 | CACATgtacccatg | 1 |
| | (SEQ ID NO: 91) | |
| MADF | ctTACCGc | 1 |
| Myb/SANT; | taGATTCtag | 1 |
| G2-like | (SEQ ID NO: 92) | |
| MYB; G2-like | atAGATTcta | 1 |
| | (SEQ ID NO: 93) | |
| SRS | ggAGAGT | 1 |

Table 17 provides the frequency of cis elements identified in SEQ ID NO: 80.

TABLE 17
Frequency of cis-acting regulator
elements identified in SEQ ID NO: 80.

| Transcription Factor Family (e.g., cis- | |
|---|---|
| acting regulatory element motif) | Frequency |
| AP2; ERF | 150 |
| GATA; tify | 106 |
| Dof | 81 |
| NF-YB; NF-YA; NF-YC | 64 |
| Trihelix | 60 |
| AT-Hook | 53 |
| WRKY | 51 |
| bZIP | 50 |
| SBP | 49 |
| bHLH | 44 |
| ZF-HD | 43 |
| TBP | 41 |
| Dehydrin | 23 |
| Myb/SANT | 20 |
| Myb/SANT; MYB; ARR-B; MYB; ARR-B | 16 |
| NAC; NAM | 15 |
| C2H2 | 14 |
| Myb/SANT; MYB | 14 |
| bZIP; Homeodomain; HD-ZIP | 12 |
| AP2; B3; RAV | 12 |
| Homeodomain; HD-ZIP | 12 |
| Homeodomain; HD-ZIP; bZIP | 12 |
| GATA | 11 |
| Homeodomain; TALE | 11 |
| AP2; RAV; B3 | 11 |
| (Others) | 9 |
| Homeodomain; bZIP; HD-ZIP | 8 |
| B3 | 8 |
| Myb/SANT; MYB-related | 8 |
| Myb/SANT; ARR-B | 7 |
| MADS box; MIKC | 7 |
| MYB-related | 6 |
| EIN3; EIL | 6 |
| MADF; Trihelix | 6 |
| TCP | 6 |
| Homeodomain | 5 |
| Sox; YABBY | 5 |
| B3; ARF | 4 |
| HSF | 4 |
| MYB | 4 |
| Homeodomain; bZIP; HD-ZIP; WOX | 4 |
| Myb/SANT; MYB; ARR-B | 3 |
| TCR; CPP | 3 |
| WOX | 3 |
| AP2; RAV | 2 |
| AP2; ERF; ERF | 2 |
| CSD | 2 |
| HD-ZIP | 2 |
| Homeodomain; HD-ZIP; HD-ZIP | 2 |
| E2F/DP | 2 |
| MYB; ARR-B | 1 |
| MYB; G2-like | 1 |
| AP2; B3 | 1 |
| GRAS | 1 |
| MADF | 1 |
| Myb/SANT; G2-like | 1 |
| AP2 | 1 |
| VOZ | 1 |
| SRS | 1 |
| Unknown | 373 |

1.-40. (canceled)

41. Cured tobacco material from a modified tobacco plant, wherein the modified tobacco plant comprises a recombinant nucleic acid molecule comprising a promoter operably linked to a heterologous polynucleotide encoding a polypeptide, wherein the promoter comprises a nucleic acid sequence at least 95% identical to a nucleic acid sequence selected from the group consisting of SEQ ID NOs: 46 and 47.

42. The cured tobacco material of claim 41, wherein the nucleic acid sequence is at least 98% identical to a nucleic acid sequence selected from the group consisting of SEQ ID NOs: 46 and 47.

43. The cured tobacco material of claim 41, wherein the nucleic acid sequence is selected from the group consisting of SEQ ID NOs: 46 and 47.

44. The cured tobacco material of claim 41, wherein the polypeptide comprises an amino acid sequence at least 95% identical to an amino acid sequence selected from the group consisting of SEQ ID NOs: 18 to 26 and 64 to 79.

45. The cured tobacco material of claim 41, wherein the polypeptide comprises an amino acid sequence selected from the group consisting of SEQ ID NOs: 18 to 26 and 64 to 79.

46. The cured tobacco material of claim 41, wherein the heterologous polynucleotide comprises a nucleic acid sequence at least 95% identical to a nucleic acid sequence selected from the group consisting of SEQ ID NOs: 27 to 35 and 48 to 63.

47. The cured tobacco material of claim 41, wherein the heterologous polynucleotide comprises a nucleic acid sequence selected from the group consisting of SEQ ID NOs: 27 to 35 and 48 to 63.

48. The cured tobacco material of claim 41, wherein the polypeptide is involved in the biosynthesis of at least one terpene.

49. The cured tobacco material of claim 48, wherein the at least one terpene is cis-abienol.

50. The cured tobacco material of claim 48, wherein the polypeptide is selected from the group consisting of geranylgeranyl diphosphate synthase, 8-hydroxy-copalyl diphosphate synthase, cis-abienol synthase, cembratrienol synthase 2a, levopimaradiene synthetase, 2-isopropylmalate synthetase, 2-oxoisovalerate dehydrogenase, and neomenthol dehydrogenase.

51. The cured tobacco material of claim 50, wherein the cis-abienol synthase is selected from the group consisting of cis-abienol synthase ISOFORM1 and cis-abienol synthase ISOFORM2.

52. The cured tobacco material of claim 41, wherein the cured tobacco material is selected from the group consisting of flue-cured tobacco material, air-cured tobacco material, fire-cured tobacco material, and sun-cured tobacco material.

53. The cured tobacco material of claim 41, wherein the cured tobacco material is selected from the group consisting of cured leaf material and cured stem material.

54. The cured tobacco material of claim 41, wherein the modified tobacco plant is of a tobacco variety selected from the group consisting of a flue-cured variety, a bright variety, a Burley variety, a Virginia variety, a Maryland variety, a dark variety, a Galpão variety, an Oriental variety, and a Turkish variety.

55. The cured tobacco material of claim 41, wherein the modified tobacco plant is a tobacco plant from a variety selected from the group consisting of: 400 (TC 225), 401 (TC 226), 401 Cherry Red (TC 227), 401 Cherry Red Free (TC 228), Cash (TC 250), Cash (TI 278), CC 101, CC 1063, CC 13, CC 143, CC 200, CC 27, CC 301, CC 33, CC 35, CC 37, CC 400, CC 500, CC 600, CC 65, CC 67, CC 700, CC 800, CC 900, Coker 139 (TC 259), Coker 139 yb1, yb2, Coker 140 (TC 260), Coker 176 (TC 262), Coker 187 (TC 263), Coker 187-Hicks (TC 265), Coker 209 (TC 267), Coker 258 (TC 270), Coker 298 (TC 272), Coker 316 (TC 273), Coker 319 (TC 274), Coker 347 (TC 275), Coker 371-Gold (TC 276), Coker 411 (TC 277), Coker 48 (TC 253), Coker 51 (TC 254), Coker 86 (TC 256), CU 263 (TC 619), CU 561, DH95-1562-1, Dixie Bright 101 (TC 290), Dixie Bright 102 (TC 291), Dixie Bright 244 (TC 292), Dixie Bright 27 (TC 288), Dixie Bright 28 (TC 289), GF 157, GF 318, GL 26H, GL 338, GL 350, GL 368, GL 395, GL 600, GL 737, GL 939, GL 939 (TC 628), Hicks (TC 310), Hicks Broadleaf (TC 311), K 149 (TC 568), K 317, K 326, K 326 (TC 319), K 340 (TC 320), K 346, K 346 (TC 569), K 358, K 394 (TC 321), K 399, K 399 (TC 322), K 730, Lonibow (TI 1573), Lonibow (TI 1613), McNair 10 (TC 330), McNair 135 (TC 337), McNair 30 (TC 334), McNair 373 (TC 338), McNair 944 (TC 339), MK94 (TI 1512), MS K 326, MS NC 71, MS NC 72, NC 100, NC 102, NC 1071 (TC 364), NC 1125-2, NC 12 (TC 346), NC 1226, NC 196, NC 2326 (TC 365), NC 27 NF (TC 349), NC 291, NC 297, NC 299, NC 37 NF (TC 350), NC 471, NC 55, NC 567 (TC 362), NC 60 (TC 352), NC 606, NC 6140, NC 71, NC 72, NC 729 (TC 557), NC 810 (TC 659), NC 82 (TC 356), NC 8640, NC 89 (TC 359), NC 92, NC 925, NC 95 (TC 360), NC 98 (TC 361), NC EX 24, NC PY 10 (TC 367), NC TG 61, Oxford 1 (TC 369), Oxford 1-181 (TC 370), Oxford 2 (TC 371), Oxford 207 (TC 632), Oxford 26 (TC 373), Oxford 3 (TC 372), Oxford 414 NF, PD 611 (TC 387), PVH 03, PVH 09, PVH 1118, PVH 1452, PVH 1600, PVH 2110, PVH 2275, R 83 (Line 256-1) (TI 1400), Reams 134, Reams 158, Reams 713, Reams 744, Reams M1, RG 11 (TC 600), RG 13 (TC 601), RG 17 (TC 627), RG 22 (TC 584), RG 8 (TC 585), RG 81 (TC 618), RG H51, RG4H 217, RGH 12, RGH 4, RGH 51, RGH 61, SC 58 (TC 400), SC 72 (TC 403), Sp. G-168, SPEIGHT 168, Speight 168 (TC 633), Speight 172 (TC 634), Speight 178, Speight 179, Speight 190, Speight 196, SPEIGHT 220, SPEIGHT 225, SPEIGHT 227, SPEIGHT 236, Speight G-10 (TC 416), Speight G-102, Speight G-108, Speight G-111, Speight G-117, Speight G-126, Speight G-15 (TC 418), Speight G-23, Speight G-28 (TC 420), Speight G-33, Speight G-41, Speight G-5, Speight G-52, Speight G-58, Speight G-70, Speight G-70 (TC 426), Speight G-80 (TC 427), Speight NF3 (TC 629), STNCB, VA 182, VA 45 (TC 559), Vesta 30 (TC 439), Vesta 33 (TC 440), Vesta 5 (TC 438), Vesta 62 (TC 441), Virginia (TI 220), Virginia (TI 273), Virginia (TI 877), Virginia 115 (TC 444), Virginia 21 (TC 443), Virginia Bright (TI 964), Virginia Bright Leaf (TC 446), Virginia Gold (TC 447), White Stem Orinoco (TC 451), 4407 LC, AA-37-1, Burley 21 (TC 7), Burley 49 (TC 10), Burley 64 (TC 11), Burley Mammoth KY 16 (TC 12), Clay 402, Clay 403, Clay 502, Clays 403, GR 10 (TC 19), GR 10 (TC 19), GR 10A (TC 20), GR 13 (TC 21), GR 14 (TC 22), GR 149 LC, GR 153, GR 17 (TC 23), GR 17B (TC 24), GR 18 (TC 25), GR 19 (TC 26), GR 2 (TC 15), GR 24 (TC 27), GR 36 (TC 28), GR 38 (TC 29), GR 38A (TC 30), GR 40 (TC 31), GR 42 (TC 32), GR 42C (TC 33), GR 43 (TC 34), GR 44 (TC 35), GR 45 (TC 36), GR 46 (TC 37), GR 48 (TC 38), GR 5 (TC 16), GR 53 (TC 39), GR 6 (TC 17), GR 9 (TC 18), GR139 NS, GR139 S, HB 04P, HB 04P LC, HB 3307P LC, HB 4108P, HB 4151P, HB 4192P, HB 4194P, HB 4196, HB 4488, HB 4488P, HB04P, HB 4488 LC, HIB 21, HPB 21, HY 403, Hybrid 403 LC, Hybrid 404 LC, Hybrid 501 LC, KDH-959 (TC 576), KDH-960 (TC 577), KT 200 LC, KT 204 LC, KT 206 LC, KT 209 LC, KT 210 LC, KT 212 LC, KT 215 LC, KY 1 (TC 52), KY 10 (TC 55), KY 12 (TC 56), KY 14 (TC 57), KY 14×L8 LC, KY 15 (TC 58), KY 16 (TC 59), KY 17 (TC 60), KY 19 (TC 61), KY 21 (TC 62), KY 22 (TC 63), KY 24 (TC 64), KY 26 (TC 65), KY 33 (TC 66), KY 34 (TC 67), KY 35 (TC 68), KY 41A (TC 69), KY 5 (TC 53), KY 52 (TC 70), KY 54 (TC 71), KY 56 (TC 72), KY 56 (TC 72), KY 57 (TC 73), KY 58 (TC 74), KY 8654 (TC 77), KY 8959, KY 9 (TC 54), KY 907 LC, KY 908 (TC 630), NBH 98 (Screened), NC 1206, NC 129, NC 2000 LC, NC 2002 LC, NC 3 LC, NC 5 LC, NC 6 LC, NC 7 LC, NC BH 129 LC, NC03-42-2, Newton 98, R 610 LC, R 630 LC, R 7-11, R 7-12 LC, RG 17, TKF 1801 LC, TKF 2002 LC, TKF 4024 LC, TKF 4028 LC, TKF 6400 LC, TKF 7002 LC, TKS 2002 LC, TN 86 (TC 82), TN 90 LC, TN 97 Hybrid LC, TN 97 LC, VA 116, VA 119, Virgin A Mutante (TI 1406), Virginia 509 (TC 84), Maryland 10 (TC 498), Maryland 14 D2 (TC 499), Maryland 201 (TC 503), Maryland 21 (TC 500), Maryland 341 (TC 504), Maryland 40, Maryland 402, Maryland 59 (TC 501), Maryland 601, Maryland 609 (TC 505), Maryland 64 (TC 502), Maryland 872 (TC 506), Maryland Mammoth (TC 507), Black Mammoth (TC 461), Black Mammoth Small Stalk (TC 641), Certified Madole (TC 463), D-534-A-1 (TC 464), DAC ULT 302, DAC ULT 303, DAC ULT 306, DAC ULT 308, DAC ULT 312, DF 300 (TC 465), DF 485 (TC 466), DF 516 (TC 467), DF 911 (TC 468), DT 508, DT 518 (Screened), DT 538 LC, DT 592, Improved Madole (TC 471), Jernigan's Madole (TC 472), KT 14LC, KT D17LC, KT D4 LC, KT D6 LC, KT D8 LC, KY 153 (TC 216), KY 157 (TC 217), KY 160, KY 160 (TC 218), KY 163 (TC 219), KY 165 (TC 220), KY 170 (TC 474), KY 171 (PhPh), KY 171 (TC 475), KY 171 LC, KY 171 NS, KY 180 (TC 573), KY 190 (TC 574), Little Crittenden, Little Crittenden (TC 476), Little Crittenden LC (certified), Little Crittenden PhPh, Lizard Tail Turtle Foot, Madole (TC 478), Madole (TC 479), MS KY 171, MS NL Madole LC, MS TN D950 LC, Nance (TC 616), Narrow Leaf Madole LC (certified), Neal Smith Madole (TC 646), Newtons VH Madole, NL Madole, NL Madole (PhPh), NL Madole (TC 484), NL Madole LC, NL Madole LC (PhPh), NL Madole NS, One Sucker (TC 224), OS 400, PD 302H, PD 312H, PD 318H, PD 7302 LC, PD 7305, PD 7309 LC, PD 7312 LC, PD 7318 LC, PD 7319 LC, Petico M PG04, PY KY 160 (TC 612), PY KY 171 (TC 613), Shirey, TI 1372, TN D94, TN D94 (TC 621), TN D950, TN D950 (PhPh), TN D950, TN D950 (TC 622), TR Madole (TC 486), VA 309, VA 309 (TC 560), VA 309 LC (certified), VA 310 (TC 487), VA 331 (TC 592), VA 355 (TC 638), VA 359, VA 359 (Screened), VA 359 (TC 639), VA 359 LC (certified), VA 403 (TC 580), VA 405 (TC 581), VA 409 (TC 562), VA 510 (TC 572), Bafra (TI 1641), Bahce (TI 1730), Bahia (TI 1416), Bahia (TI 1455), Baiano (TI 128), Basma, Basma (TI 1666), Basma Drama, Basma Hybrid (PhPh), Basma Zihna I, Bitlis (TI 1667), Bitlis (TI 1725), Bubalovac (TI 1282), Bursa (TI 1650), Bursa (TI 1668), Canik (TI 1644), Djebel 174 (TI 1492), Djebel 359 (TI 1493), Djebel 81, Dubec 566 (TI 1409), Dubec 7 (TI 1410), Dubek 566 (TI 1567), Duzce (TI 1670), Edirne (TI 1671), Ege (TI 1642), Ege-64 (TI 1672), Izmir (Akhisar) (TI 1729), Izmir (Gavurkoy) (TI 1727), Izmir Ege 64, Izmir-Incekara (TI 1674), Izmir-Ozbas (TI 1675), Jaka Dzebel (TI 1326), Kaba-Kulak, Kagoshima Maruba (TI 158), Katerini, Katerini S53, Krumovgrad 58, MS Basma, MS Katerini S53, Nevrokop 1146, Ozbas (TI 1645), Perustitza (TI 980), Prilep (TI 1291), Prilep (TI 1325), Prilep 12-2/1, Prilep 23, Samsun (TC 536), Samsun 959 (TI 1570), Samsun Evkaf (TI 1723), Samsun Holmes NN (TC 540), Samsun Maden (TI 1647), Samsun NO 15 (TC 541), Samsun-BLK SHK Tol (TC 542), Samsun-Canik (TI 1678), Samsun-Maden (TI 1679), Saribaptar 407-Izmir Region, Smyrna (TC 543), Smyrna No. 23 (TC 545), Smyrna No. 9 (TC 544), Smyrna-Blk Shk Tol (TC 546), Trabzon (TI 1649), Trabzon (TI 1682), Trapezund 161 (TI 1407), Turkish (TC 548), Turkish Angshit (TI 90), Turkish Samsum (TI 92), Turkish Tropizoid (TI 93), Turkish Varotic (TI 89), Xanthi (TI 1662), Bahai (TI 62), Beinhart 1000, Beinhart 1000 (TI 1562), Beinhart 1000-1 (TI 1561), Bergerac C, Bergerac C (TI 1529), Big Cuban (TI 1565), Castillo Negro, Blanco, Pina (TI 448), Castillo Negro, Blanco, Pina (TI 448A), Castillo Negro, Blanco, Pina (TI 449), Caujaro (TI 893), Chocoa (TI 289), Chocoa (TI 313), Connecticut 15 (TC 183), Connecticut Broadleaf, Connecticut Broadleaf (TC 186), Connecticut Shade (TC 188), Criollo Colorado (TI 1093), Enshu (TI 1586), Florida 301, Florida 301 (TC 195), PA Broadleaf (TC 119), Pennsylvania Broadleaf, Pennsylvania Broadleaf (TC 119), Petite Havana SR1, Petite Havana SR1 (TC 105), Chocoa (TI 319), Hoja Parada (TI 1089), Hoja Parado (Galpoa) (TI 1068), Perique (St. James Parrish), Perique (TC 556), Perique (TI 1374), Sylvestris (TI 984), and TI 179.

56. The cured tobacco material of claim 41, wherein the modified tobacco plant is a male sterile tobacco plant or a cytoplasmically male sterile tobacco plant.

57. The cured tobacco material of claim 41, wherein the modified tobacco plant is a female sterile tobacco plant.

58. A tobacco blend comprising the cured tobacco material of claim 41.

59. The tobacco blend of claim 58, wherein the tobacco blend further comprises tobacco material selected from the group consisting of: uncured tobacco material, fermented tobacco material, reconstituted tobacco material, and expanded tobacco material.

60. A reconstituted tobacco comprising the cured tobacco material of claim 41.


SEQUENCE LISTING (102 sequences)

SEQ ID NO: 1 - DNA, 53 bp, synthetic construct
  misc_feature 1..53: note = Description of Artificial Sequence: Synthetic primer
  source 1..53: mol_type = other DNA
ggggacaagtttgtacaaaaaagcaggcttataaatacctatgaaaattaaat

SEQ ID NO: 2 - DNA, 54 bp, synthetic construct
  misc_feature 1..54: note = Description of Artificial Sequence: Synthetic primer
  source 1..54: mol_type = other DNA
ggggaccactttgtacaagaaagctgggtaggcagttattgttatatgtgatag

SEQ ID NO: 3 - DNA, 50 bp, synthetic construct
  misc_feature 1..50: note = Description of Artificial Sequence: Synthetic primer
  source 1..50: mol_type = other DNA
ggggacaagtttgtacaaaaaagcaggctgcaataattttttatatctat

SEQ ID NO: 4 - DNA, 48 bp, synthetic construct
  misc_feature 1..48: note = Description of Artificial Sequence: Synthetic primer
  source 1..48: mol_type = other DNA
ggggaccactttgtacaagaaagctgggtactctctctctttctctcg

SEQ ID NO: 5 - DNA, 50 bp, synthetic construct
  misc_feature 1..50: note = Description of Artificial Sequence: Synthetic primer
  source 1..50: mol_type = other DNA
ggggacaagtttgtacaaaaaagcaggctatttacgagtttactatactt

SEQ ID NO: 6 - DNA, 48 bp, synthetic construct
  misc_feature 1..48: note = Description of Artificial Sequence: Synthetic primer
  source 1..48: mol_type = other DNA
ggggaccactttgtacaagaaagctgggtatttgggagggaattaaag

SEQ ID NO: 7 - DNA, 50 bp, synthetic construct
  misc_feature 1..50: note = Description of Artificial Sequence: Synthetic primer
  source 1..50: mol_type = other DNA
ggggacaagtttgtacaaaaaagcaggctattagcatcaaccgggttagc

SEQ ID NO: 8 - DNA, 51 bp, synthetic construct
  misc_feature 1..51: note = Description of Artificial Sequence: Synthetic primer
  source 1..51: mol_type = other DNA
ggggacaagtttgtacaaaaaagcaggctttcatgaatctcaatatggagg

SEQ ID NO: 9 - DNA, 52 bp, synthetic construct
  misc_feature 1..52: note = Description of Artificial Sequence: Synthetic primer
  source 1..52: mol_type = other DNA
ggggaccactttgtacaagaaagctgggtagttcaccttcactttaagctac

SEQ ID NO: 10 - DNA, 1243 bp, synthetic construct
  misc_feature 1..1243: note = Description of Artificial Sequence: Synthetic polynucleotide
  source 1..1243: mol_type = other DNA
attagcatcaaccgggttagcaattggggcattattgcgatcagcgggaggcaccttgttgctgagcaccaaattattctttttgccgtgatggccagactcaacgtcaacgttcaagtgagcagaccaaaagtttaacttttttgggatggcttgaaattaagactttaaataataagcataaaatgaggtgttatggagattcggatcaaaccaccactattatccctagccccacggttgatgccaaactgtttatttcaaaatcggataacaattacatttgtatgcggttttaagaatatgtgatttaattcaacacgaacaattaagaatatcaaataatcaaattagagataaaataatcatccaaacgaattgcgatattatggctaaccatgagcttagactgaccgatgagctcgctcttggttgaccttcgagcccagccgtgaatgaagtacataacaaatgagcaaaaactttaacaatagctaaaaggcagaaagtaaacttgtattgctttgatttacatattacaacatgtgttacaaaagaaaaacttccaccctttatatagtggagagtttcatccccagtataaatctaaaaaaggtaaaaatcttcctttgctggtaattactaattcatgatcgacatcgagtgagatttgcaccgtaatatccggttgattgcgatatcacgatcctctatctgtcatgtgtaaccgtttatcatgcctcccgagatcttagaattcattcttggaccggggtgcatgcccaataacaggcacattgttcgcccttcatgaatctcaatatggagggtctttaacctcgattataattttgtgtatatgtactcttcctctattttctcgtcgaaaaatcggagtaaacattatccccgattttaccatacacatacgttttgcctaaataaaatgacaaaagagaaagggtgacgaaaactgtcactatgcctaactaaccctcgtagataccgtcttccgcgaaccctacacacccctaaggccctaaccccttcttcaccttactttattaattacctcactcacttttgtcctcttatgtaaccgtttccagtttaattacaaaaaaattcagattactttgaagcatcacttctctccctttcatataaactcagtttttttgttcgagtctttatacatatgtctttatcataactaaagtagtagcttaaagtgaaggtgaac

SEQ ID NO: 11 - DNA, 436 bp, synthetic construct
  misc_feature 1..436: note = Description of Artificial Sequence: Synthetic polynucleotide
  source 1..436: mol_type = other DNA
ttcatgaatctcaatatggagggtctttaacctcgattataattttgtgtatatgtactcttcctctattttctcgtcgaaaaatcggagtaaacattatccccgattttaccatacacatacgttttgcctaaataaaatgacaaaagagaaagggtgacgaaaactgtcactatgcctaactaaccctcgtagataccgtcttccgcgaaccctacacacccctaaggccctaaccccttcttcaccttactttattaattacctcactcacttttgtcctcttatgtaaccgtttccagtttaattacaaaaaaattcagattactttgaagcatcacttctctccctttcatataaactcagtttttttgttcgagtctttatacatatgtctttatcataactaaagtagtagcttaaagtgaaggtgaac

SEQ ID NO: 12 - DNA, 1500 bp, synthetic construct
  misc_feature 1..1500: note = Description of Artificial Sequence: Synthetic polynucleotide
  source 1..1500: mol_type = other DNA
aaaatatttgaaagaaaatattgtagctaaatgaatatttttccttcaagttattaaaagttgtggcaatataggttaaattagccacatgtttcttgctttaatagaattttgtagctaatcattaacttttaccacgagttgaaacttaatataacaacaataaccttttaaccataataaaccatttaaatcaaatattactaaataaataactttgcttcaagttctataaaatcatggcaatagtcattacgataaaatgatataaccacgaatatattgcaacgataaattctgtaactaatcattagtttttgcgacgaggtaaattttccgtcacagtagcaatcttctaggcacattaaaaatttgaaacaaaattttgtagtcaaataaatatttatcttcttattttaagaaaataaaaatagttagataatagttactactatttgtcatgaaaatatcaatagatacaaatttaaagtgactataaatttacgagtttactatactttagtcgtacagtttgcaataatagtattttaaccacaattagttatatgtacaaaatttcataagtgaataactttttttcaatgacaaaaataagagttgctcaaacaatatcaagttacaaaaatttaattttaactgtaaaagttatatttttccaaaataacataaactatagtaattatatatagtttgaagtattaataaaatttaaatatgcaaaagttaattttaataaaccatttgtatgcctacttgtagcctctaaactattttatttgctttatttatcaaactcatattttattttattgcaccttgttagttttggacgttaattatatatatttggtgtaaaatttaaaatatattaacatttgtggagaatttatgtatgcctggttcttaactattttttttttatataactggttagagtaatttcttatatttcagtatttatttttaaataagtcctcataaattgaagactttaaaagtttttgtgtcattcctctttttatttaagaaattgaagaattccgctaaatttcatattccgctgttatttaactgtttattcccttgttaatataattggtaagaagttttaaaataaaggagttaatgatttctaggttcatggcttgcctagcttctacgagtaagcgccatcacgactcccgaggataaggaaatccgggtcgtagcattcactcacaaaaattactaaaaacaaagtttacccttctcccaaaagtaaatttcatatttggctccacataatgtgttcaatgagtcaagtgaagtacttttcattgacaaaaaaaagttgctgaaaaatgcatatctcatattttttttttttagagaaatcccatttcttgcctaaacgaaatgcctataaaagagcatatatttgcaacaacagtttgcagaaactatcaagtcaaataatcccccctttaattccctcccaaa

SEQ ID NO: 13 - DNA, 1000 bp, synthetic construct
  misc_feature 1..1000: note = Description of Artificial Sequence: Synthetic polynucleotide
  source 1..1000: mol_type = other DNA
tttacgagtttactatactttagtcgtacagtttgcaataatagtattttaaccacaattagttatatgtacaaaatttcataagtgaataactttttttcaatgacaaaaataagagttgctcaaacaatatcaagttacaaaaatttaattttaactgtaaaagttatatttttccaaaataacataaactatagtaattatatatagtttgaagtattaataaaatttaaatatgcaaaagttaattttaataaaccatttgtatgcctacttgtagcctctaaactattttatttgctttatttatcaaactcatattttattttattgcaccttgttagttttggacgttaattatatatatttggtgtaaaatttaaaatatattaacatttgtggagaatttatgtatgcctggttcttaactattttttttttatataactggttagagtaatttcttatatttcagtatttatttttaaataagtcctcataaattgaagactttaaaagtttttgtgtcattcctctttttatttaagaaattgaagaattccgctaaatttcatattccgctgttatttaactgtttattcccttgttaatataattggtaagaagttttaaaataaaggagttaatgatttctaggttcatggcttgcctagcttctacgagtaagcgccatcacgactcccgaggataaggaaatccgggtcgtagcattcactcacaaaaattactaaaaacaaagtttacccttctcccaaaagtaaatttcatatttggctccacataatgtgttcaatgagtcaagtgaagtacttttcattgacaaaaaaaagttgctgaaaaatgcatatctcatattttttttttttagagaaatcccatttcttgcctaaacgaaatgcctataaaagagcatatatttgcaacaacagtttgcagaaactatcaagtcaaataatcccccctttaattccctcccaaa

SEQ ID NO: 14 - DNA, 1500 bp, synthetic construct
  misc_feature 1..1500: note = Description of Artificial Sequence: Synthetic polynucleotide
  source 1..1500: mol_type = other DNA
accagaatcaccatacaaagctatttccagactcggaatcccaaatagacatcaatagcactgaaatgcacttcaacccaatcgtatgaaattcttccaaaaaggctaacttccacaataagtgccaaaacgatctcgggtaattccaaacccgatctggacacacacccaagtccaaaataatcatacaaacctgttaaaacattcaaatcccgattccgaggtcaattactgaaaaatcctatcctagttaattcttccaactaaaagcttccaaaatgagaattctatttccaaataaactctgaacttcccggaattcaattttgactatgcgtacaagtcataatacctgaattgaagctgcagatagcctcaaatcgctaaacaacgtgctaggtctcaaaacgccctgtcgggtcgttacattataggtgattaactacgtatatacttgtgcaagtgttctatcataattcaggccaactagtaagagtagaaacaatgaatggcacataacaaacgatcaccacgaaatgtacatgatataactcacacaaggtaggcacgctactagacaattaccaataacaacaatgcctaggacatcacaagatatgacaaatcaatccttactatcacggttgagttgtaacgtgcaagaatatttcactctttttagggcactaagatcactccaccaacatttcaagagaatccctggcactgctaaaaagccctctacactgtagtgaatttttcttagttatctaaagttaattattcatttagtattctttacattaggttcccccttctaggtcctgcacgtaactagattgaatggattggtccactctattattatagagtaattattaaatttttatttgactaggcatcactagttgcactatcaacaaagtattagttctagccttctgggtacttcatacctatgcaaatgataattttattaaaacaatagatgtacatggatataaatacctatgaaaattaaataaattataactaagaaaaaaactttaaagttcactcctaagatatcgggttattacatgaccaaacacaatttgtttatcaaatactttcaaaagaatttgccaaacgtaaattatttttctccaaagtgacttatgaattactatgttgataaaatacttttcaaagtaactaatgtttagaagtcaaggatgggcttcttttgattattgaagtttgtagcaattgtatgtagttatagtcagggtgaccaccagcatctcatatagcaatacacaagtggattagcgcattttaaatttcaattagttcatgcaaatatacacgtaatagcattataagccactttcacaacaggcagattagtggttttgaaatttcaaccaatgatatatactataaattgatcaagcacaaaccttaattgagcaacacaatttcttacagcaataactatcacatataacaataactgcc

SEQ ID NO: 15 - DNA, 500 bp, synthetic construct
  misc_feature 1..500: note = Description of Artificial Sequence: Synthetic polynucleotide
  source 1..500: mol_type = other DNA
ataaatacctatgaaaattaaataaattataactaagaaaaaaactttaaagttcactcctaagatatcgggttattacatgaccaaacacaatttgtttatcaaatactttcaaaagaatttgccaaacgtaaattatttttctccaaagtgacttatgaattactatgttgataaaatacttttcaaagtaactaatgtttagaagtcaaggatgggcttcttttgattattgaagtttgtagcaattgtatgtagttatagtcagggtgaccaccagcatctcatatagcaatacacaagtggattagcgcattttaaatttcaattagttcatgcaaatatacacgtaatagcattataagccactttcacaacaggcagattagtggttttgaaatttcaaccaatgatatatactataaattgatcaagcacaaaccttaattgagcaacacaatttcttacagcaataactatcacatataacaataactgcc

SEQ ID NO: 16 - DNA, 1500 bp, synthetic construct
  misc_feature 1..1500: note = Description of Artificial Sequence: Synthetic polynucleotide
  source 1..1500: mol_type = other DNA
taatacttattttgtaagaattgcaatattgttatttttgttatggacttaaatattaaccatgttataatcttaagtttatattattagaaaaacttagtttttgaaagactaatatgaacattagtacttatttcaaaaataagcacctagatatatgaaattactttaagtacttatttaaaataattaagtaccacacatacatacatatctctacaaactgttaaagttttctatatgagtacttattttaaaataagagcttaaatataataaattatgttaaatttcttatttaaaataataaaggaccaaacatgcataaaataaagtatgagcttaataagtcaagaagctaattgataagcattgatgccaaatgcacttactaacttttctatattgtaggaaaaatctaacttttatatttaaaatttatttttgtaaaacttctctaatttttggacaaactcttatattgattttttaatcaaagccaaaatatttatttaactatgaaaaaattttaacaactaatttattatggtaaataatattgatatggtaacttcaagcacatgacaaaaattataactaactgcagaagtttattgtctctctgaatcttgtggctatatcataacaaatacttgtagctaataagccaacgatgttctcggtttcatataatttgaattttaaaatagtttttaaatttaatatttatttcaaatcattattgtggctaacatgttataatcgcagtaatatttggagatgcaatacttatatttagctacaaaattttattgtatcagaataagtttgtagctattaagttagtttttgccacaaatttttataattgaagcaaaaatacctattcagctacagtattttgtatcgagtaatattttgtgactagaagattaatattgctacagtaatttcagacgtgtggcaaaaactcataattagctacaaaatattgtcgtagcaataattttttatatctattaatgcaattattactacatgcttttataacttgaggcaaaaatatctaatagctataacattttgttagaagtaatttttgtggctataaaattggtattgctacagtaatttcaaatgcgtggcaaaaaaatacgattaactacgaaattttattgtagcaataacntattgtagcaataaatttgtagctatttgggtaatattgctacgacagttagcaattatagcaaaaatgctaaattagctttgttaatttaattttgtagctaaacttttttatgaaattttaatttttgtggctattgttaggtattagctacaattttcatatatgtagctaagaatttgtagctatatatacataatgttgtagtggcaaattctaacattgtacgcttggctgcccttttttttttggctacaaaactctaaagtaaaggaactagaaaactcgtttggcgagagaaagagagagag

SEQ ID NO: 17 - DNA, 500 bp, synthetic construct
  misc_feature 1..500: note = Description of Artificial Sequence: Synthetic polynucleotide
  source 1..500: mol_type = other DNA
caataattttttatatctattaatgcaattattactacatgcttttataacttgaggcaaaaatatctaatagctataacattttgttagaagtaatttttgtggctataaaattggtattgctacagtaatttcaaatgcgtggcaaaaaaatacgattaactacgaaattttattgtagcaataacntattgtagcaataaatttgtagctatttgggtaatattgctacgacagttagcaattatagcaaaaatgctaaattagctttgttaatttaattttgtagctaaacttttttatgaaattttaatttttgtggctattgttaggtattagctacaattttcatatatgtagctaagaatttgtagctatatatacataatgttgtagtggcaaattctaacattgtacgcttggctgcccttttttttttggctacaaaactctaaagtaaaggaactagaaaactcgtttggcgagagaaagagagagag

SEQ ID NO: 18 - protein, 365 aa, Nicotiana sp.
MAFLATISGHENMLLSNTLNNNFIFSGKPPQRHSYSFLPKKIQARSVANSSKTFQVKEEEFSSKTEKFILPKFDFEEYMKMKAIKVNKALDDAIPMQEPIKIHEAMRYSLLAGGKRVRPILCMASCEVVGGDESLAIPAACSVEMIHTMSLIHDDLPCMDNDDLRRGKPTSHKAFGEDTAVLTGDALLSLAFEHVASKTKDVTPQRVVQAVGELGSAVGSKGLVAGQIVDIASEGKQVSLTELEYIHNHKTGKLLEAAVVCGAIIGGGNEIEVERMRNYARCLGLLFQVVDDILDVTKSSEELGKTAGKDLVTDKATYPKLMGLEKARELAGELVAKAMDELSYFDAAKAAPLYHFANYIAHRQN

SEQ ID NO: 19 - protein, 802 aa, Nicotiana tabacum
MQVIITSSHRFFCHHLHQLKSPTSLSAQKAEFKKHGPRNWLFQTEGSLLYKPVRLNCATSDASYLGNVNEYLESDHSKNSEEKDIQVSRTIQMKGLTEEIKHMLNSMEDGRLNVLAYDTAWVSFIPNTTNNGNDQRPMFPSCLQWIIDNQLSDGSWGEEIVFCIYDRLLNTLVCVIALTLWNTCLHKRNKGVMFIKENLSKLETGEVENMTSGFELVFPTLLEKAQQLDIDIPYDAPVLKDIYARREVKLTRIPKDVIHTIPTTVLFSLEGLRDDLDWQRLLKLQMPDGSFLISPASTAFAFMETNDEKCLAYLQNVVEKSNGGARQYPFDLVTRLWAIDRLQRLGISYYFAEEFKELLNHVFRYWDEENGIFSGRNSNVSDVDDTCMAIRLLRLHGYDVSPDALNNFKDGDQFVCFRGEVDGSPTHMFNLYRCSQVLFPGEKILEEAKNFTYNFLQQCLANNRCLDKWVIAKDIPGEIWYALEFPWYASLPRVEARYYIEQYGGADDIWIGKTLYRMPDVNNNVYLQAAKLDYNRCQSQHRFEWLIMQEWFEKCNFQQFGISKKYLLVSYFLAAASIFEVEKSRERLAWAKSRIICKMITSYYNDEATTWTTRNSLLMEFKVSHDPTRKNGNETKEILVLKNLRQFLRQLSEETFEDLGKDIHHQLQNAWETWLVFLREEKNACQEETELLVRTINLSGGYMTHDEILFDADYENLSNLTNKVCGKLNELQNDKVTGGSKNTNIELDMQALVKLVFGNTSSNINQDIKQTFFAVVKTFYYSAHVSEEIMNFHISKVLFQQV

SEQ ID NO: 20 - protein, 792 aa, Nicotiana sp.
MVLGLRSKIIPLPDHKLGNIKLGSVTNAICHRPCRVRCSHSTASSMEEAKERIRETFGKIELSPSSYDTAWVAMVPSRYSMNQPCFPQCLDWILENQREDGSWGLNPSHPLLVKDSLSSTLASLLALRKWRIGDNQVQRGLGFIETHGWAVDNKDQISPLGFEIIFPCMINYAEKLNLDLPLDPNLVNMMLCERELTIERALKNEFEGNMANVEYFAEGLGELCHWKEMMLRQRHNGSLFDSPATTAAALIYHQYDEKCFGYLNSILKLHDNWVPTICPTKIHSNLFLVDALQNLGVDRYFKTEVKRVLDEIYRLWLEKNEEIFSDVAHCAMAFRLLRMNNYEVSSEELEGFVDQEHFFTTSSGKLMNHVAILELHRASQVAIHERKDHILDKISTWTRNFMEQKLLDKHIPDRSKKEMEFAMRKFYGTFDRVETRRYIESYKMDSFKILKAAYRSSGINNIDLLKFSEHDFNLCQTRHKEELQQMKRWFTDCKLEQVGLSQQYLYTSYFIIAAILFEPEYADARLAYAKYAIIITAVDDFFDCFICKEELQNIIELVERWEGYSTVGFRSERVRIFFLALYKMVEEIAAKAETKQGRCVKDHLINLWIDMLKCMLVELDLWKIKSTTPSIEEYLSVACVTIGVPCFVLTSLYLLGPKLSKDVIESSEVSALCNCTAAVARLINDIHSYKREQAESSTNMVSILITQSQGTISEEEAIRQIKEMMESKRRELLGMVLQNKESQLPQVCKDLFWTTINAAYSIHTHGDGYRFPEEFKNHINDVIYKPLNQYSP

SEQ ID NO: 21 - protein, 687 aa, Nicotiana sp.
MILGLRSKIIPLPDHKLGNIKLGSVTNAICHRPCRVRCSHSTASSMEEAKERIRETFGKIELSPSSYDTAWVAMVPSRYSMNQPCFPQCLDWILENQREDGSWGLNPSHPLLVKDSLSSTLASLLALRKWRIGDNQVQRGLGFIETHGWAVDNKDQISPLGFEIIFPCMINYAEKLNLDLPLDPNLVNMMLCERELTIERALKNEFEGNMANVEYFAEGLGELCHWKEMMLRQRHNGSLFDSPATTAAALIYHQYDEKCFGYLNSILKLHDNWVPTICPTKIHSNLFLVDALQNLGVDRYFKTEVKRVLDEIYRLWLEKNEEIFSDVAHCAMAFRLLRMNNYEVSSEELEGFVDQEHFFTTSSGKLMNHVAILELHRASQVAIHERKDHILDKISTWTRNFMEQKLLDKHIPDRSKKEMEFAMRKFYGTFDRVETRRYIESYKMDSFKILKAAYRWEGYSTVGFRSERVRIFFLALYKMVEEIAAKAETKQGRCVKDHLINLWIDMLKCMLVELDLWKIKSTTPSIEEYLSVACVTIGVPCFVLTSLYLLGPKLSKDVIESSEVSALCNCTAAVARLINDIHSYKREQAESSTNMVSILITQSQGTISEEEAIRQIKEMMESKRRELLGMVLQNKESQLPQVCKDLFWTTINAAYSIHTHGDGYRFPEEFKNHINDVIYKPLNQYSP

SEQ ID NO: 22 - protein, 598 aa, Nicotiana sp.
MSQSISPLICSHFAKFQSNIWRCNTSQLRVIHSSYASFGGRRKERVRRMNRAMDLSSSSRHLADFPSTIWGDHFLSYNSEITEITTQEKNEHEMLKEIVRKMLVETPDNSTQKLVLIDTIQRLGLAYHFNDEIENSIQNIFNLSQNSEDDDEHNLYVAALRFRLARQQGYYMSSDVFKQFTNHDGKFKENHTNDVQGLLSLYEAAHMRVHDEEILEEALIFTTTHLESVIPNLSNSLKVQVTEALSHPIRKAIPRVGARKYIHIYENIGTHNDLLLKFAKLDFNMLQKLHRKELNELTSWWKDLDRANKFPYAKDRLVEAYFWTVGIYFEPQYSRSRSLVTKVVKMNSIIDDTYDAYATFDELVLFTDAIQRWDEGAMDLLPTYLRPIYQGLLDVFNEMEEVLAKEGKADHIYYAKKEMKKVAEVYFKEAEWLNANYIPKCEEYMKNGLVSSTGPMYGIISLVVMEEIITKEAFEWLTNEPLILRAASTICRLMDDMADHEVEQQRGHVASFVECYMKEYGVSKQEAYVEMRKKITNAWKDINKELLRPTAVPMFILERSLNFSRLADTFLKDDDGYTNPKSKVKDLIASLFVESVDI

SEQ ID NO: 23 - protein, 238 aa, Nicotiana sp.
MSCQYYLTTTTSSLRIFSFTPRRYAPNSSASQPHEFFKKQVLFSSNLQCNAVSRPRAQVIKRDDNVEEVDSAEEQQEEEETQEVYRSNKIKQHIYAVRLMLQSMDDGEISISAYDTAWVALVKDINGSDTPQFPSSLEWIANNQLAECSWGDKSIFLAHDRIINTLACVIALKSWNLHIDKRELGMSFIRENLSKIGDENAVHMPIGFEVAFPSLIEIGKKIGIDIPDDSHVLREIYT

SEQ ID NO: 24 - protein, 606 aa, Nicotiana sp.
MASITINHSFSRNPNISFHPQNPLIQTQALFNFKPSISKCSPIIHCAIRRRPEYTPSHIPDPNYIRIFDTTLRDGEQSPGATMTTKEKLDVARQLAKLGVDIIEAGFPASSEADLEAVKLIAKEVGNGVNEEGHVPVICGLARCNKRDIDKAWEAVKYAKKPRIHTFIATSEIHMKFKLKMSRDEVVEKARSMVAYARSIGCEDVEFSPEDAGRSDPEFLYHILGEVIKAGATTLNIPDTVGYTVPSEFGKLIADIKANTPGIGDVIISTHCQNDLGLSTANTLAGACAGARQVEVTINGIGERAGNASLEEVVMALKCRGEQVLGGLYTGINTQHILMSSKMVEEYTGLHVQPHKAIVGANAFAHESGIHQDGMLKHKDTYEIISPEDIGLNRVNESGIVLGKLSGRHALQAKMLELGYDIEGKELEDLFWRFKSVAEKKKKITDDDLIALMSDEVFQPQFVWQLENVQVTCGSLGLSTATVKLIDADGQEHVSCSVGTGPVDAAYKAVDLIVKVPVALLEYSLNAVTEGIDAIASTRVLIRGENGHTSTHALTGETVHRSFSGTGADMDIVISSVRAYIGALNKMLSFRKLVSKHSKPEGSAVV

SEQ ID NO: 25 - protein, 492 aa, Nicotiana sp.
MAILISRSRNFNHFLLSTRFRFLSRLNQTSSKTFSHHAKTSSFTTAAGGGFTKPTATFSGESAAVFRVERFQSNKTGEQLNTLYYEEDNHHQIIDENQVMDFPGGQLPITPQMKFIAESSEKRLPCYRVLDDDGYPIPGSIFEEVSKELAVKMYSSMVTLQVMDTIFYEAQRQGRLSFYLTTAGEEGINIASAAALSVDDFVLPQYREVGVILWRGFPLKDIANQLFGNKFDYGKGRQMPCHHGSNELNYLTISSPIATQIPQAAGVAYSLKMDKKEACAITYLGDGSTSEGDFHAALNFAAVLDAPVVFICRNNGWAISTPVNEQFRSDGVASKGQAYGIRSIRVDGNDVLATHSAIRAAREMAIKEQKPILVEAMTYRVAHHSTSDDSTKYRPVEEIEHWKTAKSPISRFRKWIQRNGWWNDENESELRGDTRKQVLQVMQAAEKVEKPPLTDLFTDVYDKVPLNLQEQHKFIRDAVKKSPREYPSDVPI

SEQ ID NO: 26 - protein, 321 aa, Nicotiana tabacum
MAEKITSHENTRYAVVTGGNKGIGYETCRQLAKEGIVVVLTARDERRGIEALEKLKEEYSSNKTDDDQILFHQLDVMDPASISSLVDFIKTKFGKLDILVNNAGIGGLMVEGDVVIIKDLIEGDFVTISAENGEEDGIKKSIEGIERIVTDYELTKQCLETNFYGAKRMIEAFIPLLQLSNSPRIVNVASFLGKLKLLCNQWAIGMLSDAKSLREERVDEVLNEFIKDFKEKSIEAKGWPTYFSAYKVSKASLIAYTRVLATKYPNFRINSVCPGFCKTDVNCNTGSLTAEEGAESLVKLALVPNDGPSGLFFYRKEVTSF

SEQ ID NO: 27 - DNA, 1098 bp, Nicotiana sp.
  source 1..1098: mol_type = unassigned DNA
atggcatttttggctaccatttctggccatgaaaatatgcttctttccaataccctaaacaataactttattttcagtggaaaacctccacagagacattcttatagtttcctccccaagaaaatccaggccagaagtgttgcaaactcatccaaaacatttcaagtcaaagaagaagaattctcatctaagacagagaaattcatcttgcctaagtttgactttgaagaatatatgaaaatgaaggcaattaaggtaaacaaagcactagatgatgcaataccaatgcaagagcctataaaaattcatgaagccatgagatactcacttctagctgggggaaaacgcgtccggccgatcctatgcatggcttcttgtgaagtagtaggaggggatgaatccttagctattcctgcagcttgctcggttgagatgatccacaccatgtcactcatccacgacgatcttccttgcatggacaacgatgatctacgtcgtggcaagcccacgagccacaaggctttcggggaagacactgcagttctaacaggggatgcacttttgtctttggcctttgaacatgtagcttccaagactaaagatgtgacaccccaaagagtggttcaagccgttggcgaattgggttcagccgttggctcgaaagggcttgtggcggggcagattgtggacatagctagtgagggaaaacaagtgagcctaactgaattagagtacattcacaaccataagacagggaaactattggaggctgctgtggtttgtggggcaataattgggggagggaatgagattgaggtggagagaatgaggaactatgctagatgccttggactgttgtttcaagtggtagatgatattcttgatgttactaagtcatcagaagagttgggaaagacagctggtaaagacctagtgactgataaggctacatatcctaagttgatggggctagaaaaagctcgggagctcgccggagagctggtggctaaggccatggatgagctgagctactttgatgctgccaaggcggcacctctttatcattttgctaattatattgcacatcgccagaattga

SEQ ID NO: 28 - DNA, 2409 bp, Nicotiana sp.
  source 1..2409: mol_type = unassigned DNA
atgcaagttataattacgtccagtcacagatttttctgccatcatcttcatcagctcaagagtcctacatcgttgtctgcacagaaagctgagtttaaaaaacatggaccccgaaattggttgttccaaactgaaggctcacttctatataaaccagttcgtctcaattgtgcaactagtgatgcaagttatcttggtaatgtgaatgagtacttagaatctgatcactcaaaaaactccgaagaaaaggatattcaggtaagcagaacaatacagatgaaaggtttgacagaagagatcaaacacatgttgaattcgatggaggatggaaggttaaatgtcttagcctatgacacagcttgggtttcctttattccaaatactactaataatggaaatgatcaaagacctatgtttccatcttgtcttcaatggattatagacaatcaactttctgatggttcatggggagaggagattgtattctgcatatatgatcgactcttgaacacactagtatgtgttattgcattgacattatggaacacgtgccttcataagagaaacaaaggtgtgatgtttatcaaagaaaacttaagcaagctagagacaggggaagttgaaaacatgactagtggatttgaacttgtgtttcctactctccttgaaaaagctcaacaactagatattgacattccgtatgatgctccagtcttaaaggatatttatgcaaggagagaagtaaagttaacaagaattcctaaagatgttatccatacgattccgacaacagtattgttttcattagaaggattaagggacgacttagactggcaaagacttttaaagcttcaaatgcctgatggttcattcttaatatcccctgcttccactgcctttgcattcatggaaacaaatgatgaaaagtgtttggcatatcttcagaacgttgttgaaaagagtaatggaggagcgcgacaatacccgttcgacttggtaacacgactttgggcaattgatcgattacaacgccttggaatctcttattattttgcggaagagttcaaggaacttttgaatcatgtgttcagatactgggacgaggagaatggaatttttagtggaaggaattcaaacgtttctgatgttgatgatacatgcatggctataaggttgctaaggttacatgggtatgatgttagtccagatgcgctaaacaatttcaaagatggtgatcaattcgtttgcttcagaggtgaagtggacgggtcaccaacacatatgtttaatctctatagatgttcccaagttttattcccaggagaaaagattcttgaagaggcaaagaattttacttataacttcttacagcaatgtcttgcaaacaaccgatgcttagacaaatgggtcatagctaaggacatccccggggagatatggtatgcactagaatttccatggtatgccagcttacctcgggtggaagctaggtattatatagaacagtatggcggagcagatgatatttggattggcaagacgttatacagaatgcccgatgtcaacaacaatgtttatttacaagctgcaaaattggattacaatagatgccaaagtcaacatcgctttgaatggctgattatgcaagagtggtttgagaagtgcaactttcaacaatttggaataagcaaaaagtacctcctagtttcttatttcctagctgctgcaagtatatttgaagtcgagaagtcaagagaacgccttgcatgggctaaatctcgtataatatgtaagatgattacatcttactacaatgatgaagccacaacttggacaactaggaattcattgctaatggaattcaaggtttctcatgatccgaccagaaaaaatggtaatgaaacaaaagagatcttagttctcaaaaatcttcgtcagtttttgcgccaactatcagaagaaacttttgaagacctaggcaaagacatccatcaccaactacaaaatgcttgggaaacgtggttggtgttcttgagggaggaaaagaatgcatgtcaagaagaaacagagttgctggtgcgcacaattaatctctcgggcggctatatgacacatgatgagatattattcgatgcggactacgagaatctgtccaaccttaccaataaagtttgtggcaagcttaatgagctccaaaatgacaaggtgacgggcggctcaaagaacaccaatattgaactcgacatgcaagctctcgtaaagttagtgtttggtaacacctcaagcaacatcaaccaagacattaagcaaacattttttgcagttgtgaagactttctattacagtgcgcatgttagtgaggaaataatgaactttcacatatccaaagtgctttttcagcaagtctag

SEQ ID NO: 29 - DNA, 2379 bp, Nicotiana tabacum
  source 1..2379: mol_type = unassigned DNA
atggtacttggactgagaagcaaaatcataccacttcctgatcataagttgggaaatatcaaattaggttcagtaaccaatgcaatttgccacagaccatgtagagtaagatgcagccacagtactgcttcatcaatggaagaggcaaaggagagaataagggaaacatttggaaaaatagagctatctccttcttcctatgacacagcatgggtagctatggtcccttcaagatattctatgaaccaaccatgttttcctcagtgcttagattggattcttgaaaatcaaagagaagatggatcttggggcctaaatcctagccatccattgcttgtaaaagactccctttcttccactctagcatctttgcttgcccttcgcaaatggagaattggagataaccaagtccaaagaggccttggctttattgaaacgcatggttgggcagtcgataacaaggatcagatttcacctttaggatttgaaattatatttccctgcatgatcaactatgcagagaaacttaatttggatctacctttggatcctaaccttgtaaatatgatgctctgcgaacgtgaattaacaattgaaagagccttaaagaatgaattcgaggggaatatggcaaatgtagaatattttgctgaagggctcggtgaattatgtcattggaaagagatgatgcttcgtcagagacacaacgggtcgctctttgattcaccagccactactgcagctgccttgatttaccatcagtacgatgagaaatgctttgggtacttgaactcaatcttgaaactgcacgataattgggtccccactatttgccctacaaagatacattcaaatctcttcttagttgatgcccttcaaaatcttggagtagatcggtattttaaaacagaagtcaaaagagtactagatgaaatatacaggctttggctagaaaagaatgaagaaattttttcagacgttgctcattgtgccatggcgtttcgacttttacggatgaataactatgaagtttcctcagaagaacttgaaggatttgtcgaccaagaacatttctttacaacatcaagtgggaaacttatgaatcacgttgcaattctcgaacttcaccgagcttcacaggtggctattcatgaaaggaaagatcacattttagataaaataagtacttggacaaggaattttatggagcaaaaactcttggacaagcacatccctgataggtcaaagaaggagatggaatttgctatgaggaaattttatggcacatttgatcgagtggaaactagacgttacatcgagtcatacaaaatggacagttttaagatcttaaaagcggcttacaggtcttccggtattaacaacatagacttgctaaagttctcagaacacgattttaacttgtgccaaacccgacacaaagaagaacttcaacagatgaaaaggtggttcacagattgcaaactcgaacaagtaggattatcacaacagtacttatacactagttacttcataattgctgccatactctttgaacctgaatatgctgatgctcgtctagcatatgcaaagtacgccataataataacagcggtggatgatttcttcgattgttttatttgcaaagaagaactgcaaaacatcatcgaattagtagagagatgggagggatactcaaccgtcggattccgttcagagagggttagaattttctttttggcactttacaaaatggtagaggaaattgcggcaaaggcggaaactaagcaaggtcgatgtgtcaaagatcaccttattaacttgtggattgatatgttgaagtgtatgctggtggaattggacctttggaaaattaaatcaactaccccaagcatagaggagtacttgtctgttgcatgtgtaactattggtgttccatgttttgttctcacatcactatatcttcttggaccaaaactgtccaaggatgtcatagaaagttctgaggtcagtgccttatgcaattgtacagctgctgtggcccgattgattaatgatatacacagttacaagagagaacaagcagaaagttcaacaaatatggtatcaatattaataacacaaagtcagggaactatctctgaagaagaggctataagacagataaaggaaatgatggaaagtaagagaagagagttgctagggatggttctacaaaataaagaaagccaattgccacaagtgtgcaaggatcttttttggacgacaatcaacgcagcttattctatacatacacatggcgatgggtatcgcttcccagaggaattcaagaaccatatcaacgatgtaatttacaaaccactcaatcaatattccccataa

SEQ ID NO: 30 - DNA, 2064 bp, Nicotiana tabacum
  source 1..2064: mol_type = unassigned DNA
atgatacttggactgagaagcaaaatcataccacttcctgatcataagttgggaaatatcaaattaggttcagtaaccaatgcaatttgccacagaccatgtagagtaagatgcagccacagtactgcttcatcaatggaagaggcaaaggagagaataagggaaacatttggaaaaatagagctatctccttcttcctatgacacagcatgggtagctatggtcccttcaagatattctatgaaccaaccatgttttcctcagtgcttagattggattcttgaaaatcaaagagaagatggatcttggggcctaaatcctagccatccattgcttgtaaaagactccctttcttccactctagcatctttgcttgcccttcgcaaatggagaattggagataaccaagtccaaagaggccttggctttattgaaacgcatggttgggcagtcgataacaaggatcagatttcacctttaggatttgaaattatatttccctgcatgatcaactatgcagagaaacttaatttggatctacctttggatcctaaccttgtaaatatgatgctctgcgaacgtgaattaacaattgaaagagccttaaagaatgaattcgaggggaatatggcaaatgtagaatattttgctgaagggctcggtgaattatgtcattggaaagagatgatgcttcgtcagagacacaacgggtcgctctttgattcaccagccactactgcagctgccttgatttaccatcagtacgatgagaaatgctttgggtacttgaactcaatcttgaaactgcacgataattgggtccccactatttgccctacaaagatacattcaaatctcttcttagttgatgcccttcaaaatcttggagtagatcggtattttaaaacagaagtcaaaagagtactagatgaaatatacaggctttggctagaaaagaatgaagaaattttttcagacgttgctcattgtgccatggcgtttcgacttttacggatgaataactatgaagtttcctcagaagaacttgaaggatttgtcgaccaagaacatttctttacaacatcaagtgggaaacttatgaatcacgttgcaattctcgaacttcaccgagcttcacaggtggctattcatgaaaggaaagatcacattttagataaaataagtacttggacaaggaattttatggagcaaaaactcttggacaagcacatccctgataggtcaaagaaggagatggaatttgctatgaggaaattttatggcacatttgatcgagtggaaactagacgttacatcgagtcatacaaaatggacagttttaagatcttaaaagcggcttacagatgggagggatactcaaccgtcggattccgttcagagagggttagaattttctttttggcactttacaaaatggtagaggaaattgcggcaaaggcggaaactaagcaaggtcgatgtgtcaaagatcaccttattaacttgtggattgatatgttgaagtgtatgctggtggaattggacctttggaaaattaaatcaactaccccaagcatagaggagtacttgtctgttgcatgtgtaactattggtgttccatgttttgttctcacatcactatatcttcttggaccaaaactgtccaaggatgtcatagaaagttctgaggtcagtgccttatgcaattgtacagctgctgtggcccgattgattaatgatatacacagttacaagagagaacaagcagaaagttcaacaaatatggtatcaatattaataacacaaagtcagggaactatctctgaagaagaggctataagacagataaaggaaatgatggaaagtaagagaagagagttgctagggatggttctacaaaataaagaaagccaattgccacaagtgtgcaaggatcttttttggacgacaatcaacgcagcttattctatacatacacatggcgatgggtatcgcttcccagaggaattcaagaaccatatcaacgatgtaatttacaaaccactcaatcaatattccccataa

SEQ ID NO: 31 - DNA, 1797 bp, Nicotiana sp.
  source 1..1797: mol_type = unassigned DNA
atgagtcaatcaatttctccattaatctgttctcactttgcgaaatttcagtcgaatatttggagatgcaatacttctcaactcagagttatacactcatcatatgcctcttttggagggagaagaaaagagagagtaagaagaatgaatcgagcaatggatctttcttcaagctctcgtcatttggcagattttccctcaacaatttggggtgaccattttctctcctacaattctgaaataacagaaattactacccaagagaaaaatgaacatgaaatgctaaaagaaatagttcggaaaatgttggtagaaactccagataatagtacacaaaaactagtcttgattgacacaattcaaagattgggattagcatatcatttcaatgatgagattgaaaactccattcaaaacatctttaatttgtctcaaaatagtgaagatgacgatgaacacaacctttatgttgctgctcttcgttttcgacttgcgaggcaacaaggatattacatgtcttcagatgtgttcaagcaattcactaaccatgacggaaaattcaaggaaaatcatactaatgatgttcaaggattattgagtttgtatgaagcagcacatatgagagtgcacgacgaggaaattctagaagaagctcttatctttaccacgactcatctcgagtccgtgatcccgaatttgagcaactcgcttaaggtacaagttactgaagccttaagccatcctattcgcaaagctataccaagggtgggagcaaggaaatacatacacatatatgaaaacattggaacacataatgatttacttttgaaatttgcaaagttggacttcaacatgttacaaaagcttcatcgaaaagagcttaacgagctaacaagctggtggaaagatttggatcgtgcaaacaaatttccatatgcaaaggacagattagtagaagcttacttttggacggtgggaatatattttgaacctcaatatagtcgttcaagaagtttggtaacaaaagtagtcaaaatgaactccattattgatgacacttatgatgcttatgcaacttttgatgagcttgtgcttttcacggatgcgatccaaagatgggacgaaggtgccatggatttattaccgacatatctgagacctatttatcaaggccttctcgacgttttcaatgaaatggaagaagtattggccaaagaaggtaaagcagatcacatctactatgcgaaaaaagagatgaaaaaggtggcggaagtctattttaaggaagctgaatggttgaatgctaactacattccaaaatgcgaggagtatatgaaaaatggacttgtaagctctaccggtccgatgtatggaataatttctttggttgttatggaggaaattataacaaaagaggcttttgaatggttgacaaatgaacctttgattcttcgagctgcatcaacaatttgtagattaatggatgatatggctgatcatgaagttgaacaacaaagaggacatgttgcttcatttgttgagtgctacatgaaagaatatggagtttcaaagcaagaagcatatgttgagatgcggaaaaaaatcacaaatgcgtggaaagatataaataaggaactcttgcgccctactgcagtaccaatgtttatcctcgaacgatctttaaatttttcaagattggccgatacatttttgaaagatgatgatggatacacaaatcccaaatccaaagttaaagacttgattgcttcgttgtttgtcgaatctgtcgacatatga

SEQ ID NO: 32 - DNA, 717 bp, Nicotiana sp.
  source 1..717: mol_type = unassigned DNA
atgtcttgtcaatattacttaaccacgacgacctcttctctcagaatattctccttcaccccccgccgttacgcaccgaattcttctgcaagtcaacctcatgagttctttaaaaaacaagtacttttcagttccaatctgcaatgcaatgcggtttcaagacctcgcgcacaagttatcaagcgggacgacaacgtggaagaagtagacagtgcagaagaacaacaagaagaagaagaaacacaagaggtgtacagatcaaataagataaagcaacatatttatgccgtccggttaatgttgcaaagtatggatgatggagagataagtatatcagcttatgacacagcttgggttgctcttgtgaaagacattaatggaagcgatactcctcaattcccttcaagtcttgaatggattgccaacaatcaacttgctgaatgttcgtggggtgacaagtccatctttttggctcacgatcgaatcatcaacacattggcctgtgttattgctttgaaatcttggaatttgcacattgacaaaagagaactaggaatgtcgtttatcagagagaatttaagcaagattggagatgaaaatgctgtgcatatgccaataggatttgaagtggcgtttccttcactaattgagattggaaaaaagataggcattgatattccggatgattctcatgtcttgagagagatatatacctga

SEQ ID NO: 33 - DNA, 1821 bp, Nicotiana sp.
  source 1..1821: mol_type = unassigned DNA
atggcgtctatcaccataaaccattcattttcccgtaaccctaacatctcattccatccccaaaatcctctcattcaaacccaagctctcttcaatttcaaaccatcaatctccaaatgttcccctattatccactgcgcaatccgccgtcgacccgaatataccccgagccacattcccgacccgaactacattcgcatcttcgacaccactctccgcgacggcgaacaatccccaggcgccacaatgaccacaaaagaaaaactcgacgttgcgcgtcagttagctaagcttggtgttgacataattgaagccggttttcctgcttcttctgaagctgatctcgaagctgtgaaattaatagcgaaggaagttggaaatggtgtgaatgaagagggacatgttccggtaatttgtggacttgcgaggtgtaataagagggatattgataaggcttgggaggctgtgaagtatgcgaaaaaaccgaggattcatacgtttattgcgactagtgagatacatatgaagtttaagttgaagatgagtagagatgaagttgtggagaaagctaggagtatggttgcttatgctaggagtattggttgtgaggatgttgaatttagcccagaagatgctggaagatccgatccagagttcctctatcatatccttggagaggtcatcaaagctggggcaacaacccttaacatccctgatactgttggatacactgttcccagcgaatttggaaaattgattgctgatataaaggccaataccccaggaattggagatgtgatcatctcaacacactgccagaacgatcttgggctttctactgccaacaccttagctggagcatgcgcaggtgcaagacaagtagaagtgaccatcaacggaatcggtgaaagagctggaaatgcttctttggaggaggttgtaatggccttaaaatgtcgtggagagcaagtactaggtggcctgtatacaggaattaatacacaacatatactcatgtcaagcaagatggtagaggagtacaccgggcttcatgtgcagccacacaaggccattgttggagctaatgcgtttgctcatgaaagtggcatccatcaggatggaatgttaaaacacaaagatacatatgagattatatctcctgaagatattgggcttaaccgagttaatgaatctggcatcgtccttgggaaactcagtgggcgtcatgctttgcaagccaaaatgctcgagcttggatacgatattgagggaaaagaacttgaggacctcttttggcgattcaaatctgtggccgagaagaaaaagaaaattacagatgatgacctgatagcattaatgtcagatgaagttttccagcctcaatttgtttggcaacttgaaaatgtacaggttacatgtggaagtcttggcctttctacggcaactgttaagctcattgacgctgatggtcaagagcatgtttcttgttctgttggaacggggccagttgatgcggcttataaggcagttgatctcattgtaaaggtacctgtagcactccttgaatattccttgaatgcagtcacggaaggtatagatgccatagcttcaaccagagttttaattcgtggggagaatggccatacatcaacccatgctttaactggagagactgtacaccgttcttttagtggaaccggagcagatatggatattgttatctccagtgtccgagcctatattggtgcattgaataagatgttgagtttcagaaagctggtatcgaaacacagcaaacctgaaggcagtgcagtcgtatag

SEQ ID NO: 34 - DNA, 1479 bp, Nicotiana sp.
  source 1..1479: mol_type = unassigned DNA
atggcgattttgatatcaagatcaagaaactttaaccattttcttctaagcacaaggtttcgtttcttatcacgcctaaaccaaacaagttcaaaaacattttcccaccatgccaaaacttcatcatttacaactgcagccggcggtggttttacaaagccgacggcgacattttccggcgagtctgccgccgttttccgggtagaacgtttccagtccaataaaactggagagcaactaaatacactctactatgaagaagataaccatcaccaaattattgatgaaaatcaggtcatggattttcctggagggcagcttccaattactcctcaaatgaaatttattgcagagtcatctgaaaagaggttaccttgttatagagtccttgatgatgatggctatccaattccaggcagcatttttgaggaggtgagcaaagaattggctgtaaaaatgtatagttcaatggtgacacttcaagttatggataccatattttatgaagcacaaaggcaggggaggttatctttctatctcactactgctggagaagaaggtatcaacatagcatctgctgctgctctctccgtcgatgacttcgtcttgcctcagtatagggaagtaggggttatcttatggcggggcttccccctgaaagatattgccaatcaattgttcggaaacaagtttgattatggaaaaggaaggcaaatgccatgccaccatggttctaatgagctcaattacttaactatttcttcgccaatagcgacacagattcctcaggccgcgggcgttgcttactctctgaaaatggataaaaaggaggcttgtgctattacttatcttggagatggtagcaccagtgagggtgattttcatgctgctttaaactttgcagcggttttggacgctcctgttgtctttatatgccgcaacaatggatgggccattagcactcctgtaaacgaacaatttcgaagtgatggagttgcctcaaagggtcaagcctatggaattagaagcattcgtgtagatggcaatgatgtcttggcaactcatagtgctattcgtgcagctcgcgaaatggcaattaaggaacaaaagccaatattagtagaggccatgacttatagagtagcccaccattcaacatctgatgattcaaccaagtatcgacccgtcgaagaaatagagcactggaaaacagcaaaaagtccaatatccagattcagaaaatggattcagagaaatggttggtggaatgatgaaaatgaatctgaacttcgcggagacaccagaaaacaggtattgcaagtaatgcaagcagcagagaaggtggagaaacctccattgacagatttgtttacggatgtttatgacaaagtgccattaaatcttcaagagcaacacaagtttattagggatgctgtaaagaaatctccaagagagtatccttctgatgttcctatataa

SEQ ID NO: 35 - DNA, 966 bp, Nicotiana tabacum
  source 1..966: mol_type = unassigned DNA
atggcagaaaaaatcaccagccacgagaacacaaggtatgcagtggtgacagggggaaataaaggaataggatatgaaacatgcaggcaactagcaaaggaaggaatagtggtagtgttgacagcaagggatgaaaggagaggaattgaagctctcgaaaagctcaaggaagagtactcaagcaataaaactgatgatgatcagattttatttcatcaacttgatgttatggatccagctagtatttcttctcttgtggacttcatcaaaactaaatttggaaagctcgatattctggttaacaacgcagggattggtggattaatggtagaaggagatgttgttataataaaagatttaatagaaggagacttcgtaaccatttctgctgaaaatggggaagaggatggtattaagaaatcaattgaaggtattgagcgtattgttacagattatgagttgacaaaacaatgcctggagacaaacttctatggtgcaaaaagaatgattgaagcatttattcccctccttcagctctctaactccccaagaattgttaatgtcgcttctttcttggggaagttaaagctattgtgcaaccaatgggctataggaatgctaagtgatgctaaaagcctgagagaagaaagggtggatgaagtgttgaatgaatttataaaagattttaaagagaaatcaatagaagccaaaggatggccaacttatttctcagcttacaaagtctcgaaagcatccctgattgcttacacaagggttttagctacgaaatatccaaattttcggataaattctgtgtgtcctggattttgcaaaacagacgtgaactgcaatactgggagcttaactgctgaagaaggtgctgaaagcttggtgaagcttgctttggtgccaaatgatggaccctctggtctcttcttttatagaaaggaggtcacctctttttga

SEQ ID NO: 36-40 - skipped (5 entries)

SEQ ID NO: 41 - DNA, 14 bp, synthetic construct
  misc_feature 1..14: note = Description of Artificial Sequence: Synthetic oligonucleotide
  source 1..14: mol_type = other DNA
atgtacaaaatttc

SEQ ID NO: 42 - DNA, 14 bp, synthetic construct
  misc_feature 1..14: note = Description of Artificial Sequence: Synthetic oligonucleotide
  source 1..14: mol_type = other DNA
catgtcaaacgtta

SEQ ID NO: 43-44 - skipped (2 entries)

SEQ ID NO: 45 - DNA, 1500 bp, Cannabis sp.
  source 1..1500: mol_type = unassigned DNA
tcttaaacccttatgtgtttatttcattgttttagaattcatattaggatgttaatgaaacatacttgttagtaaatctagatcctcgtaaaatatttccaagaaatataacagctactcagattatacacaaaaatcccaatagacaaaaacactgtaaaagtcatactaaaactcaaaaaaaaaaaaaaatacaaccaaaccaaatcaataaaaactaaaaaatcatcaatgggcaggcaagacttcaattgtagttttgacttgcctgagtgaatcgattccggaaaagaattgaaattgtggtcgtcgttcaagctcttggctgaatctcctgattgttcaagccctctcatcagaattatctccattgttcaagccctctcatcggaagctcctggtttttccatcaatttttggccatggtcactatggaggtaagtagattgaagaagacgaatgtgagagagaaggcaaagggtcgaggtgggcgtggtgcagtttttgaggtgggagttgtttctcggccgcaacaatccaccatgtttcttagtttttttttctttttgttctgggaatcgattgtttttgggtgtggattgttggttgataggttttgggggtggtttctgggcagtgagaaggtgagaagaagaagaagaagaagaaagttatggtttgaagaagaagaagaaggaaaatcaggtgggtgggtggggtttcgtgtgaagcagaaaaaagaaaaaaaaaacaagttattttatgatttgaaaatattatttttagtttttttatattattttaaattttttttattaaaattataatttggaccagtaatacttcaagacgtggcattttaaaaattaataaacggacagttatcttagggaccaacggactcacagaaaatgtgaccttggggactattaccgccaatttttgaggtttgggaataatctccatcaacccttaatttttggggacttttaccgcaattatccctattatacataatattttataacaagtttttttttattattttttattatttgatgaagtaattaaagaggagttttcaatttgttaatttttcagattagcttgaaaaaaggattagcttgaaaatacaattctataatcatacaatttcaaagcatagaaaaaaaattgttcaaaaaataagaaaagaaaattaagcataattctatattggctgctacattgcaatatacgtacgtacagtcataaaaatatgcacatggatgaactattacaattagaacagaagaaaagtaaatgataaagcttcttactcttgactaactcttattaagtactgttgattaatttgaaattttcaaatcaaaatacactataaatagctgagtacatgaaacgagttttccatcaattaaagctaactccatctcgtaatttatataattatatagtgatcgtttatatacatatatatcaccaaatcgtgatttcaaa

SEQ ID NO: 46 - DNA, 1500 bp, Cannabis sp.
  source 1..1500: mol_type = unassigned DNA
aatgtttacaattttaacatgtcgattattttcaattactaattttttgaaaaaaaaaatgtgtaaagttaaatataaaaatacataagtcaatatttatattttgatatattattaaaataatatatttactaaaatttaatacaaaattatatatatacaaacatatttaaaaatttgtttatattttaaataaacaacatatatgcatgtttattaaatatttaattattattaatgtttataatactagtattgatctaatatgtttattatgataatattttaatagaatcaatataatttgttgattaaaagttcataaataagtttaaaaataaaacatttatgtttattttgttttatgaaacaaagctataaacaaatgtgaaattatgtataaacattatatttatattttatattgataatgtttataaattattaatctaatatatttaatatatagttttataaataaagtatgtatatttattttattttacttttttttgttgtaagaaaaaatatgtatgtttattgtttatttatataagacttaggaaaaattaattacctcatatagattgtacagaaaattaaaattatgtgtctctaatatagaatgtataattaaatgtaaataaattattttgtattatatagtataataaaattatataatgtatacaaaattataaatttctcatatcttttcttctaatttttatttatttatttttttaatgtcaatatgtataaagtgttggactccaatataaacacttaaagttaaacttaaattgagtttaattaattcaaattttttgtcaagttgtattttttaagtagtttgtttgtgcttgtggcttgttataaaacatgctatgccctaattttgtatacataacactaaggtggcatttggttgaaagaaatgaaaacatatataaaaataaaaatgaaataaaataaaatttaaaatgtataaaaaaatcattagtttcttttaatgttatattaacatgattttttattctattttaaaatagaatagtcattccactaaaatagtgtaaaggacattccattagaatgatattctaatactttaaaatgtaaccaaataaataaataaaataaaaataatttctttttcattctattccatttcattactcattttcattccattctatttcatttaacaaaaatgataaatgaacatttttgttatcttgtagtagtacaaactacaaataatagaaaaatgaaactttaatataaggaggagacaaagtctattatttaccttggtgatcaagttttatatacaactttgaaataagtactttctttctctctcttattaacataatcctacttgagttcccacttgcatttaccttaatataatactatgtcaaccatgtgtataattagatctaacatctacttcaaatctaaagaacttatttaccaactaatcaaaatcaaaat

SEQ ID NO: 47 - DNA, 1500 bp, Cannabis sp.
  source 1..1500: mol_type = unassigned DNA
gtagtacataaatttatagctgatgataagtctcattctcagtcgaggaatatctactgtgttttggatcaactgaaaagggagcttaaaagttcatccatagctcaattttcaaccatgtatttttagtttataatagtaaagattattcagtcccgtatagagagaaaacaaatgacaaatcatatgattcagcagcaaaaaatggactttttacaactcaaaataaaacagaaatatcttactataatataatacatagggataatgtacgttgatcggtttatgttaagtacagttcgtctaattaaaaaaaaaaaaaaacagaaaccaatccaataaaattagtcgagttagtcaggttaatccgtccaaatcgatcattaatatattttttttacattcaattgctatattttaattagatgtaataacgagacaaatagatatatatttataaattacacttacagttcaattcaacacataaaagaaacatattaattttaactttatatgttttaaaaattcatataaagtatatgatatatataaatatgtatatacataaaaacccttttgattgtgttggttggattagtaagagttgaatagtggggttgatattattttcaatccacccaatataaagatcagacagattatatttttatcgaatttttcgatttgtattttttgtcagcttatctcaatttaatttaagcaagttattcagattaggcggtttaaaaaaatgaaataatagcatgaaagttttcagtcataacattaaaaaaaaaaatgtcattttgttatgatgagtggctatgaactgcagccagtttttgatggtattcatatgcaattttaatggctttcttcagcttaatgatttaattagtagataagagattcttgaatctagtactcatgatgtatttgatatccatagtgaccattgaagattgaaacagttttaacttggcttctctctcatattgttcaagcattatttttggaacatgacgaaagttgttcaggaacccatgttgtccacatcctgaatttccaagcttaagagaacaaaaaatccaaataggcattcatcactaacttcacagacatatgtcacatatctctacatttaatcaaaactgttgtatgctgatgtcttcaattcacaagtggcaaaaatgtaaggtgggaccccaaaaaatttccactagagccaaactctaaatagggaaaactctacaccacctcgtagtacccaccgaaatcccataccactcatcttttagaaaagaaaaaaaccacacttgatctcacaacaaatataaagctgacttcttttgttgtttttagaaaccatttccaccattttcctaataaactacttgatactaaaataatcaaaataagaataaaggaaaggaaaggaaataatactacaacactgcaattattttctttctcctgttaaccaagtttgaggttagg

SEQ ID NO: 48 - DNA, 1113 bp, Cannabis sativa
  source 1..1113: mol_type = unassigned DNA
atgagcactgtaaatctcacatgggttcaaacctgttccatgttcaaccaaggaggtagatccagatccttatcaactttcaatctcaatctctaccaccctttgaaaaaaacacccttttcaatccaaaccccaaaacaaaaacgacccacttcaccattttcatcaatctcagctgttctaaccgagcaagaagccgttaaagaaggcgatgaagaaaaatccatcttcaatttcaagtcttacatggtccaaaaagccaactcagtcaaccaagctttagactcagccgttttgctcagagatcccattatgatacacgagtccatgcgttactcactcctcgccggaggaaaacgagtcagacccatgctctgtctctcagcctgtgaactcgtaggcggaaaagaatccgtagccatgccggctgcctgcgccgtcgaaatgatccacaccatgtctctaatccacgacgacctcccttgtatggacaacgatgacctccgccgtggaaagcccacaaaccacaaagtcttcggagaagacgtggccgttttagccggcgatgcacttttagcctttgcttttgagcacatggcggtctctaccgttggtgttccggcagccaagattgtcagggcgattggtgagcttgctaagtcaattgggtcagaaggattagtggctggtcaagtggttgatattgattcagagggtttggctaatgttgggcttgaacaacttgagttcattcatctccataagactggggctcttctagaagcttctgttgttttgggggctattcttggtggtggtacagatgaagaagttgaaaaacttaggagctttgctaggtgtattggcttgctttttcaggttgttgatgacattcttgatgtgactaaatcttctcaagaattgggtaaaactgctgggaaagatttggtggctgataaggttacttatccaaggctaatgggtattgacaaatcaagagaatttgctgagcaattgaacacagaagccaaacagcatctttctggttttgatcccataaaggctgctcctttaattgctttggctaattatattgcttataggcaaaattga

SEQ ID NO: 49 - DNA, 2469 bp, Cannabis sativa
  source 1..2469: mol_type = unassigned DNA
atgccttctctcttctcccaatcactacttctccctttctctcaaaacactaatactctctcccttttccatcaaccaaaacttcttcctccaggtgcttcgctattggaagctaaagacaaacaagttaactttgatcgtgatattcgctcaaaatgcagcgctatatcaaaaccccgcactcacgacgtgtttcaaagtggtggtctgccagttataaagtggcacgagattgtggaggatgacatagatggagaagaagaagatactaagtggacaagatcgaatgagatcgaggaacgtgtcgcttcaatcaaatcaatgttggagagtatggatgagggagagataagcatttcagcgtacgacacagcatgggtagcccttgtggaagatattcatgggagtggcttacctcaattcccatcgagtctccaatggatcgccacacatcagctctccgacggttcttggggcgatgctgacattttctccgcacacgatcgcctcatcaacactttggcttgtgttgttgctttgaaatcttggaacctttatcccgaaaaatgtcaaaaaggtatggcctttttcaatgcaaatataagtaagcttgagagggagaatccggaacacatgcctattggtttcgaagtggctttcccttctttacttgaaatagctcgaaaattaaaccttgaagtgcctgaggattctcctgtgttaaaagtcatatatgctaggagagatttcaagctcacaaggattccgagggacataatgcacacagtgcccacgacgctactccatagcttggaaggaatggtaggtctggactgggaaaagcttttgaaactgcagtcccaagatgggtcattcttgttctcaccatcctcaactgcttttgcactcatggagaccaaagaccgaaattgcttgcaatatttaactaaagcggtccaaaggttcaacgggggtgtcccaaatgtttacccggttgacttgttcgagcacctttgggttgcggatcggttgcagcgcttgggaatatcaagattctttgagccacaaattgaggaatgtatcgattatgtattcagaaattggactgagaaaggaattggctgggcaagaaattccaaggttgaagatattgacgataccgcaatgggtttcagactactaagattgcatggtcacaaagtttctgccgatgtgttccaacactttaagaaaggtgacgattttttctgctttcggggccagtcaactcaagcagtgactgggatgtataacctttatagagcttctcagttggttttccctggagaaaaaattcttgaagatgccatggaattctcatcgaaatttctcagaaaaaaacaggcgtccaatgaattgctagataaatggatcataacaaaggacttacctggtgaggtgggtttcgcattggaggttccatggaatgcaaacttacctcgagtagagaccagattctacattgaacagtatggtggacaaaatgatgtttggattggcaagacactctacagaatgcgaaaagttaacaatgacgaatatctggagttagcaaaacttgattacaacatttgccaagctttgcattcgattgagtggcacaatttgctaaaatggtaccgagattgtaagttggaaaattatggagtgagcagaaggaacctcctcttggcctattttcttgctgcggccagtattttcgaaccggatagggccgatgagcggcttgcatgggctaaaacggcagcactgatgcaggccatccaatctcatttcgatgaccagaaagcttcttcggagcatcgtatagcttttgtctctgcttttaaaaggagttgtaacatgccatcgtatttgattacaagggtgtcgaacataagtgatacagatcatggccttcttagaacgttgatgacgactctcagccacctctctttggacacaatgatgctgtatggtcgggacatcacccaccatttacgtcaagcttgggaaaagtggctggtgaagtggcaagagggtggtgatggacattacgaagaagaagcagaattattgatccaaacaataaaccttagctcaggccgtacacttgtgaaggccctcttgctgtcaaatcctcactatgaaaaactcttcagtaccacaaacaaagtttgctgcaaaattcgtcactttcaaagacaaaggcatagggccaaggcaaatcaaaatggagaatttaacagaaacatcttaacaccagaaatagagtcagatatgcaagaggttgtgcaattggtgctacaaaaatcttcagatgacatgatcaacacaaaaattaagcagacatttctactggtggccaagtctttttattatgctgcctactgtgattctaagaccatcaatttccacattggcaaagtaatatttgagactgtggactga

SEQ ID NO: 50 - DNA, 1251 bp, Cannabis sativa
  source 1..1251: mol_type = unassigned DNA
atgctattctcaaggggatttcgtcggattccgaccaccaccttcaatgggttttcccgttggttcgtctctcaccgacccgggtactcacagtcacagaccactactcattgttctagagattcaacccacaagatttttggcggtttcgaagaaagtcgtttctggggttttgctggctctagataccaaattcatcaccagagtagctccttagttgaggaagaattagaccctttttctcttgttgcggatgaactctcacttgttgctaataggttaagggatatggtagttgctgaggtgcccaagcttgcttctgctgctgagtacttctttaaaatgggtgtggaggggaagagatttcgtccaacggtattattgttaatggcaacagcattaaatgttaaggttcccgagcctgctaaagcgttagcagatactttaacacccgagttacgtacaaggcagcaatctgttgcagaaataacagagatgatccatgtggcaagcctactacatgatgatgtcttagatgatgcagaaactagacgcggtgttggttcgttgaattgcattatgggaaacaaggtatcggtattagcaggagattttctgctttctcgagcttgtgttgccctcgcagctttaaagaataccgaggtagttacgctactagccacggttctagaacagctcgtgacaggtgaaaccatgcaaatgacatgtacatctgaacaacgttgtagtatggaatattatatgcaaaagacatattacaaaactgcatcgttaatttcgaacagttgcaagtcggttgcagtcattgctggacaaactacagaagttgcaatgctagcattcgagtacggcaaaaatttgggtttggcttatcaattgatagatgatattcttgatttcaccggcacgtcagcttccctcggaaagggttcactatctgacatccgtcatggtattgttacagctccattattgtacgccatggaagagtttcctcagttgcgcgccgtggttgagcagggatttgaaaaccccaaaaatgttgacattgcactcgactaccttggaaagagtcgcgggatccaaaaggcgagggaacttgcaataaagcatgcaaacctcgctgctgaggcaatcgagtcacttccagagagcgatgatgaagatgtaaaaagatcgagacgagcactagtagatctcacccaaagagtcgttacaagaacaaagtga

SEQ ID NO: 51 - DNA, 1770 bp, Cannabis sativa
  source 1..1770: mol_type = unassigned DNA
atgtctaccaataataataataatattaataatattatttctcgaagatcagcaaactatcaaccttcactttggcattttgattatgtacaatcactttctacccctttcaaggaaggagcatatgccaaaagagttgagaaagtaaaggaagaggtaagagtaatggtgaagagagcaaaagaggaggagaagcctttatctcaacttgagcttattgatgtaatgcaaagacttggaatctcttaccactttgagaatgaaattaatgatacattgaaagatatatataacaacaataatgtgtacaacaccaacaataatgtgtatgccaattctcttgaatttagactcctaagacaacatggttatccggtgtctcaagaaatatttagtacgtgcaaagatgaaagaggcaattttatggtgtcttccaatgatatcaaaggaatgttatctttatatgaagcttcattctatttggtagaaaatgaagatggtattttggaagagacaagagaaaaaacaaagaaatatcttgaggaatacataatcatgatcatggaaaaacaacaatcattattagatcaaaataataataatgattatgattatgattatgaactagtgagccatgcattggaacttccacttcattggagaatgttaagattggagagtaggtggtttattgatgtgtatgagaagagactagacatgaaccctactctacttaccttagctaaactagatttcaacattgtccaatcaatataccaagatgatcttaaacatgtcttcagctggtgggaaagcactaatatgggaaagaagttggaatttgcaagagatagaacaatggtgaatttcttatggacagtaggagttgcatttgagccacatttcaaaagttttagaagaatgattacaaaagtaaatgctttaataacagtaatagatgacatatatgatgtttatggtgcactagatgaattggagctcttcactaacgcagttgagagatgggatattagtgctatggatgggctccctgagtatatgaagacatgttttcttgctttatacaatttcataaatgatcttccatttgatgtgttaaaaggggaagaaggcctccatgtaataaaattccttcagaaatcgtgggcagatctttgcaaatcttatttaagagaagcaagatggtattataatggatacacaccaagatttgaagagtacattgagaatgcatggatatcaatatcaggacctgttatactatcacatttatacttttttgtagtgaatccaaacatggaagatgccttattaagtacttgctttaatggataccctaccataatacgacattcatcgatgattttacgtcttacagatgatcttgcaacttcaacggatgaattgaaaagaggcgatgttcccaaatcaatccaatgcaaaatgtacgaagatggtatatctgaagaggaagctcgtcaacgtattaagttattaataagtgaaacatggaagcttattaataaagattacataaatttggatgatgatgatgatgatggtgatgactactctccaatgttctatgagtctaataatattaataaggctttcattgaaatgtgtttaaaccttggtagaatggcacattgcatttatcaatatggagatggacatggaattcaagatcgccaaacaaaagatcatgtactatcattacttattcaccctattcctcttacccaatag

SEQ ID NO: 52 - DNA, 1872 bp, Cannabis sativa
  source 1..1872: mol_type = unassigned DNA
atgcagtgcatagcttttcaccaatttgcttcatcatcatccctccctatttggagtagtattgataatcgttttacaccaaaaacttctattacttctatttcaaaaccaaaaccaaaactaaaatcaaaatcaaacttgaaatcgagatcgagatcaagtacttgctaccccatacaatgtactgtggtcgataaccctagttctacgattactaataatagtgatcgaagatcagccaactatggacctcccatttggtcttttgattttgttcaatctcttccaatccaatataagggtgaatcttatacaagtcgattaaataagttggagaaagatgtgaaaaggatgctaattggagtggaaaactctttagcccaacttgaactaattgatacaatacaaagacttggaatatcttatcgttttgaaaatgaaatcatttctattttgaaagaaaaattcaccaataataataacaaccctaatcctattaattatgatttatatgctactgctctccaatttaggcttctacgccaatatggatttgaagtacctcaagaaattttcaataattttaaaaatcacaagacaggagagttcaaggcaaatataagtaatgatattatgggagcattgggcttatatgaagcttcattccatgggaaaaagggtgaaagtattttggaagaagcaagaattttcacaacaaaatgtctcaaaaaatacaaattaatgtcaagtagtaataataataatatgacattaatatcattattagtgaatcatgctttggagatgccacttcaatggagaatcacaagatcagaagctaaatggtttattgaagaaatatatgaaagaaaacaagacatgaatccaactttacttgagtttgccaaattggatttcaatatgctgcaatcaacatatcaagaggagctcaaagtactctctaggtggtggaaggattctaaacttggagagaaattgcctttcgttagagatagattggtggagtgtttcttatggcaagttggagtaagatttgagccacaattcagttactttagaataatggatacaaaactctatgttctattaacaataattgatgatatgcatgacatttatggaacattggaggaactacaacttttcactaatgctcttcaaagatgggatttgaaagaattagataagttaccagattatatgaagacagctttctactttacatacaatttcacaaatgaattggcatttgatgtattacaagaacatggttttgttcacattgaatacttcaagaaactgatggtagagttgtgtaaacatcatttgcaagaggcaaaatggttttatagtggatacaaaccaacattgcaagaatatgttgagaatggatggttgtctgtgggaggacaagttattcttatgcatgcatatttcgcttttacaaatcctgttaccaaagaggcattggaatgtctaaaagacggtcatcctaacatagttcgccatgcatcgataatattacgacttgcagatgatctaggaacattgtcggatgaactgaaaagaggcgatgttcctaaatcaattcaatgttatatgcacgatactggtgcttctgaagatgaagctcgtgagcacataaaatatttaataagtgaatcatggaaggagatgaataatgaagatggaaatattaactcttttttctcaaatgaatttgttcaagtttgcaaaaatcttggtagagcgtcacaattcatgtatcagtatggcgatggacatgcttctcagaataatctatcgaaagagcgcgttttagggttgattattactcctatccccatgtaa

SEQ ID NO: 53 - DNA, 1551 bp, Cannabis sativa
  source 1..1551: mol_type = unassigned DNA
atggagttccaacaaataataccctctttccaagtccttctcttttcattttttatgattattgtggtgagcatactcttgaagagagctcaaacaagcaataggtcagcttcaaaactacccccaggtccatggagactacccttcctgggaaacttgcaccaacttttgggccctttgcctcatcacacattcagagacttagccaaaaaacatggaccatttatgtacctcaaaattggacaagttccaaccatagtagtttcatcaccagagtatgctaaagaggtcatgaaagtccatgataatgctcttgcatcaagacccaataatcttgttacgcaaatcttggcatataatggtacggacattatatttgctccatatggtcagtattggaaagaggtaaaaaagatttgtgtgcaagagcttctaaccctatctagggttaaaacttttcaacccattagagaagaagagtcgtttaatattgtaaaaaatatagcttcaaaagttggctcacctatcaatcttactcaaatgttgaaaagtttgtcttatagcatcatagctagggctgcctttggcgagaaaagaagtgatcatgatgacttcgtatatattatgaaggaaactgtgaaattgtctgcagggtttgcatttggagatgtgtttccatctttgagttttcttgattggtatactataagtaaattcaaagatttgaaactaaggtcttcaagaatagtggacagaattatcaagttacatatagatgatcaagacaaggacaatcttaagaaaagtggagaagaggaagacttggttgatgttcttttgaggtttcataaaagtgaagatcctaataacttcacattaacaaaggacaatcttaaagcaattattgctagcatatttggagctgggagtgatacatcatcagtaactatggagttggctatggcagaaatgatgagaaatccaagagtgatgaaaaaagctcaagacgaggttagagaagtctttggcaaaaaagggtttttggatgaatcttcaatcaatgagatgacatacttaaaatcagttgtgaaagaaactttaaggttgcatcccccagctgccatgttatttccaagagaaagtagagaaaagtgtgatattaatggttatgaaattcctatgaaaactaagatacttgtaaatgcatgggcaattggaagagatcctaaatattggattgaacctgagagttttatgccagagaggtttcttgaaagctctattgattttaagggaaataattttgagtttatcccattcggcgctggaaggagaatatgtcccggaatatcatttgctctcaccagtattgagcttcctctagcatttttgctatatcatttcgattggaaacttcccaatggaatgaaacctgaagagttggacatgacagagagatttggtattacagtctgtagaaagaaggatttgtacttgattccttctaaatatgaaccaccttctatggccaaagcaatgaatataaagtag

SEQ ID NO: 54 - DNA, 1545 bp, Cannabis sativa
  source 1..1545: mol_type = unassigned DNA
atggatcttcaactactttcattcccaataatcctcattactttattttttatgtttatggtagtgaaaatagttttgaaaatagctcatcatcaaacaaagaactcagtttcaaagctacccccaggaccatggaaattaccattggtgggaaatatacaccaaatctttggctcttcaccccatgtttcattcagagacttagccaagaaatatgggccattcatgtacctcaaaattggacaaattccaactctaatagtttcatcaccagagtatgttaaagagatcatgagaacccatgatgttgtttttgcatctaggcctcaaactcttgctgctcagatcatggcatataattgtactgacattatattttcttcatatggtgaacattggagacaactcagaaagatttctatgcaagagcttctaagcccgggaagagttcaaacttttcgaccggttagagaagaagagttgtgtaatcttgttgaagggatcatgacatcttcaaaagatgggtcacctatcaatgttactaaaatggttacaaaatgttcttatggcatcacatctagggctgcctttggcaagaaaagcagtgatcacgacgagttcatttcgattgttgaggaagctatcgaggcagctggaggctttgaatttgcagaagtgtttcctgctttgagatttcttgattggaaaagtcgtcctatttttgagagcatcaaactaagatcttcaagaataatggaaaatatcatcaaggagcatataaaagagaaggaaatttcatttgagaaaattggaaaggatgaagatttggttgatgttcttttgaagtttcataagaatggagatgatcttgggcggttcaccctaacaaaagacaatattaaagcagtaatctttgatatctttgtagctggaagtgaaacaacatctttatctgtagattgggctatggtagaaatgatgagatatccaaaagtgatgaaaaaggctcaagaagaggtgagaaaaattattggtacaaaagggtcagtgaatgaatcatcaatcaatgagatgaaatacttaaaattagttgttaaagaaactctaaggttgcatcctccagctcctttgttacttccaagggaaagtacagaaaaatgtgacattgatggttatgagatacctaagaaaacaagagtaatagtaaatgcttgggcaattggaagagatccaaagtattggattgaacctgagaattttatgccagagaggtttatggaaagctctattgattttaagggcaacaattttgagtacattccatttggtggtggaaggagaatatgtccaggcatgttatttggtgttattaatattgagctttcactagcatatttgttataccattttgattggaaacttcctaatggaatgaaccatgaaaatttggatatgacagaattatttggtcttacaatgagaagaaaagatgatttgtatttgattcctactatttatgaccattcttctatagcaaaatcatga

SEQ ID NO: 55 - DNA, 1512 bp, Cannabis sativa
  source 1..1512: mol_type = unassigned DNA
atggatctccaactaccctctttcccagtcctcttgtcccttcttttttctctccttatggcagttaccatactcatgaaaagagctcgaaactcaaaactacctccagggccatggagacttcccttagtgggaaatctccaccagcttttattggggtattcatcatcatcctcatcttacgaggtcttcagtgacttagccaaaaaacatggacccttcatgtacctcgaaatcggacaagttccaaccgtaatagtgtcatcaccagagtacgctaaagagatcatgagaacccacgacgtcgtttttgcgtctaggccacggactcttgccgctcaaatcattggatacgattgtacggacatcgcatttgctccctatggtgattactggagacagctcagaaagatttgtatgcaagctctttttagtcccaaaagagttcaatctttggaacccattagagagaaagaggtgtttaatacgttacaacatatcattgctaattccaataaactcaattttactcaaatggtcacaaatttgtcttatagcatcgtatctcgagcagcttttggggaaaaaagcagtgatcatgatgagttcatatcgattgtggaggaagatataaaggtagctggagggtttgaatttggggagttgtttccttctttgagatttcttgattggaccagtaggcctaaatatgaaagcctcaaacagaggtcttctagaatattggaaaagatcatcaaacaacatatgattaatcagaataatgagaaaagtgaagaagagcaagacttggttgatgttcttctcaagtatcataacaaggcaaatcttgggttaacccttgacaatatcaaaggagtaatctgggacatttttgaagctggaagtgaaacaacagctgtaacagaggattgggctatggtagaattgatgagaaatccaataatgatgaaaaaggctcaagatgaggttagggaagtttttggaagaaaaggattagttgataaaacatcaatccatgagatgaaatacttaaaattaattattaaagaaactctaaggttgcatcctcctgctccttttttacttccaagggaaaatagtgaaaaatgtgaaattaatggttatgagatacctaatggaacaagagtattggtaaatgtttggggaattggaagacatgctaagtattggaatgaacctgaaagttttataccagaacggtttgatgatagctctattgacttcaaaggtaataattttgagtatattccatttggtgctggaaggagaatatgtcctggcataacatttggtgttgttagtcttgagtattctcttgctttaatgttataccactttgattggaaacttcctaatggaatgaaacctcaagatttagacatgagtgagttatttggcattgcagtaaggagaaaagatgatttgtacttaattcctacaatttatcatcagtcacctcttgcaaattaa

SEQ ID NO: 56 - DNA, 804 bp, Cannabis sativa
  source 1..804: mol_type = unassigned DNA
atgacggaacactcaccactaacccccaaaaccaagcttcatggcaaggtggcagtcgtcaccggcggagccagcggcatcggagaagccacggctcgaaagttcgctgctgatggagcacgcgccgtcgtgattgcagatatccaagacgagaaaggccaaaacgtggccgcatcaatcggcctggaacgctccacctacgtccactgcgacgtgaccgacgaggctcaagtcgcagccctcatcgactcaacggtccaaaagtacggtcaagtcgacgtgatgttcagcaacgccggggtgccttgcgagtcggatcagacgattctggatttggatctggtggcgtacgataaggtgtttgcggtgaacgcgagggggatggcggcgtgtgtgaagcacgcggcgagggcgatggtggagggtggagtgagggggagcgtgatatgcacggcgagcaatctggcgagtataggaactgagaagtacacggactacactatgtcgaagcacgccgttttggggctggtgaggtcagcgagtcttcagctgggggcgcgtggaattcgcgtgaacgcggtttcgccggggccgttgaggacgcctttgctgaaagcgtttatagataggagtgaggaggagcaggataagatgattgaggcttcgttatcgctgaagaatggaaggactccgtcggtggagaacgtggctgatgcggtgtcgtttttggcttcagatgagtctgagtttatcactggccataatcttgccgtcgacggtggttatattcatcatccaccctaa

SEQ ID NO: 57 - DNA, 1032 bp, Cannabis sativa
  source 1..1032: mol_type = unassigned DNA
atggaagaagtgagcaataaacaactgctgctgaagaactatgtttccggttatccgaaagagtcggatatggtcttagccacttccaccatcaagcttaagcttccagaaggctccaatggtgttctagtaaagaacctttatttgtcatgcgatccttacatgggaccccgaatgaagaacctcaacaatggctttttcatagagcccttcaaacttggttctcctatcactgggaatggaatttgtaaggtactaaaatctggaaatccaaacttcaaggaaggagatttggtctctggagtgacagggtgggagcaatacagtgttattgagtccacgaaatatatgtctaaaattcaaaacactgacgtgcctctgtcttactatactggattactaggaatgcctggtatgactgcttatgctggcttcttcgaggtttgctctccgaaaaagggggaatatgtgttcatttctgcagcatcaggagcagttggtcagcttgttgggcagttcgcaaagctcttgggttgttatgttgttggaagtgctggaaggaacgaaaaggttgatttgttgaagaacaaatttggctttgatgaggctttcaactacaaagaagagtctgactttgatgtagctttaaaaaggtattttccagaaggtatagatatatactttgataatgttgggggaaagctgctagatgctgttctacaaaacatgagaagccatgctcgaatttctgtttgtggaatgatctcacagtacaaccttgaacaaactgaaggtgtacataatctgacgaatcttgtgtggaaaagtgctcgaatggttggatttctggttgctgactattaccacctatacccaaaatttctcgaatatgttatgccttacttaaaagaaggaaaaattgtgtatgttgaagatatagctgaagggctaaagagtgccccaaaagctttggtagggctcttcaatggatgcaatgtgggaaaacaggtggttctagtctcttcggaatga

SEQ ID NO: 58 - DNA, 1884 bp, Cannabis sativa
  source 1..1884: mol_type = unassigned DNA
atggaacaagagtactcctcttcatttctctcaacaaagagatatgctgttgttacaggtggaaacaaggggatcgggtttgaaatatgcagacaattagcttcaaatggcatcaaggtcgtgttaactgccagagatgaaaagaggggtgttgaagctgttgagaaactgatcaaagaatcaaatttcactagtgaagacaatgtcgtttttcacaggcttgatgtcgttgaccctgacaccattgcttctttggcagattacatcaaatcccacttcgggaagcttgacattttgataaataatgcaggaattgctgggggtacacttgattcttatggttatgcacaagccactgagcttgctggtggtaattggccagagaacggcaattggaatgagataatgacccagaactatgaatcggctgaagaatgcctgaaaacaaattattatggagccaaagcaacgattgaagcacttgttccactcctccaattgtctgattcaccaagaatcgtcaatgtttcatcctctcttggtctcttacaatacataccgaatgaatgggccaaaaacatgctgagcgatgttgataagctaagagaagagcaaatagatgaggtagtgagtgagtttctgaatgatttcaaacaaggtaagttagaagccaagaagtggcctacagagatttcggggtacaaagtttcgaaagcttcgctgaacgcgtacacaaggattttggcgaagaaataccctcaaatgtgcgttaactgtgtgtgccctggctatgtcaaaactgatatcacttgcaatactgggcagttggttgctgccgaaggtgctgaaagtcccgtgatgctagccttgttgcccctcgccaagccttccggcttcttcttctccaggaagcaactctcccctttctgccattcattcatcactaaacgaaccaaaacatttttattacaaacccaagtttcatcaacaatgtctggagcttcagaaagatatgcaattgtaacaggggcaaataaggggattggactagagatagtgagacaattggccttgaatggagtcaatgtggtcttaacagcaagagatgagaaaaggggtcttgaagctttggagaaactcaaagagaaagagaaaaacctctctcacaaagtgctgtttcaccagctcgacgtggctgatccagctagcattgttgctatggttgatttcattaaaacacatttcggcaaacttgatattttggtgaacaatgctggcgttggtggaacagaagaagacatgcatgcaattgtagcttctttaaatgctaagaccccaaaagaaggtgatattaagaaaactactcaaacttatgagtcagccaaagaatgcatgcaaataaactattatggtgctaaaaaaactgctgaagagcttattccccttctccagttatctgattcaccacgaattgttaatgtttcttctaccatgggaaagctacagaatatatcaaatgactgggctaaaggtgttcttagtgatgctgagagtctcacagaggataaaattgatgaagtgataagagagtttctgaaagatttcaaagaaggttcattggaaaccaaaggctggcctagttttttgtccccatatactgtctcaaaagcagcccttactgccttcacaagggtactagcaaagaagcaccccaactttatcatcaactgtgtatgccctggatttgtgaagacagagataaacttcaacaccggtattttacagcctgaagaaggcgctgcgagtccagtaaggttagcattgcttccaaatgatgcaccttcaggcctcttctttgatcggtcacaggtttcgtctttttga

SEQ ID NO: 59 - DNA, 1587 bp, Cannabis sativa
  source 1..1587: mol_type = unassigned DNA
atgttattattattccattacaatatgtccttcttagacttaaccagtaataatctctcttcctttaccattttattagcaaccttattattctttgtgctgctatataaatcatggttctctattaaaacaaattctccaccatcgcctccaaagcttccaataattggaaacctccaccagctcgggttgtaccctcaccgaacgctgcaggcctggagcaggcgctatggccccgtcatgcagctccggctaggcagcgtgccggttctcgtcatctcctctgccaccgccgctcgtgagattatgaagactcacgacattgccttttccaaccgacccaagtcctgtgccctcgagaagctcctctacaactacagagacattgcttcggcaccttatggcgagtactggaggcagataaagagcgtttccgtgcttcatctgttgaataataaaagggttcagtcctatagagctgtaagggaagaggagaccaagctcatggttgagaagattcgaaagtcttgtgggactggggtgaatttgagcgagttgtttgtaaggctaaccaacgacgtcgtttgtagggtggccttggggagaaagtacggtgaagaaagtggtgggaagaggtttaaggagcttctgggggagtttacggagctacttgggggtttctatgtaagagactattttcctaagcttggttggttgagtcgtgtgagtggtttggatggtagaatggataaagtggctaaggagttcgatgagtttctggaaggtgttcttcatgaccatatgaatacaaataagaatgttgatgatgaacagaaagattttgtggatattttgctttggattcagagggaaaactcgcttggattttctattgataggacttccataaaggctctcatattggacacatttgcagcgggaacagacacaacctatacagtcctagaatgggcaatgactgagctcataagacatccaaacgccatgaaaaagcttcaaaacgagatcagaacaacaatccttaataagaagataactaacattgccatgccagaagaatatattaatagtgttacagaagacgacctagaaaaaatgccatacttgaaggcagttttcaaagaaactctccgtctgcatccaccaatccctttaatcgttcctcgactcacaatacaagacatgaaaataagtggatacgacgtcgcttcaggcacccaagtattcatcaatgcatgggcaatcgggagagatccgaccttgtgggaagaggaaccagacaagtttgaacctgagaggtttttgctgaagaacgctgcaattgattacaaaggacatgacttcgagttgatccctttcggggccgggaggcgaggctgccctgggattgtatttgccatggctgttaacgagcttgctttggctagtgtggtctataagtttgattgggcgttgttgagtagtggagaagaggatttggattattatcatatgactgaaaccacaggtttgactacgcatagaaagtttcctcttatggctgtgccaactgaatattatcaatga

SEQ ID NO: 60 - DNA, 1701 bp, Cannabis sativa
  source 1..1701: mol_type = unassigned DNA
atgtctccttgcgaagctacaattgatgaaaaacgccctaatatgccaaagtttactccaaccatttggggtgattatttcatgtctcatgcttcaagtcatcactcatctcttatggaaactatggagaataataacaaagagagttatgagaagattattgagatgaaggaacaagtgaagaataaattacttcatggtcttcatcctttggaaaaccctttggagacacttgaatatattgatgatattcaacgattggggttgtcttattattttgaaaatgaaattgaacaaaatttggagcaatttcataataattatcaaaatctaattgattttggtgataataacctttatgctgatgctctttgctttcggttgcttaggcaacaaggttataatattgcatgtgacatattcgacaagtacaagaatgaaaatgaaaaatttaaagaatcaatttcgagtgacattcgaggaatgttgaacttgtatgaagctgcacaaatgagagttcatggagagaaaatactagacgaagcacttatctttacaactactcatcttgaatcctcagttaaaacatgtcaattgagctctccttatctagacctagtgaaacatgccctaatgcaccctattcgaaagagcttacaaagaagagaggcaagactttacatatcactttatcatcaactaccttctcatgaggagattcttttaatacttgctaaactagatttcaacctgcttcaaaaactacatcaaaaggaactaagttacataacgaggtggtggaaggagtttgattacaaaagtaagcattcatttataaaagacagaatagtggagtgctatttctgggtttatggagtgttttttgaggcagaaacttcccaaatcagactaataatcaccaaattaattgctattctcacaataattgatgatgcttatgatagctttggtacacttgaagaactagagccttttactcaagcaatagaaaggtgggatatatgtgccatagatactctgcctgagtacatgaaaatattttacatgaaacttttggagatctacaatgaaattgaacaattttctaaggaaagatcatactgccctagctatgctaagaaaggggtgcaatctctaattagagcttattttaaggaagccaaatggttacacacaaaatatataccaacattagaagaatatatgccagttgggattgatagtgcaggatcttttatgctcatttcaatggtttttattggaatgggagatattgttacaaaacattctatggattggatattttctaatcctcaacctaaaattatacaaactatggcaatagttggaagagttatgaatgacattggctaccataagtcggagcgaaagaaatcatcaggagaaattgtggcttcaactgtggagtgttacatgaaacaatatggcgtgactggtgaagaagctatagagaaacttagccaacaagttaaagattcatggaaagatctcaatgaagatcttctcaatccaatcactatccctaggccactcttaatgcaagttctaaagcttgtacgagtgaaccatgagatttatagagaaggagatggctttacacaacccactttgctcaagaatttgattcattctctcatcatcaatccaattgacttttga

SEQ ID NO: 61 - DNA, 2469 bp, Cannabis sativa; identical to SEQ ID NO: 49
  source 1..2469: mol_type = unassigned DNA

SEQ ID NO: 62 - DNA, 1902 bp, Cannabis sativa
  source 1..1902: mol_type = unassigned DNA
atggcgaccgctattttctccaaccccaagttctcccccacaatcaccaccacctcctccaaaaaccattaccactaccaacgccgtacacatttactactccacgacaaagtccaagcttttcaagccgcctctcttaaactcaacccaaatcctcattacaagaaaccccagatcgtttcctgccagagctccggtagcgagtcaccggacacagagaaggtgcggcggccggagtacattccgaaccgtatctccgatccaaactatgtacgtatcttcgacactactctccgagacggtgagcagtcccctggggccgccctgacgtcaaaggagaagctggacattgccagacagctttccaagctcggcgttgacataatcgaggctggattccccgccgcctcgaaagatgacttcgaggccgtcaagattattgccaaagaggtcggtaacgccgttgacgccgacggctatgttcccgttatctgtggtctgtcgaggtgtaatgagaacgatattaggagggcttgggaggcggtcaagtacgccaaaaggcctaggattcatactttcattgctacaagtccaattcacatggagtacaagttgagaaagagtaaggagcaggtgattgagatcgctaggaacatggtgaagtttgctcggagtttggggtgtgatgatgttgagtttagccctgaagatgctggcaggtctgagagggaattcttgtatcagattttgggagaagttataaaggctggagcaacaactctaaacatacctgacactgttggttacaacgtgccaaaagaatttggagaattgattgctgacattaaagccaatacccctggaattgagaatgttgtcatttctacacactgtcaaaatgatcttggactttctactgcaaacacgatatcgggggcatgcgcaggtgctagacaattagaagtaacaatcaacggcattggtgaaagagccgggaatgcatctctggaggaggttgtaatggccataaaatgccgtggagatcaacaactgggaggactttatactggaatcaacacaagacacatctcaatgacaagcataatggttgaggaatacacagggttgcaagtacagccacataaggctattgttggagccaatgcttttgcacatgaaagcggtatccatcaggatggaatgcttaagcacaaaggtacatatgaaatcatatccccagaagatatagggcttgaacgaagcaatgaagctggtatagtccttggaaaactcagtggtcgccatgcattgaaacaacaacttgaggagcttggttatgagcttgaggatgagcaacttgagagtatattctggcgcttcaaatctgtggctgaacttaagaagaggataactgatgctgacctcagagcactagtttcggatgaagtttttcaaccagaagtcatctggaagttcgttgatttgcaggttacatgtggaactcttggtctttcaactgcaaccgtcaaacttattggttcagatgggaaagagcatgttgcttgttcagtaggaactggtccagtggactcggcttacaaagctgttgatctgattgtgaaggaaccagtagcactcctggagtattcaatgaatgctgttactgaaggtatagatgcaattgcaaccacccgtgtgctaatccgagaggaaacgagcgacttgtcaggtcatggttcaactgttgaacgagttactcggacatttagtgggaacgggtcaggaatggatattgtggtttcaagtgtaaaggcttacattggtgcgataaacaagatgttaggtttcaaagataggaccgttgtgaattcttctgaagagagaatacccatatctgcataa

SEQ ID NO: 63 - DNA, 1566 bp, Cannabis sativa
  source 1..1566: mol_type = unassigned DNA
atggggaggcactcttgttgttacaagcagaaactgagaaaagggttgtggtcaccagaagaagatgagaaacttcttaattatataaccaagcatggacatggctgctggagctctgtccctaagctagctggtcttcagagatgtggaaaaagttgcaggctaaggtggataaattatttgaggcctgatttgaaaagaggcccattttcacaacaagaggagaatttgataattgaacttcatgcagttcttggcaacagatggtcacagattgcagctcagttaccaggaagaacagataatgagattaaaaacttatggaattcttgcattaagaagaaactgaggcaaaaagggattgacccaaatactcataagccattatctgaggtagaaaatgacattggtaataaattggagaacaagggtaacaaagctgcaaccaataacaacaacaatgagaatattaataattctactgttagagcttcttcattaggaaacttatccaatgatcatcatcatcatcatcatcatcatctgaatctagctgaccagtcacaaccatcaatggcggccatcaatcgttacccactattggaagtctcatcctcaactccgccgacacaagaattcttcatagaaaaatcaacagataccagatcatcaccatcaatatcatcatcatcaccttgtgatttttctacctacttctctttccactcaaacaattacaatacgacgtcgtccgctgctgcagctgctgctgtttctcatcatcaagatcaaaacaacaacaacaacatggccagtttctgcttcaacattaatcaaaattcaactagacctccacaacaccatcatcataatcagatgattagtaatctcatccagccactacaacaacaagtatcaccttcatcaacaacaacagcatcatcatcatcaccaccctccaatattccacgtgtaaagccctccataagtctccccttattatctgatcaccaaaacaacagtaatagcactactactactactacaacaactactggagccgtacaaaattgggaaactagtactttcagcaacaacggaagtagtagtagtagctgcaatatcgaattacaaggtaataataataacaacaacaacaacttctttgatcacaacactaattccaccgccgcggccgccgccgccgccgctcctaataacttctcgtggggattagtcaatgaaagtactgttggtagcataaaatctgatgacccagaagacataaaatggtctgaatatctccatagcccttttcttcttggtggaggaattagtaatactaataatcaaaattcttcttcttcttcacatcttcaacccattttgtacagtaacattgtgaaaccagaatcacactttagtaatactactactgctacaggatcaaaccccacgtggcatcatcagaacgatcatcatcagctacaagcggcttcatcagaaataatgtacactaataaagatctacagagacttgctgtagcttttggacagaccctttag

SEQ ID NO: 64 - protein, 370 aa, Cannabis sativa
MSTVNLTWVQTCSMFNQGGRSRSLSTFNLNLYHPLKKTPFSIQTPKQKRPTSPFSSISAVLTEQEAVKEGDEEKSIFNFKSYMVQKANSVNQALDSAVLLRDPIMIHESMRYSLLAGGKRVRPMLCLSACELVGGKESVAMPAACAVEMIHTMSLIHDDLPCMDNDDLRRGKPTNHKVFGEDVAVLAGDALLAFAFEHMAVSTVGVPAAKIVRAIGELAKSIGSEGLVAGQVVDIDSEGLANVGLEQLEFIHLHKTGALLEASVVLGAILGGGTDEEVEKLRSFARCIGLLFQVVDDILDVTKSSQELGKTAGKDLVADKVTYPRLMGIDKSREFAEQLNTEAKQHLSGFDPIKAAPLIALANYIAYRQN

SEQ ID NO: 65 - protein, 822 aa, Cannabis sativa
MPSLFSQSLLLPFSQNTNTLSLFHQPKLLPPGASLLEAKDKQVNFDRDIRSKCSAISKPRTHDVFQSGGLPVIKWHEIVEDDIDGEEEDTKWTRSNEIEERVASIKSMLESMDEGEISISAYDTAWVALVEDIHGSGLPQFPSSLQWIATHQLSDGSWGDADIFSAHDRLINTLACVVALKSWNLYPEKCQKGMAFFNANISKLERENPEHMPIGFEVAFPSLLEIARKLNLEVPEDSPVLKVIYARRDFKLTRIPRDIMHTVPTTLLHSLEGMVGLDWEKLLKLQSQDGSFLFSPSSTAFALMETKDRNCLQYLTKAVQRFNGGVPNVYPVDLFEHLWVADRLQRLGISRFFEPQIEECIDYVFRNWTEKGIGWARNSKVEDIDDTAMGFRLLRLHGHKVSADVFQHFKKGDDFFCFRGQSTQAVTGMYNLYRASQLVFPGEKILEDAMEFSSKFLRKKQASNELLDKWIITKDLPGEVGFALEVPWNANLPRVETRFYIEQYGGQNDVWIGKTLYRMRKVNNDEYLELAKLDYNICQALHSIEWHNLLKWYRDCKLENYGVSRRNLLLAYFLAAASIFEPDRADERLAWAKTAALMQAIQSHFDDQKASSEHRIAFVSAFKRSCNMPSYLITRVSNISDTDHGLLRTLMTTLSHLSLDTMMLYGRDITHHLRQAWEKWLVKWQEGGDGHYEEEAELLIQTINLSSGRTLVKALLLSNPHYEKLFSTTNKVCCKIRHFQRQRHRAKANQNGEFNRNILTPEIESDMQEVVQLVLQKSSDDMINTKIKQTFLLVAKSFYYAAYCDSKTINFHIGKVIFETVD

SEQ ID NO: 66 - protein, 416 aa, Cannabis sativa
MLFSRGFRRIPTTTFNGFSRWFVSHRPGYSQSQTTTHCSRDSTHKIFGGFEESRFWGFAGSRYQIHHQSSSLVEEELDPFSLVADELSLVANRLRDMVVAEVPKLASAAEYFFKMGVEGKRFRPTVLLLMATALNVKVPEPAKALADTLTPELRTRQQSVAEITEMIHVASLLHDDVLDDAETRRGVGSLNCIMGNKVSVLAGDFLLSRACVALAALKNTEVVTLLATVLEQLVTGETMQMTCTSEQRCSMEYYMQKTYYKTASLISNSCKSVAVIAGQTTEVAMLAFEYGKNLGLAYQLIDDILDFTGTSASLGKGSLSDIRHGIVTAPLLYAMEEFPQLRAVVEQGFENPKNVDIALDYLGKSRGIQKARELAIKHANLAAEAIESLPESDDEDVKRSRRALVDLTQRVVTRTK

SEQ ID NO: 67 - protein, 589 aa, Cannabis sativa
MSTNNNNNINNIISRRSANYQPSLWHFDYVQSLSTPFKEGAYAKRVEKVKEEVRVMVKRAKEEEKPLSQLELIDVMQRLGISYHFENEINDTLKDIYNNNNVYNTNNNVYANSLEFRLLRQHGYPVSQEIFSTCKDERGNFMVSSNDIKGMLSLYEASFYLVENEDGILEETREKTKKYLEEYIIMIMEKQQSLLDQNNNNDYDYDYELVSHALELPLHWRMLRLESRWFIDVYEKRLDMNPTLLTLAKLDFNIVQSIYQDDLKHVFSWWESTNMGKKLEFARDRTMVNFLWTVGVAFEPHFKSFRRMITKVNALITVIDDIYDVYGALDELELFTNAVERWDISAMDGLPEYMKTCFLALYNFINDLPFDVLKGEEGLHVIKFLQKSWADLCKSYLREARWYYNGYTPRFEEYIENAWISISGPVILSHLYFFVVNPNMEDALLSTCFNGYPTIIRHSSMILRLTDDLATSTDELKRGDVPKSIQCKMYEDGISEEEARQRIKLLISETWKLINKDYINLDDDDDDGDDYSPMFYESNNINKAFIEMCLNLGRMAHCIYQYGDGHGIQDRQTKDHVLSLLIHPIPLTQ

SEQ ID NO: 68 - protein, 623 aa, Cannabis sativa
MQCIAFHQFASSSSLPIWSSIDNRFTPKTSITSISKPKPKLKSKSNLKSRSRSSTCYPIQCTVVDNPSSTITNNSDRRSANYGPPIWSFDFVQSLPIQYKGESYTSRLNKLEKDVKRMLIGVENSLAQLELIDTIQRLGISYRFENEIISILKEKFTNNNNNPNPINYDLYATALQFRLLRQYGFEVPQEIFNNFKNHKTGEFKANISNDIMGALGLYEASFHGKKGESILEEARIFTTKCLKKYKLMSSSNNNNMTLISLLVNHALEMPLQWRITRSEAKWFIEEIYERKQDMNPTLLEFAKLDFNMLQSTYQEELKVLSRWWKDSKLGEKLPFVRDRLVECFLWQVGVRFEPQFSYFRIMDTKLYVLLTIIDDMHDIYGTLEELQLFTNALQRWDLKELDKLPDYMKTAFYFTYNFTNELAFDVLQEHGFVHIEYFKKLMVELCKHHLQEAKWFYSGYKPTLQEYVENGWLSVGGQVILMHAYFAFTNPVTKEALECLKDGHPNIVRHASIILRLADDLGTLSDELKRGDVPKSIQCYMHDTGASEDEAREHIKYLISESWKEMNNEDGNINSFFSNEFVQVCKNLGRASQFMYQYGDGHASQNNLSKERVLGLIITPIPM

SEQ ID NO: 69 - protein, 516 aa, Cannabis sativa
MEFQQIIPSFQVLLFSFFMIIVVSILLKRAQTSNRSASKLPPGPWRLPFLGNLHQLLGPLPHHTFRDLAKKHGPFMYLKIGQVPTIVVSSPEYAKEVMKVHDNALASRPNNLVTQILAYNGTDIIFAPYGQYWKEVKKICVQELLTLSRVKTFQPIREEESFNIVKNIASKVGSPINLTQMLKSLSYSIIARAAFGEKRSDHDDFVYIMKETVKLSAGFAFGDVFPSLSFLDWYTISKFKDLKLRSSRIVDRIIKLHIDDQDKDNLKKSGEEEDLVDVLLRFHKSEDPNNFTLTKDNLKAIIASIFGAGSDTSSVTMELAMAEMMRNPRVMKKAQDEVREVFGKKGFLDESSINEMTYLKSVVKETLRLHPPAAMLFPRESREKCDINGYEIPMKTKILVNAWAIGRDPKYWIEPESFMPERFLESSIDFKGNNFEFIPFGAGRRICPGISFALTSIELPLAFLLYHFDWKLPNGMKPEELDMTERFGITVCRKKDLYLIPSKYEPPSMAKAMNIK

SEQ ID NO: 70 - protein, 514 aa, Cannabis sativa
MDLQLLSFPIILITLFFMFMVVKIVLKIAHHQTKNSVSKLPPGPWKLPLVGNIHQIFGSSPHVSFRDLAKKYGPFMYLKIGQIPTLIVSSPEYVKEIMRTHDVVFASRPQTLAAQIMAYNCTDIIFSSYGEHWRQLRKISMQELLSPGRVQTFRPVREEELCNLVEGIMTSSKDGSPINVTKMVTKCSYGITSRAAFGKKSSDHDEFISIVEEAIEAAGGFEFAEVFPALRFLDWKSRPIFESIKLRSSRIMENIIKEHIKEKEISFEKIGKDEDLVDVLLKFHKNGDDLGRFTLTKDNIKAVIFDIFVAGSETTSLSVDWAMVEMMRYPKVMKKAQEEVRKIIGTKGSVNESSINEMKYLKLVVKETLRLHPPAPLLLPRESTEKCDIDGYEIPKKTRVIVNAWAIGRDPKYWIEPENFMPERFMESSIDFKGNNFEYIPFGGGRRICPGMLFGVINIELSLAYLLYHFDWKLPNGMNHENLDMTELFGLTMRRKDDLYLIPTIYDHSSIAKS

SEQ ID NO: 71 - protein, 503 aa, Cannabis sativa
MDLQLPSFPVLLSLLFSLLMAVTILMKRARNSKLPPGPWRLPLVGNLHQLLLGYSSSSSSYEVFSDLAKKHGPFMYLEIGQVPTVIVSSPEYAKEIMRTHDVVFASRPRTLAAQIIGYDCTDIAFAPYGDYWRQLRKICMQALFSPKRVQSLEPIREKEVFNTLQHIIANSNKLNFTQMVTNLSYSIVSRAAFGEKSSDHDEFISIVEEDIKVAGGFEFGELFPSLRFLDWTSRPKYESLKQRSSRILEKIIKQHMINQNNEKSEEEQDLVDVLLKYHNKANLGLTLDNIKGVIWDIFEAGSETTAVTEDWAMVELMRNPIMMKKAQDEVREVFGRKGLVDKTSIHEMKYLKLIIKETLRLHPPAPFLLPRENSEKCEINGYEIPNGTRVLVNVWGIGRHAKYWNEPESFIPERFDDSSIDFKGNNFEYIPFGAGRRICPGITFGVVSLEYSLALMLYHFDWKLPNGMKPQDLDMSELFGIAVRRKDDLYLIPTIYHQSPLAN

SEQ ID NO: 72 - protein, 267 aa, Cannabis sativa
MTEHSPLTPKTKLHGKVAVVTGGASGIGEATARKFAADGARAVVIADIQDEKGQNVAASIGLERSTYVHCDVTDEAQVAALIDSTVQKYGQVDVMFSNAGVPCESDQTILDLDLVAYDKVFAVNARGMAACVKHAARAMVEGGVRGSVICTASNLASIGTEKYTDYTMSKHAVLGLVRSASLQLGARGIRVNAVSPGPLRTPLLKAFIDRSEEEQDKMIEASLSLKNGRTPSVENVADAVSFLASDESEFITGHNLAVDGGYIHHPP

SEQ ID NO: 73 - protein, 343 aa, Cannabis sativa
MEEVSNKQLLLKNYVSGYPKESDMVLATSTIKLKLPEGSNGVLVKNLYLSCDPYMGPRMKNLNNGFFIEPFKLGSPITGNGICKVLKSGNPNFKEGDLVSGVTGWEQYSVIESTKYMSKIQNTDVPLSYYTGLLGMPGMTAYAGFFEVCSPKKGEYVFISAASGAVGQLVGQFAKLLGCYVVGSAGRNEKVDLLKNKFGFDEAFNYKEESDFDVALKRYFPEGIDIYFDNVGGKLLDAVLQNMRSHARISVCGMISQYNLEQTEGVHNLTNLVWKSARMVGFLVADYYHLYPKFLEYVMPYLKEGKIVYVEDIAEGLKSAPKALVGLFNGCNVGKQVVLVSSE

SEQ ID NO: 74 - protein, 627 aa, Cannabis sativa
MEQEYSSSFLSTKRYAVVTGGNKGIGFEICRQLASNGIKVVLTARDEKRGVEAVEKLIKESNFTSEDNVVFHRLDVVDPDTIASLADYIKSHFGKLDILINNAGIAGGTLDSYGYAQATELAGGNWPENGNWNEIMTQNYESAEECLKTNYYGAKATIEALVPLLQLSDSPRIVNVSSSLGLLQYIPNEWAKNMLSDVDKLREEQIDEVVSEFLNDFKQGKLEAKKWPTEISGYKVSKASLNAYTRILAKKYPQMCVNCVCPGYVKTDITCNTGQLVAAEGAESPVMLALLPLAKPSGFFFSRKQLSPFCHSFITKRTKTFLLQTQVSSTMSGASERYAIVTGANKGIGLEIVRQLALNGVNVVLTARDEKRGLEALEKLKEKEKNLSHKVLFHQLDVADPASIVAMVDFIKTHFGKLDILVNNAGVGGTEEDMHAIVASLNAKTPKEGDIKKTTQTYESAKECMQINYYGAKKTAEELIPLLQLSDSPRIVNVSSTMGKLQNISNDWAKGVLSDAESLTEDKIDEVIREFLKDFKEGSLETKGWPSFLSPYTVSKAALTAFTRVLAKKHPNFIINCVCPGFVKTEINFNTGILQPEEGAASPVRLALLPNDAPSGLFFDRSQVSSF

SEQ ID NO: 75 - protein, 528 aa, Cannabis sativa
MLLLFHYNMSFLDLTSNNLSSFTILLATLLFFVLLYKSWFSIKTNSPPSPPKLPIIGNLHQLGLYPHRTLQAWSRRYGPVMQLRLGSVPVLVISSATAAREIMKTHDIAFSNRPKSCALEKLLYNYRDIASAPYGEYWRQIKSVSVLHLLNNKRVQSYRAVREEETKLMVEKIRKSCGTGVNLSELFVRLTNDVVCRVALGRKYGEESGGKRFKELLGEFTELLGGFYVRDYFPKLGWLSRVSGLDGRMDKVAKEFDEFLEGVLHDHMNTNKNVDDEQKDFVDILLWIQRENSLGFSIDRTSIKALILDTFAAGTDTTYTVLEWAMTELIRHPNAMKKLQNEIRTTILNKKITNIAMPEEYINSVTEDDLEKMPYLKAVFKETLRLHPPIPLIVPRLTIQDMKISGYDVASGTQVFINAWAIGRDPTLWEEEPDKFEPERFLLKNAAIDYKGHDFELIPFGAGRRGCPGIVFAMAVNELALASVVYKFDWALLSSGEEDLDYYHMTETTGLTTHRKFPLMAVPTEYYQ

SEQ ID NO: 76 - protein, 566 aa, Cannabis sativa
MSPCEATIDEKRPNMPKFTPTIWGDYFMSHASSHHSSLMETMENNNKESYEKIIEMKEQVKNKLLHGLHPLENPLETLEYIDDIQRLGLSYYFENEIEQNLEQFHNNYQNLIDFGDNNLYADALCFRLLRQQGYNIACDIFDKYKNENEKFKESISSDIRGMLNLYEAAQMRVHGEKILDEALIFTTTHLESSVKTCQLSSPYLDLVKHALMHPIRKSLQRREARLYISLYHQLPSHEEILLILAKLDFNLLQKLHQKELSYITRWWKEFDYKSKHSFIKDRIVECYFWVYGVFFEAETSQIRLIITKLIAILTIIDDAYDSFGTLEELEPFTQAIERWDICAIDTLPEYMKIFYMKLLEIYNEIEQFSKERSYCPSYAKKGVQSLIRAYFKEAKWLHTKYIPTLEEYMPVGIDSAGSFMLISMVFIGMGDIVTKHSMDWIFSNPQPKIIQTMAIVGRVMNDIGYHKSERKKSSGEIVASTVECYMKQYGVTGEEAIEKLSQQVKDSWKDLNEDLLNPITIPRPLLMQVLKLVRVNHEIYREGDGFTQPTLLKNLIHSLIINPIDF

SEQ ID NO: 77 - protein, 822 aa, Cannabis sativa; identical to SEQ ID NO: 65

SEQ ID NO: 78 - protein, 633 aa, Cannabis sativa
MATAIFSNPKFSPTITTTSSKNHYHYQRRTHLLLHDKVQAFQAASLKLNPNPHYKKPQIVSCQSSGSESPDTEKVRRPEYIPNRISDPNYVRIFDTTLRDGEQSPGAALTSKEKLDIARQLSKLGVDIIEAGFPAASKDDFEAVKIIAKEVGNAVDADGYVPVICGLSRCNENDIRRAWEAVKYAKRPRIHTFIATSPIHMEYKLRKSKEQVIEIARNMVKFARSLGCDDVEFSPEDAGRSEREFLYQILGEVIKAGATTLNIPDTVGYNVPKEFGELIADIKANTPGIENVVISTHCQNDLGLSTANTISGACAGARQLEVTINGIGERAGNASLEEVVMAIKCRGDQQLGGLYTGINTRHISMTSIMVEEYTGLQVQPHKAIVGANAFAHESGIHQDGMLKHKGTYEIISPEDIGLERSNEAGIVLGKLSGRHALKQQLEELGYELEDEQLESIFWRFKSVAELKKRITDADLRALVSDEVFQPEVIWKFVDLQVTCGTLGLSTATVKLIGSDGKEHVACSVGTGPVDSAYKAVDLIVKEPVALLEYSMNAVTEGIDAIATTRVLIREETSDLSGHGSTVERVTRTFSGNGSGMDIVVSSVKAYIGAINKMLGFKDRTVVNSSEERIPISA

SEQ ID NO: 79 - protein, 521 aa, Cannabis sativa
MGRHSCCYKQKLRKGLWSPEEDEKLLNYITKHGHGCWSSVPKLAGLQRCGKSCRLRWINYLRPDLKRGPFSQQEENLIIELHAVLGNRWSQIAAQLPGRTDNEIKNLWNSCIKKKLRQKGIDPNTHKPLSEVENDIGNKLENKGNKAATNNNNNENINNSTVRASSLGNLSNDHHHHHHHHLNLADQSQPSMAAINRYPLLEVSSSTPPTQEFFIEKSTDTRSSPSISSSSPCDFSTYFSFHSNNYNTTSSAAAAAAVSHHQDQNNNNNMASFCFNINQNSTRPPQHHHHNQMISNLIQPLQQQVSPSSTTTASSSSPPSNIPRVKPSISLPLLSDHQNNSNSTTTTTTTTTGAVQNWETSTFSNNGSSSSSCNIELQGNNNNNNNNFFDHNTNSTAAAAAAAAPNNFSWGLVNESTVGSIKSDDPEDIKWSEYLHSPFLLGGGISNTNNQNSSSSSHLQPILYSNIVKPESHFSNTTTATGSNPTWHHQNDHHQLQAASSEIMYTNKDLQRLAVAFGQTL

SEQ ID NO: 80 - DNA, 1974 bp, synthetic construct
  misc_feature 1..1974: note = Description of Artificial Sequence: Synthetic polynucleotide
  source 1..1974: mol_type = other DNA
actaaattttgattactttaaaactgtggctattctttaattatcagttgtaaatctggctatttttaaaattaactagttttacgcttgccaatttaccacaactcctcctttgttagcatgtaataatatgagggagctcacacagaccgagtaaattccctatgagcagtggtggatctagagggcaatgtgcgagttcctggattgcacagtccttgtatatatatattaaaaaatttattaaatatctataaatatttaattgtcaacccaattactaacttgagcccgatcattatagaaattcataaatttcaaagtctggaatcgcctaagttggtcaactaaaatctcctcttgagctactaatcacttctagaagagagtacacagcagtctcatttaatgcaacatcaacctactgaaaagggaaatgatcaggaccaaagtttactttaaagagtcaaaacttcataaaccgtatagatagataatatagcttgacaaaatagaccccgtggtaaaatcatcatttcaatagaaattaggaaaagcaatttgttttttccttatcagatgtcgagagtctatcggaaacaatctttatatcttttcaaggtaaagtaaagctgcgtacaccgtaccctctccaaaccccacttatgagattacgttgaattattattgttgttgttgttgttgttgatgtcaagattaacttgcttgttatctttactatttgttagatacctactatttttcatcgacacaagtaataaataattttgtcgtagattatccacaagatagctacctcctataaatattatatgtaccgaataattctccccatcatatttaagcatatgaaaaaatcacctatctttttaaatgtttcataattttcatcttactttattaaccgttagaccacgctttaatccgtaacttcacttgtatttaagagatttcaactcaataaaaaagatgacaggtggaaaaggaaaatgtcggcatgtttagttgaaaaaaaacttgtactgttattttctgtggcacattcaagatcgtattcatagtaataaaaagtttggtcctggctagctgaaaaatttggttagtagggaatgcatgcatgtaaacgccgcccttcattaaaagattgtatgctactatattttttgggatctttacgtaaaatagctgatcatatttactgtttactttttctagccatatacatagatcatacattgattatatatgattatgcacatatattatataaatcatgtatatattatacctcaaccggatatttttagttttgagtggttaagtgagcggctatttggattaattattcatattttttttggtttcccactttgtgtccggtatcctctttggagtttgattaatttggattcgtggtggaaaattcaattttggagtgtaaagctctccctaccaaacaagggcggatttaggccgtatggtgcacatgtacccatggtttttccgccaaataggtattttatgtatatatttcataaaattgatctaatattatctgttgagccccatgctccaaaaaagttgaatagtgcacctggttgaattctaagttattcatccaaaggaacatggatagattctagcttagcacttcttttttctggctttttcaatggtgcacctatgatctagaaatcctagattcgcctctgctaccataggcgactccatacccaaggctcgaaactgaaatctctgattaaagatagaggagagtacttaccgctccaatacaaccttggtgcctcgtggtaggttgtatagtacactttaataaacaagaaagcttaagcgttaaaattaacttcatttctcaagctataaataccatcatgaaacaacacaatttatactacaatacactccaagtttttttagaggaaaaaaaaaatg

SEQ ID NO: 81 - DNA, 1055 bp, synthetic construct
  misc_feature 1..1055: note = Description of Artificial Sequence: Synthetic polynucleotide
  source 1..1055: mol_type = other DNA
tattaaccgttagaccacgctttaatccgtaacttcacttgtatttaagagatttcaactcaataaaaaagatgacaggtggaaaaggaaaatgtcggcatgtttagttgaaaaaaaacttgtactgttattttctgtggcacattcaagatcgtattcatagtaataaaaagtttggtcctggctagctgaaaaatttggttagtagggaatgcatgcatgtaaacgccgcccttcattaaaagattgtatgctactatattttttgggatctttacgtaaaatagctgatcatatttactgtttactttttctagccatatacatagatcatacattgattatatatgattatgcacatatattatataaatcatgtatatattatacctcaaccggatatttttagttttgagtggttaagtgagcggctatttggattaattattcatattttttttggtttcccactttgtgtccggtatcctctttggagtttgattaatttggattcgtggtggaaaattcaattttggagtgtaaagctctccctaccaaacaagggcggatttaggccgtatggtgcacatgtacccatggtttttccgccaaataggtattttatgtatatatttcataaaattgatctaatattatctgttgagccccatgctccaaaaaagttgaatagtgcacctggttgaattctaagttattcatccaaaggaacatggatagattctagcttagcacttcttttttctggctttttcaatggtgcacctatgatctagaaatccttgattcgcctctgctaccataggcgactccatacccaaggctcgaaactgaaatctctgattaaagatagaggagagtacttaccgctccaatacaaccttggtgcctcgtggtaggttgtatagtacactttaataaacaagaaagcttaagcgttaaaattaacttcatttctcaagctataaataccatcatgaaacaacacaatttatactacaatacactccaagtttttttagaggaaaaaaaaaatg

SEQ ID NO: 82 - DNA, 476 bp, synthetic construct
  misc_feature 1..476: note = Description of Artificial Sequence: Synthetic polynucleotide
  source 1..476: mol_type = other DNA
atggtgcacatgtacccatggtttttccgccaaataggtattttatgtatatatttcataaaattgatctaatattatctgttgagccccatgctccaaaaaagttgaatagtgcacctggttgaattctaagttattcatccaaaggaacatggatagattctagcttagcacttcttttttctggctttttcaatggtgcacctatgatctagaaatccttgattcgcctctgctaccataggcgactccatacccaaggctcgaaactgaaatctctgattaaagatagaggagagtacttaccgctccaatacaaccttggtgcctcgtggtaggttgtatagtacactttaataaacaagaaagcttaagcgttaaaattaacttcatttctcaagctataaataccatcatgaaacaacacaatttatactacaatacactccaagtttttttagaggaaaaaaaaaatg

SEQ ID NO: 83 - DNA, 10 bp, synthetic construct
  misc_feature 1..10: note = Description of Artificial Sequence: Synthetic oligonucleotide
  source 1..10: mol_type = other DNA
atgtcggcat

SEQ ID NO: 84 - DNA, 10 bp, synthetic construct
  misc_feature 1..10: note = Description of Artificial Sequence: Synthetic oligonucleotide
  source 1..10: mol_type = other DNA
gaattattat

SEQ ID NO: 85 - DNA, 14 bp, synthetic construct
  misc_feature 1..14: note = Description of Artificial Sequence: Synthetic oligonucleotide
  source 1..14: mol_type = other DNA
gttgaattattatt

SEQ ID NO: 86 - DNA, 10 bp, synthetic construct
  misc_feature 1..10: note = Description of Artificial Sequence: Synthetic oligonucleotide
  source 1..10: mol_type = other DNA
tttaattatc

SEQ ID NO: 87 - DNA, 10 bp, synthetic construct
  misc_feature 1..10: note = Description of Artificial Sequence: Synthetic oligonucleotide
  source 1..10: mol_type = other DNA
catttaatgc

SEQ ID NO: 88 - DNA, 10 bp, synthetic construct
  misc_feature 1..10: note = Description of Artificial Sequence: Synthetic oligonucleotide
  source 1..10: mol_type = other DNA
gatgacaggt

SEQ ID NO: 89 - DNA, 10 bp, synthetic construct
  misc_feature 1..10: note = Description of Artificial Sequence: Synthetic oligonucleotide
  source 1..10: mol_type = other DNA
taaatatcta

SEQ ID NO: 90 - DNA, 10 bp, synthetic construct
  misc_feature 1..10: note = Description of Artificial Sequence: Synthetic oligonucleotide
  source 1..10: mol_type = other DNA
ctttaattat

SEQ ID NO: 91 - DNA, 14 bp, synthetic construct
  misc_feature 1..14: note = Description of Artificial Sequence: Synthetic oligonucleotide
  source 1..14: mol_type = other DNA
cacatgtacccatg

SEQ ID NO: 92 - DNA, 10 bp, synthetic construct
  misc_feature 1..10: note = Description of Artificial Sequence: Synthetic oligonucleotide
  source 1..10: mol_type = other DNA
tagattctag

SEQ ID NO: 93 - DNA, 10 bp, synthetic construct
  misc_feature 1..10: note = Description of Artificial Sequence: Synthetic oligonucleotide
  source 1..10: mol_type = other DNA
atagattcta

SEQ ID NO: 94 - DNA, 10 bp, synthetic construct
  misc_feature 1..10: note = Description of Artificial Sequence: Synthetic oligonucleotide
  source 1..10: mol_type = other DNA
caaagatatc

SEQ ID NO: 95 - DNA, 12 bp, synthetic construct
  misc_feature 1..12: note = Description of Artificial Sequence: Synthetic oligonucleotide
  source 1..12: mol_type = other DNA
ggattttacagt

SEQ ID NO: 96 - DNA, 11 bp, synthetic construct
  misc_feature 1..11: note = Description of Artificial Sequence: Synthetic oligonucleotide
  source 1..11: mol_type = other DNA
tataaatatct

SEQ ID NO: 97 - DNA, 10 bp, synthetic construct
  misc_feature 1..10: note = Description of Artificial Sequence: Synthetic oligonucleotide
  source 1..10: mol_type = other DNA
cctataaaaa

SEQ ID NO: 98 - DNA, 10 bp, synthetic construct
  misc_feature 1..10: note = Description of Artificial Sequence: Synthetic oligonucleotide
  source 1..10: mol_type = other DNA
caaagatatc

SEQ ID NO: 99 - DNA, 11 bp, synthetic construct
  misc_feature 1..11: note = Description of Artificial Sequence: Synthetic oligonucleotide
  source 1..11: mol_type = other DNA
atagaaatcaa

SEQ ID NO: 100 - DNA, 10 bp, synthetic construct
  misc_feature 1..10: note = Description of Artificial Sequence: Synthetic oligonucleotide
  source 1..10: mol_type = other DNA
attctctaac

SEQ ID NO: 101 - DNA, 10 bp, synthetic construct
  misc_feature 1..10: note = Description of Artificial Sequence: Synthetic oligonucleotide
  source 1..10: mol_type = other DNA
aaggataagg

SEQ ID NO: 102 - DNA, 10 bp, synthetic construct
  misc_feature 1..10: note = Description of Artificial Sequence: Synthetic oligonucleotide
  source 1..10: mol_type = other DNA
tatataaatc